SEQUENCE LISTING (65 sequences)

SEQ ID NO: 1 - protein, 31 aa, synthetic construct
  REGION 1..31: note = Synthetic polypeptide
WSGRARGRARGGAAVAVPAPAAAEAQAVASG

SEQ ID NO: 2 - protein, 34 aa, synthetic construct
  REGION 1..34: note = Synthetic polypeptide
GRGRGGPGGGPGAGLRLRCLRPRRRRRRRWRVGE

SEQ ID NO: 3 - protein, 54 aa, synthetic construct
  REGION 1..54: note = Synthetic polypeptide
GVVGAGPGAGPGRGCGCGACARGGGGAGGGEWVSEEAASWRVAVWGSAAGKRRG

SEQ ID NO: 4 - protein, 24 aa, synthetic construct
  REGION 1..24: note = Synthetic polypeptide
PSARLLSSRACYRLRLFPSLFSSG

SEQ ID NO: 5 - protein, 6 aa, synthetic construct
  REGION 1..6: note = Synthetic polypeptide
PLARDS

SEQ ID NO: 6 - DNA, 780 bp, Homo sapiens
  source 1..780: mol_type = genomic DNA
ccccatttcgctagcctcgtgagaaaacgtcatcgcacatagaaaacagacagacgtaacctacggtgtcccgctaggaaagagaggtgcgtcaaacagcgacaagttccgcccacgtaaaagatgacgcttggtgtgtcagccgtccctgctgcccggttgcttctcttttgggggcggggtctagcaagagcaggtgtgggtttaggaggtgtgtgtttttgtttttcccaccctctctccccactacttgctctcacagtactcgctgagggtgaacaagaaaagacctgataaagattaaccagaagaaaacaaggagggaaacaaccgcagcctgtagcaagctctggaactcaggagtcgcgcgctaggggccggggccggggccggggcgtggtcggggcgggcccgggggcgggcccggggcggggctgcggttgcggtgcctgcgcccgcggcggcggaggcgcaggcggtggcgagtgggtgagtgaggaggcggcatcctggcgggtggctgtttggggttcggctgccgggaagaggcgcgggtagaagcgggggctctcctcagagctcgacgcatttttactttccctctcatttctctgaccgaagctgggtgtcgggctttcgcctctagcgactggtggaattgcctgcatccgggccccgggcttcccggcggcggcggcggcggcggcggcgcagggacaagggatggggatctggcctcttccttgctttcccgccctcagtacccgagctgtctccttc

SEQ ID NO: 7 - DNA, 780 bp, Homo sapiens
  source 1..780: mol_type = genomic DNA
gaaggagacagctcgggtactgagggcgggaaagcaaggaagaggccagatccccatcccttgtccctgcgccgccgccgccgccgccgccgccgggaagcccggggcccggatgcaggcaattccaccagtcgctagaggcgaaagcccgacacccagcttcggtcagagaaatgagagggaaagtaaaaatgcgtcgagctctgaggagagcccccgcttctacccgcgcctcttcccggcagccgaaccccaaacagccacccgccaggatgccgcctcctcactcacccactcgccaccgcctgcgcctccgccgccgcgggcgcaggcaccgcaaccgcagccccgccccgggcccgcccccgggcccgccccgaccacgccccggccccggccccggcccctagcgcgcgactcctgagttccagagcttgctacaggctgcggttgtttccctccttgttttcttctggttaatctttatcaggtcttttcttgttcaccctcagcgagtactgtgagagcaagtagtggggagagagggtgggaaaaacaaaaacacacacctcctaaacccacacctgctcttgctagaccccgcccccaaaagagaagcaaccgggcagcagggacggctgacacaccaagcgtcatcttttacgtgggcggaacttgtcgctgtttgacgcacctctctttcctagcgggacaccgtaggttacgtctgtctgttttctatgtgcgatgacgttttctcacgaggctagcgaaatgggg

SEQ ID NO: 8 - protein, 33 aa, synthetic construct
  REGION 1..33: note = Synthetic polypeptide
  REPEAT 1..2: note = GA repeats
GAWSGRARGRARGGAAVAVPAPAAAEAQAVASG

SEQ ID NO: 9 - protein, 34 aa, synthetic construct
  REGION 1..34: note = Synthetic polypeptide
  REPEAT 1..2: note = AG repeats
AGAWSGRARGRARGGAAVAVPAPAAAEAQAVASG

SEQ ID NO: 10 - protein, 36 aa, synthetic construct
  REGION 1..36: note = Synthetic polypeptide
  REPEAT 1..2: note = GP repeats
GPGRGRGGPGGGPGAGLRLRCLRPRRRRRRRWRVGE

SEQ ID NO: 11 - protein, 37 aa, synthetic construct
  REGION 1..37: note = Synthetic polypeptide
  REPEAT 1..2: note = PG repeats
PGPGRGRGGPGGGPGAGLRLRCLRPRRRRRRRWRVGE

SEQ ID NO: 12 - protein, 56 aa, synthetic construct
  REGION 1..56: note = Synthetic polypeptide
  REPEAT 1..2: note = GR repeats
GRGVVGAGPGAGPGRGCGCGACARGGGGAGGGEWVSEEAASWRVAVWGSAAGKRRG

SEQ ID NO: 13 - protein, 57 aa, synthetic construct
  REGION 1..57: note = Synthetic polypeptide
  REPEAT 1..2: note = RG repeats
RGRGVVGAGPGAGPGRGCGCGACARGGGGAGGGEWVSEEAASWRVAVWGSAAGKRRG

SEQ ID NO: 14 - protein, 27 aa, synthetic construct
  REGION 1..27: note = Synthetic polypeptide
  REPEAT 1..2: note = AP repeats
APAPSARLLSSRACYRLRLFPSLFSSG

SEQ ID NO: 15 - protein, 26 aa, synthetic construct
  REGION 1..26: note = Synthetic polypeptide
PAPSARLLSSRACYRLRLFPSLFSSG

SEQ ID NO: 16 - protein, 8 aa, synthetic construct
  REGION 1..8: note = Synthetic polypeptide
  REPEAT 1..2: note = PR repeats
PRPLARDS

SEQ ID NO: 17 - protein, 9 aa, synthetic construct
  REGION 1..9: note = Synthetic polypeptide
  REPEAT 1..2: note = RP repeats
RPRPLARDS

SEQ ID NO: 18 - protein, 18 aa, synthetic construct
  REGION 1..18: note = Synthetic polypeptide
RGRGRGRGRGRGRGRGRC

SEQ ID NO: 19 - protein, 18 aa, synthetic construct
  REGION 1..18: note = Synthetic polypeptide
RPRPRPRPRPRPRPRPRC

SEQ ID NO: 20 - protein, 20 aa, synthetic construct
  REGION 1..20: note = Synthetic polypeptide
APAPAPAPAPAPAPACKKKK

SEQ ID NO: 21 - protein, 20 aa, synthetic construct
  REGION 1..20: note = Synthetic polypeptide
GPGPGPGPGPGPGPGPGCKK

SEQ ID NO: 22 - protein, 10 aa, synthetic construct
  REGION 1..10: note = Synthetic polypeptide
CRRRRWRVGE

SEQ ID NO: 23 - protein, 14 aa, synthetic construct
  REGION 1..14: note = Synthetic polypeptide
CYRLRLFPSLFSSG

SEQ ID NO: 24 - protein, 15 aa, synthetic construct
  REGION 1..15: note = Synthetic polypeptide
CRVAVWGSAAGKRRG

SEQ ID NO: 25 - protein, 10 aa, synthetic construct
  REGION 1..10: note = Synthetic polypeptide
CRPRPLARDS

SEQ ID NO: 26 - protein, 13 aa, synthetic construct
  REGION 1..13: note = Synthetic polypeptide
CSGRARGRARGGA

SEQ ID NO: 27 - protein, 32 aa, synthetic construct
  REGION 1..32: note = Synthetic polypeptide
AWSGRARGRARGGAAVAVPAPAAAEAQAVASG

SEQ ID NO: 28 - protein, 35 aa, synthetic construct
  REGION 1..35: note = Synthetic polypeptide
PGRGRGGPGGGPGAGLRLRCLRPRRRRRRRWRVGE

SEQ ID NO: 29 - protein, 55 aa, synthetic construct
  REGION 1..55: note = Synthetic polypeptide
RGVVGAGPGAGPGRGCGCGACARGGGGAGGGEWVSEEAASWRVAVWGSAAGKRRG

SEQ ID NO: 30 - protein, 25 aa, synthetic construct
  REGION 1..25: note = Synthetic polypeptide
APSARLLSSRACYRLRLFPSLFSSG

SEQ ID NO: 31 - protein, 7 aa, synthetic construct
  REGION 1..7: note = Synthetic polypeptide
RPLARDS

SEQ ID NO: 32 - protein, 99 aa, synthetic construct
  REGION 1..99: note = Synthetic polypeptide
  REPEAT 92..93: note = PR repeats
MQAIPPVARGESPTPSFGQRNERESKNASSSEESPRFYPRLFPAAEPQTATRQDAASSLTHSPPPAPPPPRAQAPQPQPRPGPAPGPAPTTPRPLARDS

SEQ ID NO: 33 - protein, 74 aa, synthetic construct
  REGION 1..74: note = Synthetic polypeptide
  REPEAT 73..74: note = GP repeats
MRGKVKMRRALRRAPASTRASSRQPNPKQPPARMPPPHSPTRHRLRLRRRGRRHRNRSPAPGPPPGPPRPRPGP

SEQ ID NO: 34 - protein, 68 aa, synthetic construct
  REGION 1..68: note = Synthetic polypeptide
  REPEAT 67..68: note = GP repeats
MRRALRRAPASTRASSRQPNPKQPPARMPPPHSPTRHRLRLRRRGRRHRNRSPAPGPPPGPPRPRPGP

SEQ ID NO: 35 - protein, 41 aa, synthetic construct
  REGION 1..41: note = Synthetic polypeptide
  REPEAT 40..41: note = GP repeats
MPPPHSPTRHRLRLRRRGRRHRNRSPAPGPPPGPPRPRPGP

SEQ ID NO: 36 - DNA, 19 bp, synthetic construct
  misc_feature 1..19: note = Synthetic polynucleotide
  source 1..19: mol_type = other DNA
agtcgctagaggcgaaagc

SEQ ID NO: 37 - DNA, 19 bp, synthetic construct
  misc_feature 1..19: note = Synthetic polynucleotide
  source 1..19: mol_type = other DNA
cgagtgggtgagtgaggag

SEQ ID NO: 38 - DNA, 42 bp, synthetic construct
  misc_feature 1..42: note = Synthetic polynucleotide
  source 1..42: mol_type = other DNA
cgactggagcacgaggacactgaagtcgctagaggcgaaagc

SEQ ID NO: 39 - DNA, 42 bp, synthetic construct
  misc_feature 1..42: note = Synthetic polynucleotide
  source 1..42: mol_type = other DNA
cgactggagcacgaggacactgacgagtgggtgagtgaggag

SEQ ID NO: 40 - DNA, 23 bp, synthetic construct
  misc_feature 1..23: note = Synthetic polynucleotide
  source 1..23: mol_type = other DNA
cgactggagcacgaggacactga

SEQ ID NO: 41 - protein, 91 aa, synthetic construct
  REGION 1..91: note = Synthetic polypeptide
MQAIPPVARGESPTPSFGQRNERESKNASSSEESPRFYPRLFPAAEPQTATRQDAASSLTHSPPPAPPPPRAQAPQPQPRPGPAPGPAPTT

SEQ ID NO: 42 - protein, 72 aa, synthetic construct
  REGION 1..72: note = Synthetic polypeptide
MRGKVKMRRALRRAPASTRASSRQPNPKQPPARMPPPHSPTRHRLRLRRRGRRHRNRSPAPGPPPGPPRPRP

SEQ ID NO: 43 - protein, 66 aa, synthetic construct
  REGION 1..66: note = Synthetic polypeptide
MRRALRRAPASTRASSRQPNPKQPPARMPPPHSPTRHRLRLRRRGRRHRNRSPAPGPPPGPPRPRP

SEQ ID NO: 44 - protein, 39 aa, synthetic construct
  REGION 1..39: note = Synthetic polypeptide
MPPPHSPTRHRLRLRRRGRRHRNRSPAPGPPPGPPRPRP

SEQ ID NO: 45 - DNA, 20 bp, synthetic construct
  misc_feature 1..20: note = Synthetic polynucleotide
  source 1..20: mol_type = other DNA
gcccacgtaaaagatgacgc

SEQ ID NO: 46 - DNA, 20 bp, synthetic construct
  misc_feature 1..20: note = Synthetic polynucleotide
  source 1..20: mol_type = other DNA
cctcctaaacccacacctgc

SEQ ID NO: 47 - DNA, 43 bp, synthetic construct
  misc_feature 1..43: note = Synthetic polynucleotide
  source 1..43: mol_type = other DNA
cgactggagcacgaggacactgacctcctaaacccacacctgc

SEQ ID NO: 48 - DNA, 23 bp, synthetic construct
  misc_feature 1..23: note = Synthetic polynucleotide
  source 1..23: mol_type = other DNA
cgactggagcacgaggacactga

SEQ ID NO: 49 - DNA, 19 bp, synthetic construct
  misc_feature 1..19: note = Synthetic polynucleotide
  source 1..19: mol_type = other DNA
gctttcgcctctagcgact

SEQ ID NO: 50 - DNA, 23 bp, synthetic construct
  misc_feature 1..23: note = Synthetic polynucleotide
  source 1..23: mol_type = other DNA
tctagcgactggtggaattgcct

SEQ ID NO: 51 - DNA, 20 bp, synthetic construct
  misc_feature 1..20: note = Synthetic polynucleotide
  source 1..20: mol_type = other DNA
ctgcggttgtttccctcctt

SEQ ID NO: 52 - DNA, 21 bp, synthetic construct
  misc_feature 1..21: note = Synthetic polynucleotide
  source 1..21: mol_type = other DNA
tttcttgttcaccctcagcga

SEQ ID NO: 53 - DNA, 21 bp, synthetic construct
  misc_feature 1..21: note = Synthetic polynucleotide
  source 1..21: mol_type = other DNA
ctgggaacggtgaaggtgaca

SEQ ID NO: 54 - DNA, 19 bp, synthetic construct
  misc_feature 1..19: note = Synthetic polynucleotide
  source 1..19: mol_type = other DNA
gggagaggactgggccatt

SEQ ID NO: 55 - DNA, 21 bp, synthetic construct
  misc_feature 1..21: note = Synthetic polynucleotide
  source 1..21: mol_type = other DNA
acgacatcgattacaaggacg

SEQ ID NO: 56 - DNA, 20 bp, synthetic construct
  misc_feature 1..20: note = Synthetic polynucleotide
  source 1..20: mol_type = other DNA
atcagcttctgctcgctatg

SEQ ID NO: 57 - protein, 106 aa, synthetic construct
  REGION 1..106: note = Synthetic polypeptide
  REPEAT 78..79: note = AP repeats
GEPPLLPLPLPGSRTPNSHPPGCRLLTHPLATACASAAAGAGTATAAPPRARPRARPDHAPAPAPAPAPAPAPAPAPAPPAPASARLLSSRACYRLRLFPSLFSSG

SEQ ID NO: 58 - protein, 119 aa, synthetic construct
  REGION 1..119: note = Synthetic polypeptide
  REPEAT 108..109: note = PR repeats
MQAIPPVARGESPTPSFGQRNERESKNASSSEESPRFYPRLFPAAEPQTATRQDAASSLTHSPPPAPPPPRAQAPQPQPRPGPAPGPAPTTPRPRPRPRPRPRPRPRPRPRPRPLARDS

SEQ ID NO: 59 - protein, 94 aa, synthetic construct
  REGION 1..94: note = Synthetic polypeptide
  REPEAT 89..90: note = GP repeats
MRGKVKMRRALRRAPASTRASSRQPNPKQPPARMPPPHSPTRHRLRLRRRGRRHRNRSPAPGPPPGPPRPRPGPGPGPGPGPGPGPGPGPGPGP

SEQ ID NO: 60 - protein, 56 aa, synthetic construct
  REGION 1..56: note = Synthetic polypeptide
  REPEAT 17..18: note = GP repeats
GPGPGPGPGPGPGPGPGPGPGPGRGRGGPGGGPGAGLRLRCLRPRRRRRRRWRVGE

SEQ ID NO: 61 - protein, 98 aa, synthetic construct
  REGION 1..98: note = Synthetic polypeptide
  REPEAT 39..40: note = GR repeats
RLTRRKQGGKQPQPVASSGTQESRARGRGRGRGRGRGRGRGRGRGVVGAGPGAGPGRGCGCGACARGGGGAGGGEWVSEEAASWRVAVWGSAAGKRRG

SEQ ID NO: 62 - protein, 65 aa, synthetic construct
  REGION 1..65: note = Synthetic polypeptide
  REPEAT 29..30: note = GA repeats
QALELRSRALGAGAGAGAGAGAGAGAGAGAGAGAWSGRARGRARGGAAVAVPAPAAAEAQAVASG

SEQ ID NO: 63 - DNA, 98334 bp, Homo sapiens
  repeat_region 51933..51938: note = GGGGCC repeats
  source 1..98334: mol_type = genomic DNA
aagcttgataatattatcaaatattagataaatgtaatattagaagaaaacttttttgaaaagatatataaaaataatttcattcaaaatttttatatttaatttaaatttttaatgaaaatatatctaagttttgtacgctttaaatgtaattatgtttgataatttaatcatttactattcgttctctattgctgccctaacaaattaccatagttcagtggcttacaaaacacaaatttattatcttaccattctgtgagtcaaaattccaaaataggtgtcactaggctaaaatgaaggactgcatttcttcctgcaggctccaggagagatctatgtcttactcttttcggcttctaaaggctgcccacattcctcgactagtggcgtccctccttcgtctctaaacccagcaacaacaggttgagtcctcatgtcacatctttcttacctttctgtcatctcatctcgctgactgctgctgggaaaaattctccacttttaagggctatcatgattagactatgcccactagataatacaagatctcagatccttaacttccatcacatctgcaaagtcgcttttgcctcataaaagagtctgaggtttagacgggagatcttaagggggctattaatatgcctaccataatcactgagaataagtacaagttaagattataatagcaatagaatatacaaacgtgaagctccaaaagaacaacaacaacaaaaaaggtgaacaggaaaaagaaactgaaaatctttaaaaaggcagtctgtttaaatctataaaaactggaaaaaaatgagagtggacaaatatctggtaagcatgatggacttaaaatttgtgactagggcattacattttttatattaatataatgaagattgaattactgatcaaaacaattaaaaagcaagagaactattctcatcaaatctgcaacacgaaaagttcagacaaaattccaacaacttcacattctgaactaaatgaggactaattaccagttcgagcaatgagaatatatgaggtcctccgtttgcactttgccagggatctgaaaacgttgggagtaggtcggcttcaccctgaagccagaccatcgacagccagttttccctcccttctccacccacaggtcttaggccctcatccttcccagcctcagaactagtctccaaagaagaggaaagttagaggagagagtaaatcgttgaataggatgaaggagatgtgggaaaaagaaaaagagaggctgcaagagagagggtcccagggataactctgctcttggaagggtggccacagtcatgtggtcccaagaggcaacaacaagcttaggaagccagagaaaccagttacaatcactgctactcttttcgattctgtgttgtttaagaaatatcacccgccaggagttctccagaaacattttccctgattccatgtaagtgctcaaccagtgaatggtaatcccattttggtttagtctgtaccatcccctattccaaaataaagggaaaaatggtgggtttatatcttaaattttctactttactaaactcaagggaaatagccaagcaaaaacgaaagctgagactcttgctaattatcctttccatagaatgtttgctaaaattccttgtcaaggaaggaataacaaagctagtccacgctctgtatagggtgtttccaattagttatactttaaagtataagtatttaacaaaatctataaattttgttaattatttacttgtagtgaaaaatgagccattctcaagcaaatcactttttattacacattccagagaataaccataaaaggacatttattatagcaaaaataaccacatctggatggaacttcaatcaccagtatttactaaataaatgcccagaaaaaaaatagttcatctttaatttcagtcatcattaataaaagctgaagtacctcttcagatcttttgatcattttctgttggattgttttctttttactgagttgcaaatgctctttatatattttggatacaaagctttatcacataggcattttgcaagtattttttccaagtttttttatcttttcatttatttaataatatctttcaaagaacgggaattttataatttttatgaagtccatttataattttttcttttatgggttggtgggggttgggggttgtgttgtcctaagaaatcttggctcaacacaaaaagattagtttctatattttcttctagaagttttatagtacgatctcagatccatttcagatgatgaataagcacataaaaaaaggatactcatcgttagtcattagagaaatgcatattaaaaccataaggaaatactactatatacatatattagataggatgaagagcaactggaatctcatacagtgctgattgaaatgcaaaatggcaaaacaactttagaaaccaatttggaagcagctgtactgacatggaattttgagctggaagaatcttagaaaaagaatactttaccacctcccccattctcttcaccctggggaactgttaaatgaggaaattgtggttcaaggaggaacttgtctatatgctttctcagctttcccgtggtaattaccatcttgataatataacgtaatgtatgtatatgttatcaaataatataatatcttcatcatatatttatcatcttcataatgttagctgtctagtggtaacttttttttgctctttattgcctccctcttttttccctctttgttgttttttgtcatacaattatgatatatgtgtatatattctcactgtaaagatgtaaacaacacaaagattattgaacaaatcacgaaagtaacccttccttcattcttaccctatccaaccctcatctcctcagaagaatacaccattttagttgtaaatgtttttctagctctttttcaatgtttctacctatatgcatgtatgtataatgtatatacatacatatatacatacatattgatatatacatatatagaggtatggttttttaacttaaatggaattgcattgtggatattgtcctatgacttgctttcaaccaaattatatgtcttggaaatacatacatatatttaaaaaatatgttatgtatatgtaacatactatatgtgcataatatatattacatagatataataaggcctaggaagaaattgtgtgcaacctctagtacatcttcctctatatctactgtacatacatacaacccattctttttttaatttttttatttttttagacagaatcttgctctgtcgcccaggctggagtgcagtggcacaatctcggctcactgcaagctccacctcctgggttcacgccattctcctgcctcagcctcccaagtagctgggaatacaggcacctgccatcaggcccagctaatttttttttgtatttttagtacagatggggtttcaccgtgttagccaggatggtctccatctcctgacctcgtgatccgcccacctcatcctcccaaagtgctgggatttacaggcgtgagccaccgcgcccagccacaactcattgcagagtagtccaaaatatggatggactgtagcttaattacttattctcccattgatagacacttaggacttttctaatttttataatttaaaaatatgctgcaattaacaaacattcttgtgtatctttttgctgtatgtatgcatatttctttagtatgggttttggaagaggaatcacaaaggaggcatagaatataaatatttttattttgaaaaatacagttgtaatttaataacccaccaaaagactctaacagtttagattcacatcaacagtgtaagaacatgtctgttttactgcatccttacccccactggttataatacttttaattaacaatcttatggatgaagaatactatcgcaatgttgttttaatgcatttttccaattactagtgagattgaacattaattcttttattttatggatcactggcttttctccttctgtgaactacctgttcacatcctctgcttttcagctcttgagctgttatctttttcttattgatttatatgagctctttatatattcaagatgttaatcatttgtattttatgtatatggcaatgattttcttccaaaccaatgcttgtcttttatttatttatttatttatttatttatttgagaccgagtctcgctctgtcgcccaggctggagtgcagtggcgcgatctcggctcactgcaagctccgcctcccgggttcacgccattctcctgcctcagcctcctgagtaggtgggactacaggcgcccgctgccacacccggctaattttttgtatttttagtagagacagggtttcaccgtgttagccaggatgctctctatctcctgacctcgtgatccgcccgcctcggccttccaaagtggtcggattacaggcatgagccaccacgcctggccaatgcttgtctttttatctctgtttatggcatctttcatactatggacatttttatttttattttttatgttgatttattcttgaattgtatacatgttaattatacctaagttattgtaatacccttaaagccaagttctacacatatatttaatttgctttcccaataggtctctgagggaacacattttttcaaatcactttgtttcatcttttttaggtgttgatcaattattaaggagtttgaaataatcatttaaacggaattcttcagatgaaaacataaagacatttatcgggtcagagcattggtcggttcacatactcaggatcagtggcctgggtgggcaggcactgggtgaatggagagctgcaggtattggaagagagcccagttggatatgtagtttccaaagatcatcaaggcagacaaccaaagggaaaccgtgggaaacacctgctttgggccatctaagatgagatgataaagtaaggaaagagttgagcccaacacagtgatagccaatctgaaagcgggcagaactgacaagaccaaacaagtaggtgaactggctgcaggcagccagccaccacagggacagcgtgtactccagggacaagctcaaggctataggtagttagttcaaggctactagggtgagaagagcaggaactgagttctataccagtgcttctcaaaactaatgtgcatcctaatcacctggaaatcttgtaaaaatgtagattctgattcagtgagtctgaagcagagcttaagatactacatgcttaacaagagcctagttgatgctgacactgctggtccctggagctctctttgagtagcaggcttctggaaggcttgtgtcactaagcacagagaagcctcacttatcaaatctgcaccaaaacaggaaaactaatgtgaagaataatgtgatgcacacgtcagagcatgaggcagttgctttgtccctgaggttgcgctccagatggcttcctaagatgcgacaggctgatcttgtgcgtgggggtcccggaggcttgggccacgggagagacaggacctcagaggctgggagacaggcagagacagaagagtgacatcctgctgcttttgaatttgcacattctgtagaataataacagcagtaaactgttacacaatatctattctcagcatcttgaagccctttcacatattgttacttccattaatggggccctttgctgctatttctacttttctcttcagctatcaacaatatggctttccacacctccatcagacagtagccagatgaaataaaatgtgccagaatgaaaacttgttcatttgtctactttttgccaagactagacaggcaggaaattgaatgtatttttacagaaaaggttttcaaaactttttcccctctgtggctcatttaggtaaactaaaaggcataagacccacctaaaacatgggttcccgctttttattggagaaagaacatagtactttaaaaaaatacataaaataataaaaaggaaagacaaagataatgaaggttgtacatggtaccaaatttttgtatcccataataacacatgagtagatcactactaagtaggttttagtgacatataggaaacattaaaatctacagaaatttgcattattttctgtcaaaaaggatcatttcacagcctttcagggggaacccattgcccacaggaactcatgcattccatgctttgaggatcactagatctaagaagccttccttggaggttctagcctccaacccttattttagtaaaagaagctccagttttatctgtttctaagtcagactaccacacaacattgggcttaaagaaaggtttccagggctaaagcagactttgaggattactaattccgagttaaatttctgtgtattatctctggatttgacttattcacactggactatcactcataaatatacataatacagagttaactatttaaatttataaagagagtattttccttttttatgagcaaaacatgctgccaactacttggaccacatactgatccataaatactgacagctttgtaattggaaataataaatacacactaatgaagcatctcaaaagggaagagccacaggtaatctgagtgattaggcattcatgttaggttaggctttgatcattgtttttaatcgcaatttcattgcagtgcatctataaatccatgtccagaagtatgaagtggttctatagtaagaataagatgctacagataatgcgactaaataagacactataggtaatgacacagattcaagtcttattgttgatgggaagaggtcaataatggatgatataatatactacagcaatgagaattattgaatgttttccagactcacttgtataattggccataacagcaaacaaaaaacaggttctgatagcaaaatgatatacagtactaacaaaggtgaatcttgaggtgaaccttctctttataagtttaaatagtttacccccgaccttttcccatagtagaacagcctaaaaagtatctttcagtagaatgctagtgcttatgaggttttcttaagatatcatttttcaattaaaatttatttcacaaaagactcacatccttgccagccttcagggtgagtgttgattcaggctgtgtccaacggcaacgatgagtgaacttctcaccctcagaatcacatgagcattcctgagatgttttatcagagtgataccaacttcattattagaatattgagtccctatttcctatattcaatgtcctttcaagccctaactttgtccgggttgaaggcaaagatccaaataatcacatttgtctttgataactgaaactgggagaactgggactgtctcaagagttctacgtgactgtaggttgcaagtactgtggttgcatctccaaatattaaccaatcccagtgacaattcaatggggtctcctgaaccatgatcctcatgtctccagtgaaggaaatgggcaaaggggattcaaaaatcccttttggaggaataggaaacttctgctttccttcatttcataacatttgcgatggaacaaaggcttttttagaatggagcaaccagatccttttttgggggaatcagcttaaatgtcccttcttctcatactacttttatctatgtgatcctattcttttctgttgtggattgaatcatgtccctcaaaaagattgaatttagagtgtgctctaaattcaatgtggagaaatttggacacagaggcagacacacagggagaaccccgtgtgacaatggaggaagaggatgcatttatgctgccacaagccaaggaacaccaaagattgtcagcagccaccagaagctaggataaaggcatggcacatcactccctctgagcccccaaaaggagccaagactgctaatactctgatctcggacttctggcctgaaacagtgagagaataaggttctgttgtttcaagctacccagcttgcggtattttgtcacagaagcacaaggaatcaagtacattttctttctcagcacttgtgataatttgattttttctttactcagtggttgtttcacacctatgtccccatcagactgtaagcttaaagagacctggatctggtctgtcttcaccactgttgattcattaccagcacagtgcctggcccatggtcactgaataaacgtttgttgagagaatgaatgtgcttaaccagaagtactattgacctattaggccaagttcaaggtgcctaacagctcagctgtgaaggatacctctcctttcagtcctctgttacatatgtccctgatagatgtgttatttgtatctcctcctggccctcaagtttgtttgagggcaggacccttttttgtatatctgtagagcttcgtagtacctaaatactactttgcatatataataaagtttcgataaatattcattaaataaagaaataaatgaaatgactaagttttctaagatgttacaactagattgaagatatttagctcattatttaacaagaaaactatggttaattatggtgtcctgtgtgaaaatggttatagtttgttttttaattaatataagcatgtatgtgcattatcagtatacacaatttgtggtatgagtgttttgtgtccctgcacacagaccacggaaatcctgagaaacaaactgccaccccagagcaggtgcctaacacagagacttttaatccttaaagtttttctataactaagcaatgttttttcaaatgcaataacactgatatgcagacatattgattgtccactcacaaagccattcctcaatatcattacaacatgcctctttgaatgtcattaaaaatagatgtctcatttttctaggacaagttggctgaagttctgcttgaaaactggtaatagaaaatacaatttctcaacccgctttggccttttaattctgttctacaaccttgccagttcactttcaaagtcaagggatgcatcttgcaaaaccatgacatcttttgagtaactccttctgttcttaacacatattcccaggagcttaataaatattgtttttgcaacttgtttagtggcaaaataatgagtccttggtgtatgcttatcctctgctttgctattagagaagatatattcagactgttttaaacaaattaattcaagggcagggaacagtcctaaaacctgttaaaattcaaatacttggtcactgtatgtgcagcatgtgtgttctagaaagtcctattattttaaaatataaattgaatcttgttgagaaattaatgtcatatgaatatattaataactgaaatgctgccaagtttacaaaaagccctcaatgaaactgtgaccttgtatagacaagggcctgtggagggacatttttaaaccatctctttttttatttcctcatgagatctacaatgtaagtgcattaaagttgatgaatgaattgcagtgcaacttttcctgcctcttttgcctttcatttgtctatatttcaagcttcactgaagtgatagattttgggctttgccacattgtcctctgattgcttccctctgctcctccttttcctagtgaatctttgttttactggtggaaaaatctacatctttgtatcttggcattttactttcacattatctcatagattttatttcaagttgctataaagttatcaacttttatttttaactaatattatttttaacaattagaaaattgttgaccaggtaattccagcactttgggaagctgaagcgggaggatcacgtgagcccaggagctcgagaccagcctgggcaatgcaaggagactgtctctacaaaatataaaaatacattagccaggtttggcggtgcatgcctggggtccagctattcaggaagctgaggtgggaggatcacttgagctggagaggttgaggctgcagtgagcagtgatcgcaccactgcactccagtctgggtgacagagggagaccctatctcgaaaaaaaggaaaagaagaggattttgctggcaagatggctgaataggaatagctccgttctgcagctcccagtgagatcaatgcagaaggcaggtgatttctgcatttccaacagaggtacctggttcatctcactgggactggttggacggtgggtgcagcccatggagggtgagcagaagtagggtggggcgttgcctcactcaggaagtgcaaggggtccctcttctagccaagtgaagccgtcagggactgtgccataagaacagtgcactctggtccaggcttttcccacagtctttgcaacccacagaccaggagataacaagcggtgcctatgccaccagggcccggggtttcaagcacaaaactgggtggccatttgggcagacatcaagctagctgcaggagtttttattttcataccccagtggtgcctggaacgccagtgagacagaaccgttcactcccctggataaggggcagaatccagggagccaagtggtctggcttggcgggtcccacacccacggcgcccagcaagctaagatccactggcttgaaactctcgcttccagcacagcagtctgaggtccacctgagacgcccgggcttggtgtggggaggggcatccaccattgctgaggcttgagtaggcggttttaccctcacggtgtaaacaaagctgcctggaaggtccagctgggcacagcccaccacagctcaccaaggccgctgtggccagagtgcccctctggattcctcctctctgggcaaggcatctctgaaaaaaaggcagcagcgccagtcagagacttatagataaaacccccatcaccctgggacagagcacctcagggaaggagtggctgtgggtgcagtttcagcagatttaaacgttcctgcctgacagctctgagagagcaacagatctcccagcacagcgttcaagctctgttaaagatcagactgcctcctcaagtgggtccctgactcccatgtctcctgattgagagacacctcccagtaggggctgacaaacacctcataaaggagagctccagctggcatctggcaggtgcccctctgggacgaagcttccagaggaaggaacaggcagcaatctttgctgttctgcagtctcagctgatgatacccagtcaaacaggtcctggagtggacctccagcaaactccagcagacctgcagcagaggggcctgaccgttagaaggaaaattaacaaatagaaaggaatagtatcaacatcaacaaaaaggacgtccactcagagaccccatccaaaagtcaccaacatcaaagaccaaaggtagataaatccacaaagatggggagaaaccagtgcaaaaaagtctgaaaattccaaaaaccagaacgcctcttctcctccaaagaatcaccactcctcactagcaaggtaacaaaactggacagagaatgagtttgacaaattcacagaattagtgttcagaaggtgggcaataacaaactcctccaagctaacggagcatgcaaggaagctaagaaccttgaaaaaagttagagcaattgctaactagaataaccagtttagagaagaacataaatgacctgatggagctgaaaaacacagcacgagaactttgtgaagcatacacaagtatcaatagccaaatcgatcacgtggaagaaaggatatcagagattaaagatcaacttaatgaaataaattgagaagacaagattagagaaaaaagaatgaaaaggaatgaacaaagcctccaagcaatataggactatgtgaaaagaccaaatctatgtttgactggtgtaccagaaagtgacggggagcatggaaccaagctggaaaacactcttcaggatattatccaggagaacgtccccaacctagcaaaacaggccaacatttaaattcaagaaatacagacaacaccacaaagatactcctcgagaagaccaaccccaagacacataatcgtcagattcaccaaggttgaaatgaagaaaaaaatgttaagggcagccagagagaaaggtcaggttacccacaaaggaagcccatcagactaacagcagatctctctgcagaaaccctacaagccagaagagagtgggggccaatattcaacatttttaaagaaaagaattttcaacccagaatttcatgtccagccaaactaagcttcataagtgaaggagaaataaaatcctttacagacaaccaaatgctgagagattttgtcaacagcaagcgtgccttacaagagctcctgaaggaagcactaaacgtggaaaggaacaatcggtaccagccactgcaaaagcacaccaaattttaaagtccattgacactatgaaaaaactgcatcaactaacaggcaaaataaccagctagcatcataatgacaggatcaaattaaccttaattaagttagccttaaatgtaaacgggctaaatgccccagttaaaagacacagactggccacctgtataaagagtaaagacccatcagtgtgctatattcaggagacccatctcacatgaaaagacacacataggctcaaaataaagggatggaggaatatttactaagcaaatgggaagcaaagaaaacaaaaagcaggggttgcaatcctagtctctgataaaacagactttaaaccaacaaagatcaaaatagacaaacaagggcattacataatggtaaagggatcaatgcaacaagaacagctaactatcctaaatatatatgcacccaatacaggagcacccagattcataaagcaagttcttagagacctacaaagagacttagactcccacacaataataatgggagactttaacactccactgtcaatattagacagatcaatgagataggaaattaacaaggatactcaggacttgaactcagttctggatcaagtggtcctaatagatacctacagaactctccaccccaaatcaacagaatttacattcttctcagcaccacatcgcacttattctaaaattcaccacatagttggaagtaaaacactcctcagcaaatgcaaaagaacggaaatcataacagtctcttagaccacagtgcagtcaaattagaactcaggattaagaaactcactcaaaaccgcacaactacatggaaactgaacctgttcctgaatgactactgggtaaataatgaaatgaagggcaaaataaagaagttctttgaaaccaatgacaacaaacacacaatgtaccagaatctctgggacacatttaaagcagtgttaagagggaaatttatagcactagatgcccaaaaaagaaagcagaaaagatctaaaatcgacaccctagcatcacaattaaaagaactagagaagcaagagcaaacaaattcaaaagctagcagaagacaataaataagatcagagcagaactgaagaggagagagacatgaaaaacccttcaaaaaaatcaatgaatccaggagctggttttttgaagagattgacaaaacagatagaccactagccagacaataaagaaggagagaagaatcaaatagatgcaataaaaaatgataaagggggtatcaccactgatcccacagaaatacaaactaccatcagagagaatactataaacaactacacaaataaactagaaaatctagaagaaatggataaattcctggacacatacaccctcccaagtctaaaccaggaagaagttgaatccctgaatagaccaataacaagttctgaaattcaggtagtaattaatagcctaccaaccaaaaaaagtccaggaccagacagattcacagccgaattctatcagaggtacaaacaggagctggtaccattccttctgaaactattccaatagaaaaagagggaatcctccctaactgattgtatgaagccagcatcatcgtgataccaaaacctggcagagacacaacaaaaaaaagaaattttcaggccaatatccctgatgaacattgatgcgaaaatcctcaataaaatactggcaagcggaatccagcagcgcatcaaaaagcttatccgccaggatcaagtcggcttcatctctgggatgcaaggctggttcaacatacgcaaatcaataaaccatcattctcagcaaattatcacaagaacagaaaaccaaacaccgcatgttctcactcataagagggagttgaacaatgagaacacgtggacccaaggaggggaacatcacatactgcggcctgtcgagggatttggggttgagggagtgatagcattaggagaaatacctaatgtaggtaacaggttgatgggtgcagcaaaccacaatgcgatgtgtatacctacctaacaaacctgcacgttctgcacatgcactccagaacttaaagtataataataaaaggcgctgcctcaggatgtaaagtgtaacaagggggctggggtgggcagcgtgggcctctgagacctttggttgcccgtgtccgcagctcgccccgcagccggctccacaatggtccgctccgtttgccacgtgcggattcgggttccagactgaaggctgcgtgttctctgccgcccacagcccaagtttattgtggcaaccgccggagcagccttccccgctgtggaggagcctggggctacccctcagcggtatttggggctggtcctgggggagctaagcagggttgtggcagcactgcctgaaagtgtgagaccagactctaatccttatggttttccatgggagttggtgatatgtgcagctgtacatggattttttgctgttctctttttttgtgtggagaagttttagatcggttgggagtcggctttatgtgggaagagaaaaaaagcttgctgtaatgctttctggactaattgaagaaaagcataaactacttgaaaaatttagccatgttcaaaaagagtatgaaggctatgaagtagagtcatctttaaagaatgccagctttgagaaggaggcaacctgtgaaaagctaaacaggtccaattctgaacttgaggatgaaatactctgtctagaaaaagagttaaaataagagaaatctaaacattctgaacaaggtgaattgatggtggatatttgcaaaaggatacagtctctagaagatgagtcaaaatccctcaaatgacaagtagctgaagccaaaatgaacttgacgatatttcaaatgaatgaagaacgactgaagatagcaataaaagatgctttgaatgaaaattctcaactccaggaaaacgagagacagcttttgcaagaagctgaggtatggaaagaacaagtgagtgaacttaataaacagaaaataacatttgaagactccaaagtacatgcagaacaagttctaaatgataaagaaaatcacatcaagactctgaacgcttgctaaaaatgaaagatcaggctgctatgcttggagaagacataacggatgatggtaacttggaattagaaatgaacagtgaatcggaaaatggtgcttacttagataatcctccgaaaggagctctgaagaaactgatttatgctgctaagttaaatgcttctttaaaaaccttacaaggagaaagaaaccaaatttatagtcagttatctgaagttgataaaggaagagcttacagagcatattaaaaatcttcagactgaacaagcatctttgcagtcagaaaacacacattttgaaagtgagaatcagaagcttcaacaaaaacttaaagtaatgattgaattttatcaagaaaatgaaatgaaactccagaggaaattaacagtagatgaaattaccggttagaaaaggaagaaaaactttctaaagtacacgaaaagatcagccgtgccactgaagagttggagacctatagaaagtgagccaaagatcttgaagaagagttggcgagaactattcattcttatcaaggatggattatttcccacgagaaaaaagcacataataattggttggcagcttggactgctgaaagaaacctcaatggtttaaggaaagaaagtgctcacaacagacaaaaattaactgaagcagagtttaaatttgaacttttagaaaaagatccttatgcacttcatgttccaaatacagcatttggcagagagcattccccatatggtccctcaccactgggtcggccttcatcctaaacaagagcttttctctgagggcccactgagactctcatctttgctaacaggaggaggaggaagaggctcaagaggtccagggaatcctctggaccatcagattaccaatgaaagaggagaatcaagatgtgacaggttaaccaatcctcacagggcttctctgacactgggtccctgtcacctccatgggaacaggaccgtaggatgatgtttcttccaccaggacaatcatatcctgattcagctcttcctccacaaaggcaagacagattttattctaattctggcacactgtctggaccagcagaactcagaaggtttaatatgacttctttggataaagtggatgggtcaatgctttcagaaatggaatccagcagaaatgataccaaagatgaccttggtaatttaaatgtgcctgattcatctctccctgctgaaaatgaagcaactggcccttacttttctcctccacctcttgctccaatcagaggtccattgtttccgggggatacaaggagcctgttcatgagaagaggacctcctttccccccacctcctccaggaaccatgtttggagcttctcaagattattttccaccaagggatttcccagatccaccacatgctccatttgcaatgagaaatgtctatccagcgaggcgtttcctccttaccttcccccaaaacctggatttttccccataaaccccacattctgaaggtagaagtgagttccctgcagggctgattctgccttcaaatgagcctgctactgaacatccagaaccacagcaagaaacctgacaatatttttgctctcttcaaaagtaattttgactgatctcattttcagtttaagtaactgctgttacttaagtgattacacttttgctcccactgaagcttaatggaattataattctcaggatagtgttttctaaataaagatgatttaaatatgaatcttatgagtaaattatttccattttatgttattctggatagtataactattttaatttgataaactaatccacgattatataaacaataatgggagttttatatatgtaatcttgcaggtagggaggctttaaattataaaggttgtgtctttatgccaagaactgtattaactgtggttgtagacaaatgtgaaagtaattttatgcttcattaaataaattttagttgatttttttttaaaaaaagaaaatggttaatctatcatttaggtgcatcatcagttgtttaaccattctctcttactgaacattgggttgtttaaaaagtgttgttatttttgaatcatggttcagtgaacaattttggacacataactttttatctgatgagttatttcctaaggatccagctcagaaactcagcacataaacctaataagaaaaaaacaatttgaagtggctaacctcttatcccaataaaaatgttgtatttatgtttggatttagatgcctttcagtggtcataccttcacctaacttttatggattctacttttaacatgtagagtgactgtttaaatcacctaaactcactgagttttaagttcctttttattcaacaagactggattgtatgttccagctcctcaaacttagttaccaaccaccatcctagagaagtgaattcacatgaggcctgtccagaagaacaatctccctttcagtgtcctcatgcatgcagtgaccagagaccaaccttgataaattatggaaaaagtacagcacattctggaagagccatgaaagatccagatcatctggtgctggataagaatattaatggacaggctgggcgcggtggctcacgcctgtaatcctagcactttgggaggccgaggcgggcggaacatgaggtcaggagatcgagaccatcctggctaacacggtgaaaccccgtctctactgaaaatacaaaaaattagccgggcatggtggcgggcgcctgtagtcccagctacacgagaggctgaggcaggagaatggcgtgaacccgggaggcagagcttgtagtgagcccagatggcgccattgcacttcagcctgggcgacagagtgagactccgtttcaaaaaaaaaaaaaaaagaatattaatggacaaaaagattaatgaaagaacatattgaagcatccaattacctggtgtctgctcaaatgaggaatcggtgagataggtcagttagcagtcaagatttataaaagagacgatggccttgggaggggctgccctactcgactttttaatggctagaagctattaagggctaagccagaacccttcagtatggttcagtgaggatcccaatttggggtccaaaagtaaatgacaactcccaggaaccattaagaataaaaatcatggagcattactgagaatttatgttatctaagtctgaggaaaattaatgttaaggaagctttcaaaagtctaatatttacaccgaattccagggcaccatgctctaagacaaagcactctggtcctgcccctctcctttcctcatgttttttggttcttgggatccttaagggtcaatgttattcttaaaatacagagcatcctggaaactaaaaaagtggaagatattcaaattctaatgaatgtactggcagtattgtagatcatggagtataacataaagacaagaatccctagcctcttccaccatactttgtaatggtaaggagaaaggatagaattttgagaagtctgggaagacaatgtatgataacatctggagaagctctgcataagttacttttgttcaggcttaagaaaaattctagcttgcccctgcactgtcatcaggtatcatgaaagtaaataaaacctttaaagattcttcaagccagcagacttctatcttctctatactatcctgtgatcctaaactcttaacagttactacgtataatttccctacatttgctactagtattttatcatacacaatattacactcaatatttcaaaagtggatgattcatctcccgaagagactgcaaaattcatgagttaagatttgagaatactattttagacaagatttagtcagattttagagagttagaaacctgtaacaattctctaacaatactgcttctccttttgtgtattaaggaatttttgtctatcaaagatagtacgaggtagaccagaagataacttgccttcaaaatgtctggaatgtaaaatggcaacagtagtatttggggacttcgtaggggatggccaatatacacccattcttagaggtactgatgatataatgtataagacaaaatcaagtggtctccatcaccatataatgtttaaaatggcaaagagggagcagaacaaacaccctttgcaaatctcttcatagaatctaccgtaataaacttgtacttgcttaaagtgtgtctcttcagtggtcttattaccactactttggggaaaatgaggctgcttaaaagattaacagacattacattttacatatctgtggcagagaaaacactatgtattcaccaaaccacttcttttccttcccagtcactcgggaagaggtcatttctttgtcccctttcatctaattgaggtgccgtgactacttctagacaggcaatgtgagcagaaggtatgcacgccacgtataggcctggtcttcaaaaatccctcagatatgatcttcttctctcgtctctttcatggacaaactacaggccatgtaataaggatggtggggttccaaactgaaagagcctggatttctgatttactgttttgagaagagttcaccagggaaacagcctggaaatacgcacaggaaaatatgcacaggaccctgtgtgagcaagatataaagatctattacatggtgccattaaggtgagagtattgtgcttatagtatccagcattaattatcctcactactacaacttctttgtatccatcatgtggaaaagtagagtatttaataaatgattattgagtttattaccttttttatattccaatcattgctaattgtacgttacctcatttcaaggtaaaggtgaccaagggctaaagcagtgctatccaaaccaagccagacatcaaaatcacacaaaaccttttgaaaatacaactttgaagatgccattcacatagatatttattcagtgggttttcaaatggaaccctggaatctacagtctttaacaaggcttcccaagttattctgatatacagcaggcaaatctgagaaccactggacaagaagaaaataaaggctatatctttcgacaacaaagacaatgccttaaacatagaatgtattcaattaaagcttgtagaaagataggtttgtgaacaggcacagggactagcctcgagcaaattaataagggcagcaatgtttttcactgaaaccattattccccctattttatttcttctggggctctgtgtttcctttctcctatcaaaatccattctaaggttggaggttgggggtatctcttgcctactccatacagcaaggaataaaattagtatttctcgaactatctgtgacagcagacccattgtaggccagtacttttgtaaaatgcaataaaaattaacttctagagaatgaaattttaaaatcacagacattcaaaatacaaattccaatttttttattattaactgtaagaaatttaaaattaaatctcaataaataaaattaaagcaaacataagatagaaaaaaataagcattatggattggcccagtctgcaaactgtatacactttgccaaacatgggcataaattactaagaagcaaaatcttccatctgtaaacatttccatttccattgacaatatgtgtgagggaaaggagggatgcttctgttttagaatgccaggcgtcagctaacaagtgacaaatacgtattgagactgagatctccccagcctctcagtagtcagcaagaacatgttgaggcctctgtttttgactaaaaaattggccagtgcatgggcaacatgcataggtcctgaatgaaaaaaatagcagcagcagaaatttaaaagaattttcacagctaggccacagtaaattctcaagcccttcatcagaagccactgtggggcctcatttatgcctttgtttttattaaattggatgtgatcttaagattcttctgtcaaaattccactagcatgtgaaggcaccaaaagtttaaaatgtaaaattaacccaagttaagctattccattattaagcaatagcagatatatttgttattatatgagaagaaagttaacagggagctaagattgatgttactgataagaaacagaaacaagactttaaaattaaataaatgaattatttatttaataagaaccaattgacagattctcgataaagactgtaagatgtcttaaaacattaggtgtatggagataacatttgtaactttgacaatttatatgatgagaaaaatcaaggaatgttattgtttattggcagagttctagaattacaattccatcattctgttttggggaagtttcccttgaagtaaatgataacagggcttgaaatagtacacctcagcattttgtttataaaactgtggaataggtaaggtttgtattgtaactgaacccaggttcagctgcttgctgctctaaagctagacataagagaggaaggttggtgggaggaaaagcgattttaatcggagaagcagcaaaccaagaagatggtgaacaatagtcacagaaccatcttaaattttaaaatttaccatagagtgttcaaaggaaaacttggtatgggaggcatgcaggaggggtgcagggggcggggtctgtgtgtcttgttccaatggctatctcagatagtcacccatctggaggtctagttggtattattttgaattcagcccagtggtggtggactgtcagtgactcctcgctaagcaggaggattctgcactcagggctccatgcatggtttgtttcaagattggcctctggaatttctcaagcaagaacataattaaataagcaggcattgccagaggggagtgtctggaaaggaaaggaatgaagagatgaaaggaaagtgggtggttaaactatatttttaaaactgaggttcccagttatagtatgtttcgcacgctccccccattttagcacccctgacagaatttagtaatctcctcatcttgtcctctacttcaggtcccctatctgtccttgtactctccagggtttccttttcttcttcacgaccttccttccctgcaattttataagctattcctatcccagtgatttagtttcagcttataaaactgtgtctttgccattgtaatcaaattgaagggcctctgcttcatggttggattctgtgaccaggagactcttacgaggagttggccaggtctctgttaggaaagcaaaaaagaacaatggaggcaattatcccattgatttcagctataaatcctattttgcctgaattgtctgaacgatgagtattctgtgaaaatgctgctctctagtgcaatagaactgcaaataatgcacatctatttcttataatctcatccaacatacccacagagattcagatctaacaaaacagaggtgatttggttattgaatcataatataaatatggggaagaggagggaaatttcaagcctgaggaaactgtagtaggagtaagtatgctgtgtttaagaggtcacagataaaattaatattaccaatccatcaataggcaattactaatagcttactacacacacaggaataaaatgtgaagacagaggaagtgtaaaatggagccgccaactctacggagttgtttgcaatttggtctggtagaaagctatgaaataaggaagtacatgattgagagctagagaatgtggcacaggctctgaacccggaccgttcaatgtagtaagctctagccacactggacacttgcaatgtggcttgtccaaactgacatgtgctttaagtataaaatataatccagatttctaagacttcaaaaaaaatggaaatatctcattaataatcttaagtttattacaggtagaaatgatagattaaataaactatattgtcaaaattcatttgatctgtttctacagtataacaaacttacttgtgtggtttgcattttatttctactggataacatggctttaaaaatggtattttagaggaaggaaagcttggtagagaatggactaatccggatccctggaagaaatggaccttgaatgggtcttgatgacttggagaggcagagagagaaaaagaaaagtcaaacatagggaattggttgataaaatgaaggtgaggggagaaggaacagagggaggagaagatccagtttgagggatattacagcgagcagcctgagaaagaaggataagaaaggagagaaaaaatgcaagggaagtaacccttcaaagccagtcagaagtttctgggttcctcagcagccagaaaagaagccgttgaaaagatctgagtaacggagattctggacgaaaactgaagttatggaagggaagtttagacatgggttattaaacgctttagcgcattagaagtttcttatgtaatcactaaattcagatcctgaaataatgccacaagaactatacagctcagccacccaattcaataagaagttacagcacagtctcacacatatccaattaaccttggcctttagtcaacatctgggttctttttgtcattttcaaatactatcacccagaggtgctatgatttatattggggaggggattaaaagaaaataagtaagttggtgataagaaaaagctttcagatgattccatctgaattaacagccctctttagttgtctaggaaagaggatgctttttcttgaaagtgctttgaaatgatgatgtgcttgttagtaaacatcaattattttcaaatcgtaatgtttgcaagtttgtcttcctgtagctcaccctttatgtaggtccagaatatgattgtcacaaatatctgggtgagcaagactatgaaatgtggtcataaagtaagtgattatttctaaactcatctttgtcactcgtagtgcttcacaaagcaccttttcctggactacaattcattttaattgatcccatcagcactatatctgtatcctgagtgacttcacaataccctctatttcaagagaaaccaatcaggttatgggtttgttagtaataaaaattaccaaggagcagtttgtggatggtaaaagcaatgcaaattctaaagagaagtcataagagcaataataagcatcctcctcacttcttggaagtgaacaattccaagctccctgaagcaacacttaacctatcatattaaacagtaatggacaaatattagaaatgttgatgtcagctttcagaatctgtgggcatcaaaacatcacttaagttctccgaagtattctctgtcaagtttccttctacagtattcttttcctactaggacagagccttaagccctagaagaataattttgcttgtgtgttaattatttgtttactggttcattccagagtgtgagctggaaaaagggggaagtgtcataaatagttttttatggcccatggtttttcaactacgtcactattggtagcagtttccactgcaggatctatttgcaaagcctaggaaattagcattaagcaagctgctaggaagacttcaacagtaactaggccacaggcctcacacatttttcctccaccccagcctcctctggagagtacttgctaaacctctgtgacacataatgaagcaaagaaagtgatagaacaacagaattacacgggcagatccttgtttcttcttctctctctaaagaattccttggactgaaaagcagtttattttggaggagtgagaaagtggtgacagaattagaagggcctgggagggcttcattttaggagacagttttaggctgaaaagagatttcatgagtgtgatttacctgaggtgacttttgggggctcttataaaaaggaagttcatgctgaatgggaggtggcttctgagatgcagattctggtgagctaagagggctcggtaaagaggaggcaggagttaagtagcgtgaactatgcagtagcagccttcttccccccttgcttggggcaggtcatcacaacccttctcaataaaggggtccaggaaccactaggaataaatgggcatttgcacttcaggtgaaacccatttgtcataactgcttggactttaagcttacaaataaaaagaaccacatatttccctttgcagcttgatttagttaatgtcattttgagaaagaaagaagacattgttatcccgtcccttttttttttttttttttttttttatgaagagactgggactcagagaagtcaagtgattttcccagaaccagaaaacacagaagtagcagagctgagatgactactccggtcttctgattccaaattccaaattcattcttctaagcgatttcccaaaacgggaaatgggtttatcttctatttatgggaagtgatagtggtattctatttagagaacttatataaaatcttactttaaaataaataatatttcaaaaagtaagcttaatttaaagaaaataatcaagaaagtctggtatatttttacaaatataccaaatgaccttgctctaaaatacatctactttccagcaagccaaagtgaaacaatttgaaataagtggcatttactgaccactccctaaagttcacacaaaagaggtagtactctaacttaaatatacaaggtgaagaaatagcttactcagcctgttgggcttcctcttctacactcttgggaaatgccctccgtgttaaccaagaattctcaggccttggagggagttttccattctcagtaaactgagattgcagttgcggaaattaagaggtatctgtccagcacttcattcccttaaggtcaggatctgtgcttttaataatgacaattagctaacatatacaattaagccatgcaaatgaagtaagagaaagctagaggagaaattcaggagccagttgccttttccagacatcttgtacaaatagtgttcaaaggactaattcaaaagatgggattcttcgcttgaacccaggaggtggagtttgcagtgagcggagatcgctccactgcactccagcctgggtgacaaagtgagaccccatccaaaaaaaaaaaaaaaaaaaaaaaaaaaagatgggattcttttttaaaaaataaattttactgcgtatttttaaggtatacaacgtgatgttataagatggatatagatagtgaaaaggtaactgtagtgaagcaaattaacatattcatcatctcacatagttatcttttatttgttttgttttgatgggatttttaagatagtagaaaggaatggtagacaataaacatttgagggaaagtggggctttgtagaactcctaaaatgacagcacgcacaaatgtccccattatgtctaaagggtaactcgttcctacttctagggacagctgagggacatcaatgtaaatttctaaatgacttcctgaactttttatttttattttttgtatttttagaggaaattataataacatcaagccacctctggaccatatcgctgctgatatcatcagcaaatggcactattcctaaatcctaagatgcacttttcccttcacatttcaacatttgtgaaactcgattgtacctacacctgattttatatacaatgcagcctttccttttcttttgtcattgcatcttacgcctgatttctccttggaattgagtaaatataatgcttacatgtgttaataagaattgaggtcactcataatttttgaaatatgccaccaaatataagcctttctacatattgttgactttgaagtcatttctttttttaactactaaacaataacactttttgttgagaaaaattgcatatgaacaagagaccaagcaggtagagagaaaaaaacttttaataatcaagagaatgttactgtgtcccaaaggctaaagtcaccttactatcaagagagaaggacaggaacagagagaaccaggtaaattacgaattgaaaattccatggttcatttatctttatttttaataattccatttgtgtgattgtgttgaccacaaggtcataatgttactcttcatactgacttctcatgtaaattataaataagtttttatgctaatgatttatggagtaagctattcatctttccgacagagagttacctacaaagaaataattattctacctctgagatgaaatatcatgaaaggagtggtttccagatattttgacttttaaaagcttaaagaatatatgtagtataaaattctaaagcaggcaaaattaatccttttagcaatcaagatagcggctacttttggtgagaaggacaaggtagtgatagagaaggggctcaggggtctttcctgaagacagtgaggtgggcaatggtattttccttgacctggatggtgattaaacagatgtgtttactttgtgataattgactaggctgtgcacctatgaactgcatacttttccatatatgtactgtattcttatacttaaaaagaagtttaaaaataaatgcaacagatataggacttcctatattactcgttgaccaaaaaaatggattcatttttctttcaggtaaaacgtactagtggttttaatattatattgaccagggagtaaatgtttaccttaggaaccttaatcttgatgttctccaaagtcattatctgttctttctgattatcagaatagagtatatctctatataaatgaaaatttctggtcattctcaaaaaataacactaagcatgaaaatcagaaatattgatcttgttttgtaatgatgtttctattgatgtgaagtagtttctagtagagttgctgtcctaacacacaaatgaaattgcactgtttggaagacacaactgtgaatgacttgcttcagtaaggaatttccaacatgatggtttagggatagaggtgctcgattcctctgtctccggttacccaggttattgaggacagggaggtcaataagtaatgccctcctcccacccatagcacaaaacagagcggggttcagagaataggtaaggctttggccagggtgttgaggagacttacatccctgggaaccagtcagaatgggggcgctgaaaacaatgttttaaattctagcacccagcaacatatgtgtgaagattaaatgtactcgtgctaaattcacttgctccattactgaatttgggtggtgtctgttaaagatgggaacaaaggcattcaggtcctggtatcttctaccactcccagcatgaacagactcatgtcagtgggtaagggatggtatttcccgagaaggctttgaactcttgtagtgggtcaaataatggccccccacttaaaaatgttcatgtccaaatccctggaagctgtgaaaaggggtttttgcacatgtaattaagtcaaagatattgaaattagatcatcctggattacataggtgggccctacatttaatgacaagtatcctcataacagaagaggagaaggtgatgtgagatttggagcagcagagattggagtgatgtggccaccaatcaaggaaaccaaggacttccagcagccaccagaagctggaagaggcaaggaaggactcttccctaaagcctttaaaggagcacagccctactaacaccttgcttttgggctctggcccgcaaaactgtgaaaggatacattgctgttatttgaagccacagttcgtagtaaatttattacagcagccctagaaactgatacaactcctaaatacacccttagcaacactgctcaacaagaagtaggcaatttcctcctgactgaaaaatactgatactgttatgggatccttgggggtgttgcttttctgtccagaaacctctgtggcggtggcacctttgcatgagttttgctcgggtccactgggcccactcatcctggcaggctgcgctcagctgacactactggcgtggatcccatgcctccaaagagactggagcgaagcggtgagggatgtgtgaggaagtgagcgtggggtctggcacacagtcaggctcaatggctgctacagcgggatgggcagcttcaggtgctggcacgggtgctggctcactgcaaggctgtggctgcaccaagcagcgcagcaacggaacgcattggtgcctggaaacttggagactccaggaacctcagggctccaaaaggcaaatcacagccctagcttcgggagctcccaggtctgggctgccaaagggctgcagctcttctctcctctctctctcttcgctcctctccctttctctcttcactcctccctctttctctcttcactcctcctgtcgcctatgaacagcgaattcaaccttccagttttcagactaggaatgctggagttgtccttgattactctgaattgttcactccgcatatgggcactgaggatacgttgatgaactacacagacaaaaaggatagaaattcctgtcaagactacattcaatagggatgaagcaggcaataatgaataaacatactaagttgaatatgactatttaaatatatataacacatatgacttgtataatgttaaatattttaagttttttaaattcttcccttcatagattttacattatagtagaagaggcatttttgttgttgttctttttgttttggattcagagggtaaatgtgcggggttgttacatgggtatattgcataatgctgatgatggtcccatcacccaggtggtaaacatagtacgtaataggtgaatttttagcccgtgcttccctctcccatctagtcgtcctgagtgtttatcgttgctacgtttatgtcaatgtgtattcaatatttagctcccacttataattgagaatatgcagtatttcgttttttgttctcgtgttaatttgtttaggataatggcctacaaagaacatgatttcattatttttatggacatgtagtatttcatggtgtatatgtaccacggtttctttatacaatcccactgttgatgggcacctaggttgattctattgctgttgtgaatagggctgcaatgaacatacaagtgcatgtatctttttggtaacaaaaattttatatttggattacccagtagaattgctgggttgaataatagttttggtttaagttctctgagaaatctccaaactgctttccacagtagctgaactaatttacatttccactagcagtgtataagcgttctcttttctccacaatcttttcaccagcatctgttatgttttggctttttaatagccttttgatgactgtgaaatggtatctcactgtggtttggatttccatttctctaatgattagtgaatgttgagcatttttttcatatgtttattggccgtttgtatgtcttcttttgataagcgtctgttcatgtcctttacacattttcaattaaaatatttgttttttgcttgctgatttaagttctttgtatattctggaaattagatctttgtcagatgcatagtttgcaaatattttctcccattctgtagcctgtttactctgttggtaatttcttttgctgtacagaaactctttaattaggtcccacttgcctatttttagttttgttgcaattattctctggaacttagccataaattgtttgccaaagccaacgtggagaaggatattttctaggttttcttctaggattttatagtttaagttttacatttaaatctttaatccatcttgagttaatttttgtatatgttgagaagcaggagtctaatttcattcttctgcatagggctagccattatcttggcaccatttattgaatagagagtcctttccttattgcttatttctgtcaattttgttgaatatcagatcgtcgtaggtgtatgggtccatttctgggttttctattctgttctatttgtctctgtgtctgtttttgtaccagaaccatgctgcttggttactgtagccttttagtatagtttgaagttgggtaatgtgatgtctctggcttcgttctttttgcttaggattgctttggctattcaggctcctttttggttccatatgaattttagaatatttttctgattctgtgaaaaatgacttgatattttgctagggatagcattggagtggtaacttgctttggacagtgtggccattttaatgatattgattattccaatccatgagcatggagtatttttatatttattcagtcatcttgatttctttcagcagtgttttgtagttcaccctgtagaacatttcacttccatggttagatgtattcctattttgtggctattgtaaatggcattgtatttttttttatttggccctaaactagaatgttattggtgtatagaattgctactgatttttgtacattgattttgtatccttaaactttactgaagttatttatcagttctaggagacttttggagaagtctttagggttttctatgtatgaaatcatatcatcagcaaagagagacagtttgacttcttcttctttttggatgccatttatttctttctcttgcctagttgctctgactaggacttccagggcaatgctgaataggagtggtgagagtgggcatccttgtcttgttccagtactcaagagaaatgcttccagcatttacctgtttagtatgatgttggctgtggtttgtcataggtggatcttattattctaaggtatattcctttgatgcctagcctgtcgagggtttttaatcatgaatggatattgaattttattgaaggttttttctgaaactattgagatgatcatatggtttttgttttttcattctgtttatgtggtgaatcacacttattgatttgttatgttgaaccagccttgcatcccaggaataaagcctacttgattgttgtgaattaactttttgatgtgcttcttgatttagtttgctcatattttgttgaggattttcgtgtttatgttaatcagagatattgtcctgaagttttcttttttcattgtgtctctggcagattttgatatcaggatgatgctggcattgtagaatgagttagggaggagcccctctccttaatattatggaatagtttcagtaagattactatcagttcttctttgtatgcttggtagaattcagttgtgaatccatctggtccagggctaaatttggttggtaggttttttattactgattcaattttggaacttgttataggtctgttcaagttttcacttccgtcctggttcaatcttgggaggttgtatgtttccaggaatttatccatttcctctagatttcctactttgtgtgcatagaggtgttcataacggtctctgaaaatctttggcatttctgtgggattggtcgtaatgtcatttttgtcatttcttgtgctttttggaacttctgtctgtttttcctcgtttttctagctagcagtctattagtcttgtttattcttatgaaaaaccaactctttgtttcactaacattttatggacttttgcatctcaattttatttagtcattatctgattttagttatgtcttttcctctgctagctgtgagattgaattgtgctctttttttctagttcctctagtgttatgttagattgtttagttgagatctttctaacctcttgatgaaggcattttagcactataaactttcctcttaacactgcttttgctacatcccaaagattttggaaagttgtgtctctattttcattaatttcaaataattttttgatttctgccttaatttcattgttcacccaacagttattcgggagcatgtggcttaatttccatgcttttgtgtagttttgagagatcttcttggtattgatttctattgttatttcactatgatttgagagtggcctttgtatgattttaattttttttaatttattgagacttgctttatgactgagcatgtggggcaatcttagaatacgttccatgtgcatatgagaagaatgtgtgttctgtcattgttggcttgagtatcctagagaggtctattaggtccaactggtcaagtgtcaagtttaattccagaattccttcgtcagttttctgcctcagtgatctgtctaatgctatcagtggagtgataaagcccccactaatattgtgctgccatctacgttttattgtaggccaataatttgttttatgaatctgagtgctccagtgttgggtgcatatatgtttagaatagttaagtctttttgttcaattgaaccttttatcattttataatgcccttctttgtccttcctgattgttgttggtttaaagtatgttttaatctgatttaagggtagcaactcctgctcttttttgtttttcatttgcatggtagatctttcttcattctttcactttgagcctgtgagtgtcattcatgtaggatgcatcttctgaaaacagcagacagttgtgtcttgtctttttatccagcttaccactttatgcattttaaagggagagtgtagactgtttacatttagggttagcattgacatgtgagattttgctcctgtcattgtgttgtttagctggttgttttgtagacttcattgtgtaataagtgtatttttattggtagcaggtttcgtctttcatttccatgtttagcaatcacttacggatttcctgtaagaatcatctggtggtaatgaatctccttggtgcttgcttgtctgagaaggattgtatttctccttcacttatgaaactcagtttggtgggatatgagttcttggttgaaatttattttctttaataatgctgaaaatataggcccccccatatcttctggcttgtaaggtttctgctgacagaactgttgctggcctgatgaggttctttttgtaggtgacctgacctttctcactagctgccttaacaattttttcttttgcattgaccttggtgaatctgatgactatgtgacttggcaatggttgtcttgtatagtgtctcacaggagttctctgtatttcttgaatttgtatgcccacctctctggtgagataggggaaattttcatggactgcatcctcagatgtatgttctaagttgcttactctctttctcaggaatgactgtgagtcatagacttggtctctttacataacctcataaatcttgaaggttttgttcatgttttaaattcttttttctttatttttgtccaaccaagttgattcaaataactggtcttcaaactctgagattctttcctcagcttggtctgttctgctgttaatgcctctgactatattatgaaatttttgaagttgatccctcaatttctgaagttcagttttgttctttcttaaaatagctatttcatctttaagctctttgatcatttttctggattccttgagttccttgtattgggtttcaatgatctcctggatcttgatgtacttccttgccatccagattctgaattctatgtatgtcatttgagtcattttaatctggttaaaatcctttgctggaggacttgtgtgtttgtctggaggtaaggagacaccagcttttttgaattgctagagttcttgagatgactctttaacatatgagggctggtgttccattaacaatagtgtacattgagtatagtcagttggcttcattctgagtgctttcaaagggccaaagctctgtacagcatctttatttgtggctagatttttgctttaggtttcacaggtgctgtatattggaaaaatgtttttggtgttgtcatttggggtgcaatccagtaggtgatgcttaagagtggtagctggcagataggctcttactcagtccacagctcttttgtattttggtgcagtcctcagtagtgctctgtggtggtagggagagatgaccccctcaccagatacattcctgggccttgggggagccctctcttattactggcactgcacctgcatttcatttattaggtgtcctgggctgcagggtgccctcaggcagaggctgcggctggaaaatagaccatacccttccctggctggccctgcacaaggaggcacaccctgttcctgagccagtccatgaacccagctgtctcacccctctcagtgttctgagagtaggggatcccccactgcttgagcaccatgagcccctcctggctacaggcagtgggggtaggtatagtctctcaacccactgtccaactgatttccagggtaacagagagctgtgcctgcccacagagttcaggcagaggccaggccattgtgctggaagctgatgctaagccttgtctgatgatggggagtgaagcaatgtaacggctccctaactgtggcttctctcagggctatggcagctggcatgagactgctccaggtccaaggcctgtgggacttcctgtggacttgagttttgcctctgcaaacactccagcaactctctatgtcagtctagaggcccagggacacggatcaggtattgggatgaaggggttctccagttcccaggatttcacaggtccctgtggaaagtgaggatcccccaggggctctcactcactcaccctttctctatgttggggagcttcccctggctccatgcccatcttgggtggccagctgcccagcttcactcttccctgttctctgtgtcccctcactcccttaattgtcctgatatcgttccttaggtgatctacttgcagaggcagtgtttactcgccacttgttttctctctgtgagagtagcacacactagctgctactcatctagcatcttgaattcttcccatctgaaaaagtttcaactgcaatcacagttaaagaaatacaaaaacaatagcactctaagttacaacttctcacctatagaattcaaaaacatccaaatgattaactaaacatttgtttggtagatctgtgggaaaacatgaattccttgtgaattactggagaaaatgaaaatgatgcaacacttatggaagaaaatttggggatttttgggggggaggggaacaatatatttaaaactataaatgcatttatcctagcaattctatgaatggggatttatcttagggtacacctgcacacttaggaaataatgtatgcagtcattcattacagaattgtttgtaatagcaacaacctgaaaagcaactcatatatccatccatcacacagggactggtttcatgactacggttcatgaatactctgcagcccttagaaagaatgaggaagtggccgggcacggtggctcatgcctgtaatcccagcactttgggaggccgaggcgggtggatcacgaggtcaggagatcaagaccatcctggctaacacggtgaaaccccgtctctactaaaaacaatacaaaaaaattagccaggcaggcgcctatagtcccagctattcgggaggctgaggccggagaatggcatgaacccgggaggcagagcttgcagtgagccgagataacgccactgcactccatccagcctgggcgacagagcgagactccgtcaaaaaaaaaaaaaaagaggaagttctctatgcgctgacatggaaggaagacagatggttgaatgaaaaaagtacataattagccataaagtgtaagactttttgtctaaaaaagaagggtgatataattgcatatttatattttcttccatttatattaagagataataaaggtacacaaattggctagaataaagtggtttcctataaagggtaagagtaattgagtggatgaagactagggttagggatagatttctcagtgtattcattttaatatatgtattcattttatatatgtactaatttttatatatgtatttattttatattttgattttcttaacataaatatattattccttcataaaattaaacttgatacatttttgattactagatatgtagaaagcattatgttcagtaccacagtaatactttcaaaccagctacaattagtatttatgagcatctatgtgccagacattgtgttctgctttggttggtgggggtagaggaggaaaggaaaccatggcttacataggagtggaagtcttgtctttcactttgcacctctctccttcagacctagcataaatatgaccttaggggaggcagaacacatatgataaagagataactagcaagagacataatagtagctaaataaatactgaaggaaaaattcaggaagaggtaggaaggatatgcctcatcacttccacctgttaagaaaaactttagacattcttgccaatattccttattgcctgtcttttgaacaaatgccattatcactagagtgaaatgatatttcattgtagttttgatttgcatttctctcatgatcggtgatgttgagcacctttttatatacctgtttgccatttgtatgtcttctcttgaaaaatgtctattcagatctttgcccatttttaaatggcgtaatacattttttcctattgagttgtttgagttctttatatattctggttattaatcccttgtcagatgaataatttgcaaatattttctcccattctgaggattaccagaggctcagaggggtaatggtggtgggggagaataaaaatggttaatgagtacaaaaatatagataggagtaataagatctagtatctgatagcacaacagggtaattacagccaacaaaaatttattgtgcatttcaaaataactaagagtataattggaatgtctgtaacacaaagaagcaataaatgcttgaggtgatgtgaggggatggatatctaatttaccttgatgtgattattacatattgtatgcctgcatcaaaatagctcatgtatcttataagtatatacacctattatgtacccattaaattttttaagaactttaaacaaatcaaatttaacagagtttaattgggcaaagaatgatttgaggatcaggcaacccccagaaacagaagaggttcaaagcaactcagtgctgtcacatggttggagaggatttatgggcagaaaagggaaagagagatacagaaaatggaagtgaggtacacaaacagctggattggttacagcttgccatttgcgttatttgaacataatctgaacagttggctgtctttgcttgaccaaaacttggtgtttggtacaagagcagattacagtctatttacacatccagttagtttacagttcactatacacgaagaagaaacctttaagcagaacttaaaatatgcaaagaggaagctttaagttaaacttaatttaacacacccaattatcaaaaaatgagtagctctgcaaaagtggattttcctggtcatctttggtacttccttaaaaaagagaaaagtagtactcacgataaaaaaaaaaaagtcctcaagtctttattttattcctttccaatttaaaatgttacatcatctgaggaaggtttttccctttgaccgctttcatagacatttcttctgcatgggttggccagaatcagaagagtaattgtaactttctgttcttgtcctacagttacaaagcggtttcactttgtaaatgctctttggatggcaggaaccaagcagccatgaaaagaggagttacacctttaaaggagtcattccatcatgactctcaggactggaacatggaatacctgaatggcctctttggcacagataggccacccttgaaaggtgttccaagctaggaactcactaccactgttacatcgatgcaactctgtgagaagtttttatctggtgatggaaaatctcatctcttcaacacactgactactaccagtctcagaaccctgtaaacaagattcattcatctcaaattgggttaaagcagtcaccctgccttacattagtttggaataaggatgtggggatggtggtagaggaggggagtggatgatgatttttttattgttatttgattctaaagaaacttctatacattttgcatttaaaataattatgtttttaacaatgtttggattaattcaaaataggatattatatcctattatattaaatatactatttaatcatcttgttgaccaaatgcaacttaaacatgtaaaatggtaaatagcataataattgtcttctaagcctgcactataaagtatttcagtggcctcattattaaaggaccaaggtgcccaaagaaacaaaatttagtaatcataaacaagagacaaacctacttcttttcccccagagttctggccacattgaaataaggtgtttgaatgcttaataagaattattttggcccacacagtggctcatgcctgtaatctcagcactttgggatgccaaggtgagcagatcacttgaggccaggagttcaagaccagcgtggccaacgtggtgaaaccccatctctactaaaaatacaaaaattagcccggtgtggtggtacacgcctatagtcccagctactcgggagactgaggtgggagaatcacttgaacccgggaggccaaggctgcaatatcgagatcacaccactgcactctagcctgggcaacagagtgagagtgagactctttctcggaaaaaaaaaaaaagaattattttgaacaaagtgctgtcacctaagttagcaaaactccaagcaaggtttttggctctgtaaggaaagaattagcctactcatttggaaatttagtggtgtttgtaatgcagaaagtgacagtgagactggaaagggattggctttggggcttgttctgctttataaataataatgaatcttctccaacatgaagtaatgtgaattaaaaaaaaaaaatctgtccttagagtacaaaattacttcataacccaatctgcatttctccactccaagcatattttctgggagttctacttagagagtgaaagctgctgtgtgtgtgataattaattttaacaaacacttggcaaactgagctggactatgtataagctaccctagactaagcatgaatttgaactgcactttttatggtgttttttccacaatgacattatttaggcatttaaagttatctgaactgcaattttttgttcttttttttttaatttgactttttaaaaaaaattattcctgaataaagaggcagtttgtaaaaactcgagaactgtgagagataattggatctttgtgtagcaaaactagaagggtgttgggtatctgctctttatcaaatggaccacttacttttcttttcttttttgccctgtgttcagaaaacaaatgtgcgtgtctcctgatttataatgtatagttcattaatggagaaagtgcttgagaattagatcctaatgtcatttcccatgcagcatcttcattcttttctaaagcactatttggtaaaaacaactgatagtcgtcagaggtgatcagcaatgtttgagcactatttcctttttatatcctgcacatggaatatggacaggcaaacaaatcatttccaagtaagaaaataaattttgagggagttaatactataatttgaaagtaataacctcctatttatccatctagtttgttgttctgtactaaattatttgtgcatgtctctgtgtctataatttatgtgaaactttgcacaatcttaaataggacaaaatagacattctgtaatttcccaggcaagctatttaaggtgactatctctctacatatttgagatgaaaaacaataacatgacaatccatcccttcttaggtttttgtaagcagacttactacctgtgactcagttttgttctcacagggtactaattaatccttcacgataataacttgtcaaattccattacttctgtaaaggcaatactttatatttgtttgtattcaaattttaaactgatgttaaatgccgtgggtgcaactgcaggttaaaaatatgtgtttgaatctcttattctttttgcttggcaatgtatgaaataactgctctttctagaaatcttgatgatgaagtggcctgttgttttgtcacctaaaaatgcaataatgttcaaattaagcttttctttattaacatcacttgattgtgtgccatatttagagcttagtgaaattttaatctacacattgattaaatacattttatttattcttgtttctaatgggaactttctttgtttctaatgggaactttcttaaattaaattacatccaacatttattaaagacctaaaacataggcaattactgtgcttagaggaaaagcgcagacgaaagtgaatcagacaagttccctgccctccggaagctttcagtctagtgatgagaaagacgtatacacaccttatgttgatttaaaaaaaaaaaaagctcttacctggttgctggcatatgaaagtgttagttacagatctgccccaaactaaaggtgtcacctcgagtaaatctctttccctttccctttcaatctcttcatctataaactaggggttgggaatacatttattaacaaacacaaattgagcgtctaccatgtgataatagtagctaaacttactgagcaattaccatggggcaggtatcaagataaaccctttatgatggtaacctcatttaatcctcaaagcaattccattttcaagaggaggaaattgaggctcaaaaatgttaagtaactcccccaaggatgcaaagtgattgagccagaattcaagactaggttggtttgactccaaaactcatgccattaaaccctattgtgtcactgcaaacaactctaatagtttcaaattattagttctattaatattatattaccattatttgcccccaaaatgtaaaatgtaaatacaaagagtttggtttttgtattactagtggaggttaaaggtgcacaatggaattattcaaactgggaaaatccaggaagacttcatggaggaggcagcatatggctgcagttaataaggtttgctcacacaaaatggagaggtgaggacatttcaggcagagagaattatatgagaggttacagagcagtaaacagtcatgcgtctgcaagatcaaagggaaagggcggtaagagagaagcttgaaagtcaagtggagccagattgtggaaaaactagagagtcatgccaaggaccttgacatatagaaaatgggaagcccctgaaaggtgaagaacatgagagtgaaatgattagtaactttttggtttaggacttgtttcttttgtgttttggttgctttcttgttttgttttgtttgtggtttttaaatttacaaccaataagaatatttagtaaggtttccaaatacatcatgaatatataaaactagcctgactcaaggataataattctgggtagttggagtgaagtttcaatcagctacgtggcatttgctaatcatctgatatgagctaacaataaaggagttaacaaataaactgtcagcctacagtccagggtctcaaatagcatgtgacatagttgagaagcagttttccatatcatacatgaaataactaaagaaactacttacaaagcactataccagtaactacaataaaatacaactatacatgcaaaataatgctgaaagctgcaagtagaggggtaaagctaggccagttgctcagggaaccattctgaagtggatttgggaagtatgtctagaaggggagccattgctgtgagagtgctgaggctcatctgctactagtcccccactactcaggcatatggtaggtcagtaacaaaaccatcattgtgcactgttctttccatctaaattccatcaaattatgaccaacctatcaaggtactagttcaaattctctcttcctctataagctagtggtcttctctaaaatttaagaagatcgtgctcatcttcctacttcttgttctctttcttctgtgttttctgaggctgcaatgaactaggaacttcctctccccagaactctgtattccaggccttagatcactcaaaactgttgcttataaagtgcagagaatcaacagagaaggaatagaggttaatgtctggtcaaagatgtgattctcttgttgaaaagttcattagcttattatttatagaatcataagtcccaggaaaaaccaaaaggaaatatatattggatcctaatgatattctctttttttcttttttcttttcccccactccattgcccaggctggagtgcagtggcataatctcagctcactgcaacctccacctcccgggttcaagggactctcctgcctcagccttccaagtagatgggattacaggcatgtgccaccacatctggctaatttttttttgtatttttagtagagatggggtttcaccatgttagtcaggctggtgttgaactcctgacctcaaatgatccaccagcctcggcctcccagtgtgctgggattgcaggcgtgagccaccacacccggcctgatattctcttgcaagggcattgtttacattgtctatcatcagaactgtagagtgttggctccaggcacagaacccctagagttttgtaaaccatttatatcacactggcaaccagaagtaactttatatactcaagaatcaagatttcacctagaagtacctcaggtaggtgttggttcattcacattccaaccaaaagataatgtaccataaagtgcataccgcctagtccgtaatgattaaggcaaccacataaaatctcattatttaaaagaaattaagtccaggcacggtggctcacacctgtaatctcagcacttcgggaggccaaggagggcagatcacctgaggttgggagtttgagaccagcctgatcaacatggagaaatcccatctctactaaaaatacaaaattagcggggcatggtggtgcatgcctataatcccagctactcaggaggctgaggcaggagaatcacttgaacccaggaggtggaggttgagatcgtgccattgcactccagcctggacaacaagagtgaaactctgtctcaaaaaagaaaaaaagaaaaagaaattaaatgcactatggtttatggagcggtattcctcctccatgtcctacataagatctttcacatgccagtcacagttaaatctaatttgctgtaatctggataaatgggagctaatcaacaagctctcagctctagctctgaatcagcagcagatattgcatttttgaaatacactaatagcaagaatgccttcctgacaacaactggcatttttgacacagcaggaagtttatctggattctgatataatagttattggaatcatacataggtacatagtttaaaaggctaataagtcatttgttattgcttttattatctctgcatagttagtaaaattgagattagaaccacttctcgaatgtactgttctaaatccttagcttgcttgatcacacatgaccctcacaatgatcctaggagaaattattctgcatgccattttgtagctggggaaactgaggcacagagaaatacagtactgcccaaaatgtcataactaatcaaaggcaaagacaatactcacaccagctctgattccagagcccactctcttaaccatatgcttttctgcttccctagttgtagagtctttttgtatgactgcattaattatatgtgaagagttcaaaaatttctatataaggtcttttaagggtgtcattctggttgaaaatggaggactaggcttctcacttgaagacatatttctgtagaaaaacctattttcatttagatgctacagttacttgatgtggttaataaaccagttaacagagtatgaaaaggataagggttaaagccctcccaagccatctttcatgctgctaatatgaatcacattactagatacttaaatatcattttctctttggttcccagaagactgcatatatgctagaatatttgtcctcctcttttaccctttcaggcaataaagtattttggaccactgtactatgttataattattgtttctctcctgatttttttgctccaatctaatgaaagacatacaagctactatactgctacacaatgactaaatacctgttggattaggtggggggaagatacacagtcactggctagaaagcatcatgcatacagagccattttcaccatatattttatttctcatgatcatgtagaatttaggctttggtgttgattatttctctcttaggaaacatagttgtttcagggttgatatcacaaaaaaacagaaaaacctattcgagaaaaggaaaattatttgtctgtaggccaaattttgaagtaggaaaacctgcttttggagttgtattcccctcccaggcacttaatccaagttccagtcttattctaaactggggatgctagtattaaccaccataggagttatctgagatgagttatcatcaacttggtaccaggttgttgtcctctggactcagtgagctctagaattgcatgaaactggcctaatttatcaaagtatgtagccttgggtaaataattcaagctctcagaggtccagttatctcctctgtaaaacatatctacatcctagggatgacaatatctacatcctagagatgtcaggaggattaagtgtaattttttttaattgtatgtatttaaaatgggcaacataatgttttgatatacacgtgtatagtgattactacagtcaagcaaattaacatatccatcatttcatagctaccttttatgtatgtgataagattatctaaaatctattctcttaccaaatttccagtatacaatattgatatggtttgatccatatccccatccaaatctcatgttcagttgcaatccccaacgttggagatggagcctggttggaggtgattggatcacaggggtggcttctaatggttcagcaccatcctttcttggtactgtatagtgagtaagttctcacgagatctggttgtttaaaagtgtgtaacacctcccccactttccctctctctgttcctcctgctcccgctatgtgaagtgccagctccctctttgccttccgccatgattgtaagttctctgaggcatccccagaagctgatgctgccatgcttcctatacagcctgcagaaccatgagtcaattaaacctcttttctttgtaaattacccagtctcaagtatttctttatagcaatgcaagaatggactaatacagaaaattgttactgagaagaagggcattgctataaagatacctgaaaatgtagaagtgactttggaaccggctaacaggcagaagttgaaacattttagagggctcagaagaagacagaaagatgagagaaagtttggaactcgctaggaacttgttgagtggttgtaaccaaaatactgatagtgatatagacagtgaagtccaggctgaggaggtctcagatggaaatgagaaatttattgggaatgagtaaaggtcaggtttgctatgctttagcaaagagcttagctgcattgttcctctgttctagggatctgtgaaatcttagacttaagaatgatgatttagggtatctggcagaagaaatttctaagcagcagagtgttcaagaagtaacctagctgcttctaatagcctatgctcataggcatgagcacagaaatgacctgaaattggaacttacacttaaaagggaagcagagcataaaagtttgtaaattttgcagcctggccatgtggtagtaaagaaaagctcgttctcaggagaggaagtcaagcaggctgcataaatttgcataactaaaaggaaggcaagggctgataaccaaaacaatggggagaaagactcataggactaacaggcattttattttattttatttttattttattattattatactttaagttttagggtacatgtgcacaatgtgcaggttagttgcatatgtatacatgtgccatgctggtgtgctgcacccattaactcgtcatttagcattaggtatatctcctaatgctatccctcccccctcccccaccccacaacagtccccagagtgtgatgttccccttcctgtgtccatgtgttctcattgttcaattcccacctatgagtgagaacatgtggtgtttggttttttgaccttgcaatagtttactgagaatgacgatttccaatttcatccatgtccctacaaaggacatgaactcatcattttttatggctgcatagtattccatggtgtatatgtgccacattttcttaatccagtctatcactgttggacatttgggttggttccaagtctttgctattgtgaatagtgccacaataaacatagtgtgcatgtgtctttatagcagcaggatttatagtcctttgggtatatacccagtgatgggatggctgggtcaaatggtatttctagttctagatccctgaggaatcgccacactgacttccacaatggttgaactagtttacagtcccaccaacagtgtaaaagtgttcctaataggcattttaggctttcatggtggtccctctcatcacaggccccgaggcctaggaggactgaatcatttcctgggccaggcctagggcccctgctccctcttacagccttgggactctgctccctgaatcccagctgctcaaaggggcccaggtactgttacagtaggtagctaatcaggcatgagtggggtaagagagaagtccccaccacccaccaggaatgtcaggcaaccatcagatgatggtcaggcagttgtcatactgcctctctaaaatagtaattggttgcagccagcaccagggagaggcaacttctcaatagatagaaacacctgaaattggtaactgggcgcttccaataagatctcaggaactgagagagtgggcttaacatgcacattaagaggcaaaatggtgaagtatgacctttgggggcattccaccggaaaagggaagaaagcctcaggtaagcatgtatacaactccagtaaacacactgcacacgctcaccttccaagtgcaagcagggcaccatgcatgcggcaagctcacccttagggaaggaccaagggaaaggggcacaagatgtcagaagtaggccagtgtataagatcctaggttcaaggtcaaacagggcacttgacctccaaggtgcccacttgggcctcttccaaatgtactttcctttcattcctgttctaaagctttttaataaacttttactcctgctctgaaacttgtcgcagtctctttttctgccttatgcctcttggtcaaattctttcttctgaggaggcaagaattgaggttgctgcagacccacatggatttgcagctggtaactcagataactttcaccagtaagaatacagttcaggctgctgcttcacagggtgccaggcataagccttggtggcttccataagctgtgaagccggcgggcgcacataatgcaagagttgaggcttaagaagctctgcctagattttagaggatgtatgaaaaagcctggatgtccagacagaagcctgttactggggtggaatcctcatggagaacatctactagggaagcaaggagaagaaatgtggggttgcagcccccacagagagtcccctggggcactgcctagcagagctatgacaagacagccaccgtcctccagaccccagaatggtagatccaccaacaacttgcaccctgcagcctggaaaagctgcaagcactcaatgctagcccatgagagcagctgtgggagatgaaccctggaaaaccacaggggtggttctgcccaaggttttgggagcccactcattgcatcagtgttccctgggtgtgagtcaaaggagattatttcagagctttaacatttaatgactgcccggctggctttcagacttgcaatggggccctatagcctctttcttttggcagatttctccctttcggaatggcagtatctgcccaatgcctatacccccattgtatctttgaagcaattaccttgtttttgattttacaggttcataggtagaagggactagcttcgtctcaggtgagacttgggactttggacttttgaatgaatgctggatcgagttaagactttggggaactgttggtaaggcacgacagtattttgcaatatgagaaggacattagatttgggaggggccagagttggaataacatggtttggatctctgtccccacccaaatctcatgttcaactgtaatccccagtgttggaggttgggcctggtgggaggtgagtggattatggggtggcttctaatggttttgtacagtcccctcttggtactatatagtgagttctgacaagatctagttgtttaaacgtatgtagcacctcccatttctctcttcccccagttcctgccatgtgaagtctggggtctccctatgccttccatcatgattttaagttccctatggcctgcccagaagctgatccagccatgcttcttgtacagcctgcagaactgtgagccattaaacttttctttataaattacccagtttcagttatttctttatagcagtgtaagaatggactaacacaattattaacgctagtcctcatgttgtacattaaatctctagatgtattagacgtaactgcaactttgtaccctaccctacaattttctttccccccaagccccccaaccaagggtctactctgtttctataaattcagttgttttttaattccacgtataagtgaagtacaactcagtgtagaaacttggtaaatgctagctacttgttataagctgtcagtcaaaataaaaatacagagatgaatctctaaattaagtgatttatttgggaagaaagaattgcaattagggcatacatgtagatcagatggtcttcggtatatccacacaacaaagaaaagggggaggttttgttaaaaaagagaaatgttacatagtgctctttgagaaaattcattggcactattaaggatctgaggagctggtgagtttcaactggtgagtgatggtggtagataaaattagagctgcagcaggtcattttagcaactattagataaaactggtctcaggtcacaacgggcagttgcagcagctggacttggagagaattacactgtgggagcagtgtcatttgtcctaagtgcttttctaccccctacccccactattttagttgggtataaaaagaatgacccaatttgtatgatcaactttcacaaagcatagaacagtaggaaaagggtctgtttctgcagaaggtgtagacgttgagagccattttgtgtatttattcctccctttcttcctcggtgaatgattaaaacgttctgtgtgatttttagtgatgaaaaagattaaatgctactcactgtagtaagtgccatctcacacttgcagatcaaaaggcacacagtttaaaaaacctttgtttttttacacatctgagtggtgtaaatgctactcatctgtagtaagtggaatctatacacctgcagaccaaaagacgcaaggtttcaaaaatctttgtgttttttacacatcaaacagaatggtacgtttttcaaaagttaaaaaaaaacaactcatccacatattgcaactagcaaaaatgacattccccagtgtgaaaatcatgcttgagagaattcttacatgtaaaggcaaaattgcgatgactttgcaggggaccgtgggattcccgcccgcagtgccggagctgtcccctaccagggtttgcagtggagttttgaatgcacttaacagtgtcttacggtaaaaacaaaatttcatccaccaattatgtgttgagcgcccactgcctaccaagcacaaacaaaaccattcaaaaccacgaaatcgtcttcactttctccagatccagcagcctcccctattaaggttcgcacacgctattgcgccaacgctcctccagagcgggtcttaagataaaagaacaggacaagttgccccgccccatttcgctagcctcgtgagaaaacgtcatcgcacatagaaaacagacagacgtaacctacggtgtcccgctaggaaagagaggtgcgtcaaacagcgacaagttccgcccacgtaaaagatgacgcttggtgtgtcagccgtccctgctgcccggttgcttctcttttgggggcggggtctagcaagagcaggtgtgggtttaggaggtgtgtgtttttgtttttcccaccctctctccccactacttgctctcacagtactcgctgagggtgaacaagaaaagacctgataaagattaaccagaagaaaacaaggagggaaacaaccgcagcctgtagcaagctctggaactcaggagtcgcgcgctaggggccggggccggggccggggcgtggtcggggcgggcccgggggcgggcccggggcggggctgcggttgcggtgcctgcgcccgcggcggcggaggcgcaggcggtggcgagtgggtgagtgaggaggcggcatcctggcgggtggctgtttggggttcggctgccgggaagaggcgcgggtagaagcgggggctctcctcagagctcgacgcatttttactttccctctcatttctctgaccgaagctgggtgtcgggctttcgcctctagcgactggtggaattgcctgcatccgggccccgggcttcccggcggcggcggcggcggcggcggcgcagggacaagggatggggatctggcctcttccttgctttcccgccctcagtacccgagctgtctccttcccggggacccgctgggagcgctgccgctgcgggctcgagaaaagggagcctcgggtactgagaggcctcgcctgggggaaggccggagggtgggcggcgcgcggcttctgcggaccaagtcggggttcgctaggaacccgagacggtccctgccggcgaggagatcatgcgggatgagatgggggtgtggagacgcctgcacaatttcagcccaagcttctagagagtggtgatgacttgcatatgagggcagcaatgcaagtcggtgtgctccccattctgtgggacatgacctggttgcttcacagctccgagatgacacagacttgcttaaaggaagtgactattgtgacttgggcatcacttgactgatggtaatcagttgtctaaagaagtgcacagattacatgtccgtgtgctcattgggtctatctggccgcgttgaacaccaccaggctttgtattcagaaacaggagggaggtcctgcactttcccaggaggggtggccctttcagatgcaatcgagattgttaggctctgggagagtagttgcctggttgtggcagttggtaaatttctattcaaacagttgccatgcaccagttgttcacaacaagggtacgtaatctgtctggcattacttctacttttgtacaaaggatcaaaaaaaaaaaagatactgttaagatatgatttttctcagactttgggaaacttttaacataatctgtgaatatcacagaaacaagactatcatataggggatattaataacctggagtcagaatacttgaaatacggtgtcatttgacacgggcattgttgtcaccacctctgccaaggcctgccactttaggaaaaccctgaatcagttggaaactgctacatgctgatagtacatctgaaacaagaacgagagtaattaccacattccagattgttcactaagccagcatttacctgctccaggaaaaaattacaagcaccttatgaagttgataaaatattttgtttggctatgttggcactccacaatttgctttcagagaaacaaagtaaaccaaggaggacttctgtttttcaagtctgccctcgggttctattctacgttaattagatagttcccaggaggactaggttagcctacctattgtctgagaaacttggaactgtgagaaatggccagatagtgatatgaacttcaccttccagtcttccctgatgttgaagattgagaaagtgttgtgaactttctggtactgtaaacagttcactgtccttgaagtggtcctgggcagctcctgttgtggaaagtggacggtttaggatcctgcttctctttgggctgggagaaaataaacagcatggttacaagtattgagagccaggttggagaaggtggcttacacctgtaatgccagagctttgggaggcggaggcaagaggatcacttgaagccaggagttcaagctcaacctgggcaacgtagaccctgtctctacaaaaaattaaaaacttagccgggcgtggtgatgtgcacctgtagtcctagctacttgggaggctgaggcaggagggtcatttgagcccaagagtttgaagttaccgagagctatgatcctgccagtgcattccagcctggatgacaaaacgagaccctgtctctaaaaaacaagaagtgagggctttatgattgtagaattttcactacaatagcagtggaccaaccacctttctaaataccaatcagggaagagatggttgattttttaacagacgtttaaagaaaaagcaaaacctcaaacttagcactctactaacagttttagcagatgttaattaatgtaatcatgtctgcatgtatgggattatttccagaaagtgtattgggaaacctctcatgaaccctgtgagcaagccaccgtctcactcaatttgaatcttggcttccctcaaaagactggctaatgtttggtaactctctggagtagacagcactacatgtacgtaagataggtacataaacaactattggttttgagctgatttttttcagctgcatttgcatgtatggatttttctcaccaaagacgatgacttcaagtattagtaaaataattgtacagctctcctgattatacttctctgtgacatttcatttcccaggctatttcttttggtaggatttaaaactaagcaattcagtatgatctttgtccttcattttctttcttattctttttgtttgtttgtttgtttgtttttttcttgaggcagagtctctctctgtcgcccaggctggagtgcagtggcgccatctcagctcattgcaacctctgccacctccgggttcaagagattctcctgcctcagcctcccgagtagctgggattacaggtgtccaccaccacacccggctaattttttgtatttttagtagaggtggggtttcaccatgttggccaggctggtcttgagctcctgacctcaggtgatccacctgcctcggcctaccaaagagctgggataacaggtgtgacccaccatgcccggcccattttttttttcttattctgttaggagtgagagtgtaactagcagtataatagttcaattttcacaacgtggtaaaagtttccctataattcaatcagattttgctccagggttcagttctgttttaggaaatacttttattttcagtttaatgatgaaatattagagttgtaatattgcctttatgattatccacctttttaacctaaaagaatgaaagaaaaatatgtttgcaatataattttatggttgtatgttaacttaattcattatgttggcctccagtttgctgttgttagttatgacagcagtagtgtcattaccatttcaattcagattacattcctatatttgatcattgtaaactgactgcttacattgtattaaaaacagtggatattttaaagaagctgtacggcttatatctagtgctgtctcttaagactattaaattgatacaacatatttaaaagtaaatattacctaaatgaatttttgaaattacaaatacacgtgttaaaactgtcgttgtgttcaaccatttctgtacatacttagagttaactgttttgccaggctctgtatgcctactcataatatgataaaagcactcatctaatgctctgtaaatagaagtcagtgctttccatcagactgaactctcttgacaagatgtggatgaaattctttaagtaaaattgtttactttgtcatacatttacagatcaaatgttagctcccaaagcaatcatatggcaaagataggtatatcatagtttgcctattagctgctttgtattgctattattataaatagacttcacagttttagacttgcttaggtgaaattgcaattctttttactttcagtcttagataacaagtcttcaattatagtacaatcacacattgcttaggaatgcatcattaggcgattttgtcattatgcaaacatcatagagtgtacttacacaaacctagatagtatagcctttatgtacctaggccgtatggtatagtctgttgctcctaggccacaaacctgtacaactgttactgtactgaatactatagacagttgtaacacagtggtaaatatttatctaaatatatgcaaacagagaaaaggtacagtaaaagtatggtataaaagataatggtatacctgtgtaggccacttaccacgaatggagcttgcaggactagaagttgctctgggtgagtcagtgagtgagtggtgaattaatgtgaaggcctagaacactgtacaccactgtagactataaacacagtacgctgaagctacaccaaatttatcttaacagtttttcttcaataaaaaattataactttttaactttgtaaactttttaattttttaacttttaaaatacttagcttgaaacacaaatacattgtatagctatacaaaaatattttttctttgtatccttattctagaagcttttttctattttctattttaaatttttttttttacttgttagtcgtttttgttaaaaactaaaacacacacactttcacctaggcatagacaggattaggatcatcagtatcactcccttccacctcactgccttccacctccacatcttgtcccactggaaggtttttaggggcaataacacacatgtagctgtcacctatgataacagtgctttctgttgaatacctcctgaaggacttgcctgaggctgttttacatttaacttaaaaaaaaaaaaagtagaaggagtgcactctaaaataacaataaaaggcatagtatagtgaatacataaaccagcaatgtagtagtttattatcaagtgttgtacactgtaataattgtatgtgctatactttaaataacttgcaaaatagtactaagaccttatgatggttacagtgtcactaaggcaatagcatattttcaggtccattgtaatctaatgggactaccatcatatatgcagtctaccattgactgaaacgttacatggcacataactgtatttgcaagaatgatttgttttacattaatatcacataggatgtacctttttagagtggtatgtttatgtggattaagatgtacaagttgagcaaggggaccaagagccctgggttctgtcttggatgtgagcgtttatgttcttctcctcatgtctgttttctcattaaattcaaaggcttgaacgggccctatttagcccttctgttttctacgtgttctaaataactaaagcttttaaattctagccatttagtgtagaactctctttgcagtgatgaaatgctgtattggtttcttggctagcatattaaatatttttatctttgtcttgatacttcaatgtcgttttaaacatcaggatcgggcttcagtattctcataaccagagagttcactgaggatacaggactgtttgcccattttttgttatggctccagacttgtggtatttccatgtctttttttttttttttttttttgaccttttagcggctttaaagtatttctgttgttaggtgttgtattacttttctaagattacttaacaaagcaccacaaactgagtggctttaaacaacagcaatttattctctcacaattctagaagctagaagtccgaaatcaaagtgttgacaggggcatgatcttcaagagagaagactctttccttgcctcttcctggcttctggtggttaccagcaatcctgagtgttcctttcttgccttgtagtttcaacaatccagtatctgccttttgtcttcacatggctgtctaccatttgtctctgtgtctccaaatctctctccttataaacacagcagttattggattaggccccactctaatccagtatgaccccattttaacatgattacacttatttctagataaggtcacattcacgtacaccaagggttaggaattgaacatatctttttgggggacacaattcaacccacaagtgtcagtctctagctgagcctttcccttcctgtttttctcctttttagttgctatgggttaggggccaaatctccagtcatactagaattgcacatggactggatatttgggaatactgcgggtctattctatgagctttagtatgtaacatttaatatcagtgtaaagaagcccttttttaagttatttctttgaatttctaaatgtatgccctgaatataagtaacaagttaccatgtcttgtaaaatgatcatatcaacaaacatttaatgtgcacctactgtgctagttgaatgtctttatcctgataggagataacaggattccacatctttgacttaagaggacaaaccaaatatgtctaaatcatttggggttttgatggatatctttaaattgctgaacctaatcattggtttcatatgtcattgtttagatatctccggagcatttggataatgtgacagttggaatgcagtgatgtcgactctttgcccaccgccatctccagctgttgccaagacagagattgctttaagtggcaaatcacctttattagcagctacttttgcttactgggacaatattcttggtcctagagtaaggcacatttgggctccaaagacagaacaggtacttctcagtgatggagaaataacttttcttgccaaccacactctaaatggagaaatccttcgaaatgcagagagtggtgctatagatgtaaagttttttgtcttgtctgaaaagggagtgattattgtttcattaatctttgatggaaactggaatggggatcgcagcacatatggactatcaattatacttccacagacagaacttagtttctacctcccacttcatagagtgtgtgttgatagattaacacatataatccggaaaggaagaatatggatgcataaggtaagtgatttttcagcttattaatcatgttaacctatctgttgaaagcttattttctggtacatataaatcttatttttttaattatatgcagtgaacatcaaacaataaatgttatttattttgcatttaccctattagatacaaatacatctggtctgatacctgtcatcttcatattaactgtggaaggtacgaaatggtagctccacattatagatgaaaagctaaagcttagacaaataaagaaacttttagaccctggattcttcttgggagcctttgactctaataccttttgtttccctttcattgcacaattctgtcttttgcttactactatgtgtaagtataacagttcaaagtaatagtttcataagctgttggtcatgtagcctttggtctctttaacctctttgccaagttcccaggttcataaaatgaggaggttgaatggaatggttcccaagagaattccttttaatcttacagaaattattgttttcctaaatcctgtagttgaatatataatgctatttacatttcagtatagttttgatgtatctaaagaacacattgaattctccttcctgtgttccagtttgatactaacctgaaagtccattaagcattaccagttttaaaaggcttttgcccaatagtaaggaaaaataatatcttttaaaagaataattttttactatgtttgcaggcttacttccttttttctcacattatgaaactcttaaaatcaggagaatcttttaaacaacatcataatgtttaatttgaaaagtgcaagtcattcttttcctttttgaaactatgcagatgttacattgactgttttctgtgaagttatctttttttcactgcagaataaaggttgttttgattttattttgtattgtttatgagaacatgcatttgttgggttaatttcctacccctgcccccattttttccctaaagtagaaagtatttttcttgtgaactaaattactacacaagaacatgtctattgaaaaataagcaagtatcaaaatgttgtgggttgtttttttaaataaattttctcttgctcaggaaagacaagaaaatgtccagaagattatcttagaaggcacagagagaatggaagatcaggtatatgcaaattgcatactgtcaaatgtttttctcacagcatgtatctgtataaggttgatggctacatttgtcaaggccttggagacatacgaataagcctttaatggagcttttatggaggtgtacagaataaactggaggaagatttccatatcttaaacccaaagagttaaatcagtaaacaaaggaaaatagtaattgcatctacaaattaatatttgctccctttttttttctgtttgcccagaataaattttggataacttgttcatagtaaaaataaaaaaaattgtctctgatatgttctttaaggtactacttctcgaacctttccctagaagtagctgtaacagaaggagagcatatgtacccctgaggtatctgtctggggtgtaggcccaggtccacacaatatttcttctaagtcttatgttgtatcgttaagactcatgcaatttacattttattccataactattttagtattaaaatttgtcagtgatatttcttaccctctcctctaggaaaatgtgccatgtttatcccttggctttgaatgcccctcaggaacagacactaagagtttgagaagcatggttacaagggtgtggcttcccctgcggaaactaagtacagactatttcactgtaaagcagagaagttcttttgaaggagaatctccagtgaagaaagagttcttcacttttacttccatttcctcttgtgggtgaccctcaatgctccttgtaaaactccaatattttaaacatggctgttttgcctttctttgcttctttttagcatgaatgagacagatgatactttaaaaaagtaattaaaaaaaaaaacttgtgaaaatacatggccataatacagaacccaatacaatgatctcctttaccaaattgttatgtttgtacttttgtagatagctttccaattcagagacagttattctgtgtaaaggtctgacttaacaagaaaagatttccctttacccaaagaatcccagtccttatttgctggtcaataagcagggtccccaggaatggggtaactttcagcaccctctaacccactagttattagtagactaattaagtaaacttatcgcaagttgaggaaacttagaaccaactaaaattctgcttttactgggattttgttttttcaaaccagaaacctttacttaagttgactactattaatgaattttggtctctcttttaagtgctcttcttaaaaatgttatcttactgctgagaagttcaagtttgggaagtacaaggaggaatagaaacttaagagattttcttttagagcctcttctgtatttagccctgtaggatttttttttttttttttttttttggtgttgttgagcttcagtgaggctattcattcacttatactgataatgtctgagatactgtgaatgaaatactatgtatgcttaaacctaagaggaaatattttcccaaaattattcttcccgaaaaggaggagttgccttttgattgagttcttgcaaatctcacaacgactttattttgaacaatactgtttggggatgatgcattagtttgaaacaacttcagttgtagctgtcatctgataaaattgcttcacagggaaggaaatttaacacggatctagtcattattcttgttagattgaatgtgtgaattgtaattgtaaacaggcatgataattattactttaaaaactaaaaacagtgaatagttagttgtggaggttactaaaggatggtttttttttaaataaaactttcagcattatgcaaatgggcatatggcttaggataaaacttccagaagtagcatcacatttaaattctcaagcaacttaataatatggggctctgaaaaactggttaaggttactccaaaaatggccctgggtctgacaaagattctaacttaaagatgcttatgaagactttgagtaaaatcatttcataaaataagtgaggaaaaacaactagtattaaattcatcttaaataatgtatgatttaaaaaatatgtttagctaaaaatgcatagtcatttgacaatttcatttatatctcaaaaaatttacttaaccaagttggtcacaaaactgatgagactggtggtggtagtgaataaatgagggaccatccatatttgagacactttacatttgtgatgtgttatactgaattttcagtttgattctatagactacaaatttcaaaattacaatttcaagatgtaataagtagtaatatcttgaaatagctctaaagggaatttttctgttttattgattcttaaaatatatgtgctgattttgatttgcatttgggtagattatacttttatgagtatggaggttaggtattgattcaagttttccttacctatttggtaaggatttcaaagtctttttgtgcttggttttcctcatttttaaatatgaaatatattgatgacctttaacaaattttttttatctcaaattttaaaggagatcttttctaaaagaggcatgatgacttaatcattgcatgtaacagtaaacgataaaccaatgattccatactctctaaagaataaaagtgagctttagggccgggcatggtcagaaatttgacaccaacctggccaacatggcgaaaccccgtctctactaaaaatacaaaaatcagccgggcatggtggcggcacctatagtcccagctacttgggaggatgagacaggagagtcacttgaacctgggaggagaggttgcagtgagctgagatcacgccattgcactccagcctgagcaatgaaagcaaaactccatctcaaaaaaaaaaaaagaaaagaaagaataaaagtgagctttggattgcatataaatcctttagacatgtagtagacttgtttgatactgtgtttgaacaaattacgaagtattttcatcaaagaatgttattgtttgatgttatttttattttttattgcccagcttctctcatattacgtgattttcttcacttcatgtcactttattgtgcagggtcagagtattattccaatgcttactggagaagtgattcctgtaatggaactgctttcatctatgaaatcacacagtgttcctgaagaaatagatgtaagtttaaatgagagcaattatacactttatgagttttttggggttatagtattattatgtatattattaatattctaattttaatagtaaggactttgtcatacatactattcacatacagtattagccactttagcaaataagcacacacaaaatcctggattttatggcaaaacagaggcatttttgatcagtgatgacaaaattaaattcattttgtttatttcattacttttataattcctaaaagtgggaggatcccagctcttataggagcaattaatatttaatgtagtgtcttttgaaacaaaactgtgtgccaaagtagtaaccattaatggaagtttacttgtagtcacaaatttagtttccttaatcatttgttgaggacgttttgaatcacacactatgagtgttaagagatacctttaggaaactattcttgttgttttctgattttgtcatttaggttagtctcctgattctgacagctcagaagaggaagttgttcttgtaaaaattgtttaacctgcttgaccagctttcacatttgttcttctgaagtttatggtagtgcacagagattgttttttggggagtcttgattctcggaaatgaaggcagtgtgttatattgaatccagacttccgaaaacttgtatattaaaagtgttatttcaacactatgttacagccagactaatttttttattttttgatgcattttagatagctgatacagtactcaatgatgatgatattggtgacagctgtcatgaaggctttcttctcaagtaagaatttttcttttcataaaagctggatgaagcagataccatcttatgctcacctatgacaagatttggaagaaagaaaataacagactgtctacttagattgttctagggacattacgtatttgaactgttgcttaaatttgtgttatttttcactcattatatttctatatatatttggtgttattccatttgctatttaaagaaaccgagtttccatcccagacaagaaatcatggccccttgcttgattctggtttcttgttttacttctcattaaagctaacagaatcctttcatattaagttgtactgtagatgaacttaagttatttaggcgtagaacaaaattattcatatttatactgatctttttccatccagcagtggagtttagtacttaagagtttgtgcccttaaaccagactccctggattaatgctgtgtacccgtgggcaaggtgcctgaattctctatacacctatttcctcatctgtaaaatggcaataatagtaatagtacctaatgtgtagggttgttataagcattgagtaagataaataatataaagcacttagaacagtgcctggaacataaaaacacttaataatagctcatagctaacatttcctatttacatttcttctagaaatagccagtatttgttgagtgcctacatgttagttcctttactagttgctttacatgtattatcttatattctgttttaaagtttcttcacagttacagattttcatgaaattttacttttaataaaagagaagtaaaagtataaagtattcacttttatgttcacagtcttttcctttaggctcatgatggagtatcagaggcatgagtgtgtttaacctaagagccttaatggcttgaatcagaagcactttagtcctgtatctgttcagtgtcagcctttcatacatcattttaaatcccatttgactttaagtaagtcacttaatctctctacatgtcaatttcttcagctataaaatgatggtatttcaataaataaatacattaattaaatgatattatactgactaattgggctgttttaaggctcaataagaaaatttctgtgaaaggtctctagaaaatgtaggttcctatacaaataaaagataacattgtgcttatagcttcggtgtttatcatataaagctattctgagttatttgaagagctcacctacttttttttgtttttagtttgttaaattgttttataggcaatgtttttaatctgttttctttaacttacagtgccatcagctcacacttgcaaacctgtggctgttccgttgtagtaggtagcagtgcagagaaagtaaataaggtagtttattttataatctagcaaatgatttgactctttaagactgatgatatatcatggattgtcatttaaatggtaggttgcaattaaaatgatctagtagtataaggaggcaatgtaatctcatcaaattgctaagacaccttgtggcaacagtgagtttgaaataaactgagtaagaatcatttatcagtttattttgatagctcggaaataccagtgtcagtagtgtataaatggttttgagaatatattaaaatcagatatataaaaaaaattactcttctatttcccaatgttatctttaacaaatctgaagatagtcatgtacttttggtagtagttccaaagaaatgttatttgtttattcatcttgatttcattgtcttcgctttccttctaaatctgtcccttctagggagctattgggattaagtggtcattgattattatactttattcagtaatgtttctgaccctttccttcagtgctacttgagttaattaaggattaatgaacagttacatttccaagcattagctaataaactaaaggattttgcacttttcttcactgaccattagttagaaagagttcagagataagtatgtgtatctttcaatttcagcaaacctaattttttaaaaaaagttttacataggaaatatgttggaaatgatactttacaaagatattcataatttttttttgtaatcagctactttgtatatttacatgagccttaatttatatttctcatataaccatttatgagagcttagtatacctgtgtcattatattgcatctacgaactagtgaccttattccttctgttacctcaaacaggtggctttccatctgtgatctccaaagccttaggttgcacagagtgactgccgagctgctttatgaagggagaaaggctccatagttggagtgtttttttttttttttttaaacatttttcccatcctccatcctcttgagggagaatagcttaccttttatcttgttttaatttgagaaagaagttgccaccactctaggttgaaaaccactcctttaacataataactgtggatatggtttgaatttcaagatagttacatgcctttttatttttcctaatagagctgtaggtcaaatattattagaatcagatttctaaatcccacccaatgacctgcttattttaaatcaaattcaataattaattctcttctttttggaggatctggacattctttgatatttcttacaacgaatttcatgtgtagacccactaaacagaagctataaaagttgcatggtcaaataagtctgagaaagtctgcagatgatataattcacctgaagagtcacagtatgtagccaaatgttaaaggttttgagatgccatacagtaaatttaccaagcattttctaaatttatttgaccacagaatccctattttaagcaacaactgttacatcccatggattccaggtgactaaagaatacttatttcttaggatatgttttattgataataacaattaaaatttcagatatctttcataagcaaatcagtggtctttttacttcatgttttaatgctaaaatattttcttttatagatagtcagaacattatgcctttttctgactccagcagagagaaaatgctccaggttatgtgaagcagaatcatcatttaaatatgagtcagggctctttgtacaaggcctgctaaaggtatagtttctagttatcacaagtgaaaccacttttctaaaatcatttttgagactctttatagacaaatcttaaatattagcatttaatgtatctcatattgacatgcccagagactgacttcctttacacagttctgcacatagactatatgtcttatggatttatagttagtatcatcagtgaaacaccatagaataccctttgtgttccaggtgggtccctgttcctacatgtctagcctcaggacttttttttttttaacacatgcttaaatcaggttgcacatcaaaaataagatcatttctttttaactaaatagatttgaattttattgaaaaaaaattttaaacatctttaagaagcttataggatttaagcaattcctatgtatgtgtactaaaatatatatatttctatatataatatatattagaaaaaaattgtatttttcttttatttgagtctactgtcaaggagcaaaacagagaaatgtaaattagcaattatttataatacttaaagggaagaaagttgttcaccttgttgaatctattattgttatttcaattatagtcccaagacgtgaagaaatagctttcctaatggttatgtgattgtctcatagtgactactttcttgaggatgtagccacggcaaaatgaaataaaaaaatttaaaaattgttgcaaatacaagttatattaggcttttgtgcattttcaataatgtgctgctatgaactcagaatgatagtatttaaatatagaaactagttaaaggaaacgtagtttctatttgagttatacatatctgtaaattagaacttctcctgttaaaggcataataaagtgcttaatacttttgtttcctcagcaccctctcatttaattatataattttagttctgaaagggacctataccagatgcctagaggaaatttcaaaactatgatctaatgaaaaaatatttaatagttctccatgcaaatacaaatcatatagttttccagaaaatacctttgacattatacaaagatgattatcacagcattataatagtaaaaaaatggaaatagcctctttcttctgttctgttcatagcacagtgcctcatacgcagtaggttattattacatggtaactggctaccccaactgattaggaaagaagtaaatttgttttataaaaatacatactcattgaggtgcatagaataattaagaaattaaaagacacttgtaattttgaatccagtgaatacccactgttaatatttggtatatctctttctagtctttttttcccttttgcatgtattttctttaagactcccacccccactggatcatctctgcatgttctaatctgcttttttcacagcagattctaagcctctttgaatatcaacacaaacttcaacaacttcatctatagatgccaaataataaattcatttttatttacttaaccacttcctttggatgcttaggtcattctgatgttttgctattgaaaccaatgctatactgaacacttctgtcactaaaactttgcacacactcatgaatagcttcttaggataaatttttagagatggatttgctaaatcagagaccattttttaaaattaaaaaacaattattcatatcgtttggcatgtaagacagtaaattttccttttattttgacaggattcaactggaagctttgtgctgcctttccggcaagtcatgtatgctccatatcccaccacacacatagatgtggatgtcaatactgtgaagcagatgccaccctgtcatgaacatatttataatcagcgtagatacatgagatccgagctgacagccttctggagagccacttcagaagaagacatggctcaggatacgatcatctacactgacgaaagctttactcctgatttgtacgtaatgctctgcctgctggtactgtagtcaagcaatatgaaattgtgtcttttacgaataaaaacaaaacagaagttgcatttaaaaagaaagaaatattaccagcagaattatgcttgaagaaacatttaatcaagcatttttttcttaaatgttcttctttttccatacaattgtgtttaccctaaaataggtaagattaacccttaaagtaaatatttaactatttgtttaataaatatatattgagctcctaggcactgttctaggtaccgggcttaatagtggccaaccagacagccccagccccagcccctacattgtgtatagtctattatgtaacagttattgaatggacttattaacaaaaccaaagaagtaattctaagtcttttttttcttgacatatgaatataaaatacagcaaaactgttaaaatatattaatggaacatttttttactttgcattttatattgttattcacttcttatttttttttaaaaaaaaaagcctgaacagtaaattcaaaaggaaaagtaatgataattaattgttgagcatggacccaacttgaaaaaaaaaatgatgatgataaatctataatcctaaaaccctaagtaaacacttaaaagatgttctgaaatcaggaaaagaattatagtatacttttgtgtttctcttttatcagttgaaaaaaggcacagtagctcatgcctgtaagaacagagctttgggagtgcaaggcaggcggatcacttgaggccaggagttccagaccagcctgggcaacatagtgaaaccccatctctacaaaaaataaaaaagaattattggaatgtgtttctgtgtgcctgtaatcctagctattccgaaagctgaggcaggaggatcttttgagcccaggagtttgaggttacagggagttatgatgtgccagtgtactccagcctggggaacaccgagactctgtcttatttaaaaaaaaaaaaaaaaaaatgcttgcaataatgcctggcacatagaaggtaacagtaagtgttaactgtaataacccaggtctaagtgtgtaaggcaatagaaaaattggggcaaataagcctgacctatgtatctacagaatcagtttgagcttaggtaacagacctgtggagcaccagtaattacacagtaagtgttaaccaaaagcatagaataggaatatcttgttcaagggacccccagccttatacatctcaaggtgcagaaagatgacttaatataggacccattttttcctagttctccagagtttttattggttcttgagaaagtagtaggggaatgttttagaaaatgaattggtccaactgaaattacatgtcagtaagtttttatatattggtaaattttagtagacatgtagaagttttctaattaatctgtgccttgaaacattttcttttttcctaaagtgcttagtattttttccgttttttgattggttacttgggagcttttttgaggaaatttagtgaactgcagaatgggtttgcaaccatttggtatttttgttttgttttttagaggatgtatgtgtattttaacatttcttaatcatttttagccagctatgtttgttttgctgatttgacaaactacagttagacagctattctcattttgctgatcatgacaaaataatatcctgaatttttaaattttgcatccagctctaaattttctaaacataaaattgtccaaaaaatagtattttcagccactagattgtgtgttaagtctattgtcacagagtcattttacttttaagtatatgtttttacatgttaattatgtttgttatttttaattttaactttttaaaataattccagtcactgccaatacatgaaaaattggtcactggaattttttttttgacttttattttaggttcatgtgtacatgtgcaggtgtgttatacaggtaaattgcgtgtcatgagggtttggtgtacaggtgatttcattacccaggtaataagcatagtacccaataggtagttttttgatcctcacccttctcccaccctcaagtaggccctggtgttgctgtttccttctttgtgtccatgtatactcagtgtttagctcccacttagaagtgagaacatgcggtagttggttttctgttcctggattagttcacttaggataatgacctctagctccatctggtttttatggctgcatagtattccatggtgtatatgtatcacattttctttatccagtctaccattgataggcatttaggttgattccctgtctttgttatcatgaatagtgctgtgatgaacatacacatgcatgtgtctttatggtagaaaaatttgtattcctttaggtacatatagaataatggggttgctagggtgaatggtagttctattttcagttatttgagaaatcttcaaactgcttttcataatagctaaactaatttacagtcccgccagcagtgtataagtgttcccttttctccacaaccttgccaacatctgtgattttttgactttttaataatagccattcctagagaattgatttgcaattctctattagtgatattaagcattttttcatatgctttttagctgtctgtatatattcttctgaaaaattttcatgtcctttgcccagtttgtagtggggtgggttgttttttgcttgttaattagttttaagttccttccagattctgcatatccctttgttggatacatggtttgcagatatttttctcccattgtgtaggttgtcttttactctgttgatagtttcttttgccatgcaggagctcgttaggtcccatttgtgtttgtttttgttgcagttgcttttggcgtcttcatcataaaatctgtgccagggcctatgtccagaatggtatttcctaggttgtcttccagggtttttacaattttagattttacgtttatgtctttaatccatcttgagttgatttttgtatatggcacaaggaaggggtccagtttcactccaattcctatggctagcaattatcccagcaccatttattgaatacggagtcctttccccattgcttgttttttgtcaactttgttgaagatcagatggttgtaagtgtgtggctttatttcttggctctctattctccattggtctatgtgtctgtttttataacagtaccctgctgttcaggttcctatagccttttagtataaaatcggctaatgtgatgcctccagctttgttctttttgcttaggattgctttggctatttgggctcctttttgggtccatattaattttaaaacagttttttctggttttgtgaaggatatcattggtagtttataggaatagcattgaatctgtagattgctttgggcagtatggccattttaacaatattaattcttcctatctatgaatatggaatgtttttccatgtgtttgtgtcatctctttatacctgatgtataaagaaaagctggtattattcctactcaatctgttccaaaaaattgaggaggaggaactcttccctaatgaggccagcatcattctgataccaaaacctggcagagacacaacagaaaaaagaaaacttcaggccaatatccttgatgaatatagatgcaaaaatcctcaacaaaatactagcaaaccaaatccagcagcacatcaaaaagctgatctactttgatcaagtaggctttatccctgggatgcaaggttggttcaacatacacaaatcaataagtgtgattcatcacataaacagagctaaaaacaaaaaccacaagattatctcaataggtagagaaaaggttgtcaataaaatttaacatcctccatgttaaaaaccttcagtaggtcaggtgtagtgactcacacctgtaatcccagcactttgggaggccaaggcgggcatatctcttaagcccaggagttcaagacgagcctaggcagcatggtgaaaccccatctctacaaaaaaaaaaaaaaaaaaaaattagcttggtatggtgacatgcacctatagtcccagctattcaggaggttgaggtgggaggattgtttgagcccgggaggcagaggttggcagcgagctgagatcatgccaccgcactccagcctgggcaacggagtgagaccctgtctcaaaaaagaaaaatcacaaacaatcctaaacaaactaggcattgaaggaacatgcctcaaaaaaataagaaccatctatgacagacccatagccaatatcttaccaaatgggcaaaagctggaagtattctccttgagaaccgtaacaagacaaggatgtccactctcaccactccttttcagcatagttctggaagtcctagccagagcaatcaggaaagagaaagaaagaaagacattcagataggaagagaagaagtcaaactatttctgtttgcaggcagtataattctgtacctagaaaatctcatagtctctgcccagaaactcctaaatctgttaaaaatttcagcaaagttttggcattctctatactccaacaccttccaaagtgagagcaaaatcaagaacacagtcccattcacaatagccgcaaaacgaataaaatacctaggaatccagctaaccagggaggtgaaagatctctatgagaattacaaaacactgctgaaagaaatcagagatgacacaaacaaatggaaatgttcttttttaacaccttgctttatctaattcacttatgatgaagatactcattcagtggaacaggtataataagtccactcgattaaatataagccttattctctttccagagcccaagaaggggcactatcagtgcccagtcaataatgacgaaatgctaatatttttcccctttacggtttctttcttctgtagtgtggtacactcgtttcttaagataaggaaacttgaactaccttcctgtttgcttctacacatacccattctctttttttgccactctggtcaggtataggatgatccctaccactttcagttaaaaactcctcctcttactaaatgttctcttaccctctggcctgagtagaacctagggaaaatggaagagaaaaagatgaaagggaggtggggcctgggaagggaataagtagtcctgtttgtttgtgtgtttgctttagcacctgctatatcctaggtgctgtgttaggcacacattattttaagtggccattatattactactactcactctggtcgttgccaaggtaggtagtactttcttggatagttggttcatgttacttacagatggtgggcttgttgaggcaaacccagtggataatcatcggagtgtgttctctaatctcactcaaatttttcttcacattttttggtttgttttggtttttgatggtagtggcttatttttgttgctggtttgttttttgtttttttttgagatggcaagaattggtagttttatttattaattgcctaagggtctctactttttttaaaagatgagagtagtaaaatagattgatagatacatacatacccttactggggactgcttatattctttagagaaaaaattacatattagcctgacaaacaccagtaaaatgtaaatatatccttgagtaaataaatgaatgtatattttgtgtctccaaatatatatatctatattcttacaaatgtgtttatatgtaatatcaatttataagaacttaaaatgttggctcaagtgagggattgtggaaggtagcattatatggccatttcaacatttgaacttttttcttttcttcattttcttcttttcttcaggaatatttttcaagatgtcttacacagagacactctagtgaaagccttcctggatcaggtaaatgttgaacttgagattgtcagagtgaatgatatgacatgttttcttttttaatatatcctacaatgcctgttctatatatttatattcccctggatcatgccccagagttctgctcagcaattgcagttaagttagttacactacagttctcagaagagtctgtgagggcatgtcaagtgcatcattacattggttgcctcttgtcctagatttatgcttcgggaattcagacctttgtttacaatataataaatattattgctatcttttaaagatataataataagatataaagttgaccacaactactgttttttgaaacatagaattcctggtttacatgtatcaaagtgaaatctgacttagcttttacagatataatatatacatatatatatcctgcaatgcttgtactatatatgtagtacaagtatatatatatgtttgtgtgtgtatatatatatagtacgagcatatatacatattaccagcattgtaggatatatatatgtttatatattaaaaaaaagttataaacttaaaaccctattatgttatgtagagtatatgttatatatgatatgtaaaatatataacatatactctatgatagagtgtaatatattttttatatatattttaacatttataaaatgatagaattaagaattgagtcctaatctgttttattaggtgctttttgtagtgtctggtctttctaaagtgtctaaatgatttttccttttgacttattaatggggaagagcctgtatattaacaattaagagtgcagcattccatacgtcaaacaacaaacattttaattcaagcattaacctataacaagtaagttttttttttttttttgagaaagggaggttgtttatttgcctgaaatgactcaaaaatatttttgaaacatagtgtacttatttaaataacatctttattgtttcattcttttaaaaaatatctacttaattacacagttgaaggaaatcgtagattatatggaacttatttcttaatatattacagtttgttataataacattctggggatcaggccaggaaactgtgtcatagataaagctttgaaataatgagatccttatgtttactagaaattttggattgagatctatgaggtctgtgacatattgcgaagttcaaggaaaattcgtaggcctggaatttcatgcttctcaagctgacataaaatccctcccactctccacctcatcatatgcacacattctactcctacccacccactccaccccctgcaaaagtacaggtatatgaatgtctcaaaaccataggctcatcttctaggagcttcaatgttatttgaagatttgggcagaaaaaattaagtaatacgaaataacttatgtatgagttttaaaagtgaagtaaacatggatgtattctgaagtagaatgcaaaatttgaatgcatttttaaagataaattagaaaacttctaaaaactgtcagattgtctgggcctggtggcttatgcctgtaatcccagcactttgggagtccgaggtgggtggatcacaaggtcaggagatcgagaccatcctgccaacatggtgaaaccccgtctctactaagtatacaaaaattagctgggcgtggcagcgtgtgcctgtaatcccagctacctgggaggctgaggcaggagaatcgcttgaacccaggaggtgtaggttgcagtgagtcaagatcgcgccactgcactttagcctggtgacagagctagactccgtctcaaaaaaaaaaaaaaatatcagattgttcctacacctagtgcttctataccacactcctgttagggggcatcagtggaaatggttaaggagatgtttagtgtgtattgtctgccaagcactgtcaacactgtcatagaaacttctgtacgagtagaatgtgagcaaattatgtgttgaaatggttcctctccctgcaggtctttcagctgaaacctggcttatctctcagaagtactttccttgcacagtttctacttgtccttcacagaaaagccttgacactaataaaatatatagaagacgatacgtgagtaaaactcctacacggaagaaaaacctttgtacattgtttttttgttttgtttcctttgtacattttctatatcataatttttgcgcttcttttttttttttttttttttttttttccattatttttaggcagaagggaaaaaagccctttaaatctcttcggaacctgaagatagaccttgatttaacagcagagggcgatcttaacataataatggctctggctgagaaaattaaaccaggcctacactcttttatctttggaagacctttctacactagtgtgcaagaacgagatgttctaatgactttttaaatgtgtaacttaataagcctattccatcacaatcatgatcgctggtaaagtagctcagtggtgtggggaaacgttcccctggatcatactccagaattctgctctcagcaattgcagttaagtaagttacactacagttctcacaagagcctgtgaggggatgtcaggtgcatcattacattgggtgtctcttttcctagatttatgcttttgggatacagacctatgtttacaatataataaatattattgctatcttttaaagatataataataggatgtaaacttgaccacaactactgtttttttgaaatacatgattcatggtttacatgtgtcaaggtgaaatctgagttggcttttacagatagttgactttctatcttttggcattctttggtgtgtagaattactgtaatacttctgcaatcaactgaaaactagagcctttaaatgatttcaattccacagaaagaaagtgagcttgaacataggatgagctttagaaagaaaattgatcaagcagatgtttaattggaattgattattagatcctactttgtggatttagtccctgggattcagtctgtagaaatgtctaatagttctctatagtccttgttcctggtgaaccacagttagggtgttttgtttattttattgttcttgctattgttgatattctatgtagttgagctctgtaaaaggaaattgtattttatgttttagtaattgttgccaactttttaaattaattttcattatttttgagccaaattgaaatgtgcacctcctgtgccttttttctccttagaaaatctaattacttggaacaagttcagatttcactggtcagtcattttcatcttgttttcttcttgctaagtcttaccatgtacctgctttggcaatcattgcaactctgagattataaaatgccttagagaatatactaactaataagatctttttttcagaaacagaaaatagttccttgagtacttccttcttgcatttctgcctatgtttttgaagttgttgctgtttgcctgcaataggctataaggaatagcaggagaaattttactgaagtgctgttttcctaggtgctactttggcagagctaagttatcttttgttttcttaatgcgtttggaccattttgctggctataaaataactgattaatataattctaacacaatgttgacattgtagttacacaaacacaaataaatattttatttaaaattctggaagtaatataaaagggaaaatatatttataagaaagggataaaggtaatagagcccttctgccccccacccaccaaatttacacaacaaaatgacatgttcgaatgtgaaaggtcataatagctttcccatcatgaatcagaaagatgtggacagcttgatgttttagacaaccactgaactagatgactgttgtactgtagctcagtcatttaaaaaatatataaatactaccttgtagtgtcccatactgtgttttttacatggtagattcttatttaagtgctaactggttattttctttggctggtttattgtactgttatacagaatgtaagttgtacagtgaaataagttattaaagcatgtgtaaacattgttatatatcttttctcctaaatggagaattttgaataaaatatatttgaaattttgcctctttcagttgttcattcagaaaaaaatactatgatatttgaagactgatcagcttctgttcagctgacagtcatgctggatctaaactttttttaaaattaattttgtcttttcaaagaaaaaatatttaaagaagctttataatataatcttatgttaaaaaaactttctgcttaactctctggatttcattttgatttttcaaattatatattaatatttcaaatgtaaaatactatttagataaattgtttttaaacattcttattattataatattaatataacctaaactgaagttattcatcccaggtatctaatacatgtatccaaagtaaaaatccaaggaatctgaacactttcatctgcaaagctaggaataggtttgacattttcactccaagaaaaagtttttttttgaaaatagaatagttgggatgagaggtttctttaaaagaagactaactgatcacattactatgattctcaaagaagaaaccaaaacttcatataatactataaagtaaatataaaatagttccttctatagtatatttctataatgctacagtttaaacagatcactcttatataatactattttgattttgatgtagaattgcacaaattgatatttctcctatgatctgcagggtatagcttaaagtaacaaaaacagtcaaccacctccatttaacacacagtaacactatgggactagttttattacttccattttacaaatgaggaaactaaagcttaaagatgtgtaatacaccgcccaaggtcacacagctggtaaaggtggatttcatcccagacagttacagtcattgccatgggcacagctcctaacttagtaactccatgtaactggtactcagtgtagctgaattgaaaggagagtaaggaagcaggttttacaggtctacttgcactattcagagcccgagtgtgaatccctgctgtgctgcttggagaagttacttaacctatgcaaggttcattttgtaaatattggaaatggagtgataatacgtacttcaccagaggatttaatgagaccttatacgatccttagttcagtacctgactagtgcttcataaatgctttttcatccaatctgacaatctccagcttgtaattggggcatttagaacatttaatatgattattggcatggtaggttaaagctgtcatcttgctgttttctatttgttctttttgttttctccttacttttggatttttttattctactatgtcttttctattgtcttattaactatactctttgatttattttagtggttgttttagggttatacctctttctaatttaccagtttataaccagtttatatactacttgacatatagcttaagaaacttactgttgttgtctttttgctgttatggtcttaacgtttttatttctacaaacattataaactccacactttattgttttttaattttacttatacagtcaattatcttttaaagatatttaaatataaacattcaaaacaccccaattaaaagtcagagattgttaataccacatgatctcacttacacacagaattgaaaaacttggaactcatagaagcagagagtaaaaacatggttaccaggtgctggggagaggcggtgggctggggagatgttggtcaaagttagacaggaggaataagttcaagagatctattgtacaacttattcagttagataggaggaataagctaaagatcaagagatctattgtacaatgtgactataaccaacaacatatattgtacacttgaaaattgctaacagtatcttttaagtgttctctctacaaataaatatgtgaggtaatgtatatattaattaactgtagtcatttcacaatgtatacttatttcaaaacatcatattgtatgctataaatatatacaacttttatttttcaattttagaaatgtccttaaaaaatcagattttcagatcagataaaaaagcaagacccaactatatgctgccaacaggaaacacaccttaaaaataaaggacgaacaaacagattaaaagtaaaaggatggagaaaagatacatcatattggtaattagaagaaaactggagtgacaatatgaaacaaaatagatttcagagcaaagaatattaccaggggtaaaaatgatcattttataatgataaaagagtcagttcagcaaaaggatataacagtcctaaatgttttttcacctcatagctgtgtcaaaatagatgaagcaaaaactgatagaactgtaagaagtagacaagtccacaattatgtttggagattttttttttttttttttttgtcgcccaggctggagtgcagtggcaggatctcagctcactgcaagctccgcctcccaggttcacgccattctcctgcttcagcctccccagtagctgggactacaggcggccaccaccacgcctggctaatttttttgtatttttagtagagacggggtttcaccgtgttagccaggatggtctcgatctcctgacctcgtgatctgcctgcctcggcctcccaaagtgctgggattacaggcatgagccactgcacgcagcctggagattttaatatcctttcaatgtttagtagaacaagaatacacaaaatcagtaaggatatagaagattagaacaagactatcaaacaatttgacttaaatgacatttgtagagcacagcagtccccaacaacaataaatcacacattctttccaagagtacatgaaacatgtaccaagatagaccgtattttgagccatgaaacaaatcttgataaatttaaaaggattcaagtcatagaaaatatgttctctgaccacaatggaattaaattattaaccaataacaaatatctgggaaaacctcaaaaacttggacaccagcgcttttaaaagactaaataatttctaaattatctgtgttggggggaaaagagaaatggattagagagcaaaaagggtatcagagtgctgtggtacgatttttatgaagagtggaacagaatctgcctttggcgtttccccactacagcccattcttcacattgataacagcatgatccttctaaaattaaatctaacgatcacttctgcttaatggctctccaacacttacagaattaggtccaaaattctagcacagtttctgttcatctttctaacctttcttcccacaggtctagctagtacgtatttcttttattgcatttattacactattcctttgcttatctatctccccacctaggctaaagaacaagattcttgtctttttcatttttgtgtctcagtgcctagcatggtgccaggcacacagcatgcttccagtaaatgttagctggatggatgtaatgagtatattaaatattaatttatttgtttttccccaaaaagaattatttcctgcaaatcaaggaaattgctttctttatataatcaaaaacttattttcccagaagattcttcattaaaaattaagcctatgcacaacctagctctaaagtttcaaagattttaggcagcaatttttcaatctttttgaagtaatacatttgaatcttttcaaatttctgtttctgcatttgtgccacaccatctcatctcttgctgaaatgtttttgttaaattaattgcttgataaattgctaagtacttttcatcagaccaattaggacaatagtaagtatccatctgtggagcgcggacattcaagaaatctgatccagtatttagaaagtcattcctgagctgagttggctcaaactggcaccttctggcatttgcttgtgggtggggaatgtggaatgctttgaaagctgaatgagtttgtcaagttttaaaattcccttatggctaaaggaaaacaacattcattgtttaaaaacaccattgtttgttttttctgcttttttgttctttggagcctgaatctgcaaaaacactcacacccagcattttgcttcatgtaccactcctaagatgtttttagagacttgaatagtgtctccgcactactttttattgtgattgttcagaatgttcataacaaatggtaaaaagtcagttttagtgctcaaattgagttttatggagaaagaccataatttatgtttgtcattgtaaattgataggagaatttttggaagtttgcgtcctagaaccagatttccaaggctcagatccttattttctcacttcctagctgtgtgaccttagacaaggtattaaacctgtctgtgctgcctcagtgtcctcatctattctttaagagtaagaatagaacctacccgatagagtcacttgaagattaagtgggttagtaaattcagaatgcttggaacagtaactagcacagaataagtgtccaataaaattgggttgcagctattatcagtattattcctgtcataatcatcatcaccattaagcaattaaatgtagagttccaaaatttgattatgaaactacagttatacagccatgattcccggtgataccacgtcagtaacaagattatttccttagcttgagccagtcactacctcattgcatgtggcagagtgtgttgccgtaggcaaatgtcattgtagggaatgaaaaaaaaattgcctgtgagctgctctccagaggcctcatcccattttcccatcgtccactttactccatctccactgccactattaggaccttatcatttcttgtctagattaattcaacagcttccttccttctagtctccatgatttcacccactagccatcccctcccctttgcccaattttctccatttatggtagagtgatctttctaataggaaactcctgacttgccttaaaaagccctcattgaggccggacgtggtggctcatgcctgtaatcccagcactttgggaggccgaggcaggtggatcacgaggtcaagagattgagaccatcgtgactaacacagtgaaaccccatctgtactaaaaatacaagaaattagccaggcgtggtggcgggtgcctgtagtcgcagctacttgggaggctgaggcaggagaatggcgtgaacccgggaggcagagcttgcagtgagccgagattgcgccactgcactccagcctgggcgacagagtgagactccgtctcaaaaaaaaaaagccctcattgacaaccttcaacccacaatccatggtgaagcacaggagccttggggatctgcccccagcacacctctccacccttgtctctcactgctcctgccttcatggagagccctgatgaactatttgtagtttcccctgactcaccttgctgttactgggcctgtgtgcgtgttgctcccactacctgcaatacgcttacccacttcacctgggtgaactttacttaggattcaccttaggtgggcatcatgttcttccaggcccctcctctaacttttagttgagagtattccagacttaaggctccatgggatagggatcttgtctatgcaccagcttattcccaactgcctggcacgtaatgcatttattaaatatatattgaattgattaccctacttggggctcttgtttgcttctacacttacagttctagcatagcacttaactcattatcatgcatcattattatgggtttgttttgtctcccattagactgtgagctccacaaggctgtgtccttgtcttatacatcattgtatttccagcttccaacatagtgcttgccatgacacaggaagtcagtaagctctgaatgaatgaatagtatctacataccattaatctgaggtttaaagtttccccaaattctgaagcaaggggatttacggacttccctgacaatttttggatgtcatcccaatgataccactaacattttaagggacagcttgcatatatacatttttctggatggcagttttttttcccacaggcttcatcagatatttctccatagccttcctcagattctcaaaggggtctctgattcccccaaaagataagaaactgtcataaaaaattatttctaaatatcaattgttaaataaaatgtttgcaaagcagcctgatgaatcatttcaggccacttgaccccgatgagttagagagtttgtgctctgcaatctgactgcttccagcagtctcactgctgctggactgtggcacttccaattggcagcagggcaagtttcttctggatgaatattctgtcataggggtcccccttccacacatacctgtaggagcagtttgaaactcatatgcatggtcttcctggttctaggcacatgagtcatttaagctgctggagccaggaccagctagtatgctagcccggcattcagaaagttaaaatttggggtcaaaactgagaaccttctttgatccaccttggccagacattttctctggcttccattaatagcctcaacattttttttttttctggcctagacccacacaggcaagagaccagagcttctctaaggagctaagggaaagcacattttaaaaataacttgagcaaatgaattcatctggcaaaagcaaccccactacgtaaaataaacctttttagtttcgcaatagcagttcctgaaaatgtaaacaacctcagggtctacatgcactgaatcatttgctgaacagaaagtccctggtccaaattctgcaagaataaacaccttacaaaactaggggtcaatgaccttcatatgggaacaaggagggtgtggggggcagcaacccaccctgaggacaatgagaaagtcttgagacttgatattcaaaatgctggctttctaaaccaaaaactggcatgagtggagggagaaggggagggtgggcacagtctatgcctcaggctcttgctcagaccctaccaggcccctgccttccctagggaaagcgagagtctactcactgtcatgaagccagaggaaggccctgcaggtttcactgtgtgttctgttgacaagatgatggttccattgaaactgtaataacatacttggccaactaagcccatacgatcgtagtaactttgtacccagtcctagcttttcaaacataatgataatatgttctttctaatgtggcccatactgttctaatgaacttatgctgagtttttctgagtactagaataatattcgccataaataatagatataattattctcatttaatatttgcgtagctcttctttaaagcagaaagtattttctcattccttactagaacctttctgtgtgaggagcactgagctagaacccatatcttagaatggtcagaatttggagaaattcagggaaaaggcactggactcatttttaaagactagaaaatgcaacctccagaaaaagattcaagagttttttactcccagagatgtaggaaagattggagtaaatcttaatattatatttcaggtaaacaaaggatcactgtcaaaatagcagcatttattgagtaatggctgtgtgccaggtactttacagtttcacatttaaccctcataataaccttgtaaagtggatatcccctcagtacatgatgagaacactgaagcttaggttaaatgattgtccaaatcggacaatcattttcaaaatctccccctttttttctcctttcttatctgcaaggcagattgccctttccctttcagtgaaacttgtgcatgaccacatgactctctttggccaatgaaacatgaacaagcagcgtttatcactttcagatggaaggctttgcatgagctttgcctccttttcactctgccacagtggccactaacattccagatagtggcgctctgcaggctaggtcctatagtgggagctatgggcagagccccctttcccacccccatcaagatgtgcatgctgcataagccatgcattaatctttgcagttttaagccactaagttttggagttatattaatcattaatcatggttctcaagagaaacagagtgggggagtggtattcattatgggaattggcttacatgattatggaagctgagtagtcccccagtctgctgtttttgagctggagaactagaggagccagtggtataattcagcccaagcctgaaggcctgagaaatgggatgggggaattgggagggtgggtgtgctagggtaggataagtcctgaagttcaaaggccagccagaaggtggatgtttcagcaccagaagagagagcaaattcgcttttcttctgcctttttgtcctctctgggccctcaatggattggatgatgccctcccacattggtaagggtggatcttctatactcagtctgctaatttcttccagaaacatcttcacagacacatccagaaataatgttttaccagctatctcggtatcccttagcctagtccatatttaaaaattaatgatcacaagcagttgtttgtttccacagcaaaacctgggtgacagaccaagtgacccagatgactagaatttgaccttcttttgttgcccacaccatactctgaactaacatgctgtgctgccttccaagtggagaatgatggctaagtatcttctacctaatttgagtcacagaaaaaaaaaaaaaaggttattaactgcagtgacaagaattgtgattccccagggggcagatcaagactgatagataagagaagtgaggaacatctggggaatgtccattgaaaatttactcagaagagaagaataattaatataataatatgatatattgaattataataaataatattttgatgtatttccttccaggcatgtttaagttatagactttgagtatattttctcaaagggggttctatgtaagagactatttcttaatatagttcctagcttggaattgctcttgctggtttaagctgagcttattttattacagacttcacaacaataacgttttccttcactagtcagtacacaagatggtcttcatttccagtttggaatcccacactatcagagcctgagacaaggactagtatgcagttagtttgtttgggaggtgattccaggaagtgggaatgagagatcagtcagcctgcaacacgaaggaggaaaagtcaatataaggatgaatttggcaattggccgtttcatgcaactggggctaaattttgcttggctctctaagaaatgtaaagaatgcctcccgtaattgctcacctcaagtatttattcattggctctcatgctccattggttgtccatgagaactttagccctccctcgctgcagcacagacactgtgctttctcctaggctgagcaagctcctgcatctgtggaaaccgtcccggggcagatagtgaaataatgactgctgcgtgcttgagatctgggaaagaggccacatcataagtgcactgaaatcagagatgtgtcaagagatgtgacacagggcatctgaggtgtctactgcaccagctataactccctaaacgctaatctcagttcttacagaggggatggatgcaagggaacagtcatgattgagagcaccgaagaagctctgtatgaaccttaggcaagtttcctaatctccaaaatgaaggtaataatacccaccatccaagatcttcgggaggaatagatgaactaatgtatgtgaaaatgtccagcacaggtcctaacccatagtaggtgctcaccaaatgttagttccctgccctccacgttgtgtgtatccggagctgcactagatgctgaggcaaatggtctcaaatgtactttaacacttaatgactgagattttttctgagctgcctacaggttattgactatattcattattaataataatatatatggccacttcaggcaactggggctaaattttgcttggctctctaagaaatgtaaagaatgcctcctgtaattgctcacctcaagtatttattcattggctctcgtgctttattggttgtccctgaggactttagccctctctcactgcagcacagacactgtgctttctcctagtttctgtggcaagtgacaggagcccacctcaaactaaagcaaaagggacttcattggctcttgtagctaggaattccagggttggcactggctttgggcactactggatgcaggaattcaaacaatgtcttcaactctttcttttggtgtttctctcagctgtgcttctcttgtcgtttctttttcccattttacagataagttcatccgtaactgagagaggtgaaaaggggatggctgcagagaactctggcttatatcatccttgcttgctgacctcaaggtccatgtataaattctcagagaagaagccctctggttggtgatgcttggaacatgccctggagggtgggccccttgaagtggagcttgctggaaccacatgggctggagcaaggcgctagggccagaagagagaggtaggcagggctgctggccaggcactcttcaccaagacaaggcaagaggaggggcatgattgaggcagtgatacagaaagcagacagtagaggtcgtggcaagtgtgccgttacttgctacctgtggttgatgggagagtcacaccacatttaggaggagagaatccatttgccacttctgacaatgccacaagaatcacatatttcatccagaggttgaatttggcccatgctgagctttaaaatacagagctgtcttggaacaatggctcagtacattcatttggtgtccaacaaagcctgcctctgttgccttccctctctctgtgtgcccttcaagatcttcattgtgctttggggagagaaagagaaaatgtcatatcagggtagctcaccccatgtgtcctggactcaggaaaagagtatcttatcaccttactcttttgttattataaaaaataaagttgaacgtcttcaaataaaataaagaagtatagaaaaaattttaaattaacctgttatgattctacctagagaaccattgtcaacatcttggtatatgtacttccagatactttcctatgaatatatacattgtagattttttaatattaaaaggctatcatgctgctttgtatacaggctttctttactgatatgtaatataatacacagacaaatatacaaatcctaagccatcaactcattgaatttttattcattgtttttaatacctgcattgtgttccattgttaggctatgtcacaacatatttaattaagcccctattgatgaatattaatttactctatttgccagttcattccagtccaacatttattgagtgtctacttacgggccaggcactcttgtattcatcaagatcaccacattatctgtatcagttatttattgccacaataaaactgcataacaaatcactccaaaatgtagcaccttaaaactacaactacttattatttctcaagagtcaatgggtcagctgagcagttctgccgataggggtcaaggtcaacacatttcaactagactacttgtaaaaaagaatgagtgtctgggtaggtgtgttcttctaaaaataaaacaaggaatgaggaaattgcaggtaggataagaggggtggttggcaaccaaaccccacaaaaggcagacaaattttaaggaaacataatgccagactcctatgtcatcatccaagtagatgcagtgaagtataacctggggcgtagtagggtaggagtggggagagcagaggagaaggaagggagattgcttttcatcacttttggattccctaataacagacatgactgccagtattaaaatttaacaaaggatatctgatcattaattttcctgtataagtcactggtgatcttcaacatctctccctcccttcctcccttccttcctcccaccctcccttccttccttctttcctcttttgctttcaacttccttttctcgtttccttttgctttctttctcttctcccttttttctgtcactctgggcgtatgtagtagtgtaaaaaggttgacagagaaatcaaatataacaggagcagggccctgagaaaagcacctggcatcctgtaggcaaaccattgtttctaaaagaagggactgagagattgaggagctcaggacattgccaaatgaacaaggcaagcacatttattcagtaccaaacaaacggaaaacggcctttccaaataactgacctataaaacagccttttcacaagagtaccgtaattactggccaacagcaacaatgaaaaacaactcccaaacaaagaaatatttctggattaaaagccatgagatctggattctaacaagctgtgctcctcaaactacaagtacaaaatctggctctaaactaacaagctatgagcctcaaactgatgactggcatgtttgggtctccatctccttcttgggggttggggtcttagagacccttttccacgccctgattctcttactagtgtgtatgctttccttttgacttctcatgctgaccgtctgagcaggagtgagaagcaatttcaaaggaaaacatcgtttatcatctgctgaaagaaaccaaaaagaacacaggaaaacaaaaagacaaggaaagggaatgaaaatgtaattcattttattaaaaagaagaattattcttctgggacactggatagaaaccttaatgagttacctagctatcataaatcctctaacagagaagagaagagaaagaaacaaagacggaagagggcaggataaaagaaagaaaaaaggaagggaaaaatgaaggaaggaagttatctattcatttctacagagactctgctgagcagtagacaagaagacttgggaaaaatttaactgaaacttttccaaaaatcttttcagagggattttttccctctgaaaagcatcattagaggctgttcaatacccaaggcaagcctctttcatattacttactgtacatgaaacactcatgcaattgaggctagccagaggccatttagaaattcaataattattcaacccaaggggctttccaaatggtgaagtagcttcttaagaggaaattaatattgagcagtatagcaaacctaattggaatcttgagaaaatagttctgtgtcgttagaacagctagaggctaaagaagatcaggttggatgataccttcatttttgtctctttccttaattatgatgtaaagggaaaaatcttgtttattttctatgccaggagggtagagggtgatttggagaggttccaagtttatcaaaatctaccttcagtctggcagtagaaaagtttacttccttcatttctttcctatagacattcaaagagagctaaggagatccaaaaacctttttttctatatttgcaatgcaaggcagttgggaattaatgactgatttgttggtgagggcagtgggcattgatcacaaaagcagtaaagctgtgtttctcaaagagagaaagtctctttgagatcttcattattttactatttagaagagaaaggggcgttatatcacgttggaagcatccatgagtcactagtctcttctctatctttctatgcctttctgtattaattactttgaaagcacaacattccaaacccattgagcacacagtggtctgatttctccacttgtgaaaggtgctaaagtctcactgtaggattaatttgggggtccaggctatgggcttgtagatatgactaccttagactttggttctcctggcaactaaccctttttggatcgtatctaagttgacctgtttcacagtgagagaactcctctccattactcagaatactgaggcagatcacaagtgtaccacacctggctaatgttaagccagacagaaacatcaggctcatctcttgagaagaagggtcgcttattaaggatacaaactattttttttttttttttttgagacagggtctcattgcccaggttagagtgcagtggtgcaatcatagctcactgcagcctcaaccacatgggtatttttaaataagaaaaaaataccatctgatagatatgaaggagcattgggtcactataaacaaaacagattctaagagcaggaagaaagagtacagtctcttttcaataatttttttttaaacttgggaaagaacactcactctattcctatagaccagaaagcagataattgtccattatgattccacatgacactatcttgttcagctgtcactgaaacaactttgaacactgtcatatgttcttcccagctcctgaactctgacctttttatgccttagttccactttcacaaaaagggattgatgtaatgtgcatttcagaggaaacgactatagacatttagtgtcattataaatgttgagaagtatgctggcagaaattatgccttaagatcatatatggattcttgtatggtttgaaattgcttaaaagatatatatgatctctaaaatgtgtgtgtatatatatatgatgtcttcttatatatctatatgtgatatatttatatatatataaatctgtgtatatcacatatataaatttgctgttatttgaattgccattacctcagtgcttaggggaagccatgcacgtttgtttcttttcagtacccagagttaattaacataagttatcacagaagctcccataagcattgagacaatttctctatacctgtgactatttaaggttttgaaaacaaaacagaagcaggtaaggaggaagtacgctttactattgaagatttattaggtacacatttagatttgtgaactcacattgcttaggatgaaagggactcttgaggatgtctgctgtttgttagtgaactgcctgtaacaattacaattagcacacacatgagcacaatgaactgggtagtcagactcagccaaaatgaatagaaatagcctcttaccaaatttactttgagtagcccttggactctgagcactgctgcccagagcaatatgactgtaggtccaagtttgtcaatgactatgcaaatgtgctttcttcgcttttactctattgtcatctgtctattacaatgttgctatggtgacacctttccaatatccctgtgcttctttggtatcctctaaggggaagctgtaatgaagtggcttggcaaaagaatcctcttggaattttttttttttcatatgctactgaaaaccagcatgattttcctcttatgggaaatgtataaagtatgagttggaaatgatggaaattaatctgtactgacttgggcaaggaatgtgaatgttattcattctgttccaaactacctgaaaatattctctttctgttcctactttccaggagataacatcttaagggacactgaagcttgtgcgtgtgtgagtagaacacgtgctgggggctcttgagctcatgagggaggggctacatgtcggtggggtgataactgtatgctggaaacaatgataggtggtgaccctggagcacttaccatgtgacaggtgttatgctaagcatgttgtatgcattccttcattgaatgacagctacctatattatcctcattttataagatgaggtaacagagcttcagaaaggttagactcagctgctatgggtctgtctgactctggtgttcttcctcttaaaaactggggcactttggaaatgagattcctcggtgatgaacagaaatattgcttagcggctgtatttttgtatctggcagttttcccatatttgagtcttatattcacaatcggtatctttacattacacaaaagtgacacagaattagagtcatttaatccagggttgatatcattaagtcatgactatttattaaatgtttcttacaatatctgagatgatattgcaaaagatgtaagtgattttagaagttctcacttcgtagttagttgcagaaacctcttttggaggagggatgttttctctatatatcctaatttctacttaatatatttccacacctctttgaagtgtgtagtaagaatggtaaaatgcagtacttcgtcatttggtacagttcaatcaatatgcattaagatgtgatcatatgggtaatagaaaaatgtgaaagatccaattctttttctccagaaggcaggaagctcatatttgatttctgttactataaactataaaaacgtttcaaatgtagtttacccgtaaccatcaccctgcaagggtgatattgctccccgccaatttacggaggagaatactgaggctttaaggttgtagatagaccaagaccacacaagtagagagtggcgggctgtgggttgagctttaaaatccaggttcatccatgactcccagtgtgttctagtaaatccactagaatctgagtattttccaatgatttatgctccgctctgtgtcaggcagttcatggtatttttcaacaatcagaaaatcctggggaaggcaaactgtttccccctctctaggtgccttggaagtggccgttgtggacccagagatcatcctttctgatctgacaccttcttcactgccctggcccagtgtcttttctgcaaggctggaagcccccttagactggtcatgtcccatctctttccggagggaagatgatcccaaagacgacttttctctccacggtgctgccataccgcaggcggccgccaggggtccccgctcggcgtccccgcgagacagtcgagccccggccggctgcgcggcgcgctgggtgcatgagggggctgctccggagcgacggcggctgcagctggagccaggcgctcgcccgtccgccggttggctcgccgggacctcgcgcaccggcggcagagtcccttgcgtggattggcaagcgacgccccacctgccccgagctcaccattttctttcgcgctggctgcagctgacccggcgaagggagccgaccgggccctgggctggaggtaaaaccccacggtgagtaagaacccgctccaagctaggggaggcggcgcagcccggtggctgctcgctcccgatctcgcccgggcgggcggcgaggtttggggcgcacctgggcgcgggtgcaagaaggtgcgggaggcggcggaccggtcttctgcccgccggccacgggcttccggggctggagtcctcttcagacccctgccggcgcctgggtttctggccggctcctcgtgtgcacttcccggcaggaacaagggtcgcccactttccaccccgggatcttgatttgtccttgatttgaaaagatataaatcaataagatcgtccttctttcggggtgcaagactccgagcccatccccagccgcggacgcctgcagggtgcgtgttgggctgtgggtggcgggaagacaaacttttacaaaagtgcgcctgggctgggggacaacgcttgggcgtcctgatcctgagggaggagtctcggcttggggcagcgtaggggaagtccgcaccgtcagccaggtcgcccccggggctgacgatgcctcacggaggtggggagcgtgtaaaggccgtacaaatcgcgcttaactttggggccaacaactgtcaaacatctggaatcccagcccctccctttccctgaactggggaagaaggtgaaaacccttcaagttttctttgattgccccttcccaccttcagacccctgctgggagggtaaagcgccgacccctggtgcctggcaagtaccagagactctaaatctctcgggatcccccccctcgcgctctttcctgaccctctcccctaaccctccccacagagatctctctacgcagccgactgagatcgtggcgaatggccttttgtttctccgcgtttcccctattgtttgcctttccaacatctggcggggcttggggagagaaggaagcccctctggtccccctccccggcccccacgccagctccggcaggggatcccagctgggaaagtggaggagcccgaccccagcgaggccgccccaccccgcccttgtggttagagggcggagggaaagttgttccttccccgcctccgctgctgcctgtggcccagggcgcatttctcagatctcagcccaggcgcgccgcaaaggctcaaatccgagaaggtgctgctttcgagacagtggaagcgcgttccgccccaatccagagcgtccagtggttggttccagaggatttcaatctctagccaaaggcgttggggctgggccgctgctagggcagtgggaggggatcggggcacctttggtaggcggaaagctgagattctggggtccacaagtttccaagggcgggagggcaggctagtcgccaaaaagagaacgaagatgcaaataacgaggaagccttatgacgttgcctggaaatagtagtgtggtggttcactccggaatgaacgtggagttctggctttgagtaccgctccaagtttaaatcccaagtcccctttcttcattgtagaaaaagaggactcagacgacgcaacacagatacggctagagcacagttcctgcttccacgtcccagagaacaagtggcttaggatggtcccgagttcccctgtgggtgcgcttgttgggttgcaggcggccctgtttccctgcacaagtcagatgcttacacattgtgttcattcttagtgtggattattgattaaagaactggggcaaaagcaaagtagctactctgagaagtcagggtccccagatggtgcccagcgagttgtcttgcctctgaggggaggctgactgagactgtgcacctgttagaacctatgctaccccatagccttgcagttgacttgctgttgccagcttttcctgtgggatccccaatgagtccctcttccaaggaagctcaattacacttttgattcctcctcaacccaggggaagaaagaggcttctgtaggaacattatgatctatgtacccactcagacattgtcagtggataccagaagcttggctctgcacagctctgagagttttccctttgcgaactcaacagaacttttgagtttccatttaacataaaagaagtgagactgctaagccaggaatgcgacacatagagcactttctctagtgatttctgggtattatatctctttaccttcccaacggtggaaccaggaaaagaaaaaaaagcaacatctttgaagtactgcaaggcactttacaaacatttcattatgaaaatgatccccaaggaaggattcctttgaaatttagcagcagcaacccagaagcaacaaaaaagaccaaagttactcaagaagtacccaaaggcatcattaacaaaataaaagagcatttcttgtcttggcctaccccgctaaggaaaacagggtaattatagtggaagttaagcttg

SEQ ID NO: 64 - DNA, 28 bp, synthetic construct
  misc_feature 1..28: note = Synthetic polynucleotide
  source 1..28: mol_type = other DNA
cccggggccggggccggggccggggccc

SEQ ID NO: 65 - DNA, 212 bp, synthetic construct
  misc_feature 1..212: note = Synthetic polynucleotide
  source 1..212: mol_type = other DNA
cccggggccggggcccggggcccggggcccggggcccggggcccggggcccggggcccggggcccggggcccggggcccggggcccggggcccggggcccggggcccggggcccggggcccggggcccggggcccggggcccggggcccggggcccggggcccggggcccggggcccggggcccggggcccggggcccggggcccggggccc

(19) United States
(12) Patent Application Publication
(10) Pub. No.: US 2024/0393348 A1
(43) Pub. Date: Nov. 28, 2024

(54) USE AND TREATMENT OF DI-AMINO ACID REPEAT-CONTAINING PROTEINS ASSOCIATED WITH ALS

(71) Applicant: University of Florida Research Foundation, Incorporated, Gainesville, FL (US)

(72) Inventors: Laura Ranum, Gainesville, FL (US); Tao Zu, Gainesville, FL (US)

(73) Assignee: University of Florida Research Foundation, Incorporated, Gainesville, FL (US)

(21) Appl. No.: 18/668,571

(22) Filed: May 20, 2024

Related U.S. Application Data
(62) Division of application No. 17/322,604, filed on May 17, 2021, now Pat. No. 12,025,622.
Application No. 17/322,604 is a division of application No. 16/851,690, filed on Apr. 17, 2020, now Pat. No. 11,035,867.
Application No. 16/851,690 is a division of application No. 16/362,908, filed on Mar. 25, 2019, now Pat. No. 10,663,475.
Application No. 16/362,908 is a division of application No. 14/775,278, filed on Sep. 11, 2015, now Pat. No. 10,295,547.
(60) Provisional application No. 61/883,219, filed on Sep. 27, 2013.
(60) Provisional application No. 61/786,258, filed on Mar. 14, 2013.

(51) Int. Cl. G01N 33/68; A61K 35/28; B01D 21/26; C07K 16/18; C12Q 1/6883

(52) U.S. Cl. CPC G01N 33/6896; A61K 35/28; B01D 21/262; C07K 16/18; C12Q 1/6883; C07K 2317/34; C12Q 2600/158; G01N 2800/2835; G01N 2800/285

(57) ABSTRACT

Disclosed herein are methods and compositions for identifying and/or treating subjects having or likely to have amyotrophic lateral sclerosis (ALS) or frontotemporal dementia (FTD). Antibodies specific for one or more di-amino acid repeat-containing proteins are also provided herein.

### CROSS-REFERENCE TO RELATED APPLICATIONS

This application claims the benefit of the filing date of U.S. Provisional Application No. 61/786,258, filed Mar. 14, 2013, and the benefit of the filing date of U.S. Provisional Application No. 61/883,219, filed Sep. 27, 2013. The entire contents of both of these referenced applications are incorporated by reference herein.

### FEDERALLY SPONSORED RESEARCH OR DEVELOPMENT

This invention was made with government support under POINS058901 and RO1NS040389 awarded by the National Institutes of Health. The Government has certain rights in the invention.

### BACKGROUND OF THE INVENTION

Expansion of a GGGGCC hexanucleotide sequence within the intron of the human C90RF72 gene is associated with both amyotrophic lateral sclerosis and frontotemporal dementia in humans. Amyotrophic lateral sclerosis (ALS) is a debilitating disease with varied etiology characterized by rapidly progressing weakness, muscle atrophy, muscle spasticity, difficulty speaking (dysarthria), difficulty swallowing (dysphagia), and difficulty breathing (dyspnea). Although the order and rate of symptoms varies from person to person, eventually most subjects are not able to walk, get out of bed on their own, or use their hands and arms. Most subjects with ALS will eventually die from respiratory failure, usually within three to five years from the onset of symptoms. Riluzole (Rilutek) is the only currently available treatment for ALS and only slows progression and increases survival to a modest extent. Frontotemporal dementia (FTD) is also a devestating group of disorders resulting from atrophy or shrinkage of the frontal and temporal lobes of the brain. This shrinkage or atrophy results in severe behavioral changes. There is currently no cure for FTD and limited medications for managing the symptoms of FTD. New methods for diagnosing and treating ALS and/or FTD would greatly benefit ALS and FTD subjects.

### SUMMARY OF THE INVENTION

Expansion of a GGGGCC hexanucleotide sequence within the intron of the human C90RF72 gene is associated with both amyotrophic lateral sclerosis and frontotemporal dementia in humans. As described herein, an expanded GGGGCC hexanucleotide repeat sequence within the intron of the C9ORF72 gene was found to be transcribed such that RNA transcripts containing the hexanucleotide repeat in both the sense and anti-sense direction were produced. These sense and anti-sense transcripts were found to be translated to produce di-amino acid repeat-containing proteins. The sense transcript (containing 5′-GGGGCC-3′ hexanucleotide repeats) was found to be translated through repeat-associated non-ATG (RAN) translation such that poly-(Gly-Ala), poly-(Gly-Pro), and poly-(Gly-Arg) proteins were produced. The anti-sense transcript (containing 5′-GGCCCC-3′ hexanucleotide repeats) was found to be translated through repeat-associated non-ATG (RAN) translation such that poly-(Pro-Ala), poly-(Pro-Arg), poly-(Gly-Pro) proteins were produced. Additionally, the anti-sense transcript was found to be translated through ATG-initiated translation to produce Met . . . poly-(Pro-Arg) and Met . . . poly-(Gly-Pro) proteins.

These di-amino acid repeat-containing proteins were found to be present in ALS subject blood samples. Accordingly, aspects of the disclosure relate to a method of detection of di-amino acid-repeat containing protein levels in sample (e.g., blood) obtained from a subject, the method comprising measuring di-amino acid-repeat-containing protein levels in the sample of the subject. In some aspects, detection of di-amino acid-repeat containing protein levels may identify (or diagnose) or aid in identification (or aid in diagnosis) of a subject having ALS or FTD or likely to develop ALS or FTD. Alternatively or additionally, detection of di-amino acid-repeat containing protein levels, e.g., in a blood sample of the subject, may identify (or diagnose) or aid in identification (or aid in diagnosis) of a subject as having a risk factor of ALS or FTD, such as an elevated level of a di-amino acid-repeat containing protein or proteins in the cerebrospinal fluid of the subject. Aspects of the disclosure also relate to treatment of a subject having ALS or FTD by decreasing or stabilizing di-amino acid-repeat-containing protein levels in the blood of the subject.

Additionally, expression of the anti-sense transcript (containing 5′-GGCCCC-3′ hexanucleotide repeats) was found to be highly elevated in subjects having the expanded GGGGCC hexanucleotide repeat compared to controls. Foci of sense and anti-sense transcripts were also detectable using fluorescent in situ hybridization (FISH) in brain and blood cells of patients having the expanded GGGGCC hexanucleotide repeat sequence within the intron of the C9ORF72 gene. Thus, other aspects of the disclosure relate to a method of detection of a hexanucleotide repeat-containing transcript, the method comprising measuring a level a hexanucleotide repeat-containing transcript and/or measuring the presence or absence of a hexanucleotide repeat-containing transcript focus. In some aspects, detection of a hexanucleotide repeat-containing transcript may identify (or diagnose) or aid in identification (or aid in diagnosis) of a subject as having ALS or FTD or likely to develop ALS or FTD. Alternatively or additionally, detection of a hexanucleotide repeat-containing transcript, e.g., in a blood sample of the subject, may identify (or diagnose) or aid in identification (or aid in diagnosis) of a subject as having a risk factor of ALS or FTD, such as an elevated level of a di-amino acid-repeat containing protein or proteins in the cerebrospinal fluid of the subject.

In some aspects, the disclosure relates to a method for identifying a subject as having ALS or FTD or likely to develop ALS or FTD, the method comprising determining, in a blood sample obtained from a subject, a level of one or more di-amino acid repeat-containing proteins selected from a poly-(Gly-Ala), poly-(Gly-Pro), poly-(Gly-Arg), poly-(Pro-Ala), poly-(Pro-Arg), Met . . . poly-(Pro-Arg) or Met . . . poly-(Gly-Pro) protein, wherein a level of the one or more di-amino acid repeat-containing proteins that is elevated compared to a control level indicates that the subject has ALS or FTD or is likely to develop ALS or FTD. In some embodiments, the level of the one or more di-amino acid repeat-containing proteins is determined by performing an assay. In some embodiments, the assay comprises an immuno-based assay. In some embodiments, the immuno-based assay comprises an isolated antibody specific for an antigen comprising a sequence as set for in Tables 1, 2, or 3. In some embodiments, the immuno-based assay comprises an isolated antibody specific for the C-terminus of the one or more di-amino acid repeat-containing protein.

In some embodiments, the method further comprises identifying the subject as having ALS or FTD or likely to develop ALS or FTD if the level of the di-amino acid repeat-containing protein is elevated compared to a control level. In some embodiments, the method further comprises treating the subject having ALS or FTD or likely to develop ALS or FTD. In some embodiments, treating comprises administering to the subject an effective amount of one or more of riluzole, baclofen, diazepam, phenytoin, trihexyphenidyl or amitriptyline. In some embodiments, treating comprises performing a procedure selected from plasmapheresis or a bone marrow transplant.

In some embodiments, the one or more di-amino acid repeat-containing proteins is selected from the poly-(Pro-Ala), poly-(Pro-Arg), Met . . . poly-(Pro-Arg) or Met . . . poly-(Gly-Pro) protein. In some embodiments, the one or more di-amino acid repeat-containing proteins is two or more di-amino acid repeat-containing proteins.

Other aspects of the disclosure relate to a method for treating a subject with ALS or FTD, the method comprising decreasing or preventing an increase in a level of one or more di-amino acid repeat-containing proteins selected from a poly-(Gly-Ala), poly-(Gly-Pro), poly-(Gly-Arg), poly-(Pro-Ala), poly-(Pro-Arg), Met . . . poly-(Pro-Arg) or Met . . . poly-(Gly-Pro) protein in the blood of the subject. In some embodiments, decreasing or preventing an increase of the level of one or more di-amino acid repeat-containing proteins comprises removing the one or more di-amino acid repeat-containing proteins from the blood of the subject. In some embodiments, the one or more di-amino acid repeat-containing proteins from the blood of the subject is removed using a procedure selected from plasmapheresis or a bone marrow transplantation. In some embodiments, the bone marrow transplantation is an allogeneic bone marrow transplantation.

In yet another aspect, the disclosure relates to an isolated antibody specific for one or more di-amino acid repeat proteins selected from a poly-(Gly-Ala), poly-(Gly-Pro), poly-(Gly-Arg), poly-(Pro-Ala), poly-(Pro-Arg), Met . . . poly-(Pro-Arg) or Met . . . poly-(Gly-Pro) protein. In some embodiments, the di-amino acid repeat protein is selected from a poly-(Pro-Ala), poly-(Pro-Arg), Met . . . poly-(Pro-Arg) or Met . . . poly-(Gly-Pro) protein. In some embodiments, the isolated antibody is specific for an antigen comprising a sequence or fragment of a sequence as set for in Tables 1, 2, or 3.

Other aspects of the disclosure relate to a method for identifying a subject as having ALS or FTD or likely to develop ALS or FTD, the method comprising determining, in a sample obtained from a subject, a level of a 5′ GGGGCC-3′ hexanucleotide repeat-containing RNA and/or a 5′-GGCCCC-3′ hexanucleotide repeat-containing RNA, wherein a level of the 5′-GGGGCC-3′ hexanucleotide repeat-containing RNA and/or a 5′-GGCCCC-3′ hexanucleotide repeat-containing RNA that is elevated compared to a control level indicates that the subject has ALS or FTD or is likely to develop ALS or FTD. In some embodiments, the level is determined by performing an assay. In some embodiments, the assay comprises a nucleic acid-based assay, such as in-situ hybridization (e.g., FISH) or RT-PCR (e.g., quantitative RT-PCR or strand specific quantitative RT-PCR). In some embodiments, the method further comprises identifying the subject as having ALS or FTD or likely to develop ALS or FTD if the level of a 5′-GGGGCC-3′ hexanucleotide repeat-containing RNA and/or a 5′-GGCCCC-3′ hexanucleotide repeat-containing RNA is elevated compared to a control level. In some embodiments, the method further comprises treating the subject having ALS or FTD or likely to develop ALS or FTD. In some embodiments, treating comprises administering to the subject an effective amount of one or more of riluzole, baclofen, diazepam, phenytoin, trihexyphenidyl or amitriptyline. In some embodiments, treating comprises performing a procedure selected from plasmapheresis or a bone marrow transplant. In some embodiments, the level of a 5′-GGGGCC-3′ hexanucleotide repeat-containing RNA and/or a 5′-GGCCCC-3′ hexanucleotide repeat-containing RNA is a level of a 5′-GGCCCC-3′ hexanucleotide repeat-containing RNA.

Yet other aspects of the disclosure relate to a method for identifying a subject as having ALS or FTD or likely to develop ALS or FTD, the method comprising determining, in a sample obtained from a subject, the presence or absence of foci containing 5′-GGGGCC-3′ hexanucleotide repeat-expansion-containing RNA and/or a 5′-GGCCCC-3′ hexanucleotide repeat-expansion-containing RNA, wherein the presence of the foci of the 5′-GGGGCC-3′ hexanucleotide repeat-expansion-containing RNA and/or a 5′-GGCCCC-3′ hexanucleotide repeat-expansion-containing RNA indicates that the subject has ALS or FTD or is likely to develop ALS or FTD. In some embodiments, presence or absence of foci or elevated C9ORF72 sense or antisense RNA levels is determined by performing an assay. In some embodiments, the assay comprises a nucleic acid-based assay, such as strand specific RT-PCR or in-situ hybridization (e.g., FISH).

Yet other aspects of the disclosure relate to transgenic mice. In some embodiments, the transgenic mouse comprises a human C9ORF72 gene and optionally human flanking sequences. In some embodiments, the transgenic mouse comprises SEQ ID NO: 63.

These and other aspects are described in more detail herein and illustrated by the non-limiting figures and examples.

### BRIEF DESCRIPTION OF THE DRAWINGS

The drawings are first described.

FIG. 1 is a drawing showing that transcripts are produced in the sense and anti-sense direction on the C9ORF72 gene, and that repeat-associated non-ATG (RAN) translation proteins are translated in all three reading-frames from both the sense and anti-sense C9ORF72 transcripts. The drawing also shows that Met . . . poly-(Pro-Arg) and Met . . . poly-(Gly-Pro) proteins are translated through ATG-initiated translation on the anti-sense transcript. CT=predicted to and/or shown to contain a c-terminal domain. *=end of protein (due to stop codon). M=Methionine.

FIG. 2A is a diagram of an expression vector for expressing RAN translation proteins in cells. CMV=cytomegalovirus promoter. 6xStop=6 stop codons, two in each frame. (GGGGCC) exp=a GGGGCC repeat sequence that extends for 4, 30, 60, or 120 repeats. (GR) HA-(GP) Flag-(GA) Myc=a HA, flag or myc tag that corresponds to the poly-(Gly-Arg), poly-(Gly-Pro), and poly-(Gly-Ala) repeat proteins, respectively. SV40 poly(a)=transcription terminator and poly A signal.

FIG. 2B is a photograph of a western blot depicting that GR and GP RAN translation proteins are expressed in cells transfected with 30, 60 or 120 GGGGCC repeat sequences.

FIG. 3 is a photograph of an immunofluorescence staining of cells expressing GP, GR, or GA RAN proteins in cells transfected with 30, 60 or 120 GGGGCC repeat sequences.

FIG. 4 is a diagram of the poly-(GR) and GR-c-terminus antigens and a series of photographs of immunofluorescence staining showing that the poly-(GR) and (GR)-c-terminal antibodies detect poly-(GR) RAN proteins.

FIG. 5 is a series of photographs of tissue from C9ORF72 ALS patients or control patients showing that poly-(GR), poly-(GP), and poly-(GA) di-amino acid-repeat-containing proteins are expressed by C9ORF72 ALS patients.

FIG. 6 is a series of photographs of tissue from C9ORF72 ALS patients or control patients showing that poly-(PA) and poly-(PR) di-amino acid-repeat-containing proteins are expressed by C9ORF72 ALS patients.

FIG. 7A is a series of photographs of immunofluorescence staining showing antibodies generated to recognize the GP repeat motif (GP) or the unique C-terminal region of the same GP-RAN proteins (GP-C) colocalize in 20% of patient cells. Cells that stain for and GP-C and GP express GP-RAN protein in the sense direction and that cells showing only GP staining express RAN-GP or Met . . . GP from the anti-sense strand.

FIG. 7B is a graph depicting the percentage of GP and GP+GP-C in patient cells.

FIG. 8 is a picture of a dot blot showing that di-amino acid repeat-containing proteins are found in the blood (PBL) and the brain (FCX, frontal cortex) of subjects with ALS, but not controls.

FIG. 9 is a photograph of a western blot showing that GP-repeat proteins are present in the brain (FCX) of subjects with ALS but not controls and that PA-repeat proteins are present in the plasma and serum of subjects with ALS but not controls.

FIG. 10 is a schematic of the RAN translation mouse model construct containing 6X stops, a CAG repeat region, tags for detecting each CAG repeat frame, and a terminator sequence.

FIG. 11 depicts two photographs showing that poly-Gln proteins accumulated in the brain of RAN translation (RANT) mice containing the construct in FIG. 10, but not in control mice.

FIG. 12 is a series of schematics, graphs and images showing that G2C4 antisense transcripts are elevated by strand specific RT-PCR and accumulate as RNA foci in C9ORF72 patient tissues. (A) Schematic diagram of C9ORF72 gene and antisense transcripts and relative location of primers for strand-specific RT-PCR and RACE primers. (B) Strand-specific RT-PCR of sense(S) and antisense (AS) transcripts (across intron 1b and exon 1) from frontal cortex of C9 (+) and C9 (−) ALS patients. (C) strand-specific qRT-PCR showing elevated antisense mRNA in C9 (+) compared to C9 (−) ALS patients. (D) In situ hybridization with G4C2-Cy3 probe showing G2C4 antisense RNA foci (arrowheads) in C9 (+) frontal cortex and peripheral blood leukocytes (PBLs) which are absent in C9 (−) cases. Nuclear foci in FCX are indicated by arrow heads. FCX=frontal cortex. PBL=peripheral blood leukocytes.

FIG. 13 is a series of schematics, graphs and images showing in vitro evidence for RAN translation of antisense G2C4 expansion and dual immunological detection strategy. (A-C) Immunoblots (B) and IF staining (C) of HEK293T cells 48 hours post-transfection with the (G2Ca) Exp-3T construct (A). (B) PR and GP expansion proteins detected by western and (C) PA, PR and GP detected by IF in transfected cells. (D) Diagram of putative proteins translated from sense and antisense transcripts. CT=C-terminal, f1-3: reading frame 1-3. (E) Abbreviated example of validation of α-PA rabbit polyclonal antibody. IF staining of HEK293T cells transfected with constructs with 5′ Flag epitope tagged PA protein and corresponding immunoblots. See FIGS. 22 and 23 for additional controls and validation of eight additional antibodies generated against repeat motifs and CT regions.

FIGS. 14A and 14B are a series of images and graphs showing in vivo evidence for RAN-translation of the G2C4 AS repeat and toxicity studies. (A) Dot blot of C9 (+) and C9 (−) frontal cortex lysates probed with α-PA. α-PA-CT, α-PR, α-PR-CT antibodies. (B) Immunoblots of C9 (+) and C9 (−) ALS frontal cortex lysates. (C) IHC detection of PA, PR and GP protein aggregates in hippocampal neurons from C9 (+) ALS patients detected with a-PA, α-PA-CT, α-PR, α-PRCT and α-GP antibodies. (D) IF staining with mouse α-GP (arrowhead) and rabbit α-GP-CT (arrow) of C9 (+) hippocampal tissue with sense inclusions positive for both antibodies (upper panel) and antisense inclusions positive for only GP repeat antibody (lower panel). (E) IF staining of larger region showing sense(S) and antisense (AS) staining. (F) Quantitation of double (sense) and single (antisense) labeled aggregates. (G-J) RAN and PR toxicity studies (G) G2C4 expansion constructs (+/−ATG-PR-3T)+/−ATG initiation codon in PR frame and 3′epitope tags. (H) Protein blots showing levels of PR and GP in cells transfected with constructs in (G). (I) LDH and (J) MTT assays of transfected HEK293T cells.

FIGS. 15A and 15B are a series of images showing in vivo evidence for RAN translation in both antisense and sense directions of C90RF72. Cytoplasmic inclusions detected by IHC using antibodies against sense (a-GR, α-GR-CT, α-GA, α-GP-CT) and antisense (α-PA, α-PA-CT, α-PR, α-PR-CT) and α-GP which recognizes GP proteins made in both the sense and antisense directions. Aggregates were found in neurons of cornu ammonis (CA) and dentate gyrus (DG) regions of the hippocampus and the motor cortex (MC) of C9 (+) ALS autopsy tissue.

FIGS. 16A and 16B are a series of images of clustered RAN protein aggregates and RAN aggregates in motor neurons. IHC showing cytoplasmic α-GP aggregates in: (A) in layer Ill of motor cortex. (B) upper motor neuron in layer V of the motor cortex; (C) lower motor neurons in the spinal cord (L-S.C). (D) in cornu ammonis, CA. (E) and dentatus gyrus, DG regions of the hippocampus. (F and G) IHC showing abundant PA and PR cytoplasmic inclusions in the pre-subiculum (PrSub) from one patient.

FIG. 17 is a series of images of clustered staining of RAN proteins. (A) Low power image of IHC staining with α-PA-CT shows variations in staining intensity (dark spots are positive) in regions I-IV with insets showing higher-power images. (B) Examples of aggregates from region I show immunoreactivity against all nine antibodies with similar staining for antibodies against repeat and unique C-terminal epitopes.

FIG. 18 is a table summarizing histopathological findings in C9ORF72 positive ALS/FTD cases and controls.

FIGS. 19A-19F are a series of images and datasets. (A) shows strand-specific RT-PCR detection of sense(S) and antisense (AS) transcripts (across intron 1) of PBLs of C9 (+) patient and normal controls. (B) is a summary of 5′ RACE products. (C) shows FISH staining of frontal cortex from a C9 (+) case showing an example of cytoplasmic RNA foci. (D) shows FISH staining of peripheral blood leukocytes showing the accumulation of antisense (AS) G₂C₄ and sense(S) G₄C₂ RNA foci in C9 (+) but not C9 (−) cells. (E) shows antisense foci specificity assay showing excess unlabeled (G₄C₂)₄ oligo blocks labeling of G4C2-Cy3 antisense (AS) but not G₂C₄-Cy3 labeled sense foci. (F) shows additional controls for antisense RNA foci showing expected DNase I resistance and RNase I sensitivity.

FIG. 20 is a series of images of in vitro evidence for RAN translation of the sense GGGGCC repeat expansion. (A) shows constructs containing varying GGGGCC repeat lengths with upstream 6X Stop cassette and 3′ tags in each reading frame. Immunoblots (B) and/or immunofluorescence staining (C) showing RAN translation occurs in all three frames (GP, GR, GA) in cells transfected with constructs containing 30, 60 and 120 repeats.

FIG. 21 is a schematic of putative protein products in sense and antisense directions for all reading frames SEQ ID NOs: 57-62, from top to bottom. Underlined sequences were used to generate polyclonal antibodies. *=Stop codon.

FIGS. 22A-22E are a series of images showing validation of dual antibodies to detect putative polyPA, polyPR, polyGP proteins by immunofluorescence and protein blot (A-D Top): Schematic diagrams of constructs expressing ATG-initiated N-terminal epitope-tagged (V5 or Flag) repeat proteins with or without endogenous C-terminal sequences. (A-D Bottom panels), co-localization of α-Flag or α-V5 staining in transfected HEK293T cells with staining using the following newly developed antibodies: (A) α-PA or α-PA-CT (antisense); (B) α-PR or α-PR-CT (C) rabbit α-GP or α-GP-CT (sense); (D) mouse α-GP. Similar staining was not seen in preimmune or pcDNA3.1 empty vector controls; (E) Corresponding immunoblots showing six of the seven antibodies tested also detect recombinant proteins by Western.

FIGS. 23A-23C are a series of images showing validation of additional sense repeat and C-terminal polyclonal antibodies. (A, B Top): Schematic diagrams of constructs expressing ATG-initiated N-terminal V5-epitope tagged GR or GA repeat proteins with endogenous C-terminal sequences. (A-B Bottom panels), co-localization of α-V5 staining in transfected HEK293T cells with α-GR, α-GR-CT and α-GP-CT respectively. Similar staining was not seen in preimmune or pcDNA3.1 empty vector controls. (C) α-GR detection of recombinant protein in Flag-GR transfected cells by protein blot.

FIG. 24 is a series of images of immunoblots of 2% soluble lysates from C9 (+) and C9 (−) ALS frontal cortices with α-GP-CT, α-GR, α-GR-CT and α-GA antibodies.

FIG. 25 is a series of images showing negative IHC staining of C9 (−) ALS/FTD hippocampal sections with antibodies against sense and antisense proteins.

FIGS. 26A-26D are a graph and a series showing images RAN translation and PR protein expression affect cell viability. (A) qRT-PCR shows expression of expansion transcripts are similar in HEK293T cells transfected with (−) ATG-PR-3T and (+) ATG-PR-3T constructs. (B-D) Bright-field microscopy images showing changes in cell morphology in cells expressing RNA and RAN proteins from (−) ATG-PR-3T constructs compared to empty vector control (pcDNA3.1) and worsening effects in (+) ATG-PR-3T cells expressing increased levels of PR protein.

FIG. 27 is a table describing primers used for RT-PCR and RACE (SEQ ID NOs: 17 of them (in order. SEQ ID NOs: 36, 37, 39, 38, 45-47, 40, 48-56).

FIG. 28 is a table describing novel sense and antisense antibodies. (in order SEQ ID NOs: 20, 23, 19, 25, 21, 21, 22, 18).

FIG. 29 is a schematic of the BAC insert used to make transgenic mice.

FIG. 30 is a series of photographs showing sense RNA foci in transgenic mice expressing a human C9ORF72 gene containing GGGGCC repeats. Exemplary foci are indicated by arrowheads.

FIG. 31 is a series of photographs showing anti-sense (AS) RNA foci in transgenic mice expressing a human C9ORF72 gene containing GGGGCC repeats. Exemplary foci are indicated by arrowheads.

### DETAILED DESCRIPTION OF THE INVENTION

Well-established rules of translational initiation have been used as a cornerstone in molecular biology to understand gene expression and to predict the consequences of disease causing mutations. In general, microsatellite expansion mutations (e.g., CAG, CTG) located in predicted coding- and non-coding regions have been thought to cause disease by protein gain-, or loss-, of-function or RNA gain-of-function mechanisms. It has been previously reported that the canonical rules of translation do not apply for CTG·CAG repeat expansions and that CAG and CUG expansion transcripts express homopolymeric expansion proteins in all three frames without an AUG start codon (see, e.g., T. Zu et al., Non-ATG-initiated translation directed by microsatellite expansions. PNAS 108, 260 (2011)). This translation independent of an AUG start codon is termed repeat-associated non-ATG (RAN) translation. RAN translation is hairpin dependent and occurs without frameshifting or RNA editing. RAN translation has been observed from trinucleotide, tetranucleotide, and pentanucleotide repeats associated with myotonic dystrophy 1, myotonic dystrophy 2, spinocerebellar ataxia type 3, spinocerebellar ataxia type 8 and Huntington disease (see PCT publication WO/2010/115033, which is incorporated herein by reference).

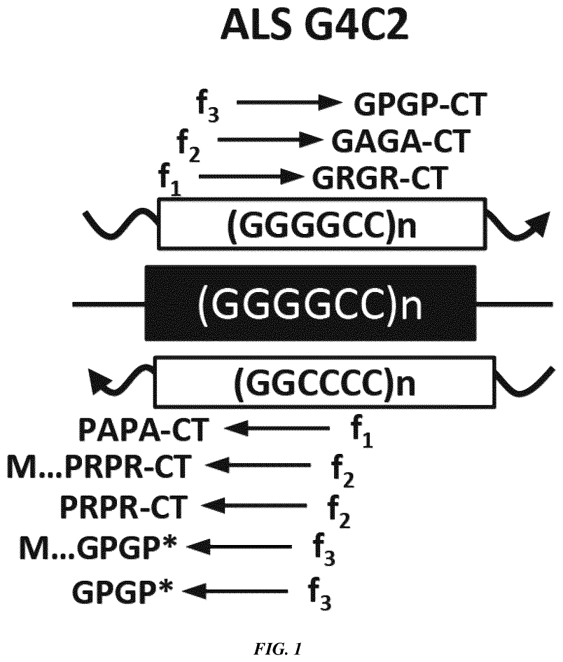

Expansion of a GGGGCC hexanucleotide repeat within the intron of the C9ORF72 gene has been previously associated with both amyotrophic lateral sclerosis and frontotemporal dementia. As described herein, it has been found that this expanded hexanucleotide repeat is contained within RNA transcripts expressed in both the sense and anti-sense direction from the C9ORF72 locus. These hexanucleotide repeat-containing transcripts were found to undergo RAN translation such that poly-(Gly-Ala), poly-(Gly-Pro), poly-(Gly-Arg), poly-(Pro-Ala), or poly-(Pro-Arg) proteins were produced, depending on the frame of the hexanucleotide repeat being read from the RNA (5′-GGGGCC-3′, 5′-GGGCCG-3′, and 5′-GGCCGG-3′ on the sense transcript, 5′ GGCCCC-3′, 5′-GCCCCG-3′, and 5′-CCCCGG-3′ on the anti-sense transcript, see FIG. 1). In addition, the anti-sense transcript was found to be translated through ATG-initiated translation to produce Met . . . poly-(Pro-Arg) and Met . . . poly-(Gly-Pro) proteins. These RAN and ATG-initiated proteins are referred to as di-amino acid-repeat-containing proteins herein. The sense and anti-sense hexanucleotide repeat-containing transcripts are referred to herein as 5′-GGGGCC-3′ hexanucleotide repeat-containing RNA (sense) and 5′-GGCCCC-3′ hexanucleotide repeat-containing RNA (anti-sense).

As further described herein, these di-amino acid-repeat-containing proteins unexpectedly were found to be present in blood samples from subjects with ALS. Additionally, expression of the anti-sense 5′-GGCCCC-3′ hexanucleotide repeat-containing RNA transcript was found to be highly elevated in subjects having a C9ORF72 gene containing the expanded GGGGCC hexanucleotide repeat sequence. Further, foci of both the sense and anti-sense hexanucleotide repeat-expansion-containing RNA transcripts were found to be present in subjects having a C9OR/72 gene containing the expanded GGGGCC hexanucleotide repeat sequence. Without wishing to be bound by theory or mechanism, it is believed that di-amino acid-repeat-containing proteins in the blood of subjects with ALS accumulate within the brain parenchyma over time, leading to neuroinflammatory changes, CNS dysfunction, and neuronal death. Accordingly, aspects of the disclosure relate to identification of a subject as having ALS or likely to develop ALS by providing novel assays for determining di-amino acid-repeat-containing protein levels in the blood of the subject and/or hexanucleotide repeat-containing RNA levels in a sample from the subject. Aspects of the disclosure also relate to treatment of a subject having ALS or FTD by decreasing or stabilizing di-amino acid-repeat-containing protein levels in the blood of the subject.

### Identification of a Subject Having ALS or FTD or Likely to Develop ALS or FTD

Aspects of the disclosure relate to identification of a subject having ALS or FTD or likely to develop ALS or FTD based on a level of one or more di-amino acid-repeat-containing proteins in a blood sample from a subject. In some embodiments, a method comprises, determining, in a blood sample obtained from a subject, a level of one or more di-amino acid-repeat-containing proteins selected from a poly-(Gly-Ala), poly-(Gly-Pro), poly-(Gly-Arg), poly-(Pro-Ala), poly-(Pro-Arg), Met . . . poly-(Pro-Arg) or Met . . . poly-(Gly-Pro) protein, wherein a level of the one or more di-amino acid-repeat-containing proteins that is elevated compared to a control level indicates that the subject has ALS or FTD or is likely to develop ALS or FTD. In some embodiments, a level of one or more di-amino acid-repeat-containing proteins is determined by performing an assay. Non-limiting assays are described herein.

Other aspects of the disclosure relate to identification of a subject having ALS or FTD or likely to develop ALS or FTD based on a level of a 5′-GGGCCC-3′ hexanucleotide repeat-containing RNA and/or a 5′-GGCCCC-3′ hexanucleotide repeat-containing RNA in a sample from a subject. In some embodiments, identification of a subject having ALS or FTD or likely to develop ALS or FTD is based on a level of a 5′-GGCCCC-3′ hexanucleotide repeat-containing RNA in a sample from a subject. The sample may be, e.g., a fluid or tissue sample obtained from the subject. In some embodiments, a method comprises, determining, in a sample obtained from a subject, a level of a 5′-GGGGCC-3′ hexanucleotide repeat-containing RNA and/or a 5′-GGCCCC-3′ hexanucleotide repeat-containing RNA, wherein a level of the hexanucleotide repeat-containing RNA that is elevated compared to a control level indicates that the subject has ALS or FTD or is likely to develop ALS or FTD. In some embodiments, a level of a hexanucleotide repeat-containing RNA is determined by performing an assay. Non-limiting assays are described herein.

Yet other aspects of the disclosure relate to identification of a subject having ALS or FTD or likely to develop ALS or FTD based on the presence or absence of RNA foci containing a 5′-GGGGCC-3′ hexanucleotide repeat-expansion-containing RNA and/or a 5′-GGCCCC-3′ hexanucleotide repeat-expansion-containing RNA in a sample from a subject, wherein the presence of the focus of the 5′-GGGGCC-3′ hexanucleotide repeat-expansion-containing RNA and/or a 5′-GGCCCC-3′ hexanucleotide repeat-expansion-containing RNA indicates that the subject has ALS or FTD or is likely to develop ALS or FTD. As used herein, a focus of a 5′-GGGGCC-3′ hexanucleotide repeat-expansion-containing RNA and/or a 5′-GGCCCC-3′ hexanucleotide repeat-expansion-containing RNA refers to an area of accumulation of the 5′-GGGGCC-3′ hexanucleotide repeat-expansion-containing RNA and/or the 5′-GGCCCC-3′ hexanucleotide repeat-expansion-containing RNA, which may be detectable using a nucleic acid-based assay, such as FISH. In some embodiments, the focus may be, e.g., 0.1 to 2 micrometers in diameter, 0.1 to 1.5 micrometers in diameter, or 0.1 to 1 micrometers in diameter. In some embodiments, the focus may be at least 0.1 micrometers in diameter. It is to be appreciated that a sample may contain more than one focus and that each focus may be a different size. For example, one focus may be 0.2 micrometers in diameter, while second focus may be I micrometer in diameter. Non-limiting examples of foci and methods detecting such foci are provided in Example 3.

It is to be understood that a subject may be identified based on a level of one or more di-amino acid-repeat-containing proteins, a level of a hexanucleotide repeat-expansion containing RNA, the presence or absence of a hexanucleotide repeat-expansion containing RNA, or any combination thereof. In some embodiments, the method further comprises identifying the subject as having ALS or FTD or likely to develop ALS or FTD if the level of the di-amino acid-repeat-containing protein or hexanucleotide repeat-containing RNA is elevated compared to a control level. In some embodiments, the method further comprises identifying the subject as having ALS or FTD or likely to develop ALS or FTD if the focus or foci of the hexanucleotide repeat-expansion-containing RNA are present in the sample. In some embodiments, the method further comprises identifying the subject as not having ALS or FTD or unlikely to develop ALS or FTD if the level of the di-amino acid-repeat-containing protein or hexanucleotide repeat-containing RNA is decreased or the same compared to a control level. In some embodiments, the method further comprises identifying the subject as not having ALS or FTD or unlikely to develop ALS or FTD if the focus or foci of the hexanucleotide repeat-expansion-containing RNA are absent in the sample.

In some embodiments, a level of one or more di-amino acid-repeat-containing proteins or the identity of a subject may be recorded. In some embodiments, recordation comprises inputting a level or identity of subject into a computer, such as a medical record database.

Other aspects of the disclosure relate to treatment of a subject identified as having ALS or FTD or likely to develop ALS or FTD. As used herein, “treat” or “treatment” refers to (a) preventing or delaying the onset of ALS or FTD; (b) reducing the severity of ALS or FTD; (c) reducing or preventing development of symptoms characteristic of ALS or FTD; (d) preventing worsening of symptoms characteristic of ALS or FTD; and/or (e) reducing or preventing recurrence of ALS or FTD symptoms in subjects that were previously symptomatic for ALS or FTD.

In some embodiments, treatment comprises administering an effective amount of a known ALS therapeutic agent, such as Riluzole (Rilutek. Sanofi-Aventis), to a subject identified as having ALS. In some embodiments, treatment comprises administering an effective amount of a known FTD therapeutic agent, such as trazodone (Desyrel, Oleptro) or a selective serotonin reuptake inhibitor (SSRI), to a subject identified as having FTD. In some embodiments, treatment comprises administering an effective amount of a therapeutic agent, such as baclofen, diazepam, phenytoin, trihexyphenidyl and/or amitriptyline, which reduces one or more symptoms of ALS or FTD in a subject identified as having ALS or FTD.

In some embodiments, treatment comprises one or more of physical therapy, occupational therapy, or speech therapy. In some embodiments, treatment comprises a method as described herein for decreasing or stabilizing di-amino acid-repeat-containing protein levels in the blood of the subject, such as bone marrow transplantation or plasmapheresis. In some embodiments, treatment comprises any combination of the above-mentioned treatments or any other treatments described herein.

An effective amount is a dosage of a therapeutic agent sufficient to provide a medically desirable result, such as treatment of ALS or FTD. The effective amount will vary with the age and physical condition of the subject being treated, the severity of ALS or FTD in the subject, the duration of the treatment, the nature of any concurrent therapy, the specific route of administration and the like factors within the knowledge and expertise of the health practitioner.

Administration of a treatment may be accomplished by any method known in the art (see, e.g., Harrison's Principle of Internal Medicine, McGraw Hill Inc.). Administration may be local or systemic. Administration may be parenteral (e.g., intravenous, subcutaneous, or intradermal) or oral. Compositions for different routes of administration are well known in the art (see, e.g., Remington's Pharmaceutical Sciences by E. W. Martin). Dosage will depend on the subject and the route of administration. Dosage can be determined by the skilled artisan.

Other aspects of the disclosure relate to methods for monitoring responsiveness to a treatment in a subject having ALS or FTD or suspected of having ALS or FTD. In some embodiments, the method comprises: determining, in a blood sample obtained from the subject at a first time point, a first level of one or more di-amino acid-repeat-containing proteins selected from a poly-(Gly-Ala), poly-(Gly-Pro), poly-(Gly-Arg), poly-(Pro-Ala), poly-(Pro-Arg), Met . . . poly-(Pro-Arg) or Met . . . poly-(Gly-Pro) protein; and determining, in a blood sample obtained from the subject at a second time point, a second level of one or more di-amino acid-repeat-containing proteins selected from a poly-(Gly-Ala), poly-(Gly-Pro), poly-(Gly-Arg), poly-(Pro-Ala), poly-(Pro-Arg), Met . . . poly-(Pro-Arg) or Met . . . poly-(Gly-Pro) protein, wherein a second level that is elevated or the same compared to a first level indicates that the subject is unresponsive or likely unresponsive to treatment and wherein a second level that is decreased compared to a first level indicates that the subject is responsive or likely responsive to treatment. In some embodiments, the first blood sample is obtained before treatment of the subject and the second blood sample is obtained during or after treatment of the subject. This method may also be performed by determining a level of a hexanucleotide repeat-containing RNA or the presence or absence of a focus or foci of a hexanucleotide repeat-expansion-containing RNA in addition to or in place of the level of di-amino acid protein.

As used herein, “elevated” means that the level of one or more di-amino acid-repeat-containing proteins or a hexanucleotide repeat-containing RNA is above a control level, such as a pre-determined threshold or a level of one or more di-amino acid-repeat-containing proteins or a hexanucleotide repeat-containing RNA in a control sample. Controls and control levels are described in detail herein. An elevated level includes a level that is, for example, 1%, 5%, 10%, 20%, 30%, 40%, 50%, 60%, 70%, 80%, 90%, 100%, 150%, 200%, 300%, 400%, 500%, or more above a control level. An elevated level also includes increasing a phenomenon from a zero state (e.g., no or undetectable di-amino acid-repeat-containing protein expression or hexanucleotide repeat-containing RNA expression) to a non-zero state (e.g., some or detectable di-amino acid-repeat-containing protein expression or hexanucleotide repeat-containing RNA).

As used herein, “decreased” means that the level of one or more di-amino acid-repeat-containing proteins or a hexanucleotide repeat-containing RNA is below a control level, such as a pre-determined threshold or a level of one or more di-amino acid-repeat-containing proteins or a hexanucleotide repeat-containing RNA in a control sample. Controls and control levels are described in detail herein. A decreased level includes a level that is, for example, 1%, 5%, 10%, 20%, 30%, 40%, 50%, 60%, 70%, 80%, 90%, 100%, 150%, 200%, 300%, 400%, 500%, or more below a control level. A decreased level also includes decreasing a phenomenon from a non-zero state (e.g., some or detectable di-amino acid-repeat-containing protein expression or hexanucleotide repeat-containing RNA) to a zero state (e.g., no or undetectable di-amino acid-repeat-containing protein expression or hexanucleotide repeat-containing RNA expression).

### Hexanucleotide Repeat-Containing RNAs and Di-Amino Acid Repeat-Containing Proteins

As described herein, an expanded GGGGCC hexanucleotide repeat sequence within the intron of the C9ORF72 gene was found to be transcribed such that RNA transcripts containing the hexanucleotide repeat in both the sense and anti-sense direction were produced. The GenBank Gene ID for the human C9ORF72 gene is 203228. Both the sense and anti-sense hexanucleotide repeat-containing transcripts were found to undergo translation independent of an AUG start codon (repeat-associated non-ATG (RAN) translation) such that poly-(Gly-Ala), poly-(Gly-Pro), poly-(Gly-Arg), poly-(Pro-Ala), or poly-(Pro-Arg) di-amino acid repeat-containing proteins were produced, depending on the frame of the hexanucleotide repeat being read (5′-GGGGCC-3′, 5′-GGGCCG-3′, and 5′-GGCCGG-3′ on the sense transcript. 5′-GGCCCC-3′, 5′-GCCCCG-3′, and 5′-CCCCGG-3′ on the anti-sense transcript, see FIG. 1). In addition, the anti-sense hexanucleotide repeat-containing transcript was found to be translated through ATG-initiated translation to produce Met . . . poly-(Pro-Arg) and Met . . . poly-(Gly-Pro) proteins.

Accordingly, aspects of the invention relate to the sense and anti-sense RNAs containing an expanded hexanucleotide repeat and uses thereof. The sense RNA is a 5′-GGGGCC-3′ hexanucleotide repeat-containing RNA and the anti-sense RNA is a 5′-GGCCCC-3′ hexanucleotide repeat-containing RNA.

The 5′-GGGGCC-3′ and 5′GGCCCC-3′ hexanucleotide repeat-containing RNAs comprise a repeat nucleic acid sequence of the formula (GGGGCC)ₓ or (GGCCCC)ₓ, respectively, where X may be at least 10, at least 20, at least 25, or at least 30, or in a range selected from 10-100,000, 10-50,000, 10-5,000, 20-1,000, 20-100,000, 20-50,000, 20-5,000, 20-1,000, 25-100,000, 25-50.000, 25-5,000, or 25-1,000. The hexanucleotide repeat-containing RNA may further comprise additional N- and/or C-terminal nucleic acids. In some embodiments, an N-terminal nucleic sequence comprises a nucleic acid sequence upstream of the 5′-GGGGCC-3′ hexanucleotide repeat within the intron of the C9ORF72 for the sense transcript or a nucleotide sequence upstream of the 5′-GGCCCC-3′ hexanucleotide repeat within the intron of the C9ORF72 for the anti-sense transcript. In some embodiments, a C-terminal nucleic acid sequence comprises a nucleotide sequence downstream of the 5′-GGGGCC-3′ hexanucleotide repeat within the intron of the C9ORF72 for the sense transcript or a nucleotide sequence downstream of the 5′-GGCCCC-3′ hexanucleotide repeat within the intron of the C90RF72 for the anti-sense transcript.

Other aspects of the invention relate to one or more di-amino acid repeat-containing proteins and uses thereof. The one or more di-amino acid repeat-containing proteins are selected from poly-(Gly-Ala), poly-(Gly-Pro), poly-(Gly-Arg), poly-(Pro-Ala), poly-(Pro-Arg), Met . . . poly-(Pro-Arg) or Met . . . poly-(Gly-Pro) proteins.

The sense 5′-GGGGCC-3′ hexanucleotide repeat-containing RNA and the anti-sense 5′-GGCCCC-3′ hexanucleotide repeat-containing RNA both encode poly-(Gly-Pro) proteins. Accordingly a poly-(Gly-Pro) protein may include a protein translated from the sense strand, the anti-sense strand, or both. It is predicted that the C-terminus of the sense and anti-sense translated poly-(Gly-Pro) proteins may differ (see Table 1). Accordingly, a sense poly-(Gly-Pro) protein may comprise the poly-(Gly-Pro) a C-terminal sequence as described in Table 1, while an anti-sense poly-(Gly-Pro) protein may comprise the repeat region with no additional C-terminal sequence. Methods described herein may comprise use of a poly-(Gly-Pro) protein translated from the sense strand, the anti-sense strand, or both. Antibodies described herein may be specific for a poly-(Gly-Pro) protein translated from the sense strand, the anti-sense strand, or both.

Each di-amino acid repeat-containing protein comprises a repeat amino acid sequence, which contains a di-amino acid repeat unit of the formula (YZ) x, where X can be from 2-10,000, 5-10,000, 2-5,000, 5-5,000, 2-1000, 5-1000, 5-500, 5-300, 5-200, 10-500, 10-300, or 10-200. The di-amino acid repeat unit for each di-amino acid repeat-containing protein is provided in Table 1.

TABLE 1
Di-Amino Acid-Repeat-Containing Proteins

| Di-Amino | | |
|---|---|---|
| Acid-Repeat- | | |
| Containing | Di-Amino Acid | |
| Protein | Repeat Unit | Predicted C-terminus |
| poly-(Gly-Ala) | (GA)ₓ or (AG)ₓ | WSGRARGRARGGAAVAVPAPAAAEAQA |
| | | VASG (SEQ ID NO: 1) or |
| | | AWSGRARGRARGGAAVAVPAPAAAEAQ |
| | | AVASG (SEQ ID NO: 27) |
| poly-(Gly-Pro) | (GP)ₓ or (PG)ₓ | GRGRGGPGGGPGAGLRLRCLRPRRRRRR |
| | | RWRVGE (SEQ ID NO: 2, sense), |
| | | PGRGRGGPGGGPGAGLRLRCLRPRRRRRR |
| | | RWRVGE (SEQ ID NO: 28, sense) |
| | | or none (anti-sense) |
| poly-(Gly-Arg) | (GR)ₓ or (RG)ₓ | GVVGAGPGAGPGRGCGCGACARGGGGA |
| | | GGGEWVSEEAASWRVAVWGSAAGKRRG |
| | | (SEQ ID NO: 3) or |
| | | RGVVGAGPGAGPGRGCGCGACARGGGG |
| | | AGGGEWVSEEAASWRVAVWGSAAGKRR |
| | | G (SEQ ID NO: 29) |
| poly-(Pro-Ala) | (AP)ₓ or (PA)ₓ | PSARLLSSRACYRLRLFPSLESSG |
| | | (SEQ ID NO: 4) OR |
| | | APSARLLSSRACYRLRLFPSLESSG |
| | | (SEQ ID NO: 30) |
| poly-(Pro-Arg) | (PR)ₓ or (RP)ₓ | PLARDS (SEQ ID NO: 5) or RPLARDS |
| | | (SEQ ID NO: 31) |
| Met . . . | (PR)ₓ | PLARDS (SEQ ID NO: 5) |
| poly-(Pro-Arg) | | |
| Met . . . | (GP)ₓ | None |
| poly-(Gly-Pro) | | |

x = number of repeats of the sequence in the parentheses

Each di-amino acid repeat-containing protein may further comprise an N- and/or C-terminal amino acid sequence that comprises a non-di-amino acid repeat sequence. In some embodiments, a N-terminal amino acid sequence comprises an amino acid sequence translated from a nucleotide sequence of a C9ORF72 RNA transcript, such as a nucleotide sequence upstream of the 5′-GGGGCC-3′ hexanucleotide repeat within the intron of the C9ORF72 for the sense transcript or a nucleotide sequence upstream of the 5′-GGCCCC-3′ hexanucleotide repeat within the intron of the C9ORF72 for the anti-sense transcript. In some embodiments, a C-terminal amino acid sequence comprises an amino acid sequence translated from a nucleotide sequence of a C9ORF72 RNA transcript, such as a nucleotide sequence downstream of the 5′-GGGGCC-3′ hexanucleotide repeat within the intron of the C90RF72 for the sense transcript or a nucleotide sequence downstream of the 5′-GGCCCC-3′ hexanucleotide repeat within the intron of the C9ORF72 for the anti-sense transcript. Such a nucleotide sequence downstream of the 5′-GGGGCC-3′ or 5′-GGCCCC-3′ hexanucleotide repeat may be translated until a stop codon or multiple stop codons are reached.

A portion of a C9ORF72 gene sequence (sense and anti-sense) is shown below. The 5′-GGGGCC-3′ or 5′-GGCCCC-3′ hexanucleotide repeat is underlined and in bold. The nucleotide sequence upstream of the 5′-GGGGCC-3′ or 5′-GGCCCC-3′ hexanucleotide repeat precedes the underlined and bolded sequence. The nucleotide sequence downstream of the 5′-GGGGCC-3′ or 5′-GGCCCC-3′ hexanucleotide repeat follows the underlined and bolded sequence. It is to be understood that this 5′-GGGGCC-3′ or 5′-GGCCCC-3′ hexanucleotide repeat can be repeated more than the number of times present in these sequences.

### C90RF72 (Partial Sequence, Sense)

(SEQ ID NO: 6)
CCCCATTTCGCTAGCCTCGTGAGAAAACGTCATCGCACATAGAAAACAG
ACAGACGTAACCTACGGTGTCCCGCTAGGAAAGAGAGGTGCGTCAAACA
GCGACAAGTTCCCCCCACGTAAAAGATGACGCTTGGTGTGTCAGCCGTC
CCTGCTGCCCGGTTGCTTCTCTTTTGGGGGCGGGGTCTAGCAAGAGCAG
GTGTGGGTTTAGGAGGTGTGTGTTTTTGTTTTTCCCACCCTCTCTCCCC
ACTACTTGCTCTCACAGTACTCGCTGAGGGTGAACAAGAAAAGACCTGA
TAAAGATTAACCAGAAGAAAACAAGGAGGGAAACAACCGCAGCCTGTAG
CAAGCTCTGGAACTCAGGAGTCGCGCGCTAGGGGCCGGGGCCGGGGCCG
GGGCGTGGTCGGGGCGGGCCCGGGGGGGGCCCGGGGCGGGGCTGCGGTT
GCGGTGCCTGCGCCCGCGGCGGCGGAGGCGCAGGCGGTGGCGAGTGGGT
GAGTGAGGAGGCGGCATCCTGGCGGGTGGCTGTTTGGGGTTCGGCTGCC
GGGAAGAGGCGCGGGTAGAAGCGGGGGCTCTCCTCAGAGCTCGACGCAT
TTTTACTTTCCCTCTCATTTCTCTGACCGAAGCTGGGTGTCGGGCTTTC
GCCTCTAGCGACTGGTGGAATTGCCTGCATCCGGGCCCCGGGCTTCCCG
GCGGCGGCGGCGGCGGCGGCGGCGCAGGGACAAGGGATGGGGATCTGGC
CTCTTCCTTGCTTTCCCGCCCTCAGTACCCGAGCTGTCTCCTTC

### C90RF72 (Partial Sequence, Anti-Sense)

(SEQ ID NO: 7)
GAAGGAGACAGCTCGGGTACTGAGGGGGGGAAAGCAAGGAAGAGGCCAG
ATCCCCATCCCTTGTCCCTGCGCCGCCGCCGCCGCCGCCGCCGCCGGGA
AGCCCGGGGCCCGGATGCAGGCAATTCCACCAGTCGCTAGAGGCGAAAG
CCCGACACCCAGCTTCGGTCAGAGAAATGAGAGGGAAAGTAAAAATGCG
TCGAGCTCTGAGGAGAGCCCCCGCTTCTACCCGCGCCTCTTCCCGGCAG
CCGAACCCCAAACAGCCACCCGCCAGGATGCCGCCTCCTCACTCACCCA
CTCGCCACCGCCTGCGCCTCCGCCGCCGCGGGCGCAGGCACCGCAACCG
CAGCCCCGCCCCGGGCCCGCCCCCGGGCCCGCCCCGACCACGCCCCGGC
CCCGGCCCCGGCCCCTAGCGCGCGACTCCTGAGTTCCAGAGCTTGCTAC
AGGCTGCGGTTGTTTCCCTCCTTGTTTTCTTCTGGTTAATCTTTATCAG
GTCTTTTCTTGTTCACCCTCAGCGAGTACTGTGAGAGCAAGTAGTGGGG
AGAGAGGGTGGGAAAAACAAAAACACACACCTCCTAAACCCACACCTGC
TCTTGCTAGACCCCGCCCCCAAAAGAGAAGCAACCGGGCAGCAGGGACG
GCTGACACACCAAGCGTCATCTTTTACGTGGGCGGAACTTGTCGCTGTT
TGACGCACCTCTCTTTCCTAGCGGGACACCGTAGGTTACGTCTGTCTGT
TTTCTATGTGCGATGACGTTTTCTCACGAGGCTAGCGAAATGGGG

In some embodiments, a Met . . . poly-(Pro-Arg) or Met . . . poly-(Gly-Pro) protein comprises an N-terminal amino acid sequence comprising an N-terminal methionine. In some embodiments, a Met . . . poly-(Pro-Arg) protein comprises an N-terminal amino acid sequence comprising MQAIPPVARGESPTPSFGQRNERESKNASSSEESPRFYPRLFPAAEPQTATRQDAASSL THSPPPAPPPPRAQAPQPQPRPGPAPGPAPTT (SEQ ID NO: 41) or a fragment thereof, wherein the sequence is N-terminal to a poly-(Pro-Arg) repeat amino acid sequence. In some embodiments, a Met . . . poly-(Gly-Pro) protein comprises an N-terminal amino acid sequence comprising

(SEQ ID NO: 42)
MRGKVKMRRALRRAPASTRASSRQPNPKQPPARMPPPHSPTRHRLRLRR
RGRRHRNRSPAPGPPPGPPRPRP,
(SEQ ID NO: 43)
MRRALRRAPASTRASSRQPNPKQPPARMPPPHSPTRHRLRLRRRGRRHR
NRSPAPGPPPGPPRPRP,
(SEQ ID NO: 44)
MPPPHSPTRHRLRLRRRGRRHRNRSPAPGPPPGPPRPRP,
or a

fragment thereof, wherein the sequence is N-terminal to a poly-(Gly-Pro) repeat amino acid sequence.
In some embodiments, a C-terminal amino acid sequence comprises a C-terminus amino acid sequence shown in Table 1 or a fragment of a C-terminus amino acid sequence shown in Table 1. It is to be understood that C-terminal amino acid sequences other than those in Table 1 are also contemplated.

Exemplary di-amino acid repeat-containing proteins may comprise a sequence provided in Table 2.

TABLE 2
(GA)ₓWSGRARGRARGGAAVAVPAPAAAEAQAVASG (SEQ ID NO: 8)
(AG)ₓAWSGRARGRARGGAAVAVPAPAAAEAQAVASG (SEQ ID NO: 9)
(GP)ₓGRGRGGPGGGPGAGLRLRCLRPRRRRRRRWRVGE (SEQ ID NO: 10)
(PG)ₓPGRGRGGPGGGPGAGLRLRCLRPRRRRRRRWRVGE (SEQ ID NO: 11)
(GP)ₓ
(PG)ₓ
(GR)ₓGVVGAGPGAGPGRGCGCGACARGGGGAGGGEWVSEEAASWRVAVWG
SAAGKRRG (SEQ ID NO: 12)
(RG)ₓRGVVGAGPGAGPGRGCGCGACARGGGGAGGGEWVSEEAASWRVAVW
GSAAGKRRG (SEQ ID NO: 13)
(AP)ₓAPSARLLSSRACYRLRLFPSLESSG (SEQ ID NO: 14)
(PA)ₓPSARLLSSRACYRLRLFPSLFSSG (SEQ ID NO: 15)
(PR)ₓPLARDS (SEQ ID NO: 16)
(RP)ₓRPLARDS (SEQ ID NO: 17)
MQAIPPVARGESPTPSFGQRNERESKNASSSEESPRFYPRLFPAAEPQTATRQDA
ASSLTHSPPPAPPPPRAQAPQPQPRPGPAPGPAPTT(PR)xPLARDS (SEQ ID NO:
32)
MRGKVKMRRALRRAPASTRASSRQPNPKQPPARMPPPHSPTRHRLRLRRRGR
RHRNRSPAPGPPPGPPRPRP(GP)x (SEQ ID NO: 33)
MRRALRRAPASTRASSROPNPKQPPARMPPPHSPTRHRLRLRRRGRRHRNRSP
APGPPPGPPRPRP(GP)x (SEQ ID NO: 34)
MPPPHSPTRHRLRLRRRGRRHRNRSPAPGPPPGPPRPRP(GP)x (SEQ ID NO: 35)
x = a number between 2-10,000, 5-10,000, 2-5,000, 5-5,000, 2-1000, 5-1000, 5-500, 5-300, 5-200, 10-500, 10-300, or 10-200.

In some embodiments, the one or more di-amino acid repeat-containing proteins are selected from the poly-(Pro-Ala), poly-(Gly-Pro), poly-(Pro-Arg), Met . . . poly-(Pro-Arg) or Met . . . poly-(Gly-Pro) protein. In some embodiments, the one or more di-amino acid repeat-containing proteins are selected from the poly-(Pro-Ala), poly-(Pro-Arg) protein, Met . . . poly-(Pro-Arg) or Met . . . poly-(Gly-Pro) protein.

In some embodiments, the one or more di-amino acid repeat-containing proteins is two or more, three or more, four or more, or five or more, or six or more, seven or more, or eight di-amino acid repeat-containing proteins.

### Subjects

Aspects of the disclosure relate to identification and treatment of a subject, such as a human, with ALS or FTD or likely to develop ALS or FTD. In some embodiments, a subject may have ALS. In some embodiments, a subject may have one or more symptoms of ALS, such as difficulty breathing, difficulty swallowing, muscle cramps, muscle contractions, muscle weakness, paralysis, speech problems, or weight loss. In some embodiments, a subject may not have any symptoms of ALS. In some embodiments, a subject may have a family history of ALS.

In some embodiments, a subject may have frontotemporal dementia (FTD). In some embodiments, a subject may have one or more symptoms of FTD, such as lethargy, aspontaneity, disinhibition, loss of empathy and other interpersonal skills, apathy, progressive nonfluent aphasia, semantic dementia, binge eating, compulsive behavior, tremor, rigidity, muscle spasums, poor coordination, difficulty swallowing, and muscle weakness. In some embodiments, a subject may not have any symptoms of FTD. In some embodiments, a subject may have a family history of FTD.

In some embodiments, a subject may have GGGGCC hexanucleotide repeats within one or both alleles of a C9ORF72 gene (NCBI Entrez Gene ID: 203228). In some embodiments. GGGGCC hexanucleotide repeats are within a promoter and/or intron of the C90RF72 gene. In some embodiments, the number of GGGGCC hexanucleotide repeats is greater than 25, 50, 100, 150, 200, 250, 300, 500, 5,000, 10,000 or more. The number of repeats may be detected using any assay known in the art, e.g., using as a nucleic acid-based assay such as a southern blot (see, e.g., Dejesus-Hernandez et al. Expanded GGGGCC hexanucleotide repeat in noncoding region of C9ORF72 causes chromosome 9p-linked FTD and ALS. Neuron 72, 245 (2011); Renton et al. A hexanucleotide repeat expansion in C9ORF72 is the cause of chromosome 9p21-linked ALS-FTD. Neuron 72, 257 (2011); and Gijselink et al. A C9orf72 promoter repeat expansion in a Flanders-Belgian cohort with disorders of the frontotemporal lobar degeneration-amyotrophic lateral sclerosis spectrum: A gene identification study. Lancet Neurol. 11, 54 (2011)).

### Controls and Control Levels

Aspects of the disclosure relate to comparison of a level of one or more di-amino acid repeat-containing proteins and/or hexanucleotide repeat-containing RNAs to a control level. In some embodiments, the control level is a level of one or more di-amino acid repeat-containing proteins and/or hexanucleotide repeat-containing RNAs in sample, such as a fluid sample or tissue sample, obtained from a healthy subject or population of healthy subjects. In some embodiments, the sample is a blood sample. As used herein, a healthy subject is a subject that is apparently free of disease and has no history of disease, such as ALS or FTD. In some embodiments, a healthy subject is a subject that has 25 or fewer GGGGCC hexanucleotide repeats within a C9ORF72 gene.

In some embodiments, a control level is a level of one or more di-amino acid repeat-containing proteins and/or hexanucleotide repeat-containing RNAs that is undetectable or below a background/noise level obtained using standard methods of detection (e.g., Western blot, qPCR, northern blot, or immunohistochemistry). Such a level could be obtained, for example, by measuring a level of one or more di-amino acid repeat-containing proteins and/or hexanucleotide repeat-containing RNAs in a sample that is known to be free of the di-amino acid repeat-containing proteins and/or hexanucleotide repeat-containing RNAs.

The disclosure also involves comparing the level of one or more di-amino acid repeat-containing proteins and/or hexanucleotide repeat-containing RNAs with a predetermined level or value, such that a control level need not be measured every time. The predetermined level or value can take a variety of forms. It can be single cut-off value, such as a median or mean. It can be established based upon comparative groups, such as where one defined group is known not to have ALS or FTD and another defined group is known to have ALS or FTD. It can be a range, for example, where the tested population is divided equally (or unequally) into groups, such as a subject that has 25 or fewer GGGGCC hexanucleotide repeats, a subject that has 25-50 GGGGCC hexanucleotide repeats, and a subject that has 50 or more GGGGCC hexanucleotide repeats.

### Samples

Aspects of the disclosure relate to determining a level of one or more di-amino acid repeat-containing proteins in a blood sample (e.g., whole blood, plasma, or serum) obtained from a subject. The blood sample may be obtained by any method known in the art, e.g., using a needle or fingerprick device. The blood may be processed before use in the methods described herein. Such processing includes, for example, addition of an anti-coagulant, removal of blood cells, and/or freezing of the blood. However, it should be appreciated that other samples may be used, such as a tissue sample (e.g., brain tissue) or other fluid samples such as saliva, or urine.

Other aspects of the disclosure relate to determining a level of hexanucleotide repeat-containing RNA in sample obtained from a subject. The sample may be a fluid or tissue sample. In some embodiments, the tissue sample is brain tissue. In some embodiments, the fluid sample is blood (e.g., whole blood, plasma, or serum), saliva, or urine. In some embodiments, the fluid sample is a blood sample (e.g., whole blood, plasma, or serum).

### Assays

Aspects of the disclosure relate to performing an assay to determine a level or presence/absence of one or more di-amino acid repeat-containing proteins and/or hexanucleotide repeat-containing RNAs. Assays known in the art for detecting proteins and RNAs (see, e.g., Molecular Cloning: A Laboratory Manual, J. Sambrook, et al., eds., Third Edition. Cold Spring Harbor Laboratory Press, Cold Spring Harbor, New York, 2001, Current Protocols in Molecular Biology, F. M. Ausubel, et al., eds., John Wiley & Sons, Inc., New York. Microarray technology is described in Microarray Methods and Protocols, R. Matson, CRC Press, 2009, or Current Protocols in Molecular Biology, F. M. Ausubel, et al., eds., John Wiley & Sons, Inc., New York) can be used alone or in combination with, techniques and compositions described herein for measuring a di-amino acid repeat-containing protein level.

Assays for detecting protein levels include, but are not limited to, immunoassays (also referred to herein as immune-based or immuno-based assays, e.g., Western blot, immunohistochemistry and ELISA assays), Mass spectrometry, and multiplex bead-based assays. Such assays for protein level detection are well-known in the art. Other examples of protein detection and quantitation methods include multiplexed immunoassays as described for example in U.S. Pat. Nos. 6,939,720 and 8,148,171, and published US Patent Application No. 2008/0255766, and protein microarrays as described for example in published US Patent Application No. 2009/0088329, all of which are incorporated herein by reference in their entirety.

Any suitable binding partner for a di-amino acid repeat-containing protein is contemplated for detection of a di-amino acid repeat-containing protein level. In some embodiments, the binding partner is any molecule that binds specifically to a di-amino acid repeat-containing protein as described herein. As described herein, “binds specifically to a di-amino acid repeat-containing protein” means that the molecule is more likely to bind to a portion of or the entirety of a di-amino acid repeat-containing protein than to a portion of or the entirety of a non-di-amino acid repeat-containing protein.

In some embodiments, the binding partner is an antibody or antigen-binding fragment thereof, such as Fab, F(ab)2, Fv, single chain antibodies, Fab and sFab fragments, F(ab′)2, Fd fragments, scFv, or dAb fragments. Methods for producing antibodies and antigen-binding fragments thereof are well known in the art (see, e.g., Sambrook et al, “Molecular Cloning: A Laboratory Manual” (2nd Ed.), Cold Spring Harbor Laboratory Press (1989); Lewin, “Genes IV”, Oxford University Press, New York, (1990), and Roitt et al., “Immunology” (2nd Ed.), Gower Medical Publishing, London, New York (1989), WO2006/040153, WO2006/122786, and WO2003/002609). Binding partners also include other peptide molecules and aptamers that bind specifically to a di-amino acid repeat-containing protein. Methods for producing peptide molecules and aptamers are well known in the art (see, e.g., published US Patent Application No. 2009/0075834, U.S. Pat. Nos. 7,435,542, 7,807,351, and 7,239,742). The binding partner may comprise a label including, but not limited to, a fluorescent, enzymatic, affinity or isotopic label.

In some embodiments, an assay comprises an immuno-based assay. In some embodiments, the immuno-based assay comprises an isolated antibody specific for one or more di-amino acid repeat-containing proteins. In some embodiments, the isolated antibody specific for one or more di-amino acid repeat-containing proteins is an isolated antibody as described herein in further detail. In some embodiments, the isolated antibody specific for one or more di-amino acid repeat-containing proteins is an isolated antibody specific for an antigen or sequence, or a fragment of an antigen or sequence described in Table 1, Table 2 or Table 3.

Accordingly, a di-amino acid repeat-containing binding partner (e.g., a di-amino acid repeat-containing-specific antibody) can be labeled with a detectable moiety.

Assays for detecting RNA include, but are not limited to, hybridization-based assays such as Northern blot analysis, RT-PCR, sequencing technology, RNA in situ hybridization (using e.g., DNA or RNA probes to hybridize to RNA molecules present in the sample as in FISH), in situ RT-PCR (e.g., as described in Nuovo G J, et al. Am J Surg Pathol. 1993, 17:683-90; Komminoth P, et al. Pathol Res Pract. 1994, 190:1017-25), and oligonucleotide microarray (e.g., by hybridization of polynucleotide sequences derived from a sample to oligonucleotides attached to a solid surface (e.g., a glass wafer) with addressable locations, such as an Affymetrix microarray (Affymetrix®, Santa Clara, CA)). Methods for designing nucleic acid binding partners, such as probes, are well known in the art. In some embodiments, the nucleic acid binding partners bind to a part of or an entire nucleic acid sequence of a hexanucleotide repeat-containing RNA provided herein.

### Treatment

As described herein, it was found that di-amino acid repeat-containing proteins were present in samples of blood from patients with ALS. Without wishing to be bound by theory or mechanism, it is believed that di-amino acid repeat-containing proteins in the blood of subjects with ALS accumulate within the brain parenchyma over time, leading to neuroinflammatory changes, CNS dysfunction, and neuronal death. Accordingly, aspects of the disclosure relate to treatment of a subject having ALS or PTD by decreasing or stabilizing di-amino acid repeat-containing protein levels in the blood of the subject.

In some embodiments, decreasing or preventing an increase of the level of one or more di-amino acid repeat-containing proteins comprises removing the one or more (e.g., 1, 2, 3, 4, 5, 6, 7 or 8) di-amino acid repeat-containing proteins from the blood of the subject. In some embodiments, the one or more di-amino acid repeat-containing proteins from the blood of the subject is removed using a procedure selected from plasmapheresis or a bone marrow transplantation. In some embodiments, it may be advantageous to decrease or prevent an increase of the level of all di-amino acid repeat-containing proteins expressed by a subject. Accordingly, in some embodiments, a method comprises decreasing or preventing an increase of the level of all forms of di-amino acid repeat-containing proteins expressed by a subject.

In some embodiments, the one or more di-amino acid repeat-containing from the blood of the subject is removed using a hematopoietic stem cell (HSC) transplantation. HSC transplantation is the transplantation of hematopoietic stem cells, usually derived from bone marrow, peripheral blood, or umbilical cord blood, into a subject. The source of hematopoietic stem cells may be allogeneic (e.g., from a donor such as a healthy subject). Methods of HSC transplantation are well known in the art (see, e.g., Bishop M R, Pavletic S Z. Hematopoietic stem cell transplantation. In: Abeloff M D, Armitage J O, Niederhuber J E, Kastan M B, McKena W G, eds. Clinical Oncology. 4th ed. Philadelphia, Pa: Elsevier Churchill Livingstone; 2008: chap 32; and Vose J M. Pavletic S Z. Hematopoietic stem cell transplantation. In: Goldman L, Schafer A I. Cecil Medicine. 24th ed. Philadelphia, Pa: Saunders Elsevier; 2011: chap 181).

In order to prepare a subject for HSC transplantation, the HSCs present in the subject may be removed or depleted so that the transplanted cells can become the dominant HSC population in the subject. HSCs in the subject may be depleted, for example, by treating the subject with a chemotherapy, radiation, or both in order to cause the HSC cells of the subject to undergo apoptosis or cell cycle arrest.

In allogeneic HSC transplantation, the HSCs are obtained from a donor. The donor is preferably a healthy subject, such as a subject that is apparently free of disease and has no history of disease, such as ALS or FTD. It is preferable that the donor is HLA-compatible with the subject receiving the transplant in order to reduce the risk of graft versus host disease. HLA-compatibility can be determined, e.g., using HLA typing. HLA typing generally involves examination of at least 8 HLA markers: two A, two B, two C, and two DRB1 markers, and optionally also two DQ markers. HLA typing can be accomplished, e.g., through a blood test. HLA allele identities can be determined using serology or a nucleic acid-based assay. Generally, a match of at least 4-6 markers between host and donor is preferred. In some embodiments, the donor is a subject that has 25 or fewer GGGGCC hexanucleotide repeats within a C9ORF72 gene.

HSCs can be obtained from a donor using any method known in the art. Exemplary methods include bone marrow harvest and leukapheresis (see, e.g., Transfusion. 2003 February; 43 (2): 259-64. Leukapheresis after high-dose chemotherapy and autologous peripheral blood progenitor cell transplantation: a novel approach to harvest a second autograft. Schwella N, Braun A, Ahrens N, Rick O, Salama A). In a bone marrow harvest, the bone marrow is typically removed from the back of one or both hip bones of the donor. Leukapheresis involves separation of HSCs from blood obtained from the donor using, e.g., continuous flow centrifugation or filtering. The growth factor G-CSF may be administered to the donor to stimulate the growth of new HSCs so that more HSCs are present in the blood. Once obtained, the allogeneic HSCs are then administered to the subject receiving the transplant. Any suitable method of administration known in the art is contemplated, e.g., by central venous catheter.

In some embodiments, during or after HSC transplantation, the subject receiving the HSC transplant may receive additional treatments and/or therapies, such as antibiotics, antifungals, antivirals, blood transfusions and/or immunosuppressive therapies. Such treatments and/or therapies may help to prevent infection and/or graft versus host disease during a HSC transplant recovery period.

In some embodiments, the HSC transplantation is bone marrow transplantation. In some embodiments, the bone marrow transplantation is an allogeneic bone marrow transplantation.

Plasmapheresis is a medical procedure that occurs outside the body (an “extracorporeal therapy”) and refers to the removal, treatment, and return of (components of) blood plasma from blood circulation. Plasmapheresis is well-known in the art and has been used to treat several diseases including Goodpasture's syndrome, myasthenia gravis, Guillain-Barre syndrome, lupus, and thrombotic thrombocytopeniarpura (see, e.g., Madore, Plasmapheresis Technical aspects and indications, Crit Care Clin 18:375-392, 2002). During plasmapheresis, blood is initially taken out of the body, e.g., through a needle or previously implanted catheter. Plasma is then separated from the blood cells, e.g., by using a cell separator. After plasma separation, the blood cells are combined with a replacement fluid and readministered to the subject. The replacement fluid may be either the separated plasma treated to remove disease-associated components or a replacement plasma (also called plasma exchange).

Exemplary procedures used to separate the plasma from the blood cells include:
1) Discontinuous flow centrifugation: One venous catheter line is used. Typically, one or more batches of blood are removed at a time and centrifuged to separate plasma from blood cells. The blood cells are then combined with the replacement fluid and returned to the subject.
2) Continuous flow centrifugation: Two venous lines are used. Plasma is continuously spun out of the blood and the separated blood cells are fed through a line that combines with a replacement fluid before return to the subject.
3) Plasma filtration: Two venous lines are used. The plasma is filtered using standard hemodialysis equipment, e.g., a parallel-plate or hollow-fiber filter. The separated blood cells are fed through a line that combines with a replacement fluid before return to the subject. The filters usually have pores of 0.2-0.6 μm diameter, sufficient to allow passage of plasma, while retaining cells. Several membrane plasma separators are commercially available (e.g., Plasmaflo from Asahi Medical Co., Ltd., Tokyo, Japan; Plasmax from Toray Industries, Tokyo, Japan; CPS-10 from Baxter. Deerfield. IL, USA; Plasmaflux from Fresenius Medical Care AG, Bad Homburg, Germany; Prisma TPE 2000 from Hospal, Lyon, France).

If the separated plasma is to be used as the replacement fluid, the separated plasma is first treated to decrease the levels of di-amino acid repeat-containing proteins present in the separated plasma. In some embodiments, decreasing the levels of di-amino acid repeat-containing proteins present in the separated plasma comprises contacting the separated plasma with one or more isolated antibodies specific for a di-amino acid repeat-containing protein as described herein, whereby the di-amino acid repeat-containing proteins present in the separated plasma bind to the one or more isolated antibodies. In some embodiments, a binding partner for the one or more isolated antibodies is contacted with the separated plasma. A binding partner for the one or more isolated antibodies may be, for example, a capture moiety such as biotin or streptavidin, protein A, or a secondary antibody specific for the one or more isolated antibodies. Such binding partners allow for the one or more isolated antibodies to be removed from the separated plasma.

In some embodiments, the one or more isolated antibodies are attached to a filter, column, and/or solid support. In such embodiments, the separated plasma is contacted with, the filter, column, and/or solid support, whereby the di-amino acid repeat-containing proteins bind to the isolated antibodies attached to the filter, column and/or solid support. Without wishing to be bound by theory, it is believed that the di-amino acid repeat-containing proteins may form aggregates in the blood. Accordingly, the di-amino acid repeat-containing proteins may be removed from the separated plasma using a filter, such that the aggregates are isolated from the separated plasma.

In some embodiments, a subject expressing one or more di-amino acid repeat-containing proteins may develop autoantibodies. In some embodiments, autoantibodies to one or more di-amino acid repeat-containing proteins may be removed from the separated plasma. Autoantibodies may be removed using any method known in the art, e.g., using a binding partner (e.g., bound to a solid support or attached to a tag) that recognizes the autoantibodies. In some embodiments, the binding partner may be one or more di-amino acid repeat-containing proteins as described herein.

If plasma exchange is to be used, the subject receives replacement plasma. Replacement plasma may be, e.g., donor plasma or a solution of albumin (e.g., 5-70% albumin in saline). An exemplary replacement plasma is 5% albumin combined with 0.9% saline in a 50%: 50% (vol:vol) solution. Medication to keep the blood from clotting (e.g., an anticoagulant such as citrate, acid-citrate dextrose or heparin) may be given to the subject or contacted with the blood of the subject during the procedure.

In some embodiments, decreasing or preventing an increase of the level of one or more di-amino acid repeat-containing proteins comprises decreasing a level of a hexanucleotide repeat-containing RNA. Decreasing a level of a hexanucleotide repeat-containing RNA may comprise administration of an effective amount of an inhibitory nucleic acid molecule such as an shRNA, an siRNA, miRNA, or an antisense nucleic acid molecule that targets the hexanucleotide repeat-containing RNA.

Methods for producing shRNAs, siRNAs, miRNAs, and antisense nucleic acid molecules are well known in the art (see e.g., Sambrook, Fritsch and Maniatis, MOLECULAR CLONING: A LABORATORY MANUAL, (Current Edition); CURRENT PROTOCOLS IN MOLECULAR BIOLOGY (F. M. Ausubel et al. eds., (Current Edition)); Oligonucleotide Synthesis (N. Gait, ed., Current Edition); Nucleic Acid Hybridization (B. Hames & S. Higgins, eds., Current Edition); Transcription and Translation (B. Hames & S. Higgins, eds., Current Edition). In some embodiments, a nucleic acid inhibitor comprises or corresponds to at least a portion of sequence of a target hexanucleotide repeat-containing RNA sequence or comprises at least a portion of a sequence complementary to a target hexanucleotide repeat-containing RNA sequence.

In some embodiments, treatment may comprise decreasing or stabilizing a level of an autoantibody to one or more di-amino acid repeat-containing proteins in a subject. A level of autoantibody may be decreased or stabilized using any method known in the art. In some embodiments, decreasing or stabilizing a level of an autoantibody comprises administration of an effective amount of atacicept, belimumab, blisibimod, BR3-Fc, rituximab, ocrelizumab, atumumab, epratuzumab, corticosteroid (e.g., prednisone), mycophenolic acid, methotrexate, cyclophosphamide, azathioprine, and/or cyclosporin. In some embodiments, decreasing or stabilizing a level of an autoantibody comprises plasmapheresis.

### Antibodies

Aspects of the disclosure relate to isolated antibodies specific for a di-amino acid repeat-containing protein (e.g., a RAN protein) selected from a poly-(Gly-Ala), poly-(Gly-Pro), poly-(Gly-Arg), poly-(Pro-Ala), poly-(Pro-Arg), Met . . . poly-(Pro-Arg) or Met . . . poly-(Gly-Pro) protein. The isolated antibody may recognize a region or regions of the di-amino acid repeat-containing protein (such as a repeat sequence or the C-terminus) or may recognize the entire di-amino acid repeat-containing protein.

An antibody that “specifically binds” to a target or an epitope is a term understood in, the art, and methods to determine such specific binding are also known in the art. A molecule is said to exhibit “specific binding” if it reacts or associates more frequently, more rapidly, with greater duration and/or with greater affinity with a particular target antigen than it does with alternative targets. An antibody “specifically binds” to a target antigen if it binds with greater affinity, avidity, more readily, and/or with greater duration than it binds to other substances. For example, an antibody that specifically binds to a poly-(Gly-Ala) protein or an epitope therein is an antibody that binds this target antigen with greater affinity, avidity, more readily, and/or with greater duration than it binds to other antigens or other epitopes in the same antigen. It is also understood by reading this definition that, for example, an antibody that specifically binds to a first target antigen may or may not specifically bind to a second target antigen. As such, “specific binding” does not necessarily require (although it can include) exclusive binding. Generally, but not necessarily, reference to binding means specific binding. In some embodiments, antibodies described herein have a suitable binding affinity to a di-amino acid repeat-containing protein (e.g., a RAN protein). As used herein, “binding affinity” refers to the apparent association constant or K_{A}. The K_{A} is the reciprocal of the dissociation constant (K_{D}). The antibody described herein may have a binding affinity (K_{D}) of at least 10⁻⁵, 10⁻⁶, 10⁻⁷, 10⁻⁸, 10⁻⁹, 10⁻¹⁰ M, or lower. An increased binding affinity corresponds to a decreased K_{D}. Higher affinity binding of an antibody to a first target relative to a second target can be indicated by a higher K_{A} (or a smaller numerical value K_{D}) for binding the first target than the K_{A} (or numerical value K_{D}) for binding the second target. In such cases, the antibody has specificity for the first target (e.g., a protein in a first conformation or mimic thereof) relative to the second target (e.g., the same protein in a second conformation or mimic thereof; or a second protein). Differences in binding affinity (e.g., for specificity or other comparisons) can be at least 1.5, 2, 3, 4, 5, 10, 15, 20, 37.5, 50, 70, 80, 91, 100, 500, 1000, 10,000 or 105 fold.

Binding affinity can be determined by a variety of methods including equilibrium dialysis, equilibrium binding, gel filtration, ELISA, surface plasmon resonance, or spectroscopy (e.g., using a fluorescence assay). Exemplary conditions for evaluating binding affinity are in, e.g., TRIS-buffer (50 mM TRIS, 150 mM NaCl, 5 mM CaCl2 at pH7.5). These techniques can be used to measure the concentration of bound binding protein as a function of target protein concentration. The concentration of bound binding protein ([Bound]) is related to the concentration of free target protein ([Free]) and the concentration of binding sites for the binding protein on the target where (N) is the number of binding sites per target molecule by the following equation:

[Bound]=[N][Free]/(Kd+[Free])

It is not always necessary to make an exact determination of K_{A}, though, since sometimes it is sufficient to obtain a quantitative measurement of affinity. e.g., determined using a method such as ELISA or FACS analysis, is proportional to K_{A}, and thus can be used for comparisons, such as determining whether a higher affinity is, e.g., 2-fold higher, to obtain a qualitative measurement of affinity, or to obtain an inference of affinity, e.g., by activity in a functional assay, e.g., an in vitro or in vivo assay.

In some embodiments, the isolated antibody is specific for a di-amino acid repeat-containing protein selected from a poly-(Pro-Ala) poly-(Pro-Arg), Met . . . poly-(Pro-Arg) or Met . . . poly-(Gly-Pro) protein.

In some embodiments, the isolated antibody is specific for an antigen comprising a di-amino acid repeat and/or C-terminus sequence or fragment thereof as defined in Table 1. In some embodiments, the isolated antibody is specific for an antigen comprising a sequence or fragment of a sequence defined in Table 2.

In some embodiments, the isolated antibody is specific for an antigen in Table 3 or in FIG. 28. In some embodiments, an antigen in Table 3 does not contain an N- and/or C-terminal modification.

TABLE 3
Di-Amino Acid Repeat-Containing Protein Antigens

| di-amino acid | | | Antigen location |
|---|---|---|---|
| repeat- | | | in di-amino acid |
| containing | | | repeat-containing |
| protein | Label | Antigen | protein |
| Poly-(Gly-Arg) | GGGGCC F1 | Ac-RGRGRGRGRGRGRGRGRC- | Repeat sequence |
| | repeat | amide (SEQ ID NO: 18) | |
| Poly-(Pro-Arg) | GGGGCC- | Ac-RPRPRPRPRPRPRPRPRC- | Repeat sequence |
| | AS F2 repeat | amide (SEQ ID NO: 19) | |
| Poly-(Pro-Ala) | GGGGCC- | H2N- | Repeat sequence |
| | AS F1 repeat | APAPAPAPAPAPAPACKKKK- | |
| | | amide (SEQ ID NO: 20) | |
| Poly-(Gly-Pro) | GGGGCC F3 | H2N- | Repeat sequence |
| | repeat | GPGPGPGPGPGPGPGPGCKK- | |
| | | amide (SEQ ID NO: 21) | |
| Poly-(Gly-Pro) | GGGGCC F3 | Ac-CRRRRWRVGE-OH (SEQ ID | C-terminus |
| | CT | NO: 22) | |
| Poly-(Pro-Ala) | GGGGCC- | Ac-CYRLRLFPSLFSSG-OH (SEQ | C-terminus |
| | AS F1 CT | ID NO: 23) | |
| Poly-(Gly-Arg) | GGGGCC F1 | Ac-CRVAVWGSAAGKRRG-OH | C-terminus |
| | CT | (SEQ ID NO: 24) | |
| Poly-(Pro-Arg) | GGGGCC- | Ac-CRPRPLARDS-OH (SEQ ID | C-terminus |
| | AS F2 CT | NO: 25) | |
| Poly-(Gly-Ala) | GGGGCC F2 | Ac-CSGRARGRARGGA-amide | C-terminus |
| | CT | (SEQ ID NO: 26) | |

F1 = reading frame 1, F2 = reading frame 2, F3 = reading frame 3, AS F1 = anti-sense reading frame 1, AS F2 = anti-sense reading frame 2, AS F3 = anti-sense reading frame 3.

An isolated antibody may be a monoclonal or polyclonal antibody, or an antigen-binding fragment thereof. An antigen-binding fragment thereof includes, for example, an Fab, F(ab)2, F(ab′)2, Fv, single chain antibody, Fab fragment, sFab fragment, Fd fragment, scFv, or dAb fragment. Methods for producing polyclonal and monoclonal antibodies and antigen-binding fragments thereof are well known in the art (see, e.g., Sambrook et al, “Molecular Cloning: A Laboratory Manual” (2nd Ed.), Cold Spring Harbor Laboratory Press (1989); Lewin, “Genes IV”, Oxford University Press, New York, (1990), and Roitt et al., “Immunology” (2nd Ed.), Gower Medical Publishing, London, New York (1989), WO2006/040153, WO2006/122786, and WO2003/002609). Also encompassed are antibodies made by recombinant means such as chimeric antibodies (variable region and constant region derived from different species) and CDR-grafted antibodies (complementary determining region derived from a different species) as described in U.S. Pat. Nos. 4,816,567 and 5,225,539, which are incorporated herein by reference in their entirety. Also encompassed are humanized antibodies, typically produced by recombinant methods, wherein the human sequences comprise part or all of the antibody. Also included are fully human antibodies, such as those produced in genetically-altered mice (see PCT Application No. 93/12227, which is incorporated herein by reference in its entirety).

In some embodiments, an isolated antibody specific for a di-amino acid repeat-containing protein is a rabbit polyclonal antibody as listed in Table 4.

TABLE 4
Di-Amino Acid Repeat-Containing Protein Rabbit
Polyclonal Antibodies

| Antigen | Animal | Titer |
|---|---|---|
| GGGGCC F1 repeat | H3147 | 1,575,500 |
| GGGGCC F1 repeat | H3148 | 1,956,500 |
| GGGGCC-AS F2 repeat | H3149 | 2,399,600 |
| GGGGCC-AS F2 repeat | H3150 | 3,225,000 |
| GGGGCC-AS F1 repeat | H3151 | 660,200 |
| GGGGCC-AS F1 repeat | H3152 | 2,082,600 |
| GGGGCC F3 repeat | H3154 | 752,300 |
| GGGGCC F3 repeat | H3155 | 590,500 |
| GGGGCC F3 CT | H3156 | 231,300 |
| GGGGCC F3 CT | H3157 | 616,700 |
| GGGGCC-AS F1 CT | H3158 | 6,300 |
| GGGGCC-AS F1 CT | H3159 | 32,800 |
| GGGGCC F1 CT | H3160 | 573,900 |
| GGGGCC F1 CT | H3161 | 363,000 |
| GGGGCC-AS F2 CT | H3162 | 2,261,700 |
| GGGGCC-AS F2 CT | H3163 | 176,300 |
| GGGGCC F2 CT | H3164 | 1,549,500 |
| GGGGCC F2 CT | H3165 | 115,700 |

Antibodies may be produced in bacterial cells, e.g., E. coli, or eukaryotic cells, such as yeast cells or mammalian cells. In one embodiment, antibodies are produced in mammalian cells. Mammalian host cells for expressing the antibodies or antigen-binding fragments thereof include Chinese Hamster Ovary (CHO cells) (including dhfr-CHO cells, described in Urlaub and Chasin, 1980, Proc. Natl. Acad. Sci. USA 77:4216-4220, used with a DHFR selectable marker, e.g., as described in Kaufman and Sharp, 1982, Mol. Biol. 159:601 621), lymphocytic cell lines, e.g., NS0 myeloma cells and SP2 cells, COS cells, and a cell from a transgenic animal, e.g., a transgenic mammal. Antibodies can also be produced by a transgenic animal. For example, U.S. Pat. No. 5,849,992 describes a method of expressing an antibody in the mammary gland of a transgenic mammal.

Isolated antibodies of the disclosure may also have a detectable label attached thereto. The label may be, for example, a fluorescent, enzymatic, affinity or isotopic label. Examples include fluorescein isothiocyanate (FITC) for detection by fluorescence, horseradish peroxidase which allows detection by cleavage of a chromogenic substrate, radioisotopes such as I¹²⁵ for detection by autoradiography and avidin/biotin for antibody detection and affinity purification of antigens and antigen-bearing cells.

Also encompassed by the disclosure are hybridoma cell lines producing a monoclonal antibody specific for a di-amino acid repeat-containing protein selected from a poly-(Gly-Ala), poly-(Oly-Pro), poly-(Gly-Arg), poly-(Pro-Ala), poly-(Pro-Arg) protein, Met . . . poly-(Pro-Arg). Met . . . poly-(Gly-Pro), a C-terminal peptide of a di-amino acid repeat-containing protein as described herein, and/or a combination of two or more thereof.

In some embodiments, an isolated antibody is an isolated auto-antibody obtained from a subject having ALS, wherein the isolated auto-antibody is specific for one or more di-amino acid repeat-containing proteins as described herein.

In some embodiments, an isolated antibody described herein is contained within a buffered solution. In some embodiments, an isolated antibody described herein is attached to a solid support (e.g., the surface of a plate or a bead).

### Transgenic Mouse

In another aspect, the disclosure relates to a transgenic mouse comprising a human C90RF72 gene comprising a GGGGCC hexanucleotide repeat sequence. In some embodiments, the mouse comprises a human C9ORF72 gene comprising a GGGGCC hexanucleotide repeat sequence and flanking human sequences on the 5′ and 3′ end of the human C9ORF72 gene. In some embodiments, the flanking human sequences on the 5′ and 3′ end are each independently at least 1kilobases (kB), at least 5 kB, at least 10 kB, at least 20 kB, at least 30 kB, at least 40 kB, or at least 50 KB in length. In some embodiments, the flanking human sequences on the 5′ and 3′ end each independently comprise a promoter capable of driving transcription of the human C90RF72 gene in the sense and anti-sense direction, respectively. Accordingly, in some embodiments, the transgenic mouse expresses both sense and anti-sense transcripts (e.g., 5′-GGGGCC-3′ and 5′GGCCCC-3′ hexanucleotide repeat-containing RNAs described herein). In some embodiments, the human C90RF72 gene and flanking sequences comprise the sequence below, wherein (GGGGCC) n indicates the location of the GGGGCC hexanucleotide repeat sequence:

Chr9: 27,527,137-27,625,470 (reverse complement)
(SEQ ID NO. 63)
AAGCTTGATAATATTATCAAATATTAGATAAATGTAATATTAGAAGAAAACTTTTTTGAAAAGATATATAAAAAT
AATTTCATTCAAAATTTTTATATTTAATTTAAATTTTTAATGAAAATATATCTAAGTTTTGTACGCTTTAAATGT
AATTATGTTTGATAATTTAATCATTTACTATTCGTTCTCTATTGCTGCCCTAACAAATTACCATAGTTCAGTGGC
TTACAAAACACAAATTTATTATCTTACCATTCTGTGAGTCAAAATTCCAAAATAGGTGTCACTAGGCTAAAATGA
AGGACTGCATTTCTTCCTGCAGGCTCCAGGAGAGATCTATGTCTTACTCTTTTCGGCTTCTAAAGGCTGCCCACA
TTCCTCGACTAGTGGCGTCCCTCCTTCGTCTCTAAACCCAGCAACAACAGGTTGAGTCCTCATGTCACATCTTTC
TTACCTTTCTGTCATCTCATCTCGCTGACTGCTGCTGGGAAAAATTCTCCACTTTTAAGGGCTATCATGATTAGA
CTATGCCCACTAGATAATACAAGATCTCAGATCCTTAACTTCCATCACATCTGCAAAGTCGCTTTTGCCTCATAA
AAGAGTCTGAGGTTTAGACGGGAGATCTTAAGGGGGCTATTAATATGCCTACCATAATCACTGAGAATAAGTACA
AGTTAAGATTATAATAGCAATAGAATATACAAACGTGAAGCTCCAAAAGAACAACAACAACAAAAAAGGTGAACA
GGAAAAAGAAACTGAAAATCTTTAAAAAGGCAGTCTGTTTAAATCTATAAAAACTGGAAAAAAATGAGAGTGGAC
AAATATCTGGTAAGCATGATGGACTTAAAATTTGTGACTAGGGCATTACATTTTTTATATTAATATAATGAAGAT
TGAATTACTGATCAAAACAATTAAAAAGCAAGAGAACTATTCTCATCAAATCTGCAACACGAAAAGTTCAGACAA
AATTCCAACAACTTCACATTCTGAACTAAATGAGGACTAATTACCAGTTCGAGCAATGAGAATATATGAGGTCCT
CCGTTTGCACTTTGCCAGGGATCTGAAAACGTTGGGAGTAGGTCGGCTTCACCCTGAAGCCAGACCATCGACAGC
CAGTTTTCCCCCCTTCTCCACCCACAGGTCTTTAGGCCCTCATCCTTCCCAGCCTCAGAACTAGTCTCCAAAGAA
GAGGAAAGTTAGAGGAGAGAGTAAATCGTTGAATAGGATGAAGGAGATGTGGGAAAAAGAAAAAGAGAGGCTGCA
AGAGAGAGGGTCCCAGGGATAACTCTGCTCTTGGAAGGGTGGCCACAGTCATGTGGTCCCAAGAGGCAACAACAA
GCTTAGGAAGCCAGAGAAACCAGTTACAATCACTGCTACTCTTTTCGATTCTGTGTTGTTTAAGAAATATCACCC
GCCAGGAGTTCTCCAGAAACATTTTCCCTGATTCCATGTAAGTGCTCAACCAGTGAATGGTAATCCCATTTTGGT
TTAGTCTGTACCATCCCCTATTCCAAAATAAAGGGAAAAATGGTGGGTTTATATCTTAAATTTTCTACTTTACTA
AACTCAAGGGAAATAGCCAAGCAAAAACGAAAGCTGAGACTCTTGCTAATTATCCTTTCCATAGAATGTTTGCTA
AAATTCCTTGTCAAGGAAGGAATAACAAAGCTAGTCCACGCTCTGTATAGGGTGTTTCCAATTAGTTATACTTTA
AAGTATAAGTATTTAACAAAATCTATAAATTTTGTTAATTATTTACTTGTAGTGAAAAATGAGCCATTCTCAAGC
AAATCACTTTTTATTACACATTCCAGAGAATAACCATAAAAGGACATTTATTATAGCAAAAATAACCACATCTGG
ATGGAACTTCAATCACCAGTATTTACTAAATAAATGCCCAGAAAAAAAATAGTTCATCTTTAATTTCAGTCATCA
TTAATAAAAGCTGAAGTACCTCTTCAGATCTTTTGATCATTTTCTGTTGGATTGTTTTCTTTTTACTGAGTTGCA
AATGCTCTTTATATATTTTGGATACAAAGCTTTATCACATAGGCATTTTGCAAGTATTTTTTCCAAGTTTTTTTA
CTTTTCATTTATTTAATAATATCTTTCAAAGAACGGGAATTTTATAATTTTTTATGAAGTCCATTTATAATTTTT
TCTTTTATGGGTTGGTGGGGGTTGGGGGTTGTGTTGTCCTAAGAAATCTTGGCTCAACACAAAAAGATTAGTTTC
TATATTTTCTTCTAGAAGTTTTATAGTACGATCTCAGATCCATTTCAGATGATGAATAAGCACATAAAAAAAGGA
TACTCATCGTTAGTCATTAGAGAAATGCATATTAAAACCATAAGGAAATACTACTATATACATATATTAGATAGG
ATGAAGAGCAACTGGAATCTCATACAGTGCTGATTGAAATGCAAAATGGCAAAACAACTTTAGAAACCAATTTGG
AAGCAGCTGTACTGACATGGAATTTTGAGCTGGAAGAATCTTAGAAAAAGAATACTTTACCACCTCCCCCATTCT
CTTCACCCTGGGGAACTGTTAAATGAGGAAATTGTGGTTCAAGGAGGAACTTGTCTATATGCTTTCTCAGCTTTC
CCGTGGTAATTACCATCTTGATAATATAACGTAATGTATGTATATGTTATCAAATAATATAATATCTTCATCATA
TATTTATCATCTTCATAATGTTAGCTGTCTAGTGGTAACTTTTTTTTGCTCTTTATTGCCTCCCTCTTTTTTCCC
TCTTTGTTGTTTTTTGTCATACAATTATGATATATGTGTATATATTCTCACTGTAAAGATGTAAACAACACAAAG
ATTATTGAACAAATCACGAAAGTAACCCTTCCTTCATTCTTACCCTATCCAACCCTCATCTCCTCAGAAGAATAC
ACCATTTTAGTTGTAAATGTTTTTCTAGCTCTTTTTCAATGTTTCTACCTATATGCATGTATGTATAATGTATAT
ACATACATATATACATACATATTGATATATACATATATAGAGGTATGGTTTTTTAACTTAAATGGAATTGCATTG
TGGATATTGTCCTATGACTTGCTTTCAACCAAATTATATGTCTTGGAAATACATACATATATTTAAAAAATATGT
TATGTATATGTAACATACTATATGTGCATAATATATATTACATAGATATAATAAGGCCTAGGAAGAAATTGTGTG
CAACCTCTAGTACATCTTCCTCTATATCTACTGTACATACATACAACCCATTCTTTTTTTAATTTTTTTATTTTT
TTAGACAGAATCTTGCTCTGTCGCCCAGGCTGGAGTGCAGTGGCACAATCTCGGCTCACTGCAAGCTCCACCTCC
TGGGTTCACGCCATTCTCCTGCCTCAGCCTCCCAAGTAGCTGGGAATACAGGCACCTGCCATCAGGCCCAGCTAA
TTTTTTTTTGTATTTTTAGTACAGATGGGGTTTCACCGTGTTAGCCAGGATGGTCTCCATCTCCTGACCTCGTGA
TCCGCCCACCTCATCCTCCCAAAGTGCTGGGATTTACAGGCGTGAGCCACCGCGCCCAGCCACAACTCATTGCAG
AGTAGTCCAAAATATGGATGGACTGTAGCTTAATTACTTATTCTCCCATTGATAGACACTTAGGACTTTTCTAAT
TTTTATAATTTAAAAATATGCTGCAATTAACAAACATTCTTGTGTATCTTTTTGCTGTATGTATGCATATTTCTT
TAGTATGGGTTTTGGAAGAGGAATCACAAAGGAGGCATAGAATATAAATATTTTTATTTTGAAAAATACAGTTGT
AATTTAATAACCCACCAAAAGACTCTAACAGTTTAGATTCACATCAACAGTGTAAGAACATGTCTGTTTTACTGC
ATCCTTACCCCCACTGGTTATAATACTTTTAATTAACAATCTTATGGATGAAGAATACTATCGCAATGTTGTTTT
AATGCATTTTTCCAATTACTAGTGAGATTGAACATTAATTCTTTTATTTTATGGATCACTGGCTTTTCTCCTTCT
GTGAACTACCTGTTCACATCCTCTGCTTTTCAGCTCTTGAGCTGTTATCTTTTTCTTATTGATTTATATGAGCTC
TTTATATATTCAAGATGTTAATCATTTGTATTTTATGTATATGGCAATGATTTTCTTCCAAACCAATGCTTGTCT
TTTATTTATTTATTTATTTATTTATTTATTTGAGACCGAGTCTCGCTCTGTCGCCCAGGCTGGAGTGCAGTGGCG
CGATCTCGGCTCACTGCAAGCTCCGCCTCCCGGGTTCACGCCATTCTCCTGCCTCAGCCTCCTGAGTAGGTGGGA
CTACAGGCGCCCGCTGCCACACCCGGCTAATTTTTTGTATTTTTAGTAGAGACAGGGTTTCACCGTGTTAGCCAG
GATGCTCTCTATCTCCTGACCTCGTGATCCGCCCGCCTCGGCCTTCCAAAGTGGTCGGATTACAGGCATGAGCCA
CCACGCCTGGCCAATGCTTGTCTTTTTATCTCTGTTTATGGCATCTTTCATACTATGGACATTTTTATTTTTATT
TTTTATGTTGATTTATTCTTGAATTGTATACATGTTAATTATACCTAAGTTATTGTAATACCCTTAAAGCCAAGT
TCTACACATATATTTAATTTGCTTTCCCAATAGGTCTCTGAGGGAACACATTTTTTCAAATCACTTTGTTTCATC
TTTTTTAGGTGTTGATCAATTATTAAGGAGTTTGAAATAATCATTTAAACGGAATTCTTCAGATGAAAACATAAA
GACATTTATCGGGTCAGAGCATTGGTCGGTTCACATACTCAGGATCAGTGGCCTGGGTGGGCAGGCACTGGGTGA
ATGGAGAGCTGCAGGTATTGGAAGAGAGCCCAGTTGGATATGTAGTTTCCAAAGATCATCAAGGCAGACAACCAA
AGGGAAACCGTGGGAAACACCTGCTTTGGGCCATCTAAGATGAGATGATAAAGTAAGGAAAGAGTTGAGCCCAAC
ACAGTGATAGCCAATCTGAAAGCGGGCAGAACTGACAAGACCAAACAAGTAGGTGAACTGGCTGCAGGCAGCCAG
CCACCACAGGGACAGCGTGTACTCCAGGGACAAGCTCAAGGCTATAGGTAGTTAGTTCAAGGCTACTAGGGTGAG
AAGAGCAGGAACTGAGTTCTATACCAGTGCTTCTCAAAACTAATGTGCATCCTAATCACCTGGAAATCTTGTAAA
AATGTAGATTCTGATTCAGTGAGTCTGAAGCAGAGCTTAAGATACTACATGCTTAACAAGAGCCTAGTTGATGCT
GACACTGCTGGTCCCTGGAGCTCTCTTTGAGTAGCAGGCTTCTGGAAGGCTTGTGTCACTAAGCACAGAGAAGCC
TCACTTATCAAATCTGCACCAAAACAGGAAAACTAATGTGAAGAATAATGTGATGCACACGTCAGAGCATGAGGC
AGTTGCTTTGTCCCTGAGGTTGCGCTCCAGATGGCTTCCTAAGATGCGACAGGCTGATCTTGTGCGTGGGGGTCC
CGGAGGCTTGGGCCACGGGAGAGACAGGACCTCAGAGGCTGGGAGACAGGCAGAGACAGAAGAGTGACATCCTGC
TGCTTTTGAATTTGCACATTCTGTAGAATAATAACAGCAGTAAACTGTTACACAATATCTATTCTCAGCATCTTG
AAGCCCTTTTCACATATTGTTACTTCCATTAATGGGGCCCTTTGCTGCTATTCTACTTTTCTCTTCAGCTATCAA
CAATATGGCTTTCCACACCTCCATCAGACAGTAGCCAGATGAAATAAAATGTGCCAGAATGAAAACTTGTTCATT
TGTCTACTTTTTGCCAAGACTAGACAGGCAGGAAATTGAATGTATTTTTACAGAAAAGGTTTTCAAAACTTTTTC
CCCTCTGTGGCTCATTTAGGTAAACTAAAAGGCATAAGACCCACCTAAAACATGGGTTCCCGCTTTTTATTGGAG
AAAGAACATAGTACTTTAAAAAAATACATAAAATAATAAAAAGGAAAGACAAAGATAATGAAGGTTGTACATGGT
ACCAAATTTTTGTATCCCATAATAACACATGAGTAGATCACTACTAAGTAGGTTTTAGTGACATATAGGAAACAT
TAAAATCTACAGAAATTTGCATTATTTTCTGTCAAAAAGGATCATTTCACAGCCTTTCAGGGGGAACCCATTGCC
CACAGGAACTCATGCATTCCATGCTTTGAGGATCACTAGATCTAAGAAGCCTTCCTTGGAGGTTCTAGCCTCCAA
CCCTTATTTTAGTAAAAGAAGCTCCAGTTTTATCTGTTTCTAAGTCAGACTACCACACAACATTGGGCTTAAAGA
AAGGTTTCCAGGGCTAAAGCAGACTTTGAGGATTACTAATTCCGAGTTAAATTTCTGTGTATTATCTCTGGATTT
GACTTATTCACACTGGACTATCACTCATAAATATACATAATACAGAGTTAACTATTTAAATTTATAAAGAGAGTA
TTTTCCTTTTTTATGAGCAAAACATGCTGCCAACTACTTGGACCACATACTGATCCATAAATACTGACAGCTTTG
TAATTGGAAATAATAAATACACACTAATGAAGCATCTCAAAAGGGAAGAGCCACAGGTAATCTGAGTGATTAGGC
ATTCATGTTAGGTTAGGCTTTGATCATTGTTTTTAATCGCAATTTCATTGCAGTGCATCTATAAATCCATGTCCA
GAAGTATGAAGTGGTTCTATAGTAAGAATAAGATGCTACAGATAATGCGACTAAATAAGACACTATAGGTAATGA
CACAGATTCAAGTCTTATTGTTGATGGGAAGAGGTCAATAATGGATGATATAATATACTACAGCAATGAGAATTA
TTGAATGTTTTCCAGACTCACTTGTATAATTGGCCATAACAGCAAACAAAAAACAGGTTCTGATAGCAAAATGAT
ATACAGTACTAACAAAGGTGAATCTTGAGGTGAACCTTCTCTTTATAAGTTTAAATAGTTTACCCCCGACCTTTT
CCCATAGTAGAACAGCCTAAAAAGTATCTTTCAGTAGAATGCTAGTGCTTATGAGGTTTTCTTAAGATATCATTT
TTCAATTAAAATTTATTTCACAAAAGACTCACATCCTTGCCAGCCTTCAGGGTGAGTGTTGATTCAGGCTGTGTC
CAACGGCAACGATGAGTGAACTTCTCACCCTCAGAATCACATGAGCATTCCTGAGATGTTTTATCAGAGTGATAC
CAACTTCATTATTAGAATATTGAGTCCCTATTTCCTATATTCAATGTCCTTTCAAGCCCTAACTTTGTCCGGGTT
GAAGGCAAAGATCCAAATAATCACATTTGTCTTTGATAACTGAAACTGGGAGAACTGGGACTGTCTCAAGAGTTC
TACGTGACTGTAGGTTGCAAGTACTGTGGTTGCATCTCCAAATATTAACCAATCCCAGTGACAATTCAATGGGGT
CTCCTGAACCATGATCCTCATGTCTCCAGTGAAGGAAATGGGCAAAGGGGATTCAAAAATCCCTTTTGGAGGAAT
AGGAAACTTCTGCTTTCCTTCATTTCATAACATTTGCGATGGAACAAAGGCTTTTTTAGAATGGAGCAACCAGAT
CCTTTTTTGGGGGAATCAGCTTAAATGTCCCTTCTTCTCATACTACTTTTATCTATGTGATCCTATTCTTTTCTG
TTGTGGATTGAATCATGTCCCTCAAAAAGATTGAATTTAGAGTGTGCTCTAAATTCAATGTGGAGAAATTTGGAC
ACAGAGGCAGACACACAGGGAGAACCCCGTGTGACAATGGAGGAAGAGGATGCATTTATGCTGCCACAAGCCAAG
GAACACCAAAGATTGTCAGCAGCCACCAGAAGCTAGGATAAAGGCATGGCACATCACTCCCTCTGAGCCCCCAAA
AGGAGCCAAGACTGCTAATACTCTGATCTCGGACTTCTGGCCTGAAACAGTGAGAGAATAAGGTTCTGTTGTTTC
AAGCTACCCAGCTTGCGGTATTTTGTCACAGAAGCACAAGGAATCAAGTACATTTTCTTTCTCAGCACTTGTGAT
AATTTGATTTTTTCTTTACTCAGTGGTTGTTTCACACCTATGTCCCCATCAGACTGTAAGCTTAAAGAGACCTGG
ATCTGGTCTGTCTTCACCACTGTTGATTCATTACCAGCACAGTGCCTGGCCCATGGTCACTGAATAAACGTTTGT
TGAGAGAATGAATGTGCTTAACCAGAAGTACTATTGACCTATTAGGCCAAGTTCAAGGTGCCTAACAGCTCAGCT
GTGAAGGATACCTCTCCTTTCAGTCCTCTGTTACATATGTCCCTGATAGATGTGTTATTTGTATCTCCTCCTGGC
CCTCAAGTTTGTTTGAGGGCAGGACCCTTTTTTGTATATGTGTAGAGCTTCGTAGTACCTAAATACTACTTTGCA
TATATAATAAAGTTTCGATAAATATTCATTAAATAAAGAAATAAATGAAATGACTAAGTTTTCTAAGATGTTACA
ACTAGATTGAAGATATTTAGCTCATTATTTAACAAGAAAACTATGGTTAATTATGGTGTCCTGTGTGAAAATGGT
TATAGTTTGTTTTTTAATTAATATAAGCATGTATGTGCATTATCAGTATACACAATTTGTGGTATGAGTGTTTTG
TGTCCCTGCACACAGACCACGGAAATCCTGAGAAACAAACTGCCACCCCAGAGCAGGTGCCTAACACAGAGACTT
TTAATCCTTAAAGTTTTTCTATAACTAAGCAATGTTTTTTCAAATGCAATAACACTGATATGCAGACATATTGAT
TGTCCACTCACAAAGCCATTCCTCAATATCATTACAACATGCCTCTTTGAATGTCATTAAAAATAGATGTCTCAT
TTTTCTAGGACAAGTTGGCTGAAGTTCTGCTTGAAAACTGGTAATAGAAAATACAATTTCTCAACCCGCTTTGGC
CTTTTAATTCTGTTCTACAACCTTGCCAGTTCACTTTCAAAGTCAAGGGATGCATCTTGCAAAACCATGACATCT
TTTGAGTAACTCCTTCTGTTCTTAACACATATTCCCAGGAGCTTAATAAATATTGTTTTTGCAACTTGTTTAGTG
GCAAAATAATGAGTCCTTGGTGTATGCTTATCCTCTGCTTTGCTATTAGAGAAGATATATTCAGACTGTTTTAAA
CAAATTAATTCAAGGGCAGGGAACAGTCCTAAAACCTGTTAAAATTCAAATACTTGGTCACTGTATGTGCAGCAT
GTGTGTTCTAGAAAGTCCTATTATTTTAAAATATAAATTGAATCTTGTTGAGAAATTAATGTCATATGAATATAT
TAATAACTGAAATGCTGCCAAGTTTACAAAAAGCCCTCAATGAAACTGTGACCTTGTATAGACAAGGGCCTGTGG
AGGGACATTTTTAAACCATCTCTTTTTTTATTTCCTCATGAGATCTACAATGTAAGTGCATTAAAGTTGATGAAT
GAATTGCAGTGCAACTTTTCCTGCCTCTTTTGCCTTTCATTTGTCTATATTTCAAGCTTCACTGAAGTGATAGAT
TTTGGGCTTTGCCACATTGTCCTCTGATTGCTTCCCTCTGCTCCTCCTTTTCCTAGTGAATCTTTGTTTTACTGG
TGGAAAAATCTACATCTTTGTATCTTGGCATTTTACTTTCACATTATCTCATAGATTTTATTTCAAGTTGCTATA
AAGTTATCAACTTTTATTTTTAACTAATATTATTTTTAACAATTAGAAAATTGTTGACCAGGTAATTCCAGCACT
TTGGGAAGCTGAAGCGGGAGGATCACGTGAGCCCAGGAGCTCGAGACCAGCCTGGGCAATGCAAGGAGACTGTCT
CTACAAAATATAAAAATACATTAGCCAGGTTTGGCGGTGCATGCCTGGGGTCCAGCTATTCAGGAAGCTGAGGTG
GGAGGATCACTTGAGCTGGAGAGGTTGAGGCTGCAGTGAGCAGTGATCGCACCACTGCACTCCAGTCTGGGTGAC
AGAGGGAGACCCTATCTCGAAAAAAAGGAAAAGAAGAGGATTTTGCTGGCAAGATGGCTGAATAGGAATAGCTCC
GTCTGCAGCTCCCAGTTGAGATCAATGCAGAAGGCAGGTGATTTCTGCATTTCCAACAGAGGTACCTGGTTCATC
TCACTGGGACTGGTTGGACGGTGGGTGCAGCCCATGGAGGGTGAGCAGAAGTAGGGTGGGGCGTTGCCTCACTCA
GGAAGTGCAAGGGGTCCCTCTTCTAGCCAAGTGAAGCCGTCAGGGACTGTGCCATAAGAACAGTGCACTCTGGTC
CAGGCTTTTCCCACAGTCTTTGCAACCCACAGACCAGGAGATAACAAGCGGTGCCTATGCCACCAGGGCCCGGGG
TTTCAAGCACAAAACTGGGTGGCCATTTGGGCAGACATCAAGCTAGCTGCAGGAGTTTTTATTTTCATACCCCAG
TGGTGCCTGGAACGCCAGTGAGACAGAACCGTTCACTCCCCTGGATAAGGGGCAGAATCCAGGGAGCCAAGTGGT
CTGGCTTGGCGGGTCCCACACCCACGGCGCCCAGCAAGCTAAGATCCACTGGCTTGAAACTCTCGCTTCCAGCAC
AGCAGTCTGAGGTCCACCTGAGACGCCCGGGCTTGGTGTGGGGAGGGGCATCCACCATTGCTGAGGCTTGAGTAG
GCGGTTTTACCCTCACGGTGTAAACAAAGCTGCCTGGAAGGTCCAGCTGGGCACAGCCCACCACAGCTCACCAAG
GCCGCTGTGGCCAGAGTGCCCCTCTGGATTCCTCCTCTCTGGGCAAGGCATCTCTGAAAAAAAGGCAGCAGCGCC
AGTCAGAGACTTATAGATAAAACCCCCATCACCCTGGGACAGAGCACCTCAGGGAAGGAGTGGCTGTGGGTGCAG
TTTCAGCAGATTTAAACGTTCCTGCCTGACAGCTCTGAGAGAGCAACAGATCTCCCAGCACAGCGTTCAAGCTCT
TTTAAAGATCAGACTGCCTCCTCAAGTGGGTCCCTGACTCCCATGTCTCCTGATTGAGAGACACCTCCCAGTAGG
GGCTGACAAACACCTCATAAAGGAGAGCTCCAGCTGGCATCTGGCAGGTGCCCCTCTGGGACGAAGCTTCCAGAG
GAAGGAACAGGCAGCAATCTTTGCTGTTCTGCAGTCTCAGCTGATGATACCCAGTCAAACAGGTCCTGGAGTGGA
CCTCCAGCAAACTCCAGCAGACCTGCAGCAGAGGGGCCTGACCGTTAGAAGGAAAATTAACAAATAGAAAGGAAT
AGTATCAACATCAACAAAAAGGACGTCCACTCAGAGACCCCATCCAAAAGTCACCAACATCAAAGACCAAAGGTA
GATAAATCCACAAAGATGGGGAGAAACCAGTGCAAAAAAGTCTGAAAATTCCAAAAACCAGAACGCCTCTTCTCC
TCCAAAGAATCACCACTCCTCACTAGCAAGGTAACAAAACTGGACAGAGAATGAGTTTGACAAATTCACAGAATT
AGTGTTCAGAAGGTGGGCAATAACAAACTCCTCCAAGCTAACGGAGCATGCAAGGAAGCTAAGAACCTTGAAAAA
AGTTAGAGCAATTGCTAACTAGAATAACCAGTTTAGAGAAGAACATAAATGACCTGATGGAGCTGAAAAACACAG
CACGAGAACTTTGTGAAGCATACACAAGTATCAATAGCCAAATCGATCACGTGGAAGAAAGGATATCAGAGATTA
AAGATCAACTTAATGAAATAAATTGAGAAGACAAGATTAGAGAAAAAAGAATGAAAAGGAATGAACAAAGCCTCC
AAGCAATATAGGACTATGTGAAAAGACCAAATCTATGTTTGACTGGTGTACCAGAAAGTGACGGGGAGCATGGAA
CCAAGCTGGAAAACACTCTTCAGGATATTATCCAGGAGAACGTCCCCAACCTAGCAAAACAGGCCAACATTTAAA
TTCAAGAAATACAGACAACACCACAAAGATACTCCTCGAGAAGACCAACCCCAAGACACATAATCGTCAGATTCA
CCAAGGTTGAAATGAAGAAAAAAATGTTAAGGGCAGCCAGAGAGAAAGGTCAGGTTACCCACAAAGGAAGCCCAT
CAGACTAACAGCAGATCTCTCTGCAGAAACCCTACAAGCCAGAAGAGAGTGGGGGCCAATATTCAACATTTTTAA
AGAAAAGAATTTTCAACCCAGAATTTCATGTCCAGCCAAACTAAGCTTCATAAGTGAAGGAGAAATAAAATCCTT
TACAGACAACCAAATGCTGAGAGATTTTGTCAACAGCAAGCGTGCCTTACAAGAGCTCCTGAAGGAAGCACTAAA
CGTGGAAAGGAACAATCGGTACCAGCCACTGCAAAAGCACACCAAATTTTAAAGTCCATTGACACTATGAAAAAA
CTGCATCAACTAACAGGCAAAATAACCAGCTAGCATCATAATGACAGGATCAAATTAACCTTAATTAAGTTAGCC
TTAAATGTAAACGGGCTAAATGCCCCAGTTAAAAGACACAGACTGGCCACCTGTATAAAGAGTAAAGACCCATCA
GTGTGCTATATTCAGGAGACCCATCTCACATGAAAAGACACACATAGGCTCAAAATAAAGGGATGGAGGAATATT
TACTAAGCAAATGGGAAGCAAAGAAAACAAAAAGCAGGGGTTGCAATCCTAGTCTCTGATAAAACAGACTTTAAA
CCAACAAAGATCAAAATAGACAAACAAGGGCATTACATAATGGTAAAGGGATCAATGCAACAAGAACAGCTAACT
ATCCTAAATATATATGCACCCAATACAGGAGCACCCAGATTCATAAAGCAAGTTCTTAGAGACCTACAAAGAGAC
TTAGACTCCCACACAATAATAATGGGAGACTTTAACACTCCACTGTCAATATTAGACAGATCAATGAGATAGGAA
ATTAACAAGGATACTCAGGACTTGAACTCAGTTCTGGATCAAGTGGTCCTAATAGATACCTACAGAACTCTCCAC
CCCAAATCAACAGAATTTACATTCTTCTCAGCACCACATCGCACTTATTCTAAAATTCACCACATAGTTGGAAGT
AAAACACTCCTCAGCAAATGCAAAAGAACGGAAATCATAACAGTCTCTTAGACCACAGTGCAGTCAAATTAGAAC
TCAGGATTAAGAAACTCACTCAAAACCGCACAACTACATGGAAACTGAACCTGTTCCTGAATGACTACTGGGTAA
ATAATGAAATGAAGGGCAAAAAAAGAAGTTCTTTTGAAACCAATGACAACAAACACACAATGTACCAGAATCTCT
GGGACACATTTAAAGCAGTGTTAAGAGGGAAATTTATAGCACTAGATGCCCAAAAAAGAAAGCAGAAAAGATCTA
AAATCGACACCCTAGCATCACAATTAAAAGAACTAGAGAAGCAAGAGCAAACAAATTCAAAAGCTAGCAGAAGAC
AATAAATAAGATCAGAGCAGAACTGAAGAGGAGAGAGACATGAAAAACCCTTCAAAAAAATCAATGAATCCAGGA
GCTGGTTTTTTGAAGAGATTGACAAAACAGATAGACCACTAGCCAGACAATAAAGAAGGAGAGAAGAATCAAATA
GATGCAATAAAAAATGATAAAGGGGGTATCACCACTGATCCCACAGAAATACAAACTACCATCAGAGAGAATACT
ATAAACAACTACACAAATAAACTAGAAAATCTAGAAGAAATGGATAAATTCCTGGACACATACACCCTCCCAAGT
CTAAACCAGGAAGAAGTTGAATCCCTGAATAGACCAATAACAAGTTGTGAAATTCAGGTAGTAATTAATAGCCTA
CCAACCAAAAAAAGTCCAGGACCAGACAGATTCACAGCCGAATTCTATCAGAGGTACAAACAGGAGCTGGTACCA
TTCCTTCTGAAACTATTCCAATAGAAAAAGAGGGAATCCTCCCTAACTGATTGTATGAAGCCAGCATCATCGTGA
TACCAAAACCTGGCAGAGACACAACAAAAAAAAGAAATTTTCAGGCCAATATCCCTGATGAACATTGATGCGAAA
ATCCTCAATAAAATACTGGCAAGCGGAATCCAGCAGCGCATCAAAAAGCTTATCCGCCAGGATCAAGTCGGCTTC
ATCTCTGGGATGCAAGGCTGGTTCAACATACGCAAATCAATAAACCATCATTCTCAGCAAATTATCACAAGAACA
GAAAACCAAACACCGCATGTTTCTCACTCATAAGAGGGAGTTGAACAATGAGAACACGTGGACCCAAGGAGGGAA
CATCACATACTGCGGCCTGTCGAGGGATTTGGGGTTGAGGGAGTGATAGCATTAGGAGAAATACCTAATGTAGGT
AACAGGTTGATGGGTGCAGCAAACCACAATGCGATGTGTATACCTACCTAACAAACCTGCACGTTCTGCACATGC
ACTCCAGAACTTAAAGTATAATAATAAAAGGCGCTGCCTCAGGATGTAAAGTGTAACAAGGGGGCTGGGGTGGGC
AGCGTGGGCCTCTGAGACCTTTGGTTGCCCGTGTCCGCAGCTCGCCCCGCAGCCGGCTCCACAATGGTCCGCTCC
GTTTGCCACGTGCGGATTCGGGTTCCAGACTGAAGGCTGCGTGTTCTCTGCCGCCCACAGCCCAAGTTTATTGTG
GCAACCGCCGGAGCAGCCTTCCCCGCTGTGGAGGAGCCTGGGGCTACCCCTCAGCGGTATTTGGGGCTGGTCCTG
GGGGAGCTAAGCAGGGTTGTGGCAGCACTGCCTGAAAGTGTGAGACCAGACTCTAATCCTTATGGTTTTCCATGG
GAGTTGGTGATATGTGCAGCTGTACATGGATTTTTTGCTGTTCTCTTTTTTTGTGTGGAGAAGTTTTAGATCGGT
TGGGAGTCGGCTTTATGTGGGAAGAGAAAAAAAGCTTGCTGTAATGCTTTCTGGACTAATTGAAGAAAAGCATAA
ACTACTTGAAAAATTTAGCCATGTTCAAAAAGAGTATGAAGGCTATGAAGTAGAGTCATCTTTAAAGAATGCCAG
CTTTGAGAAGGAGGCAACCTGTGAAAAGCTAAACAGGTCCAATTCTGAACTTGAGGATGAAATACTCTGTCTAGA
AAAAGAGTTAAAATAAGAGAAATCTAAACATTCTGAACAAGGTGAATTGATGGTGGATATTTGCAAAAGGATACA
GTCTCTAGAAGATGAGTCAAAATCCCTCAAATGACAAGTAGCTGAAGCCAAAATGAACTTGACGATATTTCAAAT
GAATGAAGAACGACTGAAGATAGCAATAAAAGATGCTTTGAATGAAAATTCTCAACTCCAGGAAAACGAGAGACA
GCTTTTGCAAGAAGCTGAGGTATGGAAAGAACAAGTGAGTGAACTTAATAAACAGAAAATAACATTTGAAGACTC
CAAAGTACATGCAGAACAAGTTCTAAATGATAAAGAAAATCACATCAAGACTCTGAACGCTTGCTAAAAATGAAA
GATCAGGCTGCTATGCTTGGAGAAGACATAACGGATGATGGTAACTTGGAATTAGAAATGAACAGTGAATCGGAA
AATGGTGCTTACTTAGATAATCCTCCGAAAGGAGCTCTGAAGAAACTGATTTATGCTGCTAAGTTAAATGCTTCT
TTAAAAACCTTACAAGGAGAAAGAAACCAAATTTATAGTCAGTTATCTGAAGTTGATAAAGGAAGAGCTTACAGA
GCATATTAAAAATCTTCAGACTGAACAAGCATCTTTGCAGTCAGAAAACACACATTTTGAAAGTGAGAATCAGAA
GCTTCAACAAAAACTTAAAGTAATGATTGAATTTTATCAAGAAAATGAAATGAAACTCCAGAGGAAATTAACAGT
AGATGAAATTACCGGTTAGAAAAGGAAGAAAAACTTTCTAAAGTACACGAAAAGATCAGCCGTGCCACTGAAGAG
TTGGAGACCTATAGAAAGTGAGCCAAAGATCTTGAAGAAGAGTTGGCGAGAACTATTCATTCTTATCAAGGATGG
ATTATTTCCCACGAGAAAAAAGCACATAATAATTGGTTGGCAGCTTGGACTGCTGAAAGAAACCTCAATGGTTTA
AGGAAAGAAAGTGCTCACAACAGACAAAAATTAACTGAAGCAGAGTTTAAATTTGAACTTTTAGAAAAAGATCCT
TATGCACTTCATGTTCCAAATACAGCATTTGGCAGAGAGCATTCCCCATATGGTCCCTCACCACTGGGTCGGCCT
TCATCCTAAACAAGAGCTTTTCTCTGAGGGCCCACTGAGACTCTCATCTTTGCTAACAGGAGGAGGAGGAAGAGG
CTCAAGAGGTCCAGGGAATCCTCTGGACCATCAGATTACCAATGAAAGAGGAGAATCAAGATGTGACAGGTTAAC
CAACCTCACAGGGCTTCTCTGACACTGGGTCCCTGTCACCTCCATGGGAACAGGACCGTAGGATGATGTTTTCTT
CCACCAGGACAATCATATCCTGATTCAGCTCTTCCTCCACAAAGGCAAGACAGATTTTATTCTAATTCTGGCACA
CTGTCTGGACCAGCAGAACTCAGAAGGTTTAATATGACTTCTTTGGATAAAGTGGATGGGTCAATGCTTTCAGAA
ATGGAATCCAGCAGAAATGATACCAAAGATGACCTTGGTAATTTAAATGTGCCTGATTCATCTCTCCCTGCTGAA
AATGAAGCAACTGGCCCTTACTTTTCTCCTCCACCTCTTGCTCCAATCAGAGGTCCATTGTTTCCGGGGGATACA
AGGAGCCTGTTCATGAGAAGAGGACCTCCTTTCCCCCCACCTCCTCCAGGAACCATGTTTGGAGCTTCTCAAGAT
TATTTTCCACCAAGGGATTTCCCAGATCCACCACATGCTCCATTTGCAATGAGAAATGTCTATCCAGCGAGGCGT
TTCCTCCTTACCTTCCCCCAAAACCTGGATTTTTCCCCATAAACCCCACATTCTGAAGGTAGAAGTGAGTTCCCT
GCAGGGCTGATTCTGCCTTCAAATGAGCCTGCTACTGAACATCCAGAACCACAGCAAGAAACCTGACAATATTTT
TGCTCTCTTCAAAAGTAATTTTGACTGATCTCATTTTCAGTTTAAGTAACTGCTGTTACTTAAGTGATTACACTT
TTGCTCCCACTGAAGCTTAATGGAATTATAATTCTCAGGATAGTGTTTTCTAAATAAAGATGATTTAAATATGAA
TCTTATGAGTAAATTATTTCCATTTTATGTTATTCTGGATAGTATAACTATTTTAATTTGATAAACTAATCCACG
ATTATATAAACAATAATGGGAGTTTTATATATGTAATCTTGCAGGTAGGGAGGCTTTAAATTATAAAGGTTGTGT
CTTTATGCCAAGAACTGTATTAACTGTGGTTGTAGACAAATGTGAAAGTAATTTTATGCTTCATTAAATAAATTT
TAGTTGATTTTTTTTTAAAAAAAGAAAATGGTTAATCTATCATTTAGGTGCATCATCAGTTGTTTAACCATTCTC
TCTTACTGAACATTGGGTTGTTTAAAAAGTGTTGTTATTTTTGAATCATGGTTCAGTGAACAATTTTGGACACAT
AACTTTTTATCTGATGAGTTATTTCCTAAGGATCCAGCTCAGAAACTCAGCACATAAACCTAATAAGAAAAAAAC
AATTTGAAGTGGCTAACCTCTTATCCCAATAAAAATGTTGTATTTATGTTTGGATTTAGATGCCTTTCAGTGGTC
ATACCTTCACCTAACTTTTATGGATTCTACTTTTAACATGTAGAGTGACTGTTTAAATCACCTAAACTCACTGAG
TTTTAAGTTCCTTTTTATTCAACAAGACTGGATTGTATGTTCCAGCTCCTCAAACTTAGTTACCAACCACCATCC
TAGAGAAGTGAATTCACATGAGGCCTGTCCAGAAGAACAATCTCCCTTTCAGTGTCCTCATGCATGCAGTGACCA
GAGACCAACCTTGATAAATTATGGAAAAAGTACAGCACATTCTGGAAGAGCCATGAAAGATCCAGATCATCTGGT
GCTGGATAAGAATATTAATGGACAGGCTGGGCGCGGTGGCTCACGCCTGTAATCCTAGCACTTTGGGAGGCCGAG
GCGGGCGGAACATGAGGTCAGGAGATCGAGACCATCCTGGCTAACACGGTGAAACCCCGTCTCTACTGAAAATAC
AAAAAATTAGCCGGGCATGGTGGCGGGCGCCTGTAGTCCCAGCTACACGAGAGGCTGAGGCAGGAGAATGGCGTG
AACCCGGGAGGCAGAGCTTGTAGTGAGCCCAGATGGCGCCATTGCACTTCAGCCTGGGCGACAGAGTGAGACTCC
GTTTCAAAAAAAAAAAAAAAAGAATATTAATGGACAAAAAGATTAATGAAAGAACATATTGAAGCATCCAATTAC
CTGGTGTCTGCTCAAATGAGGAATCGGTGAGATAGGTCAGTTAGCAGTCAAGATTTATAAAAGAGACGATGGCCT
TGGGAGGGGCTGCCCTACTCGACTTTTTAATGGCTAGAAGCTATTAAGGGCTAAGCCAGAACCCTTCAGTATGGT
TCAGTGAGGATCCCAATTTGGGGTCCAAAAGTAAATGACAACTCCCAGGAACCATTAAGAATAAAAATCATGGAG
CATTACTGAGAATTTATGTTATCTAAGTCTGAGGAAAATTAATGTTAAGGAAGCTTTCAAAAGTCTAATATTTAC
ACCGAATTCCAGGGCACCATGCTCTAAGACAAAGCACTCTGGTCCTGCCCCTCTCCTTTCCTCATGTTTTTTGGT
TCTTGGGATCCTTAAGGGTCAATGTTATTCTTAAAATACAGAGCATCCTGGAAACTAAAAAAGTGGAAGATATTC
AAATTCTAATGAATGTACTGGCAGTATTGTAGATCATGGAGTATAACATAAAGACAAGAATCCCTAGCCTCTTCC
ACCATACTTTGTAATGGTAAGGAGAAAGGATAGAATTTTGAGAAGTCTGGGAAGACAATGTATGATAACATCTGG
AGAAGCTCTGCATAAGTTACTTTTGTTCAGGCTTAAGAAAAATTCTAGCTTGCCCCTGCACTGTCATCAGGTATC
ATGAAAGTAAAAAAACCTTTAAAGATTCTTCAAGCCAGCAGACTTCTATCTTTCTCTATACTATCCTGTGATCCT
AAACTCTTAACAGTTACTACGTATAATTTCCCTACATTTGCTACTAGTATTTTATCATACACAATATTACACTCA
ATATTTCAAAAGTGGATGATTCATCTCCCGAAGAGACTGCAAAATTCATGAGTTAAGATTTGAGAATACTATTTT
AGACAAGATTTAGTCAGATTTTAGAGAGTTAGAAACCTGTAACAATTCTCTAACAATACTGCTTCTCCTTTTGTG
TATTAAGGAATTTTTGTCTATCAAAGATAGTACGAGGTAGACCAGAAGATAACTTGCCTTCAAAATGTCTGGAAT
GTAAAATGGCAACAGTAGTATTTGGGGACTTCGTAGGGGATGGCCAATATACACCCATTCTTAGAGGTACTGATG
ATATAATGTATAAGACAAAATCAAGTGGTCTCCATCACCATATAATGTTTAAAATGGCAAAGAGGGAGCAGAACA
AACACCCTTTGCAAATCTCTTCATAGAATCTACCGTAATAAACTTGTACTTGCTTAAAGTGTGTCTCTTCAGTGG
TCTTATTACCACTACTTTGGGGAAAATGAGGCTGCTTAAAAGATTAACAGACATTACATTTTACATATCTGTGGC
AGAGAAAACACTATGTATTCACCAAACCACTTCTTTTCCTTCCCAGTCACTCGGGAAGAGGTCATTTCTTTGTCC
CCTTTCATCTAATTGAGGTGCCGTGACTACTTCTAGACAGGCAATGTGAGCAGAAGGTATGCACGCCACGTATAG
GCCTGGTCTTCAAAAATCCCTCAGATATGATCTTCTTCTCTCGTCTCTTTCATGGACAAACTACAGGCCATGTAA
TAAGGATGGTGGGGTTCCAAACTGAAAGAGCCTGGATTTCTGATTTACTGTTTTGAGAAGAGTTCACCAGGGAAA
CAGCCTGGAAATACGCACAGGAAAATATGCACAGGACCCTGTGTGAGCAAGATATAAAGATCTATTACATGGTGC
CATTAAGGTGAGAGTATTGTGCTTATAGTATCCAGCATTAATTATCCTCACTACTACAACTTCTTTGTATCCATC
ATGTGGAAAAGTAGAGTATTTAATAAATGATTATTGAGTTTATTACCTTTTTTATATTCCAATCATTGCTAATTG
TACGTTACCTCATTTCAAGGTAAAGGTGACCAAGGGCTAAAGCAGTGCTATCCAAACCAAGCCAGACATCAAAAT
CACACAAAACCTTTTGAAAATACAACTTTGAAGATGCCATTCACATAGATATTTATTCAGTGGGTTTTCAAATGG
AACCCTGGAATCTACAGTCTTTAACAAGGCTTCCCAAGTTATTCTGATATACAGCAGGCAAATCTGAGAACCACT
GGACAAGAAGAAAATAAAGGCTATATCTTTCGACAACAAAGACAATGCCTTAAACATAGAATGTATTCAATTAAA
GCTTGTAGAAAGATAGGTTTGTGAACAGGCACAGGGACTAGCCTCGAGCAAATTAATAAGGGCAGCAATGTTTTT
CACTGAAACCATTATTCCCCCTATTTTATTTCTTCTGGGGCTCTGTGTTTCCTTTCTCCTATCAAAATCCATTCT
AAGGTTGGAGGTTGGGGGTATCTCTTGCCTACTCCATACAGCAAGGAATAAAATTAGTATTTCTCGAACTATCTG
TGACAGCAGACCCATTGTAGGCCAGTACTTTTGTAAAATGCAATAAAAATTAACTTCTAGAGAATGAAATTTTAA
AATCACAGACATTCAAAATACAAATTCCAATTTTTTTATTATTAACTGTAAGAAATTTAAAATTAAATCTCAATA
AATAAAATTAAAGCAAACATAAGATAGAAAAAAATAAGCATTATGGATTGGCCCAGTCTGCAAACTGTATACACT
TTGCCAAACATGGGCATAAATTACTAAGAAGCAAAATCTTCCATCTGTAAACATTTCCATTTCCATTGACAATAT
GTGTGAGGGAAAGGAGGGATGCTTCTGTTTTAGAATGCCAGGCGTCAGCTAACAAGTGACAAATACGTATTGAGA
CTGAGATCTCCCCAGCCTCTCAGTAGTCAGCAAGAACATGTTGAGGCCTCTGTTTTTGACTAAAAAATTGGCCAG
TGCATGGGCAACATGCATAGGTCCTGAATGAAAAAAATAGCAGCAGCAGAAATTTAAAAGAATTTTCACAGCTAG
GCCACAGTAAATTCTCAAGCCCTTCATCAGAAGCCACTGTGGGGCCTCATTTATGCCTTTGTTTTTATTAAATTG
GATGTGATCTTAAGATTCTTCTGTCAAAATTCCACTAGCATGTGAAGGCACCAAAAGTTTAAAATGTAAAATTAA
CCCAAGTTAAGCTATTCCATTATTAAGCAATAGCAGATATATTTGTTATTATATGAGAAGAAAGTTAACAGGGAG
CTAAGATTGATGTTACTGATAAGAAACAGAAACAAGACTTTAAAATTAAATAAATGAATTATTTATTTAATAAGA
ACCAATTGACAGATTCTCGATAAAGACTGTAAGATGTCTTAAAACATTAGGTGTATGGAGATAACATTTGTAACT
TTGACAATTTATATGATGAGAAAAATCAAGGAATGTTATTGTTTATTGGCAGAGTTCTAGAATTACAATTCCATC
ATTCTGTTTTGGGGAAGTTTCCCTTGAAGTAAATGATAACAGGGCTTGAAATAGTACACCTCAGCATTTTGTTTA
TAAAACTGTGGAATAGGTAAGGTTTGTATTGTAACTGAACCCAGGTTCAGCTGCTTGCTGCTCTAAAGCTAGACA
TAAGAGAGGAAGGTTGGTGGGAGGAAAAGCGATTTTAATCGGAGAAGCAGCAAACCAAGAAGATGGTGAACAATA
GTCACAGAACCATCTTAAATTTTAAAATTTACCATAGAGTGTTCAAAGGAAAACTTGGTATGGGAGGCATGCAGG
AGGGGTGCAGGGGGCGGGGTCTGTGTGTCTTGTTCCAATGGCTATCTCAGATAGTCACCCATCTGGAGGTCTAGT
TGGTATTATTTTGAATTCAGCCCAGTGGTGGTGGACTGTCAGTGACTCCTCGCTAAGCAGGAGGATTCTGCACTC
AGGGCTCCATGCATGGTTTGTTTCAAGATTGGCCTCTGGAATTTCTCAAGCAAGAACATAATTAAATAAGCAGGC
ATTGCCAGAGGGGAGTGTCTGGAAAGGAAAGGAATGAAGAGATGAAAGGAAAGTGGGTGGTTAAACTATATTTTT
AAAACTGAGGTTCCCAGTTATAGTATGTTTCGCACGCTCCCCCCATTTTAGCACCCCTGACAGAATTTAGTAATC
TCCTCATCTTGTCCTCTACTTCAGGTCCCCTATCTGTCCTTGTACTCTCCAGGGTTTCCTTTTCTTCTTCACGAC
CTTCCTTCCCTGCAATTTTATAAGCTATTCCTATCCCAGTGATTTAGTTTCAGCTTATAAAACTGTGTCTTTGCC
ATTGTAATCAAATTGAAGGGCCTCTGCTTCATGGTTGGATTCTGTGACCAGGAGACTCTTACGAGGAGTTGGCCA
GGTCTCTGTTAGGAAAGCAAAAAAGAACAATGGAGGCAATTATCCCATTGATTTCAGCTATAAATCCTATTTTGC
CTGAATTGTCTGAACGATGAGTATTCTGTGAAAATGCTGCTCTCTAGTGCAATAGAACTGCAAATAATGCACATC
TATTTCTTATAATCTCATCCAACATACCCACAGAGATTCAGATCTAACAAAACAGAGGTGATTTGGTTATTGAAT
CATAATATAAATATGGGGAAGAGGAGGGAAATTTCAAGCCTGAGGAAACTGTAGTAGGAGTAAGTATGCTGTGTT
TAAGAGGTCACAGATAAAATTAATATTACCAATCCATCAATAGGCAATTACTAATAGCTTACTACACACACAGGA
ATAAAATGTGAAGACAGAGGAAGTGTAAAATGGAGCCGCCAACTCTACGGAGTTGTTTGCAATTTGGTCTGGTAG
AAAGCTATGAAATAAGGAAGTACATGATTGAGAGCTAGAGAATGTGGCACAGGCTCTGAACCCGGACCGTTCAAT
GTAGTAAGCTCTAGCCACACTGGACACTTGCAATGTGGCTTGTCCAAACTGACATGTGCTTTAAGTATAAAATAT
AATCCAGATTTCTAAGACTTCAAAAAAAATGGAAATATCTCATTAATAATCTTAAGTTTATTACAGGTAGAAATG
ATAGATTAAATAAACTATATTGTCAAAATTCATTTGATCTGTTTCTACAGTATAACAAACTTACTTGTGTGGTTT
GCATTTTATTTCTACTGGATAACATGGCTTTAAAAATGGTATTTTAGAGGAAGGAAAGCTTGGTAGAGAATGGAC
TAATCCGGATCCCTGGAAGAAATGGACCTTGAATGGGTCTTGATGACTTGGAGAGGCAGAGAGAGAAAAAGAAAA
GTCAAACATAGGGAATTGGTTGATAAAATGAAGGTGAGGGGAGAAGGAACAGAGGGAGGAGAAGATCCAGTTTGA
GGGATATTACAGCGAGCAGCCTGAGAAAGAAGGATAAGAAAGGAGAGAAAAAATGCAAGGGAAGTAACCCTTCAA
AGCCAGTCAGAAGTTTCTGGGTTCCTCAGCAGCCAGAAAAGAAGCCGTTGAAAAGATCTGAGTAACGGAGATTCT
GGACGAAAACTGAAGTTATGGAAGGGAAGTTTAGACATGGGTTATTAAACGCTTTAGCGCATTAGAAGTTTCTTA
TGTAATCACTAAATTCAGATCCTGAAATAATGCCACAAGAACTATACAGCTCAGCCACCCAATTCAATAAGAAGT
TACAGCACAGTCTCACACATATCCAATTAACCTTGGCCTTTAGTCAACATCTGGGTTCTTTTTGTCATTTTCAAA
TACTATCACCCAGAGGTGCTATGATTTATATTGGGGAGGGGATTAAAAGAAAATAAGTAAGTTGGTGATAAGAAA
AAGCTTTCAGATGATTCCATCTGAATTAACAGCCCTCTTTAGTTGTCTAGGAAAGAGGATGCTTTTTCTTGAAAG
TGCTTTGAAATGATGATGTGCTTGTTAGTAAACATCAATTATTTTCAAATCGTAATGTTTGCAAGTTTGTCTTCC
TGTAGCTCACCCTTTATGTAGGTCCAGAATATGATTGTCACAAATATCTGGGTGAGCAAGACTATGAAATGTGGT
CATAAAGTAAGTGATTATTTCTAAACTCATCTTTGTCACTCGTAGTGCTTCACAAAGCACCTTTTCCTGGACTAC
AATTCATTTTAATTGATCCCATCAGCACTATATCTGTATCCTGAGTGACTTCACAATACCCTCTATTTCAAGAGA
AACCAATCAGGTTATGGGTTTGTTAGTAATAAAAATTACCAAGGAGCAGTTTGTGGATGGTAAAAGCAATGCAAA
TTCTAAAGAGAAGTCATAAGAGCAATAATAAGCATCCTCCTCACTTCTTGGAAGTGAACAATTCCAAGCTCCCTG
AAGCAACACTTAACCTATCATATTAAACAGTAATGGACAAATATTAGAAATGTTGATGTCAGCTTTCAGAATCTG
TGGGCATCAAAACATCACTTAAGTTCTCCGAAGTATTCTCTGTCAAGTTTCCTTCTACAGTATTCTTTTCCTACT
AGGACAGAGCCTTAAGCCCTAGAAGAATAATTTTGCTTGTGTGTTAATTATTTGTTTACTGGTTCATTCCAGAGT
GTGAGCTGGAAAAAGGGGGAAGTGTCATAAATAGTTTTTTATGGCCCATGGTTTTTCAACTACGTCACTATTGGT
AGCAGTTTCCACTGCAGGATCTATTTGCAAAGCCTAGGAAATTAGCATTAAGCAAGCTGCTAGGAAGACTTCAAC
AGTAACTAGGCCACAGGCCTCACACATTTTTCCTCCACCCCAGCCTCCTCTGGAGAGTACTTGCTAAACCTCTGT
GACACATAATGAAGCAAAGAAAGTGATAGAACAACAGAATTACACGGGCAGATCCTTGTTTCTTCTTCTCTCTCT
AAAGAATTCCTTGGACTGAAAAGCAGTTTATTTTGGAGGAGTGAGAAAGTGGTGACAGAATTAGAAGGGCCTGGG
AGGGCTTCATTTTAGGAGACAGTTTTAGGCTGAAAAGAGATTTCATGAGTGTGATTTACCTGAGGTGACTTTTGG
GGGCTCTTATAAAAAGGAAGTTCATGCTGAATGGGAGGTGGCTTCTGAGATGCAGATTCTGGTGAGCTAAGAGGG
CTCGGTAAAGAGGAGGCAGGAGTTAAGTAGCGTGAACTATGCAGTAGCAGCCTTCTTCCCCCCTTGCTTGGGGCA
GGTCATCACAACCCTTCTCAATAAAGGGGTCCAGGAACCACTAGGAATAAATGGGCATTTGCACTTCAGGTGAAA
CCCATTTGTCATAACTGCTTGGACTTTAAGCTTACAAATAAAAAGAACCACATATTTCCCTTTGCAGCTTGATTT
AGTTAATGTCATTTTGAGAAAGAAAGAAGACATTGTTATCCCGTCCCTTTTTTTTTTTTTTTTTTTTTTTTATGA
AGAGACTGGGACTCAGAGAAGTCAAGTGATTTTCCCAGAACCAGAAAACACAGAAGTAGCAGAGCTGAGATGACT
ACTCCGGTCTTCTGATTCCAAATTCCAAATTCATTCTTCTAAGCGATTTCCCAAAACGGGAAATGGGTTTATCTT
CTATTTATGGGAAGTGATAGTGGTATTCTATTTAGAGAACTTATATAAAATCTTACTTTAAAATAAATAATATTT
CAAAAAGTAAGCTTAATTTAAAGAAAATAATCAAGAAAGTCTGGTATATTTTTACAAATATACCAAATGACCTTG
CTCTAAAATACATCTACTTTCCAGCAAGCCAAAGTGAAACAATTTGAAATAAGTGGCATTTACTGACCACTCCCT
AAAGTTCACACAAAAGAGGTAGTACTCTAACTTAAATATACAAGGTGAAGAAATAGCTTACTCAGCCTGTTGGGC
TTCCTCTTCTACACTCTTGGGAAATGCCCTCCGTGTTAACCAAGAATTCTCAGGCCTTGGAGGGAGTTTTCCATT
CTCAGTAAACTGAGATTGCAGTTGCGGAAATTAAGAGGTATCTGTCCAGCACTTCATTCCCTTAAGGTCAGGATC
TGTGCTTTTAATAATGACAATTAGCTAACATATACAATTAAGCCATGCAAATGAAGTAAGAGAAAGCTAGAGGAG
AAATTCAGGAGCCAGTTGCCTTTTCCAGACATCTTGTACAAATAGTGTTCAAAGGACTAATTCAAAAGATGGGAT
TCTTCGCTTGAACCCAGGAGGTGGAGTTTGCAGTGAGCGGAGATCGCTCCACTGCACTCCAGCCTGGGTGACAAA
GTGAGACCCCATCCAAAAAAAAAAAAAAAAAAAAAAAAAAAAGATGGGATTCTTTTTTAAAAAATAAATTTTACT
GCGTATTTTTAAGGTATACAACGTGATGTTATAAGATGGATATAGATAGTGAAAAGGTAACTGTAGTGAAGCAAA
TTAACATATTCATCATCTCACATAGTTATCTTTTATTTGTTTTGTTTTGATGGGATTTTTAAGATAGTAGAAAGG
AATGGTAGACAATAAACATTTGAGGGAAAGTGGGGCTTTGTAGAACTCCTAAAATGACAGCACGCACAAATGTCC
CCATTATGTCTAAAGGGTAACTCGTTCCTACTTCTAGGGACAGCTGAGGGACATCAATGTAAATTTCTAAATGAC
TTCCTGAACTTTTTATTTTTATTTTTTGTATTTTTAGAGGAAATTATAATAACATCAAGCCACCTCTGGACCATA
TCGCTGCTGATATCATCAGCAAATGGCACTATTCCTAAATCCTAAGATGCACTTTTCCCTTCACATTTCAACATT
TGTGAAACTCGATTGTACCTACACCTGATTTTATATACAATGCAGCCTTTCCTTTTCTTETGTCATTGCATCTTA
CGCCTGATTTCTCCTTGGAATTGAGTAAATATAATGCTTACATGTGTTAATAAGAATTGAGGTCACTCATAATTT
TTGAAATATGCCACCAAATATAAGCCTTTCTACATATTGTTGACTTTGAAGTCATTTCTTTTTTTAACTACTAAA
CAATAACACTTTTTGTTGAGAAAAATTGCATATGAACAAGAGACCAAGCAGGTAGAGAGAAAAAAACTTTTAATA
ATCAAGAGAATGTTACTGTGTCCCAAAGGCTAAAGTCACCTTACTATCAAGAGAGAAGGACAGGAACAGAGAGAA
CCAGGTAAATTACGAATTGAAAATTCCATGGTTCATTTATCTTTATTTTTAATAATTCCATTTGTGTGATTGTGT
TGACCACAAGGTCATAAGTTACTCTTCATACTGACTTCTCATGTAAATTATAAATAAGTTTTTTATGCTAATGAT
TTATGGAGTAAGCTATTCATCTTTCCGACAGAGAGTTACCTACAAAGAAATAATTATTCTACCTCTGAGATGAAA
TATCATGAAAGGAGTGGTTTCCAGATATTTTGACTTTAAAAGCTTAAAGAATATATGTAGTTATAAAATTCTAAA
GCAGGCAAAATTAATCCTTTTAGCAATCAAGATAGCGGCTACTTTTGGTGAGAAGGACAAGGTAGTGATAGAGAA
GGGGCTCAGGGGTCTTTCCTGAAGACAGTGAGGTGGGCAATGGTATTTTCCTTGACCTGGATGGTGATTAAACAG
ATGTGTTTACTTTGTGATAATTGACTAGGCTGTGCACCTATGAACTGCATACTTTTCCATATATGTACTGTATTC
TTATACTTAAAAAGAAGTTTAAAAATAAATGCAACAGATATAGGACTTCCTATATTACTCGTTGACCAAAAAAAT
GGATTCATTTTTCTTTCAGGTAAAACGTACTAGTGGTTTTAATATTATATTGACCAGGGAGTAAATGTTTACCTT
AGGAACCTTAATCTTGATGTTCTCCAAAGTCATTATCTGTTCTTTCTGATTATCAGAATAGAGTATATCTCTATA
TAAATGAAAATTTCTGGTCATTCTCAAAAAATAACACTAAGCATGAAAATCAGAAATATTGATCTTGTTTTGTAA
TGATGTTTCTATTGATGTGAAGTAGTTTCTAGTAGAGTTGCTGTCCTAACACACAAATGAAATTGCACTGTTTGG
AAGACACAACTGTGAATGACTTGCTTCAGTAAGGAATTTCCAACATGATGGTTTAGGGATAGAGGTGCTCGATTC
CTCTGTCTCCGGTTACCCAGGTTATTGAGGACAGGGAGGTCAATAAGTAATGCCCTCCTCCCACCCATAGCACAA
AACAGAGCGGGGTTCAGAGAATAGGTAAGGCTTTGGCCAGGGTGTTGAGGAGACTTACATCCCTGGGAACCAGTC
AGAATGGGGGCGCTGAAAACAATGTTTTAAATTCTAGCACCCAGCAACATATGTGTGAAGATTAAATGTACTCGT
GCTAAATTCACTTGCTCCATTACTGAATTTGGGTGGTGTCTGTTAAAGATGGGAACAAAGGCATTCAGGTCCTGG
TATCTTCTACCACTCCCAGCATGAACAGACTCATGTCAGTGGGTAAGGGATGGTATTTCCCGAGAAGGCTTTGAA
CTCTTGTAGTGGGTCAAATAATGGCCCCCCACTTAAAAATGTTCATGTCCAAATCCCTGGAAGCTGTGAAAAGGG
GTTTTTGCACATGTAATTAAGTCAAAGATATTGAAATTAGATCATCCTGGATTACATAGGTGGGCCCTACATTTA
ATGACAAGTATCCTCATAACAGAAGAGGAGAAGGTGATGTGAGATTTGGAGCAGCAGAGATTGGAGTGATGTGGC
CACCAATCAAGGAAACCAAGGACTTCCAGCAGCCACCAGAAGCTGGAAGAGGCAAGGAAGGACTCTTCCCTAAAG
CCTTTAAAGGAGCACAGCCCTACTAACACCTTGCTTTTGGGCTCTGGCCCGCAAAACTGTGAAAGGATACATTGC
TGTTATTTGAAGCCACAGTTCGTAGTAAATTTATTACAGCAGCCCTAGAAACTGATACAACTCCTAAATACACCC
TTAGCAACACTGCTCAACAAGAAGTAGGCAATTTCCTCCTGACTGAAAAATACTGATACTGTTATGGGATCCTTG
GGGGTGTTGCTTTTCTGTCCAGAAACCTCTGTGGCGGTGGCACCTTTGCATGAGTTTTGCTCGGGTCCACTGGGC
CCACTCATCCTGGCAGGCTGCGCTCAGCTGACACTACTGGCGTGGATCCCATGCCTCCAAAGAGACTGGAGCGAA
GCGGTGAGGGATGTGTGAGGAAGTGAGCGTGGGGTCTGGCACACAGTCAGGCTCAATGGCTGCTACAGCGGGATG
GGCAGCTTCAGGTGCTGGCACGGGTGCTGGCTCACTGCAAGGCTGTGGCTGCACCAAGCAGCGCAGCAACGGAAC
GCATTGGTGCCTGGAAACTTGGAGACTCCAGGAACCTCAGGGCTCCAAAAGGCAAATCACAGCCCTAGCTTCGGG
AGCTCCCAGGTCTGGGCTGCCAAAGGGCTGCAGCTCTTCTCTCCTCTCTCTCTCTTCGCTCCTCTCCCTTTCTCT
CTTCACTCCTCCCTCTTTCTCTCTTCACTCCTCCTGTCGCCTATGAACAGCGAATTCAACCTTCCAGTTTTCAGA
CTAGGAATGCTGGAGTTGTCCTTGATTACTCTGAATTGTTCACTCCGCATATGGGCACTGAGGATACGTTGATGA
ACTACACAGACAAAAAGGATAGAAATTCCTGTCAAGACTACATTCAATAGGGATGAAGCAGGCAATAATGAATAA
ACATACTAAGTTGAATATGACTATTTAAATATATATAACACATATGACTTGTATAATGTTAAATATTTTAAGTTT
TTTAAATTCTTCCCTTCATAGATTTTACATTATAGTAGAAGAGGCATTTTTGTTGTTGTTCTTTTTGTTTTGGAT
TCAGAGGGTAAATGTGCGGGGTTGTTACATGGGTATATTGCATAATGCTGATGATGGTCCCATCACCCAGGTGGT
AAACATAGTACGTAATAGGTGAATTTTTAGCCCGTGCTTCCCTCTCCCATCTAGTCGTCCTGAGTGTTTATCGTT
GCTACGTTTATGTCAATGTGTATTCAATATTTAGCTCCCACTTATAATTGAGAATATGCAGTATTTCGTTTTTTG
TTCTCGTGTTAATTTGTTTAGGATAATGGCCTACAAAGAACATGATTTCATTATTTTTATGGACATGTAGTATTT
CATGGTGTATATGTACCACGGTTTCTTTATACAATCCCACTGTTGATGGGCACCTAGGTTGATTCTATTGCTGTT
GTGAATAGGGCTGCAATGAACATACAAGTGCATGTATCTTTTTGGTAACAAAAATTTTATATTTGGATTACCCAG
TAGAATTGCTGGGTTGAATAATAGTTTTGGTTTAAGTTCTCTGAGAAATCTCCAAACTGCTTTCCACAGTAGCTG
AACTAATTTACATTTCCACTAGCAGTGTATAAGCGTTCTCTTTTCTCCACAATCTTTTCACCAGCATCTGTTATG
TTTTGGCTTTTTAATAGCCTTTTGATGACTGTGAAATGGTATCTCACTGTGGTTTGGATTTCCATTTCTCTAATG
ATTAGTGAATGTTGAGCATTTTTTTCATATGTTTATTGGCCGTTTGTATGTCTTCTTTTGATAAGCGTCTGTTCA
TGTCCTTTACACATTTTCAATTAAAATATTTGTTTTTTGCTTGCTGATTTAAGTTCTTTGTATATTCTGGAAATT
AGATCTTTGTCAGATGCATAGTTTGCAAATATTTTCTCCCATTCTGTAGCCTGTTTACTCTGTTGGTAATTTCTT
TTGCTGTACAGAAACTCTTTAATTAGGTCCCACTTGCCTATTTTTAGTTTTGTTGCAATTATTCTCTGGAACTTA
GCCATAAATTGTTTGCCAAAGCCAACGTGGAGAAGGATATTTTCTAGGTTTTCTTCTAGGATTTTATAGTTTAAG
TTTTACATTTAAATCTTTAATCCATCTTGAGTTAATTTTTGTATATGTTGAGAAGCAGGAGTCTAATTTCATTCT
TCTGCATAGGGCTAGCCATTATCTTGGCACCATTTATTGAATAGAGAGTCCTTTCCTTATTGCTTATTTCTGTCA
ATTTTGTTGAATATCAGATCGTCGTAGGTGTATGGGTCCATTTCTGGGTTTTCTATTCTGTTCTATTTGTCTCTG
TGTCTGTTTTTGTACCAGAACCATGCTGCTTGGTTACTGTAGCCTTTTAGTATAGTTTGAAGTTGGGTAATGTGA
TGTCTCTGGCTTCGTTCTTTTTGCTTAGGATTGCTTTGGCTATTCAGGCTCCTTTTTGGTTCCATATGAATTTTA
GAATATTTTTCTGATTCTGTGAAAAATGACTTGATATTTTGCTAGGGATAGCATTGGAGTGGTAACTTGCTTTGG
ACAGTGTGGCCATTTTAATGATATTGATTATTCCAATCCATGAGCATGGAGTATTTTTATATTTATTCAGTCATC
TTGATTTCTTTCAGCAGTGTTTTGTAGTTCACCCTGTAGAACATTTCACTTCCATGGTTAGATGTATTCCTATTT
TGTGGCTATTGTAAATGGCATTGTATTTTTTTTTATTTGGCCCTAAACTAGAATGTTATTGGTGTATAGAATTGC
TACTGATTTTTGTACATTGATTTTGTATCCTTAAACTTTACTGAAGTTATTTATCAGTTCTAGGAGACTTTTGGA
GAAGTCTTTAGGGTTTTCTATGTATGAAATCATATCATCAGCAAAGAGAGACAGTTTGACTTCTTCTTCTTTTTG
GATGCCATTTATTTCTTTCTCTTGCCTAGTTGCTCTGACTAGGACTTCCAGGGCAATGCTGAATAGGAGTGGTGA
GAGTGGGCATCCTTGTCTTGTTCCAGTACTCAAGAGAAATGCTTCCAGCATTTACCTGTTTAGTATGATGTTGGC
TGTGGTTTGTCATAGGTGGATCTTATTATTCTAAGGTATATTCCTTTGATGCCTAGCCTGTCGAGGGTTTTTAAT
CATGAATGGATATTGAATTTTATTGAAGGTTTTTTCTGAAACTATTGAGATGATCATATGGTTTTTGTTTTTTCA
TTCTGTTTATGTGGTGAATCACACTTATTGATTTGTTATGTTGAACCAGCCTTGCATCCCAGGAATAAAGCCTAC
TTGATTGTTGTGAATTAACTTTTTGATGTGCTTCTTGATTTAGTTTGCTCATATTTTGTTGAGGATTTTCGTGTT
TATGTTAATCAGAGATATTGTCCTGAAGTTTTCTTTTTTCATTGTGTCTCTGGCAGATTTTGATATCAGGATGAT
GCTGGCATTGTAGAATGAGTTAGGGAGGAGCCCCTCTCCTTAATATTATGGAATAGTTTCAGTAAGATTACTATC
AGTTCTTCTTTGTATGCTTGGTAGAATTCAGTTGTGAATCCATCTGGTCCAGGGCTAAATTTGGTTGGTAGGTTT
TTATTACTGATTCAATTTTGGAACTTTGTTATAGGTCTGTTCAAGTTTTCACTTCCGTCCTGGTTCAATCTTGGG
AGGTTGTATGTTTCCAGGAATTTATCCATTTCCTCTAGATTTCCTACTTTGTGTGCATAGAGGTGTTCATAACGG
TCTCTGAAAATCTTTGGCATTTCTGTGGGATTGGTCGTAATGTCATTTTTGTCATTTCTTGTGCTTTTTGGAACT
TCTGTCTGTTTTTCCTCGTTTTTCTAGCTAGCAGTCTATTAGTCTTGTTTATTCTTATGAAAAACCAACTCTTTG
TTTCACTAACATTTTATGGACTTTTGCATCTCAATTTTATTTAGTCATTATCTGATTTTAGTTATGTCTTTTCCT
CTGCTAGCTGTGAGATTGAATTGTGCTCTTTTTTTCTAGTTCCTCTAGTGTTATGTTAGATTGTTTAGTTGAGAT
CTTTCTAACCTCTTGATGAAGGCATTTTAGCACTATAAACTTTCCTCTTAACACTGCTTTTGCTACATCCCAAAG
ATTTTGGAAAGTTGTGTCTCTATTTTCATTAATTTCAAATAATTTTTTGATTTCTGCCTTAATTTCATTGTTCAC
CCAACAGTTATTCGGGAGCATGTGGCTTAATTTCCATGCTTTTGTGTAGTTTTGAGAGATCTTCTTGGTATTGAT
TTCTATTGTTATTTCACTATGATTTGAGAGTGGCCTTTGTATGATTTTAATTTTTTTTAATTTATTGAGACTTGC
TTTATGACTGAGCATGTGGGGCAATCTTAGAATACGTTCCATGTGCATATGAGAAGAATGTGTGTTCTGTCATTG
TTGGCTTGAGTATCCTAGAGAGGTCTATTAGGTCCAACTGGTCAAGTGTCAAGTTTAATTCCAGAATTCCTTCGT
CAGTTTTCTGCCTCAGTGATCTGTCTAATGCTATCAGTGGAGTGATAAAGCCCCCACTAATATTGTGCTGCCATC
TACGTTTTATTGTAGGCCAATAATTTGTTTTATGAATCTGAGTGCTCCAGTGTTGGGTGCATATATGTTTAGAAT
AGTTAAGTCTTTTTGTTCAATTGAACCTTTTATCATTTTATAATGCCCTTCTTTGTCCTTCCTGATTGTTGTTGG
TTTAAAGTATGTTTTAATCTGATTTAAGGGTAGCAACTCCTGCTCTTTTTTGTTTTTCATTTGCATGGTAGATCT
TTCTTCATTCTTTCACTTTGAGCCTGTGAGTGTCATTCATGTAGGATGCATCTTCTGAAAACAGCAGACAGTTGT
GTCTTGTCTTTTTATCCAGCTTACCACTTTATGCATTTTAAAGGGAGAGTGTAGACTGTTTACATTTAGGGTTAG
CATTGACATGTGAGATTTTGCTCCTGTCATTGTGTTGTTTAGCTGGTTGTTTTGTAGACTTCATTGTGTAATAAG
TGTATTTTTATTGGTAGCAGGTTTCGTCTTTCATTTCCATGTTTAGCAATCACTTACGGATTTCCTGTAAGAATC
ATCTGGTGGTAATGAATCTCCTTGGTGCTTGCTTGTCTGAGAAGGATTGTATTTCTCCTTCACTTATGAAACTCA
GTTTGGTGGGATATGAGTTCTTGGTTGAAATTTATTTTCTTTAATAATGCTGAAAATATAGGCCCCCCCATATCT
TCTGGCTTGTAAGGTTTCTGCTGACAGAACTGTTGCTGGCCTGATGAGGTTCTTTTTGTAGGTGACCTGACCTTT
CTCACTAGCTGCCTTAACAATTTTTTCTTTTGCATTGACCTTGGTGAATCTGATGACTATGTGACTTGGCAATGG
TTGTCTTGTATAGTGTCTCACAGGAGTTCTCTGTATTTCTTGAATTTGTATGCCCACCTCTCTGGTGAGATAGGG
GAAATTTTCATGGACTGCATCCTCAGATGTATGTTCTAAGTTGCTTACTCTCTTTCTCAGGAATGACTGTGAGTC
ATAGACTTGGTCTCTTTACATAACCTCATAAATCTTGAAGGTTTTGTTCATGTTTTAAATTCTTTTTTCTTTATT
TTTGTCCAACCAAGTTGATTCAAATAACTGGTCTTCAAACTCTGAGATTCTTTCCTCAGCTTGGTCTGTTCTGCT
GTTAATGCCTCTGACTATATTATGAAATTTTTGAAGTTGATCCCTCAATTTCTGAAGTTCAGTTTTGTTCTTTCT
TAAAATAGCTATTTCATCTTTAAGCTCTTTGATCATTTTTCTGGATTCCTTGAGTTCCTTGTATTGGGTTTCAAT
GATCTCCTGGATCTTGATGTACTTCCTTGCCATCCAGATTCTGAATTCTATGTATGTCATTTGAGTCATTTTAAT
CTGGTTAAAATCCTTTGCTGGAGGACTTGTGTGTTTGTCTGGAGGTAAGGAGACACCAGCTTTTTTGAATTGCTA
GAGTTCTTGAGATGACTCTTTAACATATGAGGGCTGGTGTTCCATTAACAATAGTGTACATTGAGTATAGTCAGT
TGGCTTCATTCTGAGTGCTTTCAAAGGGCCAAAGCTCTGTACAGCATCTTTATTTGTGGCTAGATTTTTGCTTTA
GGTTTCACAGGTGCTGTATATTGGAAAAATGTTTTTGGTGTTGTCATTTGGGGTGCAATCCAGTAGGTGATGCTT
AAGAGTGGTAGCTGGCAGATAGGCTCTTACTCAGTCCACAGCTCTTTTGTATTTTGGTGCAGTCCTCAGTAGTGC
TCTGTGGTGGTAGGGAGAGATGACCCCCTCACCAGATACATTCCTGGGCCTTGGGGGAGCCCTCTCTTATTACTG
GCACTGCACCTGCATTTCATTTATTAGGTGTCCTGGGCTGCAGGGTGCCCTCAGGCAGAGGCTGCGGCTGGAAAA
TAGACCATACCCTTCCCTGGCTGGCCCTGCACAAGGAGGCACACCCTGTTCCTGAGCCAGTCCATGAACCCAGCT
GTCTCACCCCTCTCAGTGTTCTGAGAGTAGGGGATCCCCCACTGCTTGAGCACCATGAGCCCCTCCTGGCTACAG
GCAGTGGGGGTAGGTATAGTCTCTCAACCCACTGTCCAACTGATTTCCAGGGTAACAGAGAGCTGTGCCTGCCCA
CAGAGTTCAGGCAGAGGCCAGGCCATTGTGCTGGAAGCTGATGCTAAGCCTTGTCTGATGATGGGGAGTGAAGCA
ATGTAACGGCTCCCTAACTGTGGCTTCTCTCAGGGCTATGGCAGCTGGCATGAGACTGCTCCAGGTCCAAGGCCT
GTGGGACTTCCTGTGGACTTGAGTTTTGCCTCTGCAAACACTCCAGCAACTCTCTATGTCAGTCTAGAGGCCCAG
GGACACGGATCAGGTATTGGGATGAAGGGGTTCTCCAGTTCCCAGGATTTCACAGGTCCCTGTGGAAAGTGAGGA
TCCCCCAGGGGCTCTCACTCACTCACCCTTTCTCTATGTTGGGGAGCTTCCCCTGGCTCCATGCCCATCTTGGGT
GGCCAGCTGCCCAGCTTCACTCTTCCCTGTTCTCTGTGTCCCCTCACTCCCTTAATTGTCCTGATATCGTTCCTT
AGGTGATCTACTTGCAGAGGCAGTGTTTACTCGCCACTTGTTTTCTCTCTGTGAGAGTAGCACACACTAGCTGCT
ACTCATCTAGCATCTTGAATTCTTCCCATCTGAAAAAGTTTCAACTGCAATCACAGTTAAAGAAATACAAAAACA
ATAGCACCTAAGTTACAACTTCTCACCTATAGAATTCAAAAACATCCAAATGATTAACTAAACATTTTGTTTGGT
AGATCTGTGGGAAAACATGAATTCCTTGTGAATTACTGGAGAAAATGAAAATGATGCAACACTTATGGAAGAAAA
TTTGGGGATTTTTGGGGGGGAGGGGAACAATATATTTAAAACTATAAATGCATTTATCCTAGCAATTCTATGAAT
GGGGATTTATCTTAGGGTACACCTGCACACTTAGGAAATAATGTATGCAGTCATTCATTACAGAATTGTTTGTAA
TAGCAACAACCTGAAAAGCAACTCATATATCCATCCATCACACAGGGACTGGTTTCATGACTACGGTTCATGAAT
ACTCTGCAGCCCTTAGAAAGAATGAGGAAGTGGCCGGGCACGGTGGCTCATGCCTGTAATCCCAGCACTTTGGGA
GGCCGAGGCGGGTGGATCACGAGGTCAGGAGATCAAGACCATCCTGGCTAACACGGTGAAACCCCGTCTCTACTA
AAAACAATACAAAAAAATTAGCCAGGCAGGCGCCTATAGTCCCAGCTATTCGGGAGGCTGAGGCCGGAGAATGGC
ATGAACCCGGGAGGCAGAGCTTGCAGTGAGCCGAGATAACGCCACTGCACTCCATCCAGCCTGGGCGACAGAGCG
AGACTCCGTCAAAAAAAAAAAAAAAGAGGAAGTTCTCTATGCGCTGACATGGAAGGAAGACAGATGGTTGAATGA
AAAAAGTACATAATTAGCCATAAAGTGTAAGACTTTTTGTCTAAAAAAGAAGGGTGATATAATTGCATATTTATA
TTTTCTTCCATTTATATTAAGAGATAATAAAGGTACACAAATTGGCTAGAATAAAGTGGTTTCCTATAAAGGGTA
AGAGTAATTGAGTGGATGAAGACTAGGGTTAGGGATAGATTTTCAGTTGTATTCATTTTAATATATGTATTCATT
TTATATATGTACTAATTTTTATATATGTATTTATTTTATATTTTGATTTTCTTAACATAAATATATTATTCCTTC
ATAAAATTAAACTTGATACATTTTTGATTACTAGATATGTAGAAAGCATTATGTTCAGTACCACAGTAATACTTT
CAAACCAGCTACAATTAGTATTTATGAGCATCTATGTGCCAGACATTGTGTTCTGCTTTGGTTGGTGGGGGTAGA
GGAGGAAAGGAAACCATGGCTTACATAGGAGTGGAAGTCTTGTCTTTCACTTTGCACCTCTCTCCTTCAGACCTA
GCATAAATATGACCTTAGGGGAGGCAGAACACATATGATAAAGAGATAACTAGCAAGAGACATAATAGTAGCTAA
ATAAATACTGAAGGAAAAATTCAGGAAGAGGTAGGAAGGATATGCCTCATCACTTCCACCTGTTAAGAAAAACTT
TAGACATTCTTGCCAATATTCCTTATTGCCTGTCTTTTGAACAAATGCCATTATCACTAGAGTGAAATGATATTT
CATTGTAGTTTTGATTTGCATTTCTCTCATGATCGGTGATGTTGAGCACCTTTTTATATACCTGTTTGCCATTTG
TATGTCTTCTCTTGAAAAATGTCTATTCAGATCTTTGCCCATTTTTAAATGGCGTAATACATTTTTTCCTATTGA
GTTGTTTGAGTTCTTTATATATTCTGGTTATTAATCCCTTGTCAGATGAATAATTTGCAAATATTTTCTCCCATT
CTGAGGATTACCAGAGGCTCAGAGGGGTAATGGTGGTGGGGGAGAATAAAAATGGTTAATGAGTACAAAAATATA
GATAGGAGTAATAAGATCTAGTATCTGATAGCACAACAGGGTAATTACAGCCAACAAAAATTTTATTGTGCATTT
AAAATAACTAAGAGTATAATTGGAATGTCTGTAACACAAAGAAGCAATAAATGCTTGAGGTGATGTGAGGGGATG
GATATCTAATTTACCTTGATGTGATTATTACATATTGTATGCCTGCATCAAAATAGCTCATGTATCTTATAAGTA
TATACACCTATTATGTACCCATTAAATTTTTTAAGAACTTTAAACAAATCAAATTTAACAGAGTTTAATTGGGCA
AAGAATGATTTGAGGATCAGGCAACCCCCAGAAACAGAAGAGGTTCAAAGCAACTCAGTGCTGTCACATGGTTGG
AGAGGATTTATGGGCAGAAAAGGGAAAGAGAGATACAGAAAATGGAAGTGAGGTACACAAACAGCTGGATTGGTT
ACAGCTTGCCATTTGCGTTATTTGAACATAATCTGAACAGTTGGCTGTCTTTGCTTGACCAAAACTTGGTGTTTG
GTACAAGAGCAGATTACAGTCTATTTACACATCCAGTTAGTTTTACAGTTCACTATACACGAAGAAGAAACCTTA
AGCAGAACTTAAAATATGCAAAGAGGAAGCTTTAAGTTAAACTTAATTTAACACACCCAATTATCAAAAAATGAG
TAGCTCTGCAAAAGTGGATTTTCCTGGTCATCTTTGGTACTTCCTTAAAAAAGAGAAAAGTAGTACTCACGATAA
AAAAAAAAAAGTCCTCAAGTCTTTATTTTATTCCTTTCCAATTTAAAATGTTACATCATCTGAGGAAGGTTTTTC
CCTTTGACCGCTTTCATAGACATTTCTTCTGCATGGGTTGGCCAGAATCAGAAGAGTAATTGTAACTTTCTGTTC
TTGTCCTACAGTTACAAAGCGGTTTCACTTTGTAAATGCTCTTTGGATGGCAGGAACCAAGCAGCCATGAAAAGA
GGAGTTACACCTTTAAAGGAGTCATTCCATCATGACTCTCAGGACTGGAACATGGAATACCTGAATGGCCTCTTT
GGCACAGATAGGCCACCCTTGAAAGGTGTTCCAAGCTAGGAACTCACTACCACTGTTACATCGATGCAACTCTGT
GAGAAGTTTTTATCTGGTGATGGAAAATCTCATCTCTTCAACACACTGACTACTACCAGTCTCAGAACCCTGTAA
ACAAGATTCATTCATCTCAAATTGGGTTAAAGCAGTCACCCTGCCTTACATTAGTTTGGAATAAGGATGTGGGGA
TGGTGGTAGAGGAGGGGAGTGGATGATGATTTTTTTATTGTTATTTGATTCTAAAGAAACTTCTATACATTTTGC
ATTTAAAATAATTATGTTTTTAACAATGTTTGGATTAATTCAAAATAGGATATTATATCCTATTATATTAAATAT
ACTATTTAATCATCTTGTTGACCAAATGCAACTTAAACATGTAAAATGGTAAATAGCATAATAATTGTCTTCTAA
GCCTGCACTATAAAGTATTTCAGTGGCCTCATTATTAAAGGACCAAGGTGCCCAAAGAAACAAAATTTAGTAATC
ATAAACAAGAGACAAACCTACTTCTTTTCCCCCAGAGTTCTGGCCACATTGAAATAAGGTGTTTGAATGCTTAAT
AAGAATTATTTTGGCCCACACAGTGGCTCATGCCTGTAATCTCAGCACTTTGGGATGCCAAGGTGAGCAGATCAC
TTGAGGCCAGGAGTTCAAGACCAGCGTGGCCAACGTGGTGAAACCCCATCTCTACTAAAAATACAAAAATTAGCC
CGGTGTGGTGGTACACGCCTATAGTCCCAGCTACTCGGGAGACTGAGGTGGGAGAATCACTTGAACCCGGGAGGC
CAAGGCTGCAATATCGAGATCACACCACTGCACTCTAGCCTGGGCAACAGAGTGAGAGTGAGACTCTTTCTCGGA
AAAAAAAAAAAAGAATTATTTTGAACAAAGTGCTGTCACCTAAGTTAGCAAAACTCCAAGCAAGGTTTTTGGCTC
TGTAAGGAAAGAATTAGCCTACTCATTTGGAAATTTAGTGGTGTTTGTAATGCAGAAAGTGACAGTGAGACTGGA
AAGGGATTGGCTTTGGGGCTTGTTCTGCTTTATAAATAATAATGAATCTTCTCCAACATGAAGTAATGTGAATTA
AAAAAAAAAAATCTGTCCTTAGAGTACAAAATTACTTCATAACCCAATCTGCATTTCTCCACTCCAAGCATATTT
TCTGGGAGTTCTACTTAGAGAGTGAAAGCTGCTGTGTGTGTGATAATTAATTTTAACAAACACTTGGCAAACTGA
GCTGGACTATGTATAAGCTACCCTAGACTAAGCATGAATTTGAACTGCACTTTTTATGGTGTTTTTTCCACAATG
ACATTATTTAGGCATTTAAAGTTATCTGAACTGCAATTTTTTGTTCTTTTTTTTTTAATTTGACTTTTTAAAAAA
AATTATTCCTGAATAAAGAGGCAGTTTGTAAAAACTCGAGAACTGTGAGAGATAATTGGATCTTTGTGTAGCAAA
ACTAGAAGGGTGTTGGGTATCTGCTCTTTATCAAATGGACCACTTACTTTTCTTTTCTTTTTTGCCCTGTGTTCA
GAAAACAAATGTGCGTGTCTCCTGATTTATAATGTATAGTTCATTAATGGAGAAAGTGCTTGAGAATTAGATCCT
AATGTCATTTCCCATGCAGCATCTTCATTCTTTTCTAAAGCACTATTTGGTAAAAACAACTGATAGTCGTCAGAG
GTGATCACCAATGTTTGAGCACTATTTCCTTTTTATATCCTGCACATGGAATATGGACAGGCAAACAAATCATTT
CCAAGTAAGAAAATAAATTTTGAGGGAGTTAATACTATAATTTGAAAGTAATAACCTCCTATTTATCCATCTAGT
TTGTTGTTCTGTACTAAATTATTTGTGCATGTCTCTGTGTCTATAATTTATGTGAAACTTTGCACAATCTTAAAT
AGGACAAAATAGACATTCTGTAATTTCCCAGGCAAGCTATTTAAGGTGACTATCTCTCTACATATTTGAGATGAA
AAACAATAACATGACAATCCATCCCTTCTTAGGTTTTTGTAAGCAGACTTACTACCTGTGACTCAGTTTTGTTCT
CACAGGGTACTAATTAATCCTTCACGATAATAACTTGTCAAATCCATTACTTCTGTAAAGGCAATACTTTTATAT
TTGTTTGTATTCAAATTTTAAACTGATGTTAAATGCCGTGGGTGCAACTGCAGGTTAAAAATATGTGTTTGAATC
TCTTATTCTTTTTGCTTGGCAATGTATGAAATAACTGCTCTTTCTAGAAATCTTGATGATGAAGTGGCCTGTTGT
TTTGTCACCTAAAAATGCAATAATGTTCAAATTAAGCTTTTCTTTATTAACATCACTTGATTGTGTGCCATATTT
AGAGCTTAGTGAAATTTTAATCTACACATTGATTAAATACATTTTATTTATTCTTGTTTCTAATGGGAACTTTCT
TTGTTTCTAATGGGAACTTTCTTAAATTAAATTACATCCAACATTTATTAAAGACCTAAAACATAGGCAATTACT
GTGCTTAGAGGAAAAGCGCAGACGAAAGTGAATCAGACAAGTTTCCCTGCCCTCCGGAAGCTTCAGTCTAGTGAT
GAGAAAGACGTATACACACCTTATGTTGATTTAAAAAAAAAAAAAGCTCTTACCTGGTTGCTGGCATATGAAAGT
GTTAGTTACAGATCTGCCCCAAACTAAAGGTGTCACCTCGAGTAAATCTCTTTCCCTTTCCCTTTCAATCTCTTC
ATCTATAAACTAGGGGTTGGGAATACATTTATTAACAAACACAAATTGAGCGTCTACCATGTGATAATAGTAGCT
AAACTTACTGAGCAATTACCATGGGGCAGGTATCAAGATAAACCCTTTATGATGGTAACCTCATTTAATCCTCAA
AGCAATTCCATTTTCAAGAGGAGGAAATTGAGGCTCAAAAATGTTAAGTAACTCCCCCAAGGATGCAAAGTGATT
GAGCCAGAATTCAAGACTAGGTTGGTTTGACTCCAAAACTCATGCCATTAAACCCTATTGTGTCACTGCAAACAA
CTCTAATAGTTTCAAATTATTAGTTCTATTAATATTATATTACCATTATTTGCCCCCAAAATGTAAAATGTAAAT
ACAAAGAGTTTGGTTTTTGTATTACTAGTGGAGGTTAAAGGTGCACAATGGAATTATTCAAACTGGGAAAATCCA
GGAAGACTTCATGGAGGAGGCAGCATATGGCTGCAGTTAATAAGGTTTGCTCACACAAAATGGAGAGGTGAGGAC
ATTTCAGGCAGAGAGAATTATATGAGAGGTTACAGAGCAGTAAACAGTCATGCGTCTGCAAGATCAAAGGGAAAG
GGCGGTAAGAGAGAAGCTTGAAAGTCAAGTGGAGCCAGATTGTGGAAAAACTAGAGAGTCATGCCAAGGACCTTG
ACATATAGAAAATGGGAAGCCCCTGAAAGGTGAAGAACATGAGAGTGAAATGATTAGTAACTTTTTGGTTTAGGA
CTTGTTTCTTTTGTGTTTTGGTTGCTTTCTTGTTTTGETTTGTTTGTGGTTTTTAAATTTACAACCAATAAGAAT
ATTTAGTAAGGTTTCCAAATACATCATGAATATATAAAACTAGCCTGACTCAAGGATAATAATTCTGGGTAGTTG
GAGTGAAGTTTCAATCAGCTACGTGGCATTTGCTAATCATCTGATATGAGCTAACAATAAAGGAGTTAACAAATA
AACTGTCAGCCTACAGTCCAGGGTCTCAAATAGCATGTGACATAGTTGAGAAGCAGTTTTCCATATCATACATGA
AATAACTAAAGAAACTACTTACAAAGCACTATACCAGTAACTACAATAAAATACAACTATACATGCAAAATAATG
CTGAAAGCTGCAAGTAGAGGGGTAAAGCTAGGCCAGTTGCTCAGGGAACCATTCTGAAGTTGGATTGGGAAGTAT
GTCTAGAAGGGGAGCCATTGCTGTGAGAGTGCTGAGGCTCATCTGCTACTAGTCCCCCACTACTCAGGCATATGG
TAGGTCAGTAACAAAACCATCATTGTGCACTGTTCTTTCCATCTAAATTCCATCAAATTATGACCAACCTATCAA
GGTACTAGTTCAAATTCTCTCTTCCTCTATAAGCTAGTGGTCTTCTCTAAAATTTAAGAAGATCGTGCTCATCTT
CCTACTTCTTGTTCTCTTTCTTCTGTGTTTTCTGAGGCTGCAATGAACTAGGAACTTCCTCTCCCCAGAACTCTG
TATTCCAGGCCTTAGATCACTCAAAACTGTTGCTTATAAAGTGCAGAGAATCAACAGAGAAGGAATAGAGGTTAA
TGTCTGGTCAAAGATGTGATTCTCTTGTTGAAAAGTTCATTAGCTTATTATTTATAGAATCATAAGTCCCAGGAA
AAACCAAAAGGAAATATATATTGGATCCTAATGATATTCTCTTTTTTTCTTTTTTCTTTTCCCCCACTCCATTGC
CCAGGCTGGAGTGCAGTGGCATAATCTCAGCTCACTGCAACCTCCACCTCCCGGGTTCAAGGGACTCTCCTGCCT
CAGCCTTCCAAGTAGATGGGATTACAGGCATGTGCCACCACATCTGGCTAATTTTTTTTTGTATTTTTAGTAGAG
ATGGGGTTTCACCATGTTAGTCAGGCTGGTGTTGAACTCCTGACCTCAAATGATCCACCAGCCTCGGCCTCCCAG
TGTGCTGGGATTGCAGGCGTGAGCCACCACACCCGGCCTGATATTCTCTTGCAAGGGCATTGTTTACATTGTCTA
TCATCAGAACTGTAGAGTGTTGGCTCCAGGCACAGAACCCCTAGAGTTTTGTAAACCATTTATATCACACTGGCA
ACCAGAAGTAACTTTATATACTCAAGAATCAAGATTTCACCTAGAAGTACCTCAGGTAGGTGTTGGTTCATTCAC
ATTCCAACCAAAAGATAATGTACCATAAAGTGCATACCGCCTAGTCCGTAATGATTAAGGCAACCACATAAAATC
TCATTATTTAAAAGAAATTAAGTCCAGGCACGGTGGCTCACACCTGTAATCTCAGCACTTCGGGAGGCCAAGGAG
GGCAGATCACCTGAGGTTGGGAGTTTGAGACCAGCCTGATCAACATGGAGAAATCCCATCTCTACTAAAAATACA
AAATTAGCGGGGCATGGTGGTGCATGCCTATAATCCCAGCTACTCAGGAGGCTGAGGCAGGAGAATCACTTGAAC
CCAGGAGGTGGAGGTTGAGATCGTGCCATTGCACTCCAGCCTGGACAACAAGAGTGAAACTCTGTCTCAAAAAAG
AAAAAAAGAAAAAGAAATTAAATGCACTATGGTTTATGGAGCGGTATTCCTCCTCCATGTCCTACATAAGATCTT
TCACATGCCAGTCACAGTTAAATCTAATTTGCTGTAATCTGGATAAATGGGAGCTAATCAACAAGCTCTCAGCTC
TAGCTCTGAATCAGCAGCAGATATTGCATTTTTGAAATACACTAATAGCAAGAATGCCTTCCTGACAACAACTGG
CATTTTTGACACAGCAGGAAGTTTATCTGGATTCTGATATAATAGTTATTGGAATCATACATAGGTACATAGTTT
AAAAGGCTAATAAGTCATTTGTTATTGCTTTTATTATCTCTGCATAGTTAGTAAAATTGAGATTAGAACCACTTC
TCGAATGTACTGTTCTAAATCCTTAGCTTGCTTGATCACACATGACCCTCACAATGATCCTAGGAGAAATTATTC
TGCATGCCATTTTGTAGCTGGGGAAACTGAGGCACAGAGAAATACAGTACTGCCCAAAATGTCATAACTAATCAA
AGGCAAAGACAATACTCACACCAGCTCTGATTCCAGAGCCCACTCTCTTAACCATATGCTTTTCTGCTTCCCTAG
TTGTAGAGTCTTTTTGTATGACTGCATTAATTATATGTGAAGAGTTCAAAAATTTCTATATAAGGTCTTTTAAGG
GTGTCATTCTGGTTGAAAATGGAGGACTAGGCTTCTCACTTGAAGACATATTTCTGTAGAAAAACCTATTTTCAT
TTAGATGCTACAGTTACTTGATGTGGTTAATAAACCAGTTAACAGAGTATGAAAAGGATAAGGGTTAAAGCCCTC
CCAAGCCATCTTTCATGCTGCTAATATGAATCACATTACTAGATACTTAAATATCATTTTCTCTTTGGTTCCCAG
AAGACTGCATATATGCTAGAATATTTGTCCTCCTCTTTTACCCTTTCAGGCAATAAAGTATTTTGGACCACTGTA
CTATGTTATAATTATTGTTTCTCTCCTGATTTTTTTGCTCCAATCTAATGAAAGACATACAAGCTACTATACTGC
TACACAATGACTAAATACCTGTTGGATTAGGTGGGGGGAAGATACACAGTCACTGGCTAGAAAGCATCATGCATA
CAGAGCCATTTTCACCATATATTTTATTTCTCATGATCATGTAGAATTTAGGCTTTGGTGTTGATTATTTCTCTC
TTAGGAAACATAGTTGTTTCAGGGTTGATATCACAAAAAAACAGAAAAACCTATTCGAGAAAAGGAAAATTATTT
GTCTGTAGGCCAAATTTTGAAGTAGGAAAACCTGCTTTTGGAGTTGTATTCCCCTCCCAGGCACTTAATCCAAGT
TCCAGTCTTATTCTAAACTGGGGATGCTAGTATTAACCACCATAGGAGTTATCTGAGATGAGTTATCATCAACTT
GGTACCAGGTTGTTGTCCTCTGGACTCAGTGAGCTCTAGAATTGCATGAAACTGGCCTAATTTATCAAAGTATGT
AGCCTTGGGTAAATAATTCAAGCTCTCAGAGGTCCAGTTATCTCCTCTGTAAAACATATCTACATCCTAGGGATG
ACAATATCTACATCCTAGAGATGTCAGGAGGATTAAGTTGTAATTTTTTTAATTGTATGTATTTAAAATGGGCAA
CATAATGTTTTGATATACACGTGTATAGTGATTACTACAGTCAAGCAAATTAACATATCCATCATTTCATAGCTA
CCTTTTATGTATGTGATAAGATTATCTAAAATCTATTCTCTTACCAAATTTCCAGTATACAATATTGATATGGTT
TGATCCATATCCCCATCCAAATCTCATGTTCAGTTGCAATCCCCAACGTTGGAGATGGAGCCTGGTTGGAGGTGA
TTGGATCACAGGGGTGGCTTCTAATGGTTCAGCACCATCCTTTCTTGGTACTGTATAGTGAGTAAGTTCTCACGA
GATCTGGTTGTTTAAAAGTGTGTAACACCTCCCCCACTTTCCCTCTCTCTGTTCCTCCTGCTCCCGCTATGTGAA
GTGCCAGCTCCCTCTTTGCCTTCCGCCATGATTGTAAGTTCTCTGAGGCATCCCCAGAAGCTGATGCTGCCATGC
TTCCTATACAGCCTGCAGAACCATGAGTCAATTAAACCTCTTTTCTTTGTAAATTACCCAGTCTCAAGTATTTCT
TTATAGCAATGCAAGAATGGACTAATACAGAAAATTGTTACTGAGAAGAAGGGCATTGCTATAAAGATACCTGAA
AATGTAGAAGTGACTTTGGAACCGGCTAACAGGCAGAAGTTGAAACATTTTAGAGGGCTCAGAAGAAGACAGAAA
GATGAGAGAAAGTTTGGAACTCGCTAGGAACTTGTTGAGTGGTTGTAACCAAAATACTGATAGTGATATAGACAG
TGAAGTCCAGGCTGAGGAGGTCTCAGATGGAAATGAGAAATTTATTGGGAATGAGTAAAGGTCAGGTTTGCTATG
CTTTAGCAAAGAGCTTAGCTGCATTGTTCCTCTGTTCTAGGGATCTGTGAAATCTTAGACTTAAGAATGATGATT
TAGGGTATCTGGCAGAAGAAATTTCTAAGCAGCAGAGTGTTCAAGAAGTAACCTAGCTGCTTCTAATAGCCTATG
CTCATAGGCATGAGCACAGAAATGACCTGAAATTGGAACTTACACTTAAAAGGGAAGCAGAGCATAAAAGTTTGT
AAATTTTGCAGCCTGGCCATGTGGTAGTAAAGAAAAGCTCGTTCTCAGGAGAGGAAGTCAAGCAGGCTGCATAAA
TTTGCATAACTAAAAGGAAGGCAAGGGCTGATAACCAAAACAATGGGGAGAAAGACTCATAGGACTAACAGGCAT
TTTATTTTTATTTATTTTTATTTTATTATTATTATACTTTAAGTTTTAGGGTACATGTGCACAATGTGCAGGTTA
GTTGCATATGTATACATGTGCCATGCTGGTGTGCTGCACCCATTAACTCGTCATTTAGCATTAGGTATATCTCCT
AATGCTATCCCTCCCCCCTCCCCCACCCCACAACAGTCCCCAGAGTGTGATGTTCCCCTTCCTGTGTCCATGTGT
TCTCATTGTTCAATTCCCACCTATGAGTGAGAACATGTGGTGTTTGGTTTTTTGACCTTGCAATAGTTTACTGAG
AATGACGATTTCCAATTTCATCCATGTCCCTACAAAGGACATGAACTCATCATTTTTTATGGCTGCATAGTATTC
CATGGTGTATATGTGCCACATTTTCTTAATCCAGTCTATCACTGTTGGACATTTGGGTTGGTTCCAAGTCTTTGC
TATTGTGAATAGTGCCACAATAAACATAGTGTGCATGTGTCTTTATAGCAGCAGGATTTATAGTCCTTTGGGTAT
ATACCCAGTGATGGGATGGCTGGGTCAAATGGTATTTCTAGTTCTAGATCCCTGAGGAATCGCCACACTGACTTC
CACAATGGTTGAACTAGTTTACAGTCCCACCAACAGTGTAAAAGTGTTCCTAATAGGCATTTTAGGCTTTCATGG
TGGTCCCTCTCATCACAGGCCCCGAGGCCTAGGAGGACTGAATCATTTCCTGGGCCAGGCCTAGGGCCCCTGCTC
CCTCTTACAGCCTTGGGACTCTGCTCCCTGAATCCCAGCTGCTCAAAGGGGCCCAGGTACTGTTACAGTAGGTAG
CTAATCAGGCATGAGTGGGGTAAGAGAGAAGTCCCCACCACCCACCAGGAATGTCAGGCAACCATCAGATGATGG
TCAGGCAGTTGTCATACTGCCTCTCTAAAATAGTAATTGGTTGCAGCCAGCACCAGGGAGAGGCAACTTCTCAAT
AGATAGAAACACCTGAAATTGGTAACTGGGCGCTTCCAATAAGATCTCAGGAACTGAGAGAGTGGGCTTAACATG
CACATTAAGAGGCAAAATGGTGAAGTATGACCTTTGGGGGCATTCCACCGGAAAAGGGAAGAAAGCCTCAGGTAA
GCATGTATACAACTCCAGTAAACACACTGCACACGCTCACCTTCCAAGTGCAAGCAGGGCACCATGCATGCGGCA
AGCTCACCCTTAGGGAAGGACCAAGGGAAAGGGGCACAAGATGTCAGAAGTAGGCCAGTGTATAAGATCCTAGGT
TCAAGGTCAAACAGGGCACTTGACCTCCAAGGTGCCCACTTGGGCCTCTTCCAAATGTACTTTCCTTTCATTCCT
GTTCTAAAGCTTTTTAATAAACTTTTACTCCTGCTCTGAAACTTGTCGCAGTCTCTTTTTCTGCCTTATGCCTCT
TGGTCAAATTCTTTCTTCTGAGGAGGCAAGAATTGAGGTTGCTGCAGACCCACATGGATTTGCAGCTGGTAACTC
AGATAACTTTCACCAGTAAGAATACAGTTCAGGCTGCTGCTTCACAGGGTGCCAGGCATAAGCCTTGGTGGCTTC
CATAAGCTGTGAAGCCGGGGGGCGCACATAATGCAAGAGTTGAGGCTTAAGAAGCTCTGCCTAGATTTTAGAGGA
TGTATGAAAAAGCCTGGATGTCCAGACAGAAGCCTGTTACTGGGGTGGAATCCTCATGGAGAACATCTACTAGGG
AAGCAAGGAGAAGAAATGTGGGGTTGCAGCCCCCACAGAGAGTCCCCTGGGGCACTGCCTAGCAGAGCTATGACA
AGACAGCCACCGTCCTCCAGACCCCAGAATGGTAGATCCACCAACAACTTGCACCCTGCAGCCTGGAAAAGCTGC
AAGCACTCAATGCTAGCCCATGAGAGCAGCTGTGGGAGATGAACCCTGGAAAACCACAGGGGTGGTTCTGCCCAA
GGTTTTGGGAGCCCACTCATTGCATCAGTGTTCCCTGGGTGTGAGTCAAAGGAGATTATTTCAGAGCTTTAACAT
TTAATGACTGCCCGGCTGGCTTTCAGACTTGCAATGGGGCCCTATAGCCTCTTTCTTTTGGCAGATTTCTCCCTT
TCGGAATGGCAGTATCTGCCCAATGCCTATACCCCCATTGTATCTTTGAAGCAATTACCTTGTTTTTGATTTTAC
AGGTTCATAGGTAGAAGGGACTAGCTTCGTCTCAGGTGAGACTTGGGACTTTGGACTTTTGAATGAATGCTGGAT
CGAGTTAAGACTTTGGGGAACTGTTGGTAAGGCACGACAGTATTTTGCAATATGAGAAGGACATTAGATTTGGGA
GGGGCCAGAGTTGGAATAACATGGTTTGGATCTCTGTCCCCACCCAAATCTCATGTTCAACTGTAATCCCCAGTG
TTGGAGGTTGGGCCTGGTGGGAGGTGAGTGGATTATGGGGTGGCTTCTAATGGTTTTGTACAGTCCCCTCTTGGT
ACTATATAGTGAGTTCTGACAAGATCTAGTTGTTTAAACGTATGTAGCACCTCCCATTTCTCTCTTCCCCCAGTT
CCTGCCATGTGAAGTCTGGGGTCTCCCTATGCCTTCCATCATGATTTTAAGTTCCCTATGGCCTGCCCAGAAGCT
GATCCAGCCATGCTTCTTGTACAGCCTGCAGAACTGTGAGCCATTAAACTTTTCTTTATAAATTACCCAGTTTCA
GTTATTTCTTTATAGCAGTGTAAGAATGGACTAACACAATTATTAACGCTAGTCCTCATGTTGTACATTAAATCT
CTAGATGTATTAGACGTAACTGCAACTTTGTACCCTACCCTACAATTTTCTTTCCCCCCAAGCCCCCCAACCAAG
GGTCTACTCTGTTTCTATAAATTCAGTTGTTTTTTAATTCCACGTATAAGTGAAGTACAACTCAGTGTAGAAACT
TGGTAAATGCTAGCTACTTGTTATAAGCTGTCAGTCAAAATAAAAATACAGAGATGAATCTCTAAATTAAGTGAT
TTATTTGGGAAGAAAGAATTGCAATTAGGGCATACATGTAGATCAGATGGTCTTCGGTATATCCACACAACAAAG
AAAAGGGGGAGGTTTTGTTAAAAAAGAGAAATGTTACATAGTGCTCTTTGAGAAAATTCATTGGCACTATTAAGG
ATCTGAGGAGCTGGTGAGTTTCAACTGGTGAGTGATGGTGGTAGATAAAATTAGAGCTGCAGCAGGTCATTTTAG
CAACTATTAGATAAAACTGGTCTCAGGTCACAACGGGCAGTTGCAGCAGCTGGACTTGGAGAGAATTACACTGTG
GGAGCAGTGTCATTTGTCCTAAGTGCTTTTCTACCCCCTACCCCCACTATTTTAGTTGGGTATAAAAAGAATGAC
CCAATTTGTATGATCAACTTTCACAAAGCATAGAACAGTAGGAAAAGGGTCTGTTTCTGCAGAAGGTGTAGACGT
TGAGAGCCATTTTGTGTATTTATTCCTCCCTTTCTTCCTCGGTGAATGATTAAAACGTTCTGTGTGATTTTTAGT
GATGAAAAAGATTAAATGCTACTCACTGTAGTAAGTGCCATCTCACACTTGCAGATCAAAAGGCACACAGTTTAA
AAAACCTTTGTTTTTTTACACATCTGAGTGGTGTAAATGCTACTCATCTGTAGTAAGTGGAATCTATACACCTGC
AGACCAAAAGACGCAAGGTTTCAAAAATCTTTGTGTTTTTTACACATCAAACAGAATGGTACGTTTTTCAAAAGT
TAAAAAAAAACAACTCATCCACATATTGCAACTAGCAAAAATGACATTCCCCAGTGTGAAAATCATGCTTGAGAG
AATTCTTACATGTAAAGGCAAAATTGCGATGACTTTGCAGGGGACCGTGGGATTCCCGCCCGCAGTGCCGGAGCT
GTCCCCTACCAGGGTTTGCAGTGGAGTTTTGAATGCACTTAACAGTGTCTTACGGTAAAAACAAAATTTCATCCA
CCAATTATGTGTTGAGCGCCCACTGCCTACCAAGCACAAACAAAACCATTCAAAACCACGAAATCGTCTTCACTT
TCTCCAGATCCAGCAGCCTCCCCTATTAAGGTTCGCACACGCTATTGCGCCAACGCTCCTCCAGAGCGGGTCTTA
AGATAAAAGAACAGGACAAGTTGCCCCGCCCCATTTCGCTAGCCTCGTGAGAAAACGTCATCGCACATAGAAAAC
AGACAGACGTAACCTACGGTGTCCCGCTAGGAAAGAGAGGTGCGTCAAACAGCGACAAGTTCCGCCCACGTAAAA
GATGACGCTTGGTGTGTCAGCCGTCCCTGCTGCCCGGTTGCTTCTCTTTTGGGGGGGGGGTCTAGCAAGAGCAGG
TGTGGGTTTAGGAGGTGTGTGTTTTTGTTTTTCCCACCCTCTCTCCCCACTACTTGCTCTCACAGTACTCGCTGA
GGGTGAACAAGAAAAGACCTGATAAAGATTAACCAGAAGAAAACAAGGAGGGAAACAACCGCAGCCTGTAGCAAG
CTCTGGAACTCAGGAGTCGCGCGCTAGGGGCC(GGGGCC)ₙ
GGGGCCGGGGCGTGGTCGGGGCGGGCCCGGGGGCGGG
CCCGGGGCGGGGCTGCGGTTGCGGTGCCTGCGCCCGCGGGGGGGGAGGCGCAGGCGGTGGCGAGTGGGTGAGTGA
GGAGGCGGCATCCTGGCGGGTGGCTGTTTGGGGTTCGGCTGCCGGGAAGAGGCGCGGGTAGAAGCGGGGGCTCTC
CTCAGAGCTCGACGCATTTTTACTTTCCCTCTCATTTCTCTGACCGAAGCTGGGTGTCGGGCTTTCGCCTCTAGC
GACTGGTGGAATTGCCTGCATCCGGGCCCCGGGCTTCCCGGGGGGGGCGGCGGCGGCGGCGGCGCAGGGACAAGG
GATGGGGATCTGGCCTCTTCCTTGCTTTCCCGCCCTCAGTACCCGAGCTGTCTCCTTCCCGGGGACCCGCTGGGA
GCGCTGCCGCTGCGGGCTCGAGAAAAGGGAGCCTCGGGTACTGAGAGGCCTCGCCTGGGGGAAGGCCGGAGGGTG
GGCGGCGCGCGGCTTCTGCGGACCAAGTCGGGGTTCGCTAGGAACCCGAGACGGTCCCTGCCGGCGAGGAGATCA
TGCGGGATGAGATGGGGGTGTGGAGACGCCTGCACAATTTCAGCCCAAGCTTCTAGAGAGTGGTGATGACTTGCA
TATGAGGGCAGCAATGCAAGTCGGTGTGCTCCCCATTCTGTGGGACATGACCTGGTTGCTTCACAGCTCCGAGAT
GACACAGACTTGCTTAAAGGAAGTGACTATTGTGACTTGGGCATCACTTGACTGATGGTAATCAGTTGTCTAAAG
AAGTGCACAGATTACATGTCCGTGTGCTCATTGGGTCTATCTGGCCGCGTTGAACACCACCAGGCTTTGTATTCA
GAAACAGGAGGGAGGTCCTGCACTTTCCCAGGAGGGGGGCCCTTTTCAGATGCAATCGAGATTGTTAGGCTCTGG
GAGAGTAGTTGCCTGGTTGTGGCAGTTGGTAAATTTCTATTCAAACAGTTGCCATGCACCAGTTGTTCACAACAA
GGGTACGTAATCTGTCTGGCATTACTTCTACTTTTGTACAAAGGATCAAAAAAAAAAAAGATACTGTTAAGATAT
GATTTTTCTCAGACTTTGGGAAACTTTTAACATAATCTGTGAATATCACAGAAACAAGACTATCATATAGGGGAT
ATTAATAACCTGGAGTCAGAATACTTGAAATACGGTGTCATTTGACACGGGCATTGTTGTCACCACCTCTGCCAA
GGCCTGCCACTTTAGGAAAACCCTGAATCAGTTGGAAACTGCTACATGCTGATAGTACATCTGAAACAAGAACGA
GAGTAATTACCACATTCCAGATTGTTCACTAAGCCAGCATTTACCTGCTCCAGGAAAAAATTACAAGCACCTTAT
GAAGTTGATAAAATATTTTGTTTGGCTATGTTGGCACTCCACAATTTGCTTTCAGAGAAACAAAGTAAACCAAGG
AGGACTTCTGTTTTTCAAGTCTGCCCTCGGGTTCTATTCTACGTTAATTAGATAGTTCCCAGGAGGACTAGGTTA
GCCTACCTATTGTCTGAGAAACTTGGAACTGTGAGAAATGGCCAGATAGTGATATGAACTTCACCTTCCAGTCTT
CCCTGATGTTGAAGATTGAGAAAGTGTTGTGAACTTTCTGGTACTGTAAACAGTTCACTGTCCTTGAAGTGGTCC
TGGGCAGCTCCTGTTGTGGAAAGTGGACGGTTTAGGATCCTGCTTCTCTTTGGGCTGGGAGAAAATAAACAGCAT
GGTTACAAGTATTGAGAGCCAGGTTGGAGAAGGTGGCTTACACCTGTAATGCCAGAGCTTTGGGAGGCGGAGGCA
AGAGGATCACTTGAAGCCAGGAGTTCAAGCTCAACCTGGGCAACGTAGACCCTGTCTCTACAAAAAATTAAAAAC
TTAGCCGGGCGTGGTGATGTGCACCTGTAGTCCTAGCTACTTGGGAGGCTGAGGCAGGAGGGTCATTTGAGCCCA
AGAGTTTGAAGTTACCGAGAGCTATGATCCTGCCAGTGCATTCCAGCCTGGATGACAAAACGAGACCCTGTCTCT
AAAAAACAAGAAGTGAGGGCTTTATGATTGTAGAATTTTCACTACAATAGCAGTGGACCAACCACCTTTCTAAAT
ACCAATCAGGGAAGAGATGGTTGATTTTTTAACAGACGTTTAAAGAAAAAGCAAAACCTCAAACTTAGCACTCTA
CTAACAGTTTTAGCAGATGTTAATTAATGTAATCATGTCTGCATGTATGGGATTATTTCCAGAAAGTGTATTGGG
AAACCTCTCATGAACCCTGTGAGCAAGCCACCGTCTCACTCAATTTGAATCTTGGCTTCCCTCAAAAGACTGGCT
AATGTTTGGTAACTCTCTGGAGTAGACAGCACTACATGTACGTAAGATAGGTACATAAACAACTATTGGTTTTGA
GCTGATTTTTTTCAGCTGCATTTGCATGTATGGATTTTTCTCACCAAAGACGATGACTTCAAGTATTAGTAAAAT
AATTGTACAGCTCTCCTGATTATACTTCTCTGTGACATTTCATTTTCCCAGGCTATTTCTTTTGGTAGGATTAAA
ACTAAGCAATTCAGTATGATCTTTGTCCTTCATTTTCTTTCTTATTCTTTTTGTTTGTTTGTTTGTTTGTTTTTT
TCTTGAGGCAGAGTCTCTCTCTGTCGCCCAGGCTGGAGTGCAGTGGCGCCATCTCAGCTCATTGCAACCTCTGCC
ACCTCCGGGTTCAAGAGATTCTCCTGCCTCAGCCTCCCGAGTAGCTGGGATTACAGGTGTCCACCACCACACCCG
GCTAATTTTTTGTATTTTTTAGTAGAGGGGGGTTTCACCATGTTGGCCAGGCTGGTCTTGAGCTCCTGACCTCAG
GTGATCCACCTGCCTCGGCCTACCAAAGAGCTGGGATAACAGGTGTGACCCACCATGCCCGGCCCATTTTTTTTT
TCTTATTCTGTTAGGAGTGAGAGTGTAACTAGCAGTATAATAGTTCAATTTTCACAACGTGGTAAAAGTTTCCCT
ATAATTCAATCAGATTTTGCTCCAGGGTTCAGTTCTGTTTTAGGAAATACTTTTATTTTCAGTTTAATGATGAAA
TATTAGAGTTGTAATATTGCCTTTATGATTATCCACCTTTTTAACCTAAAAGAATGAAAGAAAAATATGTTTGCA
ATATAATTTTATGGTTGTATGTTAACTTAATTCATTATGTTGGCCTCCAGTTTGCTGTTGTTAGTTATGACAGCA
GTAGTGTCATTACCATTTCAATTCAGATTACATTCCTATATTTGATCATTGTAAACTGACTGCTTACATTGTATT
AAAAACAGTGGATATTTTAAAGAAGCTGTACGGCTTATATCTAGTGCTGTCTCTTAAGACTATTAAATTGATACA
ACATATTTAAAAGTAAATATTACCTAAATGAATTTTTGAAATTACAAATACACGTGTTAAAACTGTCGTTGTGTT
CAACCATTTCTGTACATACTTAGAGTTAACTGTTTTGCCAGGCTCTGTATGCCTACTCATAATATGATAAAAGCA
CTCATCTAATGCTCTGTAAATAGAAGTCAGTGCTTTCCATCAGACTGAACTCTCTTGACAAGATGTGGATGAAAT
TCTTTAAGTAAAATTGTTTACTTTGTCATACATTTACAGATCAAATGTTAGCTCCCAAAGCAATCATATGGCAAA
GATAGGTATATCATAGTTTGCCTATTAGCTGCTTTGTATTGCTATTATTATAAATAGACTTCACAGTTTTAGACT
TGCTTAGGTGAAATTGCAATTCTTTTTACTTTCAGTCTTAGATAACAAGTCTTCAATTATAGTACAATCACACAT
TGCTTAGGAATGCATCATTAGGCGATTTTGTCATTATGCAAACATCATAGAGTGTACTTACACAAACCTAGATAG
TATAGCCTTTATGTACCTAGGCCGTATGGTATAGTCTGTTGCTCCTAGGCCACAAACCTGTACAACTGTTACTGT
ACTGAATACTATAGACAGTTGTAACACAGTGGTAAATATTTATCTAAATATATGCAAACAGAGAAAAGGTACAGT
AAAAGTATGGTATAAAAGATAATGGTATACCTGTGTAGGCCACTTACCACGAATGGAGCTTGCAGGACTAGAAGT
TGCTCTGGGTGAGTCAGTGAGTGAGTGGTGAATTAATGTGAAGGCCTAGAACACTGTACACCACTGTAGACTATA
AACACAGTACGCTGAAGCTACACCAAATTTATCTTAACAGTTTTTCTTCAATAAAAAATTATAACTTTTTAACTT
TGTAAACTTTTTAATTTTTTAACTTTTAAAATACTTAGCTTGAAACACAAATACATTGTATAGCTATACAAAAAT
ATTTTTTCTTTGTATCCTTATTCTAGAAGCTTTTTTCTATTTTCTATTTTAAATTTTTTTTTTTACTTGTTAGTC
GTTTTTGTTAAAAACTAAAACACACACACTTTCACCTAGGCATAGACAGGATTAGGATCATCAGTATCACTCCCT
TCCACCTCACTGCCTTCCACCTCCACATCTTGTCCCACTGGAAGGTTTTTAGGGGCAATAACACACATGTAGCTG
TCACCTATGATAACAGTGCTTTCTGTTGAATACCTCCTGAAGGACTTGCCTGAGGCTGTTTTACATTTAACTTAA
AAAAAAAAAAAGTAGAAGGAGTGCACTCTAAAATAACAATAAAAGGCATAGTATAGTGAATACATAAACCAGCAA
TGTAGTAGTTTATTATCAAGTGTTGTACACTGTAATAATTGTATGTGCTATACTTTAAATAACTTGCAAAATAGT
ACTAAGACCTTATGATGGTTACAGTGTCACTAAGGCAATAGCATATTTTCAGGTCCATTGTAATCTAATGGGACT
ACCATCATATATGCAGTCTACCATTGACTGAAACGTTACATGGCACATAACTGTATTTGCAAGAATGATTTGTTT
TACATTAATATCACATAGGATGTACCTTTTTAGAGTGGTATGTTTATGTGGATTAAGATGTACAAGTTGAGCAAG
GGGACCAAGAGCCCTGGGTTCTGTCTTGGATGTGAGCGTTTATGTTCTTCTCCTCATGTCTGTTTTCTCATTAAA
TTCAAAGGCTTGAACGGGCCCTATTTAGCCCTTCTGTTTTCTACGTGTTCTAAATAACTAAAGCTTTTAAATTCT
AGCCATTTAGTGTAGAACTCTCTTTGCAGTGATGAAATGCTGTATTGGTTTCTTGGCTAGCATATTAAATATTTT
TATCTTTGTCTTGATACTTCAATGTCGTTTTAAACATCAGGATCGGGCTTCAGTATTCTCATAACCAGAGAGTTC
ACTGAGGATACAGGACTGTTTGCCCATTTTTTGTTATGGCTCCAGACTTGTGGTATTTCCATGTCTTTTTTTTTT
TTTTTTTTTTTGACCTTTTAGCGGCTTTAAAGTATTTCTGTTGTTAGGTGTTGTATTACTTTTCTAAGATTACTT
AACAAAGCACCACAAACTGAGTGGCTTTAAACAACAGCAATTTATTCTCTCACAATTCTAGAAGCTAGAAGTCCG
AAATCAAAGTGTTGACAGGGGCATGATCTTCAAGAGAGAAGACTCTTTCCTTGCCTCTTCCTGGCTTCTGGTGGT
TACCAGCAATCCTGAGTGTTCCTTTCTTGCCTTGTAGTTTCAACAATCCAGTATCTGCCTTTTGTCTTCACATGG
CTGTCTACCATTTGTCTCTGTGTCTCCAAATCTCTCTCCTTATAAACACAGCAGTTATTGGATTAGGCCCCACTC
TAATCCAGTATGACCCCATTTTAACATGATTACACTTATTTCTAGATAAGGTCACATTCACGTACACCAAGGGTT
AGGAATTGAACATATCTTTTTGGGGGACACAATTCAACCCACAAGTTGTCAGTCTCTAGCTGAGCCTTTCCCTCC
TGTTTTTCTCCTTTTTAGTTGCTATGGGTTAGGGGCCAAATCTCCAGTCATACTAGAATTGCACATGGACTGGAT
ATTTGGGAATACTGCGGGTCTATTCTATGAGCTTTAGTATGTAACATTTAATATCAGTGTAAAGAAGCCCTTTTT
TAAGTTATTTCTTTGAATTTCTAAATGTATGCCCTGAATATAAGTAACAAGTTACCATGTCTTGTAAAATGATCA
TATCAACAAACATTTAATGTGCACCTACTGTGCTAGTTGAATGTCTTTATCCTGATAGGAGATAACAGGATTCCA
CATCTTTGACTTAAGAGGACAAACCAAATATGTCTAAATCATTTGGGGTTTTGATGGATATCTTTAAATTGCTGA
ACCTAATCATTGGTTTCATATGTCATTGTTTAGATATCTCCGGAGCATTTGGATAATGTGACAGTTGGAATGCAG
TGATGTCGACTCTTTGCCCACCGCCATCTCCAGCTGTTGCCAAGACAGAGATTGCTTTAAGTGGCAAATCACCTT
TATTAGCAGCTACTTTTGCTTACTGGGACAATATTCTTGGTCCTAGAGTAAGGCACATTTGGGCTCCAAAGACAG
AACAGGTACTTCTCAGTGATGGAGAAATAACTTTTCTTGCCAACCACACTCTAAATGGAGAAATCCTTCGAAATG
CAGAGAGTGGTGCTATAGATGTAAAGTTTTTTGTCTTGTCTGAAAAGGGAGTGATTATTGTTTCATTAATCTTTG
ATGGAAACTGGAATGGGGATCGCAGCACATATGGACTATCAATTATACTTCCACAGACAGAACTTAGTTTCTACC
TCCCACTTCATAGAGTGTGTGTTGATAGATTAACACATATAATCCGGAAAGGAAGAATATGGATGCATAAGGTAA
GTGATTTTTCAGCTTATTAATCATGTTAACCTATCTGTTGAAAGCTTATTTTCTGGTACATATAAATCTTATTTT
TTTAATTATATGCAGTGAACATCAAACAATAAATGTTATTTATTTTGCATTTACCCTATTAGATACAAATACATC
TGGTCTGATACCTGTCATCTTCATATTAACTGTGGAAGGTACGAAATGGTAGCTCCACATTATAGATGAAAAGCT
AAAGCTTAGACAAATAAAGAAACTTTTAGACCCTGGATTCTTCTTGGGAGCCTTTGACTCTAATACCTTTTGTTT
CCCTTTCATTGCACAATTCTGTCTTTTGCTTACTACTATGTGTAAGTATAACAGTTCAAAGTAATAGTTTCATAA
GCTGTTGGTCATGTAGCCTTTGGTCTCTTTAACCTCTTTGCCAAGTTCCCAGGTTCATAAAATGAGGAGGTTGAA
TGGAATGGTTCCCAAGAGAATTCCTTTTAATCTTACAGAAATTATTGTTTTCCTAAATCCTGTAGTTGAATATAT
AATGCTATTTACATTTCAGTATAGTTTTGATGTATCTAAAGAACACATTGAATTCTCCTTCCTGTGTTCCAGTTT
GATACTAACCTGAAAGTCCATTAAGCATTACCAGTTTTAAAAGGCTTTTGCCCAATAGTAAGGAAAAATAATATC
TTTTAAAAGAATAATTTTTTTTTACTATGTTTGCAGGCTTACTTCCTTTCTCACATTATGAAACTCTTAAAATCA
GGAGAATCTTTTAAACAACATCATAATGTTTAATTTGAAAAGTGCAAGTCATTCTTTTCCTTGTTGAAACTATGC
AGATGTTACATTGACTGTTTTCTGTGAAGTTATCTTTTTTTCACTGCAGAATAAAGGTTGTTTTGATTTTATTTT
GTATTGTTTATGAGAACATGCATTTGTTGGGTTAATTTCCTACCCCTGCCCCCATTTTTTCCCTAAAGTAGAAAG
TATTTTTCTTGTGAACTAAATTACTACACAAGAACATGTCTATTGAAAAATAAGCAAGTATCAAAATGTTGTGGG
TTGTTTTTTTAAATAAATTTTCTCTTGCTCAGGAAAGACAAGAAAATGTCCAGAAGATTATCTTAGAAGGCACAG
AGAGAATGGAAGATCAGGTATATGCAAATTGCATACTGTCAAATGTTTTTCTCACAGCATGTATCTGTATAAGGT
TGATGGCTACATTTGTCAAGGCCTTGGAGACATACGAATAAGCCTTTAATGGAGCTTTTATGGAGGTGTACAGAA
TAAACTGGAGGAAGATTTCCATATCTTAAACCCAAAGAGTTAAATCAGTAAACAAAGGAAAATAGTAATTGCATC
TACAAATTAATATTTGCTCCCTTTTTTTTTCTGTTTGCCCAGAATAAATTTTGGATAACTTGTTCATAGTAAAAA
TAAAAAAAATTGTCTCTGATATGTTCTTTAAGGTACTACTTCTCGAACCTTTCCCTAGAAGTAGCTGTAACAGAA
GGAGAGCATATGTACCCCTGAGGTATCTGTCTGGGGTGTAGGCCCAGGTCCACACAATATTTCTTCTAAGTCTTA
TGTTGTATCGTTAAGACTCATGCAATTTACATTTTATTCCATAACTATTTTAGTATTAAAATTTGTCAGTGATAT
TTCTTACCCTCTCCTCTAGGAAAATGTGCCATGTTTATCCCTTGGCTTTGAATGCCCCTCAGGAACAGACACTAA
GAGTTTGAGAAGCATGGTTACAAGGGTGTGGCTTCCCCTGCGGAAACTAAGTACAGACTATTTCACTGTAAAGCA
GAGAAGTTCTTTTGAAGGAGAATCTCCAGTGAAGAAAGAGTTCTTCACTTTTACTTCCATTTCCTCTTGTGGGTG
ACCCTCAATGCTCCTTGTAAAACTCCAATATTTAAACATGGCTGTTTTGCCTTTCTTTGCTTCTTTTTAGCATTG
AATGAGACAGATGATACTTTAAAAAAGTAATTAAAAAAAAAAACTTGTGAAAATACATGGCCATAATACAGAACC
CAATACAATGATCTCCTTTACCAAATTGTTATGTTTGTACTTTTGTAGATAGCTTTCCAATTCAGAGACAGTTAT
TCTGTGTAAAGGTCTGACTTAACAAGAAAAGATTTCCCTTTACCCAAAGAATCCCAGTCCTTATTTGCTGGTCAA
TAAGCAGGGTCCCCAGGAATGGGGTAACTTTCAGCACCCTCTAACCCACTAGTTATTAGTAGACTAATTAAGTAA
ACTTATCGCAAGTTGAGGAAACTTAGAACCAACTAAAATTCTGCTTTTACTGGGATTTTGTTTTTTCAAACCAGA
AACCTTTACTTAAGTTGACTACTATTAATGAATTTTGGTCTCTCTTTTAAGTGCTCTTCTTAAAAATGTTATCTT
ACTGCTGAGAAGTTCAAGTTTGGGAAGTACAAGGAGGAATAGAAACTTAAGAGATTTTCTTTTAGAGCCTCTTCT
GTATTTAGCCCTGTAGGATTTTTTTTTTTTTTTTTTTTTTTGGTGTTGTTGAGCTTCAGTGAGGCTATTCATTCA
CTTATACTGATAATGTCTGAGATACTGTGAATGAAATACTATGTATGCTTAAACCTAAGAGGAAATATTTTCCCA
AAATTATTCTTCCCGAAAAGGAGGAGTTGCCTTTTGATTGAGTTCTTGCAAATCTCACAACGACTTTATTTTGAA
CAATACTGTTTGGGGATGATGCATTAGTTTGAAACAACTTCAGTTGTAGCTGTCATCTGATAAAATTGCTTCACA
GGGAAGGAAATTTAACACGGATCTAGTCATTATTCTTGTTAGATTGAATGTGTGAATTGTAATTGTAAACAGGCA
TGATAATTATTACTTTAAAAACTAAAAACAGTGAATAGTTAGTTGTGGAGGTTACTAAAGGATGGTTTTTTTTTA
AATAAAACTTTCAGCATTATGCAAATGGGCATATGGCTTAGGATAAAACTTCCAGAAGTAGCATCACATTTAAAT
TCTCAAGCAACTTAATAATATGGGGCTCTGAAAAACTGGTTAAGGTTACTCCAAAAATGGCCCTGGGTCTGACAA
AGATTCTAACTTAAAGATGCTTATGAAGACTTTGAGTAAAATCATTTCATAAAATAAGTGAGGAAAAACAACTAG
TATTAAATTCATCTTAAATAATGTATGATTTAAAAAATATGTTTAGCTAAAAATGCATAGTCATTTGACAATTTC
ATTTATATCTCAAAAAATTTACTTAACCAAGTTGGTCACAAAACTGATGAGACTGGTGGTGGTAGTGAATAAATG
AGGGACCATCCATATTTGAGACACTTTACATTTGTGATGTGTTATACTGAATTTTCAGTTTGATTCTATAGACTA
CAAATTTCAAAATTACAATTTCAAGATGTAATAAGTAGTAATATCTTGAAATAGCTCTAAAGGGAATTTTTCTGT
TTATTGATTCTTAAAATATATGTGCTGATTTTGATTTGCATTTGGGTAGATTTATACTTTTATGAGTATGGAGGT
TAGGTATTGATTCAAGTTTTCCTTACCTATTTGGTAAGGATTTCAAAGTCTTTTTGTGCTTGGTTTTCCTCATTT
TTAAATATGAAATATATTGATGACCTTTAACAAATTTTTTTTATCTCAAATTTTAAAGGAGATCTTTTCTAAAAG
AGGCATGATGACTTAATCATTGCATGTAACAGTAAACGATAAACCAATGATTCCATACTCTCTAAAGAATAAAAG
TGAGCTTTAGGGCCGGGCATGGTCAGAAATTTGACACCAACCTGGCCAACATGGCGAAACCCCGTCTCTACTAAA
AATACAAAAATCAGCCGGGCATGGTGGCGGCACCTATAGTCCCAGCTACTTGGGAGGATGAGACAGGAGAGTCAC
TTGAACCTGGGAGGAGAGGTTGCAGTGAGCTGAGATCACGCCATTGCACTCCAGCCTGAGCAATGAAAGCAAAAC
TCCATCTCAAAAAAAAAAAAAGAAAAGAAAGAATAAAAGTGAGCTTTGGATTGCATATAAATCCTTTAGACATGT
AGTAGACTTGTTTGATACTGTGTTTGAACAAATTACGAAGTATTTTCATCAAAGAATGTTATTGTTTGATGTTAT
TTTTATTTTTTATTGCCCAGCTTCTCTCATATTACGTGATTTTCTTCACTTCATGTCACTTTATTGTGCAGGGTC
AGAGTATTATTCCAATGCTTACTGGAGAAGTGATTCCTGTAATGGAACTGCTTTCATCTATGAAATCACACAGTG
TTCCTGAAGAAATAGATGTAAGTTTAAATGAGAGCAATTATACACTTTATGAGTTETTTGGGGTTATAGTATTAT
TATGTATATTATTAATATTCTAATTTTAATAGTAAGGACTTTGTCATACATACTATTCACATACAGTATTAGCCA
CTTTAGCAAATAAGCACACACAAAATCCTGGATTTTATGGCAAAACAGAGGCATTTTTGATCAGTGATGACAAAA
TTAAATTCATTTTGTTTATTTCATTACTTTTATAATTCCTAAAAGTGGGAGGATCCCAGCTCTTATAGGAGCAAT
TAATATTTAATGTAGTGTCTTTTGAAACAAAACTGTGTGCCAAAGTAGTAACCATTAATGGAAGTTTACTTGTAG
TCACAAATTTAGTTTCCTTAATCATTTGTTGAGGACGTTTTGAATCACACACTATGAGTGTTAAGAGATACCTTT
AGGAAACTATTCTTGTTGTTTTCTGATTTTGTCATTTAGGTTAGTCTCCTGATTCTGACAGCTCAGAAGAGGAAG
TTGTTCTTGTAAAAATTGTTTAACCTGCTTGACCAGCTTTCACATTTGTTCTTCTGAAGTTTATGGTAGTGCACA
GAGATTGTTTTTTGGGGAGTCTTGATTCTCGGAAATGAAGGCAGTGTGTTATATTGAATCCAGACTTCCGAAAAC
TTGTATATTAAAAGTGTTATTTCAACACTATGTTACAGCCAGACTAATTTTTTTATTTTTTGATGCATTTTAGAT
AGCTGATACAGTACTCAATGATGATGATATTGGTGACAGCTGTCATGAAGGCTTTCTTCTCAAGTAAGAATTTTT
TTTTTCATAAAAGCTGGATGAAGCAGATACCATCTTATGCTCACCTATGACAAGATTTGGAAGAAAGAAAATAAC
AGACTGTCTACTTAGATTGTTCTAGGGACATTACGTATTTGAACTGTTGCTTAAATTTGTGTTATTTTTCACTCA
TTATATTTCTATATATATTTGGTGTTATTCCATTTGCTATTTAAAGAAACCGAGTTTCCATCCCAGACAAGAAAT
CATGGCCCCTTGCTTGATTCTGGTTTCTTGTTTTACTTCTCATTAAAGCTAACAGAATCCTTTCATATTAAGTTG
TACTGTAGATGAACTTAAGTTATTTAGGCGTAGAACAAAATTATTCATATTTATACTGATCTTTTTCCATCCAGC
AGTGGAGTTTAGTACTTAAGAGTTTGTGCCCTTAAACCAGACTCCCTGGATTAATGCTGTGTACCCGTGGGCAAG
GTGCCTGAATTCTCTATACACCTATTTCCTCATCTGTAAAATGGCAATAATAGTAATAGTACCTAATGTGTAGGG
TTGTTATAAGCATTGAGTAAGATAAATAATATAAAGCACTTAGAACAGTGCCTGGAACATAAAAACACTTAATAA
TAGCTCATAGCTAACATTTCCTATTTACATTTCTTCTAGAAATAGCCAGTATTTGTTGAGTGCCTACATGTTAGT
TCCTTTACTAGTTGCTTTACATGTATTATCTTATATTCTGTTTTAAAGTTTCTTCACAGTTACAGATTTTCATGA
AATTTTACTTTTAATAAAAGAGAAGTAAAAGTATAAAGTATTCACTTTATGTTTCACAGTCTTTTCCTTTAGGCT
CATGATGGAGTATCAGAGGCATGAGTGTGTTTAACCTAAGAGCCTTAATGGCTTGAATCAGAAGCACTTTAGTCC
TGTATCTGTTCAGTGTCAGCCTTTCATACATCATTTTAAATCCCATTTGACTTTAAGTAAGTCACTTAATCTCTC
TACATGTCAATTTCTTCAGCTATAAAATGATGGTATTTCAATAAATAAATACATTAATTAAATGATATTATACTG
ACTAATTGGGCTGTTTTAAGGCTCAATAAGAAAATTTCTGTGAAAGGTCTCTAGAAAATGTAGGTTCCTATACAA
ATAAAAGATAACATTGTGCTTATAGCTTCGGTGTTTATCATATAAAGCTATTCTGAGTTATTTGAAGAGCTCACC
TACTTTTTTTTGTTTTTAGTTTGTTAAATTGTTTTATAGGCAATGTTTTTAATCTGTTTTCTTTAACTTACAGTG
CCATCAGCTCACACTTGCAAACCTGTGGCTGTTCCGTTGTAGTAGGTAGCAGTGCAGAGAAAGTAAATAAGGTAG
TTTATTTTATAATCTAGCAAATGATTTGACTCTTTAAGACTGATGATATATCATGGATTGTCATTTAAATGGTAG
GTTGCAATTAAAATGATCTAGTAGTATAAGGAGGCAATGTAATCTCATCAAATTGCTAAGACACCTTGTGGCAAC
AGTGAGTTTGAAATAAACTGAGTAAGAATCATTTATCAGTTTATTTTGATAGCTCGGAAATACCAGTGTCAGTAG
TGTATAAATGGTTTTGAGAATATATTAAAATCAGATATATAAAAAAAATTACTCTTCTATTTCCCAATGTTATCT
TTAACAAATCTGAAGATAGTCATGTACTTTTGGTAGTAGTTCCAAAGAAATGTTATTTGTTTATTCATCTTGATT
TCATTGTCTTCGCTTTCCTTCTAAATCTGTCCCTTCTAGGGAGCTATTGGGATTAAGTGGTCATTGATTATTATA
CTTTATTCAGTAATGTTTCTGACCCTTTCCTTCAGTGCTACTTGAGTTAATTAAGGATTAATGAACAGTTACATT
TCCAAGCATTAGCTAATAAACTAAAGGATTTTGCACTTTTCTTCACTGACCATTAGTTAGAAAGAGTTCAGAGAT
AAGTATGTGTATCTTTCAATTTCAGCAAACCTAATTTTTTAAAAAAAGTTTTACATAGGAAATATGTTGGAAATG
ATACTTTACAAAGATATTCATAATTTTTTTTTGTAATCAGCTACTTTGTATATTTACATGAGCCTTAATTTATAT
TTCTCATATAACCATTTATGAGAGCTTAGTATACCTGTGTCATTATATTGCATCTACGAACTAGTGACCTTATTC
CTTCTGTTACCTCAAACAGGTGGCTTTCCATCTGTGATCTCCAAAGCCTTAGGTTGCACAGAGTGACTGCCGAGC
TGCTTTATGAAGGGAGAAAGGCTCCATAGTTGGAGTGTTTTTTTTTTTTTTTTTAAACATTTTTCCCATCCTCCA
TCCTCTTGAGGGAGAATAGCTTACCTTTTTATCTTGTTTAATTTGAGAAAGAAGTTGCCACCACTCTAGGTTGAA
AACCACTCCTTTAACATAATAACTGTGGATATGGTTTGAATTTCAAGATAGTTACATGCCTTTTTATTTTTCCTA
ATAGAGCTGTAGGTCAAATATTATTAGAATCAGATTTCTAAATCCCACCCAATGACCTGCTTATTTTAAATCAAA
TTCAATAATTAATTCTCTTCTTTTTGGAGGATCTGGACATTCTTTGATATTTCTTACAACGAATTTCATGTGTAG
ACCCACTAAACAGAAGCTATAAAAGTTGCATGGTCAAATAAGTCTGAGAAAGTCTGCAGATGATATAATTCACCT
GAAGAGTCACAGTATGTAGCCAAATGTTAAAGGTTTTGAGATGCCATACAGTAAATTTACCAAGCATTTTCTAAA
TTTATTTGACCACAGAATCCCTATTTTAAGCAACAACTGTTACATCCCATGGATTCCAGGTGACTAAAGAATACT
TATTTCTTAGGATATGTTTTATTGATAATAACAATTAAAATTTCAGATATCTTTCATAAGCAAATCAGTGGTCTT
TTTACTTCATGTTTTAATGCTAAAATATTTTCTTTTATAGATAGTCAGAACATTATGCCTTTTTCTGACTCCAGC
AGAGAGAAAATGCTCCAGGTTATGTGAAGCAGAATCATCATTTAAATATGAGTCAGGGCTCTTTGTACAAGGCCT
GCTAAAGGTATAGTTTCTAGTTATCACAAGTGAAACCACTTTTCTAAAATCATTTTTGAGACTCTTTATAGACAA
ATCTTAAATATTAGCATTTAATGTATCTCATATTGACATGCCCAGAGACTGACTTCCTTTACACAGTTCTGCACA
TAGACTATATGTCTTATGGATTTATAGTTAGTATCATCAGTGAAACACCATAGAATACCCTTTGTGTTCCAGGTG
GGTCCCTGTTCCTACATGTCTAGCCTCAGGACTTTTTTTTTTTTAACACATGCTTAAATCAGGTTGCACATCAAA
AATAAGATCATTTCTTTTTAACTAAATAGATTTGAATTTTATTGAAAAAAAATTTTAAACATCTTTAAGAAGCTT
ATAGGATTTAAGCAATTCCTATGTATGTGTACTAAAATATATATATTTCTATATATAATATATATTAGAAAAAAA
TTGTATTTTTCTTTTATTTGAGTCTACTGTCAAGGAGCAAAACAGAGAAATGTAAATTACCAATTATTTATAATA
CTTAAAGGGAAGAAAGTTGTTCACCTTGTTGAATCTATTATTGTTATTTCAATTATAGTCCCAAGACGTGAAGAA
ATAGCTTTCCTAATGGTTATGTGATTGTCTCATAGTGACTACTTTCTTGAGGATGTAGCCACGGCAAAATGAAAT
AAAAAAATTTAAAAATTGTTGCAAATACAAGTTATATTAGGCTTTTGTGCATTTTCAATAATGTGCTGCTATGAA
CTCAGAATGATAGTATTTAAATATAGAAACTAGTTAAAGGAAACGTAGTTTCTATTTGAGTTATACATATCTGTA
AATTAGAACTTCTCCTGTTAAAGGCATAATAAAGTGCTTAATACTTTTGTTTCCTCAGCACCCTCTCATTTAATT
ATATAATTTTAGTTCTGAAAGGGACCTATACCAGATGCCTAGAGGAAATTTCAAAACTATGATCTAATGAAAAAA
TATTTAATAGTTCTCCATGCAAATACAAATCATATAGTTTTCCAGAAAATACCTTTGACATTATACAAAGATGAT
TATCACAGCATTATAATAGTAAAAAAATGGAAATAGCCTCTTTCTTCTGTTCTGTTCATAGCACAGTGCCTCATA
CGCAGTAGGTTATTATTACATGGTAACTGGCTACCCCAACTGATTAGGAAAGAAGTAAATTTGTTTTATAAAAAT
ACATACTCATTGAGGTGCATAGAATAATTAAGAAATTAAAAGACACTTGTAATTTTGAATCCAGTGAATACCCAC
TGTTAATATTTGGTATATCTCTTTCTAGTCTTTTTTTCCCTTTTGCATGTATTTTCTTTAAGACTCCCACCCCCA
CTGGATCATCTCTGCATGTTCTAATCTGCTTTTTTCACAGCAGATTCTAAGCCTCTTTGAATATCAACACAAACT
TCAACAACTTCATCTATAGATGCCAAATAATAAATTCATTTTTATTTACTTAACCACTTCCTTTGGATGCTTAGG
TCATTCTGATGTTTTGCTATTGAAACCAATGCTATACTGAACACTTCTGTCACTAAAACTTTGCACACACTCATG
AATAGCTTCTTAGGATAAATTTTTAGAGATGGATTTGCTAAATCAGAGACCATTTTTTAAAATTAAAAAACAATT
ATTCATATCGTTTGGCATGTAAGACAGTAAATTTTCCTTTTATTTTGACAGGATTCAACTGGAAGCTTTGTGCTG
CCTTTCCGGCAAGTCATGTATGCTCCATATCCCACCACACACATAGATGTGGATGTCAATACTGTGAAGCAGATG
CCACCCTGTCATGAACATATTTATAATCAGCGTAGATACATGAGATCCGAGCTGACAGCCTTCTGGAGAGCCACT
TCAGAAGAAGACATGGCTCAGGATACGATCATCTACACTGACGAAAGCTTTACTTCCTGATTGTACGTAATGCTC
TGCCTGCTGGTACTGTAGTCAAGCAATATGAAATTGTGTCTTTTACGAATAAAAACAAAACAGAAGTTGCATTTA
AAAAGAAAGAAATATTACCAGCAGAATTATGCTTGAAGAAACATTTAATCAAGCATTTTTTTCTTAAATGTTCTT
CTTTTTCCATACAATTGTGTTTACCCTAAAATAGGTAAGATTAACCCTTAAAGTAAATATTTAACTATTTGTTTA
ATAAATATATATTGAGCTCCTAGGCACTGTTCTAGGTACCGGGCTTAATAGTGGCCAACCAGACAGCCCCAGCCC
CAGCCCCTACATTGTGTATAGTCTATTATGTAACAGTTATTGAATGGACTTATTAACAAAACCAAAGAAGTAATT
CTAAGTCTTTTTTTTCTTGACATATGAATATAAAATACAGCAAAACTGTTAAAATATATTAATGGAACATTTTTT
TACTTTGCATTTTATATTGTTATTCACTTCTTATTTTTTTTTAAAAAAAAAAGCCTGAACAGTAAATTCAAAAGG
AAAAGTAATGATAATTAATTGTTGAGCATGGACCCAACTTGAAAAAAAAAATGATGATGATAAATCTATAATCCT
AAAACCCTAAGTAAACACTTAAAAGATGTTCTGAAATCAGGAAAAGAATTATAGTATACTTTTGTGTTTCTCTTT
TATCAGTTGAAAAAAGGCACAGTAGCTCATGCCTGTAAGAACAGAGCTTTGGGAGTGCAAGGCAGGCGGATCACT
TGAGGCCAGGAGTTCCAGACCAGCCTGGGCAACATAGTGAAACCCCATCTCTACAAAAAAAAAAAAGAATTTATT
GGAATGTGTTTCTGTGTGCCTGTAATCCTAGCTATTCCGAAAGCTGAGGCAGGAGGATCTTTTGAGCCCAGGAGT
TTGAGGTTACAGGGAGTTATGATGTGCCAGTGTACTCCAGCCTGGGGAACACCGAGACTCTGTCTTATTTAAAAA
AAAAAAAAAAAAAATGCTTGCAATAATGCCTGGCACATAGAAGGTAACAGTAAGTGTTAACTGTAATAACCCAGG
TCTAAGTGTGTAAGGCAATAGAAAAATTGGGGCAAATAAGCCTGACCTATGTATCTACAGAATCAGTTTGAGCTT
AGGTAACAGACCTGTGGAGCACCAGTAATTACACAGTAAGTGTTAACCAAAAGCATAGAATAGGAATATCTTGTT
CAAGGGACCCCCAGCCTTATACATCTCAAGGTGCAGAAAGATGACTTAATATAGGACCCATTTTTTCCTAGTTCT
CCAGAGTTTTTATTGGTTCTTGAGAAAGTAGTAGGGGAATGTTTTAGAAAATGAATTGGTCCAACTGAAATTACA
TGTCAGTAAGTTTTTATATATTGGTAAATTTTAGTAGACATGTAGAAGTTTTCTAATTAATCTGTGCCTTGAAAC
ATTTTCTTTTTTCCTAAAGTGCTTAGTATTTTTTCCGTTTTTTGATTGGTTACTTGGGAGCTTTTTTGAGGAAAT
TTAGTGAACTGCAGAATGGGTTTGCAACCATTTGGTATTTTTGTTTTGTTTTTTAGAGGATGTATGTGTATTTTA
ACATTTCTTAATCATTTTTAGCCAGCTATGTTTGTTTTGCTGATTTGACAAACTACAGTTAGACAGCTATTCTCA
TTTGCTGATCATGACAAAATAATATCCTGAATTTTTTAAATTTTGCATCCAGCTCTAAATTTTCTAAACATAAAA
TTGTCCAAAAAATAGTATTTTCAGCCACTAGATTGTGTGTTAAGTCTATTGTCACAGAGTCATTTTACTTTTAAG
TATATGTTTTTACATGTTAATTATGTTTGTTATTTTTAATTTTAACTTTTTAAAATAATTCCAGTCACTGCCAAT
ACATGAAAAATTGGTCACTGGAATTTTTTTTTTGACTTTTATTTTAGGTTCATGTGTACATGTGCAGGTGTGTTA
TACAGGTAAATTGCGTGTCATGAGGGTTTGGTGTACAGGTGATTTCATTACCCAGGTAATAAGCATAGTACCCAA
TAGGTAGTTTTTTGATCCTCACCCTTCTCCCACCCTCAAGTAGGCCCTGGTGTTGCTGTTTCCTTCTTTGTGTCC
ATGTATACTCAGTGTTTAGCTCCCACTTAGAAGTGAGAACATGCGGTAGTTGGTTTTCTGTTCCTGGATTAGTTC
ACTTAGGATAATGACCTCTAGCTCCATCTGGTTTTTATGGCTGCATAGTATTCCATGGTGTATATGTATCACATT
TTCTTTATCCAGTCTACCATTGATAGGCATTTAGGTTGATTCCCTGTCTTTGTTATCATGAATAGTGCTGTGATG
AACATACACATGCATGTGTCTTTATGGTAGAAAAATTTGTATTCCTTTAGGTACATATAGAATAATGGGGTTGCT
AGGGTGAATGGTAGTTCTATTTTCAGTTATTTGAGAAATCTTCAAACTGCTTTTCATAATAGCTAAACTAATTTA
CAGTCCCGCCAGCAGTGTATAAGTGTTCCCTTTTCTCCACAACCTTGCCAACATCTGTGATTTTTTGACTTTTTA
ATAATAGCCATTCCTAGAGAATTGATTTGCAATTCTCTATTAGTGATATTAAGCATTTTTTCATATGCTTTTTAG
CTGTCTGTATATATTCTTCTGAAAAATTTTCATGTCCTTTGCCCAGTTTGTAGTGGGGGGGTTGTTTTTTTGCTT
TTAATTAGTTTTAAGTTCCTTCCAGATTCTGCATATCCCTTTGTTGGATTACATGGTTTGCAGATATTTTTCTCC
CATTGTGTAGGTTGTCTTTTACTCTGTTGATAGTTTCTTTTGCCATGCAGGAGCTCGTTAGGTCCCATTTGTGTT
TGTTTTTGTTGCAGTTGCTTTTGGCGTCTTCATCATAAAATCTGTGCCAGGGCCTATGTCCAGAATGGTATTTCC
TAGGTTGTCTTCCAGGGTTTTTACAATTTTAGATTTTACGTTTATGTCTTTAATCCATCTTGAGTTGATTTTTGT
ATATGGCACAAGGAAGGGGTCCAGTTTCACTCCAATTCCTATGGCTAGCAATTATCCCAGCACCATTTATTGAAT
ACGGAGTCCTTTCCCCATTGCTTGTTTTTTGTCAACTTTGTTGAAGATCAGATGGTTGTAAGTGTGTGGCTTTAT
TTCTTGGCTCTCTATTCTCCATTGGTCTATGTGTCTGTTTTTATAACAGTACCCTGCTGTTCAGGTTCCTATAGC
CTTTTAGTATAAAATCGGCTAATGTGATGCCTCCAGCTTTGTTCTTTTTGCTTAGGATTGCTTTGGCTATTTGGG
CTCCTTTTTGGGTCCATATTAATTTTAAAACAGTTTTTTCTGGTTTTGTGAAGGATATCATTGGTAGTTTATAGG
AATAGCATTGAATCTGTAGATTGCTTTGGGCAGTATGGCCATTTTAACAATATTAATTCTTCCTATCTATGAATA
TGGAATGTTTTTCCATGTGTTTGTGTCATCTCTTTATACCTGATGTATAAAGAAAAGCTGGTATTATTCCTACTC
AATCTGTTCCAAAAAATTGAGGAGGAGGAACTCTTCCCTAATGAGGCCAGCATCATTCTGATACCAAAACCTGGC
AGAGACACAACAGAAAAAAGAAAACTTCAGGCCAATATCCTTGATGAATATAGATGCAAAAATCCTCAACAAAAT
ACTAGCAAACCAAATCCAGCAGCACATCAAAAAGCTGATCTACTTTGATCAAGTAGGCTTTATCCCTGGGATGCA
AGGTTGGTTCAACATACACAAATCAATAAGTGTGATTCATCACATAAACAGAGCTAAAAACAAAAACCACAAGAT
TATCTCAATAGGTAGAGAAAAGGTTGTCAATAAAATTTAACATCCTCCATGTTAAAAACCTTCAGTAGGTCAGGT
GTAGTGACTCACACCTGTAATCCCAGCACTTTGGGAGGCCAAGGCGGGCATATCTCTTAAGCCCAGGAGTTCAAG
ACGAGCCTAGGCAGCATGGTGAAACCCCATCTCTACAAAAAAAAAAAAAAAAAAAAATTAGCTTGGTATGGTGAC
ATGCACCTATAGTCCCAGCTATTCAGGAGGTTGAGGTGGGAGGATTGTTTGAGCCCGGGAGGCAGAGGTTGGCAG
CGAGCTGAGATCATGCCACCGCACTCCAGCCTGGGCAACGGAGTGAGACCCTGTCTCAAAAAAGAAAAATCACAA
ACAATCCTAAACAAACTAGGCATTGAAGGAACATGCCTCAAAAAAATAAGAACCATCTATGACAGACCCATAGCC
AATATCTTACCAAATGGGCAAAAGCTGGAAGTATTCTCCTTGAGAACCGTAACAAGACAAGGATGTCCACTCTCA
CCACTCCTTTTCAGCATAGTTCTGGAAGTCCTAGCCAGAGCAATTCAGGAAAGAGAAAAAAGAAAGACATTCAGA
TAGGAAGAGAAGAAGTCAAACTATTTCTGTTTGCAGGCAGTATAATTCTGTACCTAGAAAATCTCATAGTCTCTG
CCCAGAAACTCCTAAATCTGTTAAAAATTTCAGCAAAGTTTTGGCATTCTCTATACTCCAACACCTTCCAAAGTG
AGAGCAAAATCAAGAACACAGTCCCATTCACAATAGCCGCAAAACGAATAAAATACCTAGGAATCCAGCTAACCA
GGGAGGTGAAAGATCTCTATGAGAATTACAAAACACTGCTGAAAGAAATCAGAGATGACACAAACAAATGGAAAT
GTTCTTTTTTAACACCTTGCTTTATCTAATTCACTTATGATGAAGATACTCATTCAGTGGAACAGGTATAATAAG
TCCACTCGATTAAATATAAGCCTTATTCTCTTTCCAGAGCCCAAGAAGGGGCACTATCAGTGCCCAGTCAATAAT
GACGAAATGCTAATATTTTTCCCCTTTACGGTTTCTTTCTTCTGTAGTGTGGTACACTCGTTTCTTAAGATAAGG
AAACTTGAACTACCTTCCTGTTTGCTTCTACACATACCCATTCTCTTTTTTTGCCACTCTGGTCAGGTATAGGAT
GATCCCTACCACTTTCAGTTAAAAACTCCTCCTCTTACTAAATGTTCTCTTACCCTCTGGCCTGAGTAGAACCTA
GGGAAAATGGAAGAGAAAAAGATGAAAGGGAGGGGGGCCTGGGAAGGGAATAAGTAGTCCTGTTTTGTTTGTGTG
TTTGCTTTAGCACCTGCTATATCCTAGGTGCTGTGTTAGGCACACATTATTTTAAGTGGCCATTATATTACTACT
ACTCACTCTGGTCGTTGCCAAGGTAGGTAGTACTTTCTTGGATAGTTGGTTCATGTTACTTACAGATGGTGGGCT
TGTTGAGGCAAACCCAGTGGATAATCATCGGAGTGTGTTCTCTAATCTCACTCAAATTTTTCTTCACATTTTTTG
GTTTGTTTTGGTTTTTGATGGTAGTGGCTTATTTTTGTTGCTGGTTTGTTTTTTGTTTTTTTTTGAGATGGCAAG
AATTGGTAGTTTATTTATTAATTGCCTAAGGGTCTTCTACTTTTTTTAAAAGATGAGAGTAGTAAAATAGATTGA
TAGATACATACATACCCTTACTGGGGACTGCTTATATTCTTTAGAGAAAAAATTACATATTAGCCTGACAAACAC
CAGTAAAATGTAAATATATCCTTGAGTAAATAAATGAATGTATATTTTGTGTCTCCAAATATATATATCTATATT
CTTACAAATGTGTTTATATGTAATATCAATTTATAAGAACTTAAAATGTTGGCTCAAGTGAGGGATTGTGGAAGG
TAGCATTATATGGCCATTTCAACATTTGAACTTTTTTCTTTTCTTCATTTTCTTCTTTTCTTCAGGAATATTTTT
CAAGATGTCTTACACAGAGACACTCTAGTGAAAGCCTTCCTGGATCAGGTAAATGTTGAACTTGAGATTGTCAGA
GTGAATGATATGACATGTTTTCTTTTTTAATATATCCTACAATGCCTGTTCTATATATTTATATTCCCCTGGATC
ATGCCCCAGAGTTCTGCTCAGCAATTGCAGTTAAGTTAGTTACACTACAGTTCTCAGAAGAGTCTGTGAGGGCAT
GTCAAGTGCATCATTACATTGGTTGCCTCTTGTCCTAGATTTATGCTTCGGGAATTCAGACCTTTGTTTACAATA
TAATAAATATTATTGCTATCTTTTAAAGATATAATAATAAGATATAAAGTTGACCACAACTACTGTTTTTTGAAA
CATAGAATTCCTGGTTTACATGTATCAAAGTGAAATCTGACTTAGCTTTTACAGATATAATATATACATATATAT
ATCCTGCAATGCTTGTACTATATATGTAGTACAAGTATATATATATGTTTGTGTGTGTATATATATATAGTACGA
GCATATATACATATTACCAGCATTGTAGGATATATATATGTTTATATATTAAAAAAAAGTTATAAACTTAAAACC
CTATTATGTTATGTAGAGTATATGTTATATATGATATGTAAAATATATAACATATACTCTATGATAGAGTGTAAT
ATATTTTTTATATATATTTTAACATTTATAAAATGATAGAATTAAGAATTGAGTCCTAATCTGTTTTATTAGGTG
CTTTTTGTAGTGTCTGGTCTTTCTAAAGTGTCTAAATGATTTTTCCTTTTGACTTATTAATGGGGAAGAGCCTGT
ATATTAACAATTAAGAGTGCAGCATTCCATACGTCAAACAACAAACATTTTAATTCAAGCATTAACCTATAACAA
GTAAGTTTTTTTTTTTTTTTGAGAAAGGGAGGTTGTTTATTTGCCTGAAATGACTCAAAAATATTTTTTGAAACA
TAGTGTACTTATTTAAATAACATCTTTATTGTTTCATTCTTTTAAAAAATATCTACTTAATTACACAGTTGAAGG
AAATCGTAGATTATATGGAACTTATTTCTTAATATATTACAGTTTGTTATAATAACATTCTGGGGATCAGGCCAG
GAAACTGTGTCATAGATAAAGCTTTGAAATAATGAGATCCTTATGTTTACTAGAAATTTTGGATTGAGATCTATG
AGGTCTGTGACATATTGCGAAGTTCAAGGAAAATTCGTAGGCCTGGAATTTCATGCTTCTCAAGCTGACATAAAA
TCCCTCCCACTCTCCACCTCATCATATGCACACATTCTACTCCTACCCACCCACTCCACCCCCTGCAAAAGTACA
GGTATATGAATGTCTCAAAACCATAGGCTCATCTTCTAGGAGCTTCAATGTTATTTGAAGATTTGGGCAGAAAAA
ATTAAGTAATACGAAATAACTTATGTATGAGTTTTAAAAGTGAAGTAAACATGGATGTATTCTGAAGTAGAATGC
AAAATTTGAATGCATTTTTAAAGATAAATTAGAAAACTTCTAAAAACTGTCAGATTGTCTGGGCCTGGTGGCTTA
TGCCTGTAATCCCAGCACTTTGGGAGTCCGAGGTGGGTGGATCACAAGGTCAGGAGATCGAGACCATCCTGCCAA
CATGGTGAAACCCCGTCTCTACTAAGTATACAAAAATTAGCTGGGCGTGGCAGCGTGTGCCTGTAATCCCAGCTA
CCTGGGAGGCTGAGGCAGGAGAATCGCTTGAACCCAGGAGGTGTAGGTTGCAGTGAGTCAAGATCGCGCCACTGC
ACTTTAGCCTGGTGACAGAGCTAGACTCCGTCTCAAAAAAAAAAAAAAATATCAGATTGTTCCTACACCTAGTGC
TTCTATACCACACTCCTGTTAGGGGGCATCAGTGGAAATGGTTAAGGAGATGTTTAGTGTGTATTGTCTGCCAAG
CACTGTCAACACTGTCATAGAAACTTCTGTACGAGTAGAATGTGAGCAAATTATGTGTTGAAATGGTTCCTCTCC
CTGCAGGTCTTTCAGCTGAAACCTGGCTTATCTCTCAGAAGTACTTTCCTTGCACAGTTTCTACTTGTCCTTCAC
AGAAAAGCCTTGACACTAATAAAATATATAGAAGACGATACGTGAGTAAAACTCCTACACGGAAGAAAAACCTTT
GTACATTGTTTTTTTGTTTTGTTTCCTTTGTACATTTTCTATATCATAATTTTTGCGCTTCTTTTTTTTTTTTTT
TTTTTTTTTTTTCCATTATTTTTAGGCAGAAGGGAAAAAAGCCCTTTAAATCTCTTCGGAACCTGAAGATAGACC
TTGATTTAACAGCAGAGGGCGATCTTAACATAATAATGGCTCTGGCTGAGAAAATTAAACCAGGCCTACACTCTT
TTATCTTTGGAAGACCTTTCTACACTAGTGTGCAAGAACGAGATGTTCTAATGACTTTTTAAATGTGTAACTTAA
TAAGCCTATTCCATCACAATCATGATCGCTGGTAAAGTAGCTCAGTGGTGTGGGGAAACGTTCCCCTGGATCATA
CTCCAGAATTCTGCTCTCAGCAATTGCAGTTAAGTAAGTTACACTACAGTTCTCACAAGAGCCTGTGAGGGGATG
TCAGGTGCATCATTACATTGGGTGTCTCTTTTCCTAGATTTATGCTTTTGGGATACAGACCTATGTTTACAATAT
AATAAATATTATTGCTATCTTTTAAAGATATAATAATAGGATGTAAACTTGACCACAACTACTGTTTTTTTGAAA
TACATGATTCATGGTTTACATGTGTCAAGGTGAAATCTGAGTTGGCTTTTACAGATAGTTGACTTTCTATCTTTT
GGCATTCTTTGGTGTGTAGAATTACTGTAATACTTCTGCAATCAACTGAAAACTAGAGCCTTTAAATGATTTCAA
TTCCACAGAAAGAAAGTGAGCTTGAACATAGGATGAGCTTTAGAAAGAAAATTGATCAAGCAGATGTTTAATTGG
AATTGATTATTAGATCCTACTTTGTGGATTTAGTCCCTGGGATTCAGTCTGTAGAAATGTCTAATAGTTCTCTAT
AGTCCTTGTTCCTGGTGAACCACAGTTAGGGTGTTTTGTTTATTTTATTGTTCTTGCTATTGTTGATATTCTATG
TAGTTGAGCTCTGTAAAAGGAAATTGTATTTTATGTTTTAGTAATTGTTGCCAACTTTTTAAATTAATTTTCATT
ATTTTTGAGCCAAATTGAAATGTGCACCTCCTGTGCCTTTTTTCTCCTTAGAAAATCTAATTACTTGGAACAAGT
TCAGATTTCACTGGTCAGTCATTTTCATCTTGTTTTCTTCTTGCTAAGTCTTACCATGTACCTGCTTTGGCAATC
ATTGCAACTCTGAGATTATAAAATGCCTTAGAGAATATACTAACTAATAAGATCTTTTTTTCAGAAACAGAAAAT
AGTTCCTTGAGTACTTCCTTCTTGCATTTCTGCCTATGTTTTTGAAGTTGTTGCTGTTTGCCTGCAATAGGCTAT
AAGGAATAGCAGGAGAAATTTTACTGAAGTGCTGTTTTCCTAGGTGCTACTTTGGCAGAGCTAAGTTATCTTTTG
TTTTCTTAATGCGTTTGGACCATTTTGCTGGCTATAAAATAACTGATTAATATAATTCTAACACAATGTTGACAT
TGTAGTTTTACACAAACACAAATAAATATTTTTATTTTAAAATTCTGGAAGTAATATAAAAGGGAAAATATATTT
GAAAGGGATAAAGGTAATAGAGCCCTTCTGCCCCCCACCCACCAAATTTACACAACAAAATGACATGTTCGAATG
TGAAAGGTCATAATAGCTTTCCCATCATGAATCAGAAAGATGTGGACAGCTTGATGTTTTAGACAACCACTGAAC
TAGATGACTGTTGTACTGTAGCTCAGTCATTTAAAAAATATATAAATACTACCTTGTAGTGTCCCATACTGTGTT
TTTTACATGGTAGATTCTTATTTAAGTGCTAACTGGTTATTTTCTTTGGCTGGTTTATTGTACTGTTATACAGAA
TGTAAGTTGTACAGTGAAATAAGTTATTAAAGCATGTGTAAACATTGTTATATATCTTTTCTCCTAAATGGAGAA
TTTTGAATAAAATATATTTGAAATTTTGCCTCTTTCAGTTGTTCATTCAGAAAAAAATACTATGATATTTGAAGA
CTGATCAGCTTCTGTTCAGCTGACAGTCATGCTGGATCTAAACTTTTTTTAAAATTAATTTTGTCTTTTCAAAGA
AAAAATATTTAAAGAAGCTTTTATAATATAATCTTATGTTTTAAAAAAACTTTCTGCTTTTAACTCTCTGGATTT
TGATTTTTCAAATTATATATTAATATTTCAAATGTAAAATACTATTTAGATAAATTGTTTTTAAACATTCTTATT
ATTATAATATTAATATAACCTAAACTGAAGTTATTCATCCCAGGTATCTAATACATGTATCCAAAGTAAAAATCC
AAGGAATCTGAACACTTTCATCTGCAAAGCTAGGAATAGGTTTGACATTTTCACTCCAAGAAAAAGTTTTTTTTT
GAAAATAGAATAGTTGGGATGAGAGGTTTCTTTAAAAGAAGACTAACTGATCACATTACTATGATTCTCAAAGAA
GAAACCAAAACTTCATATAATACTATAAAGTAAATATAAAATAGTTCCTTCTATAGTATATTTCTATAATGCTAC
AGTTTAAACAGATCACTCTTATATAATACTATTTTGATTTTGATGTAGAATTGCACAAATTGATATTTCTCCTAT
GATCTGCAGGGTATAGCTTAAAGTAACAAAAACAGTCAACCACCTCCATTTAACACACAGTAACACTATGGGACT
AGTTTTATTACTTCCATTTTACAAATGAGGAAACTAAAGCTTAAAGATGTGTAATACACCGCCCAAGGTCACACA
GCTGGTAAAGGTGGATTTCATCCCAGACAGTTACAGTCATTGCCATGGGCACAGCTCCTAACTTAGTAACTCCAT
GTAACTGGTACTCAGTGTAGCTGAATTGAAAGGAGAGTAAGGAAGCAGGTTTTACAGGTCTACTTGCACTATTCA
GAGCCCGAGTGTGAATCCCTGCTGTGCTGCTTGGAGAAGTTACTTAACCTATGCAAGGTTCATTTTGTAAATATT
GGAAATGGAGTGATAATACGTACTTCACCAGAGGATTTAATGAGACCTTATACGATCCTTAGTTCAGTACCTGAC
TAGTGCTTCATAAATGCTTTTTCATCCAATCTGACAATCTCCAGCTTGTAATTGGGGCATTTAGAACATTTAATA
TGATTATTGGCATGGTAGGTTAAAGCTGTCATCTTGCTGTTTCTATTTGTTCTTTTTTGTTTTCTCCTTACTTTT
GGATTTTTTTATTCTACTATGTCTTTTCTATTGTCTTATTAACTATACTCTTTGATTTATTTTAGTGGTTGTTTT
AGGGTTATACCTCTTTCTAATTTACCAGTTTATAACCAGTTTATATACTACTTGACATATAGCTTAAGAAACTTA
CTGTTGTTGTCTTTTTGCTGTTATGGTCTTAACGTTTTTATTTCTACAAACATTATAAACTCCACACTTTATTGT
TTTTTAATTTTACTTATACAGTCAATTATCTTTTAAAGATATTTAAATATAAACATTCAAAACACCCCAATTAAA
AGTCAGAGATTGTTAATACCACATGATCTCACTTACACACAGAATTGAAAAACTTGGAACTCATAGAAGCAGAGA
GTAAAAACATGGTTACCAGGTGCTGGGGAGAGGCGGTGGGCTGGGGAGATGTTGGTCAAAGTTAGACAGGAGGAA
TAAGTTCAAGAGATCTATTGTACAACTTATTCAGTTAGATAGGAGGAATAAGCTAAAGATCAAGAGATCTATTGT
ACAATGTGACTATAACCAACAACATATATTGTACACTTGAAAATTGCTAACAGTATCTTTTAAGTGTTCTCTCTA
CAAATAAATATGTGAGGTAATGTATATATTAATTAACTGTAGTCATTTCACAATGTATACTTATTTCAAAACATC
ATATTGTATGCTATAAATATATACAACTTTTTATTTTCAATTTTAGAAATGTCCTTAAAAAATCAGATTTTCAGA
TCAGATAAAAAAGCAAGACCCAACTATATGCTGCCAACAGGAAACACACCTTAAAAATAAAGGACGAACAAACAG
ATTAAAAGTAAAAGGATGGAGAAAAGATACATCATATTGGTAATTAGAAGAAAACTGGAGTGACAATATGAAACA
AAATAGATTTCAGAGCAAAGAATATTACCAGGGGTAAAAATGATCATTTTATAATGATAAAAGAGTCAGTTCAGC
AAAAGGATATAACAGTCCTAAATGTTTTTTCACCTCATAGCTGTGTCAAAATAGATGAAGCAAAAACTGATAGAA
CTGTAAGAAGTAGACAAGTCCACAATTATGTTTGGAGATTTTTTTTTTTTTTTTTTTTGTCGCCCAGGCTGGAGT
GCAGTGGCAGGATCTCAGCTCACTGCAAGCTCCGCCTCCCAGGTTCACGCCATTCTCCTGCTTCAGCCTCCCCAG
TAGCTGGGACTACAGGCGGCCACCACCACGCCTGGCTAATTTTTTTGTATTTTTAGTAGAGACGGGGTTTCACCG
TGTTAGCCAGGATGGTCTCGATCTCCTGACCTCGTGATCTGCCTGCCTCGGCCTCCCAAAGTGCTGGGATTACAG
GCATGAGCCACTGCACGCAGCCTGGAGATTTTAATATCCTTTCAATGTTTAGTAGAACAAGAATACACAAAATCA
GTAAGGATATAGAAGATTAGAACAAGACTATCAAACAATTTGACTTAAATGACATTTGTAGAGCACAGCAGTCCC
CAACAACAATAAATCACACATTCTTTCCAAGAGTACATGAAACATGTACCAAGATAGACCGTATTTTGAGCCATG
AAACAAATCTTGATAAATTTAAAAGGATTCAAGTCATAGAAAATATGTTCTCTGACCACAATGGAATTAAATTAT
TAACCAATAACAAATATCTGGGAAAACCTCAAAAACTTGGACACCAGCGCTTTTAAAAGACTAAATAATTTCTAA
ATTATGTGTGTTGGGGGGAAAAGAGAAATGGATTAGAGAGCAAAAAGGGTATCAGAGTGCTGTGGTACGATTTTT
ATGAAGAGTGGAACAGAATCTGCCTTTGGCGTTTCCCCACTACAGCCCATTCTTCACATTGATAACAGCATGATC
CTTCTAAAATTAAATCTAACGATCACTTCTGCTTAATGGCTCTCCAACACTTACAGAATTAGGTCCAAAATTCTA
GCACAGTTTCTGTTCATCTTTCTAACCTTTCTTCCCACAGGTCTAGCTAGTACGTATTTCTTTTATTGCATTTAT
TACACTATTCCTTTGCTTATCTATCTCCCCACCTAGGCTAAAGAACAAGATTCTTGTCTTTTTCATTTTTGTGTC
TCAGTGCCTAGCATGGTGCCAGGCACACAGCATGCTTCCAGTAAATGTTAGCTGGATGGATGTAATGAGTATATT
AAATATTAATTTATTTGTTTTTCCCCAAAAAGAATTATTTCCTGCAAATCAAGGAAATTGCTTTCTTTATATAAT
CAAAAACTTATTTTCCCAGAAGATTCTTCATTAAAAATTAAGCCTATGCACAACCTAGCTCTAAAGTTTCAAAGA
TTTTAGGCAGCAATTTTTCAATCTTTTTGAAGTAATACATTTGAATCTTTTCAAATTTCTGTTTCTGCATTTGTG
CCACACCATCTCATCTCTTGCTGAAATGTTTTTGTTAAATTAATTGCTTGATAAATTGCTAAGTACTTTTCATCA
GACCAATTAGGACAATAGTAAGTATCCATCTGTGGAGCGCGGACATTCAAGAAATCTGATCCAGTATTTAGAAAG
TCATTCCTGAGCTGAGTTGGCTCAAACTGGCACCTTCTGGCATTTGCTTGTGGGTGGGGAATGTGGAATGCTTTG
AAAGCTGAATGAGTTTGTCAAGTTTTAAAATTCCCTTATGGCTAAAGGAAAACAACATTCATTGTTTAAAAACAC
CATTGTTTGTTTTTTCTGCTTTTTTGTTCTTTGGAGCCTGAATCTGCAAAAACACTCACACCCAGCATTTTGCTT
CATGTACCACTCCTAAGATGTTTTTAGAGACTTGAATAGTGTCTCCGCACTACTTTTTATTGTGATTGTTCAGAA
TGTTCATAACAAATGGTAAAAAGTCAGTTTTAGTGCTCAAATTGAGTTTTATGGAGAAAGACCATAATTTATGTT
TGTCATTGTAAATTGATAGGAGAATTTTTGGAAGTTTGCGTCCTAGAACCAGATTTCCAAGGCTCAGATCCTTAT
TTTCTCACTTCCTAGCTGTGTGACCTTAGACAAGGTATTAAACCTGTCTGTGCTGCCTCAGTGTCCTCATCTATT
CTTTAAGAGTAAGAATAGAACCTACCCGATAGAGTCACTTGAAGATTAAGTGGGTTAGTAAATTCAGAATGCTTG
GAACAGTAACTAGCACAGAATAAGTGTCCAATAAAATTGGGTTGCAGCTATTATCAGTATTATTCCTGTCATAAT
CATCATCACCATTAAGCAATTAAATGTAGAGTTCCAAAATTTGATTATGAAACTACAGTTATACAGCCATGATTC
CCGGTGATACCACGTCAGTAACAAGATTATTTCCTTAGCTTGAGCCAGTCACTACCTCATTGCATGTGGCAGAGT
GTGTTGCCGTAGGCAAATGTCATTGTAGGGAATGAAAAAAAAATTGCCTGTGAGCTGCTCTCCAGAGGCCTCATC
CCATTTTCCCATCGTCCACTTTACTCCATCTCCACTGCCACTATTAGGACCTTATCATTTCTTGTCTAGATTAAT
TCAACAGCTTCCTTCCTTCTAGTCTCCATGATTTCACCCACTAGCCATCCCCTCCCCTTTGCCCAATTTTCTCCA
TTTATGGTAGAGTGATCTTTCTAATAGGAAACTCCTGACTTGCCTTAAAAAGCCCTCATTGAGGCCGGACGTGGT
GGCTCATGCCTGTAATCCCAGCACTTTGGGAGGCCGAGGCAGGTGGATCACGAGGTCAAGAGATTGAGACCATCG
TGACTAACACAGTGAAACCCCATCTGTACTAAAAATACAAGAAATTAGCCAGGCGTGGTGGGGGGTGCCTGTAGT
CGCAGCTACTTGGGAGGCTGAGGCAGGAGAATGGCGTGAACCCGGGAGGCAGAGCTTGCAGTGAGCCGAGATTGC
GCCACTGCACTCCAGCCTGGGCGACAGAGTGAGACTCCGTCTCAAAAAAAAAAAGCCCTCATTGACAACCTTCAA
CCCACAATCCATGGTGAAGCACAGGAGCCTTGGGGATCTGCCCCCAGCACACCTCTCCACCCTTGTCTCTCACTG
CTCCTGCCTTCATGGAGAGCCCTGATGAACTATTTGTAGTTTCCCCTGACTCACCTTGCTGTTACTGGGCCTGTG
TGCGTGTTGCTCCCACTACCTGCAATACGCTTACCCACTTCACCTGGGTGAACTTTACTTAGGATTCACCTTAGG
TGGGCATCATGTTCTTCCAGGCCCCTCCTCTAACTTTTAGTTGAGAGTATTCCAGACTTAAGGCTCCATGGGATA
GGGATCTTGTCTATGCACCAGCTTATTCCCAACTGCCTGGCACGTAATGCATTTATTAAATATATATTGAATTGA
TTACCCTACTTGGGGCTCTTGTTTGCTTCTACACTTACAGTTCTAGCATAGCACTTAACTCATTATCATGCATCA
TTATTATGGGTTTGTTTTGTCTCCCATTAGACTGTGAGCTCCACAAGGCTGTGTCCTTGTCTTATACATCATTGT
ATTTCCAGCTTCCAACATAGTGCTTGCCATGACACAGGAAGTCAGTAAGCTCTGAATGAATGAATAGTATCTACA
TACCATTAATCTGAGGTTTAAAGTTTCCCCAAATTCTGAAGCAAGGGGATTTACGGACTTCCCTGACAATTTTTG
GATGTCATCCCAATGATACCACTAACATTTTAAGGGACAGCTTGCATATATACATTTTTCTGGATGGCAGTTTTT
TTTCCCACAGGCTTCATCAGATATTTCTCCATAGCCTTCCTCAGATTCTCAAAGGGGTCTCTGATTCCCCCAAAA
GATAAGAAACTGTCATAAAAAATTATTTCTAAATATCAATTGTTAAATAAAATGTTTGCAAAGCAGCCTGATGAA
TCATTTCAGGCCACTTGACCCCGATGAGTTAGAGAGTTTGTGCTCTGCAATCTGACTGCTTCCAGCAGTCTCACT
GCTGCTGGACTGTGGCACTTCCAATTGGCAGCAGGGCAAGTTTCTTCTGGATGAATATTCTGTCATAGGGGTCCC
CCTTCCACACATACCTGTAGGAGCAGTTTGAAACTCATATGCATGGTCTTCCTGGTTCTAGGCACATGAGTCATT
TAAGCTGCTGGAGCCAGGACCAGCTAGTATGCTAGCCCGGCATTCAGAAAGTTAAAATTTGGGGTCAAAACTGAG
AACCTTCTTTGATCCACCTTGGCCAGACATTTTCTCTGGCTTCCATTAATAGCCTCAACATTTTTTTTTTTTCTG
GCCTAGACCCACACAGGCAAGAGACCAGAGCTTCTCTAAGGAGCTAAGGGAAAGCACATTTTAAAAATAACTTGA
GCAAATGAATTCATCTGGCAAAAGCAACCCCACTACGTAAAATAAACCTTTTTAGTTTCGCAATAGCAGTTCCTG
AAAATGTAAACAACCTCAGGGTCTACATGCACTGAATCATTTGCTGAACAGAAAGTCCCTGGTCCAAATTCTGCA
AGAATAAACACCTTACAAAACTAGGGGTCAATGACCTTCATATGGGAACAAGGAGGGTGTGGGGGGCAGCAACCC
ACCCTGAGGACAATGAGAAAGTCTTGAGACTTGATATTCAAAATGCTGGCTTTCTAAACCAAAAACTGGCATGAG
TGGAGGGAGAAGGGGAGGGTGGGCACAGTCTATGCCTCAGGCTCTTGCTCAGACCCTACCAGGCCCCTGCCTTCC
CTAGGGAAAGCGAGAGTCTACTCACTGTCATGAAGCCAGAGGAAGGCCCTGCAGGTTTCACTGTGTGTTCTGTTG
ACAAGATGATGGTTCCATTGAAACTGTAATAACATACTTGGCCAACTAAGCCCATACGATCGTAGTAACTTTGTA
CCCAGTCCTAGCTTTTCAAACATAATGATAATATGTTCTTTCTAATGTGGCCCATACTGTTCTAATGAACTTATG
CTGAGTTTTTCTGAGTACTAGAATAATATTCGCCATAAATAATAGATATAATTATTCTCATTTAATATTTGCGTA
GCTCTTCTTTAAAGCAGAAAGTATTTTCTCATTCCTTACTAGAACCTTTCTGTGTGAGGAGCACTGAGCTAGAAC
CCATATCTTAGAATGGTCAGAATTTGGAGAAATTCAGGGAAAAGGCACTGGACTCATTTTTAAAGACTAGAAAAT
GCAACCTCCAGAAAAAGATTCAAGAGTTTTTTACTCCCAGAGATGTAGGAAAGATTGGAGTAAATCTTAATATTA
TATTTCAGGTAAACAAAGGATCACTGTCAAAATAGCAGCATTTATTGAGTAATGGCTGTGTGCCAGGTACTTTAC
AGTTTCACATTTAACCCTCATAATAACCTTGTAAAGTGGATATCCCCTCAGTACATGATGAGAACACTGAAGCTT
AGGTTAAATGATTGTCCAAATCGGACAATCATTTTCAAAATCTCCCCCTTTTTTTCTCCTTTCTTATCTGCAAGG
CAGATTGCCCTTTCCCTTTCAGTGAAACTTGTGCATGACCACATGACTCTCTTTGGCCAATGAAACATGAACAAG
CAGCGTTTATCACTTTCAGATGGAAGGCTTTGCATGAGCTTTGCCTCCTTTTCACTCTGCCACAGTGGCCACTAA
CATTCCAGATAGTGGCGCTCTGCAGGCTAGGTCCTATAGTGGGAGCTATGGGCAGAGCCCCCTTTCCCACCCCCA
TCAAGATGTGCATGCTGCATAAGCCATGCATTAATCTTTGCAGTTTTAAGCCACTAAGTTTTGGAGTTATATTAA
TCATTAATCATGGTTCTCAAGAGAAACAGAGTGGGGGAGTGGTATTCATTATGGGAATTGGCTTACATGATTATG
GAAGCTGAGTAGTCCCCCAGTCTGCTGTTTTGAGCTGGAGAACTAGAGGAGCCAGTTGGTATAATTCAGCCCAAG
CCTGAAGGCCTGAGAAATGGGATGGGGGAATTGGGAGGGTGGGTGTGCTAGGGTAGGATAAGTCCTGAAGTTCAA
AGGCCAGCCAGAAGGTGGATGTTTCAGCACCAGAAGAGAGAGCAAATTCGCTTTTCTTCTGCCTTTTTGTCCTCT
CTGGGCCCTCAATGGATTGGATGATGCCCTCCCACATTGGTAAGGGTGGATCTTCTATACTCAGTCTGCTAATTT
CTTCCAGAAACATCTTCACAGACACATCCAGAAATAATGTTTTACCAGCTATCTCGGTATCCCTTAGCCTAGTCC
ATATTTAAAAATTAATGATCACAAGCAGTTGTTTGTTTCCACAGCAAAACCTGGGTGACAGACCAAGTGACCCAG
ATGACTAGAATTTGACCTTTCTTTGTTGCCCACACCATACTCTGAACTAACATGCTGTGCTGCCTTCCAAGTGGA
GAATGATGGCTAAGTATCTTCTACCTAATTTGAGTCACAGAAAAAAAAAAAAAAGGTTATTAACTGCAGTGACAA
GAATTGTGATTCCCCAGGGGGCAGATCAAGACTGATAGATAAGAGAAGTGAGGAACATCTGGGGAATGTCCATTG
AAAATTTACTCAGAAGAGAAGAATAATTAATATAATAATATGATATATTGAATTATAATAAATAATATTTTGATG
TATTTCCTTCCAGGCATGTTTAAGTTATAGACTTTGAGTATATTTTCTCAAAGGGGGTTCTATGTAAGAGACTAT
TTCTTAATATAGTTCCTAGCTTGGAATTGCTCTTGCTGGTTTAAGCTGAGCTTATTTTATTACAGACTTCACAAC
AATAACGTTTTCCTTCACTAGTCAGTACACAAGATGGTCTTCATTTCCAGTTTGGAATCCCACACTATCAGAGCC
TGAGACAAGGACTAGTATGCAGTTAGTTTGTTTGGGAGGTGATTCCAGGAAGTGGGAATGAGAGATCAGTCAGCC
TGCAACACGAAGGAGGAAAAGTCAATATAAGGATGAATTTGGCAATTGGCCGTTTCATGCAACTGGGGCTAAATT
TTGCTTGGCTCTCTAAGAAATGTAAAGAATGCCTCCCGTAATTGCTCACCTCAAGTATTTATTCATTGGCTCTCA
TGCTCCATTGGTTGTCCATGAGAACTTTAGCCCTCCCTCGCTGCAGCACAGACACTGTGCTTTCTCCTAGGCTGA
GCAAGCTCCTGCATCTGTGGAAACCGTCCCGGGGCAGATAGTGAAATAATGACTGCTGCGTGCTTGAGATCTGGG
AAAGAGGCCACATCATAAGTGCACTGAAATCAGAGATGTGTCAAGAGATGTGACACAGGGCATCTGAGGTGTCTA
CTGCACCAGCTATAACTCCCTAAACGCTAATCTCAGTTCTTACAGAGGGGATGGATGCAAGGGAACAGTCATGAT
TGAGAGCACCGAAGAAGCTCTGTATGAACCTTAGGCAAGTTTCCTAATCTCCAAAATGAAGGTAATAATACCCAC
CATCCAAGATCTTCGGGAGGAATAGATGAACTAATGTATGTGAAAATGTCCAGCACAGGTCCTAACCCATAGTAG
GTGCTCACCAAATGTTAGTTCCCTGCCCTCCACGTTGTGTGTATCCGGAGCTGCACTAGATGCTGAGGCAAATGG
TCTCAAATGTACTTTAACACTTAATGACTGAGATTTTTTCTGAGCTGCCTACAGGTTATTGACTATATTCATTAT
TAATAATAATATATATGGCCACTTCAGGCAACTGGGGCTAAATTTTGCTTGGCTCTCTAAGAAATGTAAAGAATG
CCTCCTGTAATTGCTCACCTCAAGTATTTATTCATTGGCTCTCGTGCTTTATTGGTTGTCCCTGAGGACTTTAGC
CCTCTCTCACTGCAGCACAGACACTGTGCTTTCTCCTAGTTTCTGTGGCAAGTGACAGGAGCCCACCTCAAACTA
AAGCAAAAGGGACTTCATTGGCTCTTGTAGCTAGGAATTCCAGGGTTGGCACTGGCTTTGGGCACTACTGGATGC
AGGAATTCAAACAATGTCTTCAACTCTTTCTTTTGGTGTTTCTCTCAGCTGTGCTTCTCTTGTCGTTTCTTTTTC
CCATTTTACAGATAAGTTCATCCGTAACTGAGAGAGGTGAAAAGGGGATGGCTGCAGAGAACTCTGGCTTATATC
ATCCTTGCTTGCTGACCTCAAGGTCCATGTATAAATTCTCAGAGAAGAAGCCCTCTGGTTGGTGATGCTTGGAAC
ATGCCCTGGAGGGTGGGCCCCTTGAAGTGGAGCTTGCTGGAACCACATGGGCTGGAGCAAGGCGCTAGGGCCAGA
AGAGAGAGGTAGGCAGGGCTGCTGGCCAGGCACTCTTCACCAAGACAAGGCAAGAGGAGGGGCATGATTGAGGCA
GTGATACAGAAAGCAGACAGTAGAGGTCGTGGCAAGTGTGCCGTTACTTGCTACCTGTGGTTGATGGGAGAGTCA
CACCACATTTAGGAGGAGAGAATCCATTTGCCACTTCTGACAATGCCACAAGAATCACATATTTCATCCAGAGGT
TGAATTTGGCCCATGCTGAGCTTTAAAATACAGAGCTGTCTTGGAACAATGGCTCAGTACATTCATTTGGTGTCC
AACAAAGCCTGCCTCTGTTGCCTTCCCTCTCTCTGTGTGCCCTTCAAGATCTTCATTGTGCTTTGGGGAGAGAAA
GAGAAAATGTCATATCAGGGTAGCTCACCCCATGTGTCCTGGACTCAGGAAAAGAGTATCTTATCACCTTACTCT
TTTGTTATTATAAAAAAAAAGTTGAACGTCTTCAAATAAAATAAAGAAGTATAGAAAAAATTTTTAAATTAACCT
GTTATGATTCTACCTAGAGAACCATTGTCAACATCTTGGTATATGTACTTCCAGATACTTTCCTATGAATATATA
CATTGTAGATTTTTTAATATTAAAAGGCTATCATGCTGCTTTGTATACAGGCTTTCTTTACTGATATGTAATATA
ATACACAGACAAATATACAAATCCTAAGCCATCAACTCATTGAATTTTATTCATTGTTTTTTAATACCTGCATTG
TGTTCCATTGTTAGGCTATGTCACAACATATTTTAATTAAGCCCCTATTGATGAATATTAATTACTCTATTTGCC
AGTTCATTCCAGTCCAACATTTATTGAGTGTCTACTTACGGGCCAGGCACTCTTGTATTCATCAAGATCACCACA
TTATCTGTATCAGTTATTTATTGCCACAATAAAACTGCATAACAAATCACTCCAAAATGTAGCACCTTAAAACTA
CAACTACTTATTATTTCTCAAGAGTCAATGGGTCAGCTGAGCAGTTCTGCCGATAGGGGTCAAGGTCAACACATT
TCAACTAGACTACTTGTAAAAAAGAATGAGTGTCTGGGTAGGTGTGTTCTTCTAAAAATAAAACAAGGAATGAGG
AAATTGCAGGTAGGATAAGAGGGGGGTTTGGCAACCAAACCCCACAAAAGGCAGACAAATTTTAAGGAAACATAA
TGCCAGACTCCTATGTCATCATCCAAGTAGATGCAGTGAAGTATAACCTGGGGCGTAGTAGGGTAGGAGTGGGGA
GAGCAGAGGAGAAGGAAGGGAGATTGCTTTTCATCACTTTTGGATTCCCTAATAACAGACATGACTGCCAGTATT
AAAATTTAACAAAGGATATCTGATCATTAATTTTCCTGTATAAGTCACTGGTGATCTTCAACATCTCTCCCTCCC
TTCCTCCCTTCCTTCCTCCCACCCTCCCTTCCTTCCTTCTTTCCTCTTTTGCTTTCAACTTCCTTTTCTCGTTTC
CTTTTGCTTTCTTTCTCTTCTCCCTTTTTTCTGTCACTCTGGGCGTATGTAGTAGTGTAAAAAGGTTGACAGAGA
AATCAAATATAACAGGAGCAGGGCCCTGAGAAAAGCACCTGGCATCCTGTAGGCAAACCATTGTTTCTAAAAGAA
GGGACTGAGAGATTGAGGAGCTCAGGACATTGCCAAATGAACAAGGCAAGCACATTTATTCAGTACCAAACAAAC
GGAAAACGGCCTTTCCAAATAACTGACCTATAAAACAGCCTTTTCACAAGAGTACCGTAATTACTGGCCAACAGC
AACAATGAAAAACAACTCCCAAACAAAGAAATATTTCTGGATTAAAAGCCATGAGATCTGGATTCTAACAAGCTG
TGCTCCTCAAACTACAAGTACAAAATCTGGCTCTAAACTAACAAGCTATGAGCCTCAAACTGATGACTGGCATGT
TTGGGTCTCCATCTCCTTCTTGGGGGTTGGGGTCTTAGAGACCCTTTTCCACGCCCTGATTCTCTTACTAGTGTG
TATGCTTTCCTTTTGACTTCTCATGCTGACCGTCTGAGCAGGAGTGAGAAGCAATTTCAAAGGAAAACATCGTTT
ATCATCTGCTGAAAGAAACCAAAAAGAACACAGGAAAACAAAAAGACAAGGAAAGGGAATGAAAATGTAATTCAT
TTTATTAAAAAGAAGAATTATTCTTCTGGGACACTGGATAGAAACCTTAATGAGTTACCTAGCTATCATAAATCC
TCTAACAGAGAAGAGAAGAGAAAGAAACAAAGACGGAAGAGGGCAGGATAAAAGAAAGAAAAAAGGAAGGGAAAA
ATGAAGGAAGGAAGTTATCTATTCATTTCTACAGAGACTCTGCTGAGCAGTAGACAAGAAGACTTGGGAAAAATT
TAACTGAAACTTTTCCAAAAATCTTTTCAGAGGGATTTTTTCCCTCTGAAAAGCATCATTAGAGGCTGTTCAATA
CCCAAGGCAAGCCTCTTTCATATTACTTACTGTACATGAAACACTCATGCAATTGAGGCTAGCCAGAGGCCATTT
AGAAATTCAATAATTATTCAACCCAAGGGGCTTTCCAAATGGTGAAGTAGCTTCTTAAGAGGAAATTAATATTGA
GCAGTATAGCAAACCTAATTGGAATCTTGAGAAAATAGTTCTGTGTCGTTAGAACAGCTAGAGGCTAAAGAAGAT
CAGGTTGGATGATACCTTCATTTTTGTCTCTTTCCTTAATTATGATGTAAAGGGAAAAATCTTGTTTATTTTCTA
TGCCAGGAGGGTAGAGGGTGATTTGGAGAGGTTCCAAGTTTATCAAAATCTACCTTCAGTCTGGCAGTAGAAAAG
TTTACTTCCTTCATTTCTTTCCTATAGACATTCAAAGAGAGCTAAGGAGATCCAAAAACCTTTTTTTCTATATTT
GCAATGCAAGGCAGTTGGGAATTAATGACTGATTTGTTGGTGAGGGCAGTGGGCATTGATCACAAAAGCAGTAAA
GCTGTGTTTCTCAAAGAGAGAAAGTCTCTTTGAGATCTTCATTATTTTACTATTTAGAAGAGAAAGGGGCGTTAT
ATCACGTTGGAAGCATCCATGAGTCACTAGTCTCTTCTCTATCTTTCTATGCCTTTCTGTATTAATTACTTTGAA
AGCACAACATTCCAAACCCATTGAGCACACAGTGGTCTGATTTCTCCACTTGTGAAAGGTGCTAAAGTCTCACTG
TAGGATTAATTTGGGGGTCCAGGCTATGGGCTTGTAGATATGACTACCTTAGACTTTGGTTCTCCTGGCAACTAA
CCCTTTTTGGATCGTATCTAAGTTGACCTGTTTCACAGTGAGAGAACTCCTCTCCATTACTCAGAATACTGAGGC
AGATCACAAGTGTACCACACCTGGCTAATGTTAAGCCAGACAGAAACATCAGGCTCATCTCTTGAGAAGAAGGGT
CGCTTATTAAGGATACAAACTATTTTTTTTTTTTTTTTTTGAGACAGGGTCTCATTGCCCAGGTTAGAGTGCAGT
GGTGCAATCATAGCTCACTGCAGCCTCAACCACATGGGTATTTTTAAATAAGAAAAAAATACCATCTGATAGATA
TGAAGGAGCATTGGGTCACTATAAACAAAACAGATTCTAAGAGCAGGAAGAAAGAGTACAGTCTCTTTTCAATAA
TTTTTTTTTAAACTTGGGAAAGAACACTCACTCTATTCCTATAGACCAGAAAGCAGATAATTGTCCATTATGATT
CCACATGACACTATCTTGTTCAGCTGTCACTGAAACAACTTTGAACACTGTCATATGTTCTTCCCAGCTCCTGAA
CTCTGACCTTTTTATGCCTTAGTTCCACTTTCACAAAAAGGGATTGATGTAATGTGCATTTCAGAGGAAACGACT
ATAGACATTTAGTGTCATTATAAATGTTGAGAAGTATGCTGGCAGAAATTATGCCTTAAGATCATATATGGATTC
TTGTATGGTTTGAAATTGCTTAAAAGATATATATGATCTCTAAAATGTGTGTGTATATATATATGATGTCTTCTT
ATATATCTATATGTGATATATTTATATATATATAAATCTGTGTATATCACATATATAAATTTGCTGTTATTTGAA
TTGCCATTACCTCAGTGCTTAGGGGAAGCCATGCACGTTTGTTTCTTTTCAGTACCCAGAGTTAATTAACATAAG
TTATCACAGAAGCTCCCATAAGCATTGAGACAATTTCTCTATACCTGTGACTATTTAAGGTTTTGAAAACAAAAC
AGAAGCAGGTAAGGAGGAAGTACGCTTTACTATTGAAGATTTATTAGGTACACATTTAGATTTGTGAACTCACAT
TGCTTAGGATGAAAGGGACTCTTGAGGATGTCTGCTGTTTGTTAGTGAACTGCCTGTAACAATTACAATTAGCAC
ACACATGAGCACAATGAACTGGGTAGTCAGACTCAGCCAAAATGAATAGAAATAGCCTCTTACCAAATTTACTTT
GAGTAGCCCTTGGACTCTGAGCACTGCTGCCCAGAGCAATATGACTGTAGGTCCAAGTTTGTCAATGACTATGCA
AATGTGCTTTCTTCGCTTTTACTCTATTGTCATCTGTCTATTACAATGTTGCTATGGTGACACCTTTCCAATATC
CCTGTGCTTCTTTGGTATCCTCTAAGGGGAAGCTGTAATGAAGTGGCTTGGCAAAAGAATCCTCTTGGAATTTTT
TTTTTTTCATATGCTACTGAAAACCAGCATGATTTTCCTCTTATGGGAAATGTATAAAGTATGAGTTGGAAATGA
TGGAAATTAATCTGTACTGACTTGGGCAAGGAATGTGAATGTTATTCATTCTGTTCCAAACTACCTGAAAATATT
CTCTTTCTGTTCCTACTTTCCAGGAGATAACATCTTAAGGGACACTGAAGCTTGTGCGTGTGTGAGTAGAACACG
TGCTGGGGGCTCTTGAGCTCATGAGGGAGGGGCTACATGTCGGTGGGGTGATAACTGTATGCTGGAAACAATGAT
AGGGGTGACCCTGGAGCACTTACCATGTGACAGGTGTTATGCTAAGCATGTTTGTATGCATTCCTTCATTGAATG
ACACCTACCTATATTATCCTCATTTTATAAGATGAGGTAACAGAGCTTCAGAAAGGTTAGACTCAGCTGCTATGG
GTCTGTCTGACTCTGGTGTTCTTCCTCTTAAAAACTGGGGCACTTTGGAAATGAGATTCCTCGGTGATGAACAGA
AATATTGCTTAGCGGCTGTATTTTTGTATCTGGCAGTTTTCCCATATTTGAGTCTTATATTCACAATCGGTATCT
TTACATTACACAAAAGTGACACAGAATTAGAGTCATTTAATCCAGGGTTGATATCATTAAGTCATGACTATTTAT
TAAATGTTTCTTACAATATCTGAGATGATATTGCAAAAGATGTAAGTGATTTTAGAAGTTCTCACTTCGTAGTTA
GTTGCAGAAACCTCTTTTGGAGGAGGGATGTTTTCTCTATATATCCTAATTTCTACTTAATATATTTCCACACCT
CTTTGAAGTGTGTAGTAAGAATGGTAAAATGCAGTACTTCGTCATTTGGTACAGTTCAATCAATATGCATTAAGA
TGTGATCATATGGGTAATAGAAAAATGTGAAAGATCCAATTCTTTTTCTCCAGAAGGCAGGAAGCTCATATTTGA
TTTCTGTTACTATAAACTATAAAAACGTTTCAAATGTAGTTTACCCGTAACCATCACCCTGCAAGGGTGATATTG
CTCCCCGCCAATTTACGGAGGAGAATACTGAGGCTTTAAGGTTGTAGATAGACCAAGACCACACAAGTAGAGAGT
GGCGGGCTGTGGGTTGAGCTTTAAAATCCAGGTTCATCCATGACTCCCAGTGTGTTCTAGTAAATCCACTAGAAT
CTGAGTATTTTCCAATGATTTATGCTCCGCTCTGTGTCAGGCAGTTCATGGTATTTTTCAACAATCAGAAAATCC
TGGGGAAGGCAAACTGTTTCCCCCTCTCTAGGTGCCTTGGAAGTGGCCGTTGTGGACCCAGAGATCATCCTTTCT
GATCTGACACCTTCTTCACTGCCCTGGCCCAGTGTCTTTTCTGCAAGGCTGGAAGCCCCCTTAGACTGGTCATGT
CCCATCTCTTTCCGGAGGGAAGATGATCCCAAAGACGACTTTTCTCTCCACGGTGCTGCCATACCGCAGGCGGCC
GCCAGGGGTCCCCGCTCGGCGTCCCCGCGAGACAGTCGAGCCCCGGCCGGCTGCGCGGCGCGCTGGGTGCATGAG
GGGGCTGCTCCGGAGCGACGGCGGCTGCAGCTGGAGCCAGGCGCTCGCCCGTCCGCCGGTTGGCTCGCCGGGACC
TCGCGCACCGGCGGCAGAGTCCCTTGCGTGGATTGGCAAGCGACGCCCCACCTGCCCCGAGCTTCACCATTTCTT
TCGCGCTGGCTGCAGCTGACCCGGCGAAGGGAGCCGACCGGGCCCTGGGCTGGAGGTAAAACCCCACGGTGAGTA
AGAACCCGCTCCAAGCTAGGGGAGGCGGCGCAGCCCGGTGGCTGCTCGCTCCCGATCTCGCCCGGGGGGGGGGGG
AGGTTTGGGGCGCACCTGGGCGCGGGTGCAAGAAGGTGCGGGAGGCGGCGGACCGGTCTTCTGCCCGCCGGCCAC
GGGCTTCCGGGGCTGGAGTCCTCTTCAGACCCCTGCCGGCGCCTGGGTTTCTGGCCGGCTCCTCGTGTGCACTTC
CCGGCAGGAACAAGGGTCGCCCACTTTCCACCCCGGGATCTTGATTTGTCCTTGATTTGAAAAGATATAAATCAA
TAAGATCGTCCTTCTTTCGGGGTGCAAGACTCCGAGCCCATCCCCAGCCGCGGACGCCTGCAGGGTGCGTGTTGG
GCTGTGGGTGGCGGGAAGACAAACTTTTACAAAAGTGCGCCTGGGCTGGGGGACAACGCTTGGGCGTCCTGATCC
TGAGGGAGGAGTCTCGGCTTGGGGCAGCGTAGGGGAAGTCCGCACCGTCAGCCAGGTCGCCCCCGGGGCTGACGA
TGCCTCACGGAGGTGGGGAGCGTGTAAAGGCCGTACAAATCGCGCTTAACTTTGGGGCCAACAACTGTCAAACAT
CTGGAATCCCAGCCCCTCCCTTTCCCTGAACTGGGGAAGAAGGTGAAAACCCTTCAAGTTTTCTTTGATTGCCCC
TTCCCACCTTCAGACCCCTGCTGGGAGGGTAAAGCGCCGACCCCTGGTGCCTGGCAAGTACCAGAGACTCTAAAT
CTCTCGGGATCCCCCCCCTCGCGCTCTTTCCTGACCCTCTCCCCTAACCCTCCCCACAGAGATCTCTCTACGCAG
CCGACTGAGATCGTGGCGAATGGCCTTTTGTTTCTCCGCGTTTCCCCTATTGTTTGCCTTTCCAACATCTGGCGG
GGCTTGGGGAGAGAAGGAAGCCCCTCTGGTCCCCCTCCCCGGCCCCCACGCCAGCTCCGGCAGGGGATCCCAGCT
GGGAAAGTGGAGGAGCCCGACCCCAGCGAGGCCGCCCCACCCCGCCCTTGTGGTTAGAGGGCGGAGGGAAAGTTG
TTCCTTCCCCGCCTCCGCTGCTGCCTGTGGCCCAGGGCGCATTTCTCAGATCTCAGCCCAGGCGCGCCGCAAAGG
CTCAAATCCGAGAAGGTGCTGCTTTCGAGACAGTGGAAGCGCGTTCCGCCCCAATCCAGAGCGTCCAGTGGTTGG
TTCCAGAGGATTTCAATCTCTAGCCAAAGGCGTTGGGGCTGGGCCGCTGCTAGGGCAGTGGGAGGGGATCGGGGC
ACCTTTGGTAGGCGGAAAGCTGAGATTCTGGGGTCCACAAGTTTCCAAGGGCGGGAGGGCAGGCTAGTCGCCAAA
AAGAGAACGAAGATGCAAATAACGAGGAAGCCTTATGACGTTGCCTGGAAATAGTAGTGTGGTGGTTCACTCCGG
AATGAACGTGGAGTTCTGGCTTTGAGTACCGCTCCAAGTTTAAATCCCAAGTCCCCTTTCTTCATTGTAGAAAAA
GAGGACTCAGACGACGCAACACAGATACGGCTAGAGCACAGTTCCTGCTTCCACGTCCCAGAGAACAAGTGGCTT
AGGATGGTCCCGAGTTCCCCTGTGGGTGCGCTTGTTGGGTTGCAGGCGGCCCTGTTTCCCTGCACAAGTCAGATG
CTTACACATTGTGTTCATTCTTAGTGTGGATTATTGATTAAAGAACTGGGGCAAAAGCAAAGTAGCTACTCTGAG
AAGTCAGGGTCCCCAGATGGTGCCCAGCGAGTTGTCTTGCCTCTGAGGGGAGGCTGACTGAGACTGTGCACCTGT
TAGAACCTATGCTACCCCATAGCCTTGCAGTTGACTTGCTGTTGCCAGCTTTTCCTGTGGGATCCCCAATGAGTC
CCTCTTCCAAGGAAGCTCAATTACACTTTTGATTCCTCCTCAACCCAGGGGAAGAAAGAGGCTTCTGTAGGAACA
TTATGATCTATGTACCCACTCAGACATTGTCAGTGGATACCAGAAGCTTGGCTCTGCACAGCTCTGAGAGTTTTC
CCTTTGCGAACTCAACAGAACTTTTGAGTTTCCATTTAACATAAAAGAAGTGAGACTGCTAAGCCAGGAATGCGA
CACATAGAGCACTTTCTCTAGTGATTTCTGGGTATTATATCTCTTTACCTTCCCAACGGTGGAACCAGGAAAAGA
AAAAAAAGCAACATCTTTGAAGTACTGCAAGGCACTTTACAAACATTTCATTATGAAAATGATCCCCAAGGAAGG
ATTCCTTTGAAATTTAGCAGCAGCAACCCAGAAGCAACAAAAAAGACCAAAGTTACTCAAGAAGTACCCAAAGGC
ATCATTAACAAAATAAAAGAGCATTTCTTGTCTTGGCCTACCCCGCTAAGGAAAACAGGGTAATTATAGTGGAAG
TTAAGCTTG

In some embodiments, the human C9ORF72 gene and flanking sequences comprise a sequence that is, e.g., at least 75, 80, 85, 90, 95, 96, 97, 98, or 99% identical to the sequence above. As used herein, the term “percent sequence identity” or “percent identity” or “% identity” is the identity fraction times 100. The “identity fraction” for a sequence optimally aligned with a reference sequence is the number of nucleotide matches in the optimal alignment, divided by the total number of nucleotides in the reference sequence, e.g. the total number of nucleotides in the full length of the entire reference sequence.

In some embodiments, the number of GGGGCC hexanucleotide repeats (e.g., (GGGGCC)ₙ in SEQ ID NO: 63) is 300-800, 300-700, 400-600, or 500-600. In some embodiments, the number of GGGGCC hexanucleotide repeats (e.g., (GGGGCC)ₙ in SEQ ID NO: 63) is 500-600. In some embodiments, the number of GGGGCC hexanucleotide repeats (e.g., (GGGGCC)ₙ in SEQ ID NO: 63) is greater than 300, 400, 500, 600, 700 or 800. In some embodiments, the number of GGGGCC hexanucleotide repeats (e.g., (GGGGCC)ₙ in SEQ ID NO: 63) is greater than 500. In some embodiments, the transgenic mouse is an FVB, balb-C or C57B/6 strain mouse. In some embodiments, the transgenic mouse is an FVB strain mouse. In some embodiments, the mouse can be used to screen for therapies for the treatment of ALS or FTD, e.g., a therapy described herein or a candidate therapeutic agent.

A transgenic mouse as described herein can be made using any method known in the art or described herein, e.g., Example 4 (see also, e.g., PCT Publication Number WO2001010199 and WO2013022715; and US Publication Number US20110113496 and 20060031954, each of which are incorporated by reference herein). For example, a transgenic mouse described herein may be produced by introducing transgenes (e.g., the human C9ORF72 gene, optionally with flanking sequences) into the germline of the mouse. Embryonal target cells at various developmental stages can be used to introduce transgenes. Different methods are used depending on the stage of development of the embryonal target cell. The specific line(s) of any animal used to practice this disclosure are selected for general good health, good embryo yields, good pronuclear visibility in the embryo, and good reproductive fitness. In addition, the haplotype is a significant factor. For example, when, transgenic mice are to be produced, strains such as C57BL/6 or FVB lines are often used (Jackson Laboratory, Bar Harbor, Me.). The line(s) may themselves be transgenics, and/or may be knockouts (e.g., obtained from animals which have one or more genes partially or completely suppressed). The transgene construct may be introduced into a single stage embryo. The zygote is the preferred target for micro-injection. The use of zygotes as a target for gene transfer has a major advantage in that in most cases the injected DNA will be incorporated into the host gene before the first cleavage (Brinster et al. (1985) PNAS 82:4438-4442). Normally, fertilized embryos are incubated in suitable media until the pronuclei appear. At about this time, the nucleotide sequence comprising the transgene is introduced into the female or male pronucleus as described below. In some species such as mice, the male pronucleus is preferred. It is most preferred that the exogenous genetic material be added to the male DNA complement of the zygote prior to its being processed by the ovum nucleus or the zygote female pronucleus. It is thought that the ovum nucleus or female pronucleus release molecules which affect the male DNA complement, perhaps by replacing the protamines of the male DNA with histones, thereby facilitating the combination of the female and male DNA complements to form the diploid zygote. Thus, the exogenous genetic material should be added to the male complement of DNA or any other complement of DNA prior to its being affected by the female pronucleus. For example, the exogenous genetic material is added to the early male pronucleus, as soon as possible after the formation of the male pronucleus, which is when the male and female pronuclei are well separated and both are located close to the cell membrane.

Alternatively, the exogenous genetic material could be added to the nucleus of the sperm after it has been induced to undergo decondensation. Sperm containing the exogenous genetic material can then be added to the ovum or the decondensed sperm could be added to the ovum with the transgene constructs being added as soon as possible thereafter. Any technique which allows for the addition of the exogenous genetic material into nucleic genetic material can be utilized so long as it is not destructive to the cell, nuclear membrane, or other existing cellular or genetic structures. Introduction of the transgene nucleotide sequence into the embryo may be accomplished by any means known in the art such as, for example, microinjection, electroporation, or lipofection. The exogenous genetic material is preferentially inserted into the nucleic genetic material by microinjection. Microinjection of cells and cellular structures is known and is used in the art. In the mouse, the male pronucleus reaches the size of approximately 20 micrometers in diameter which allows reproducible injection of 1 −2pl of DNA solution. Following introduction of the transgene nucleotide sequence into the embryo, the embryo may be incubated in vitro for varying amounts of time, or reimplanted into the surrogate host, or both. In vitro incubation to maturity is within the scope of this invention. One common method in to incubate the embryos in vitro for about 1-7 days, depending on the species, and then reimplant them into the surrogate host.

Transgenic offspring of the surrogate host may be screened for the presence and/or expression of the transgene by any suitable method. Screening is often accomplished by Southern blot or Northern blot analysis, using a probe that is complementary to at least a portion of the transgene. Western blot analysis using an antibody against the protein encoded by the transgene may be employed as an alternative or additional method for screening for the presence of the transgene product. Typically, DNA is prepared from tail tissue and analyzed by Southern analysis or PCR for the transgene. Alternatively, the tissues or cells believed to express the transgene at the highest levels are tested for the presence and expression of the transgene using Southern analysis or PCR, although any tissues or cell types may be used for this analysis.

Alternative or additional methods for evaluating the presence of the transgene include, without limitation, suitable biochemical assays such as enzyme and/or immunological assays, histological stains for particular marker or enzyme activities, flow cytometric analysis, and the like. Analysis of the blood may also be useful to detect the presence of the transgene product in the blood, as well as to evaluate the effect of the transgene on the levels of various types of blood cells and other blood constituents.

Progeny of the transgenic animals may be obtained by mating the transgenic animal with a suitable partner, or by in vitro fertilization of eggs and/or sperm obtained from the transgenic animal. Where mating with a partner is to be performed, the partner may or may not be transgenic and/or a knockout; where it is transgenic, it may contain the same or a different transgene, or both. Alternatively, the partner may be a parental line. Where in vitro fertilization is used, the fertilized embryo may be implanted into a surrogate host or incubated in vitro, or both. Using either method, the progeny may be evaluated for the presence of the transgene using methods described above, or other appropriate methods.

Aspects of the disclosure also relate to polynucleotides, e.g., a bacterial artificial chromosome (BAC) vector, comprising SEQ ID NO: 63.

Without further elaboration, it is believed that one skilled in the art can, based on the above description, utilize the present disclosure to its fullest extent. The following specific embodiments are, therefore, to be construed as merely illustrative, and not limitative of the remainder of the disclosure in any way whatsoever. All publications cited herein are incorporated by reference for the purposes or subject matter referenced herein.

### Examples

### Example 1

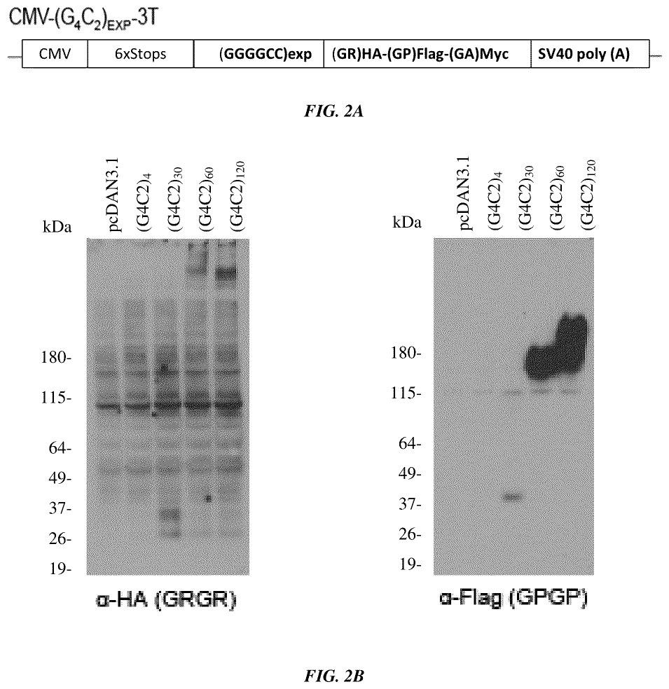

A construct containing a CMV promoter, a (GGGGCC) expansion motif containing either 4, 30, 60, or 120 repeats of GGGGCC, and an HA, FLAG, or MYC tag were transfected into cells (FIG. 2A). It was shown by western blot that poly-(GR) and poly-(GP) proteins were produced in cells transfected with constructs containing 30, 60 or 120 repeats of GGGGCC (FIG. 2B). It was further shown using immunofluorescence of cells that GP-flag, GR-HA, and GA-Myc proteins were expressed in cells transfected with constructs containing 30, 60 or 120 repeats of GGGGCC (FIG. 3). These results show that GGGGCC repeat regions are capable of initiating translation independent of an AUG start codon (repeat-associated non-ATG (RAN) translation), and that poly-(GP), -(GR), and (GA)-repeat proteins are produced.

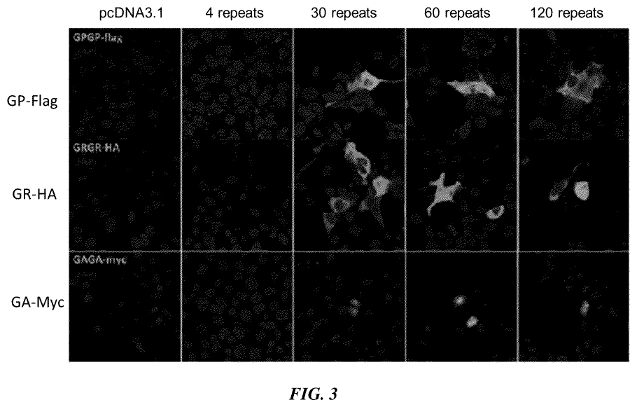

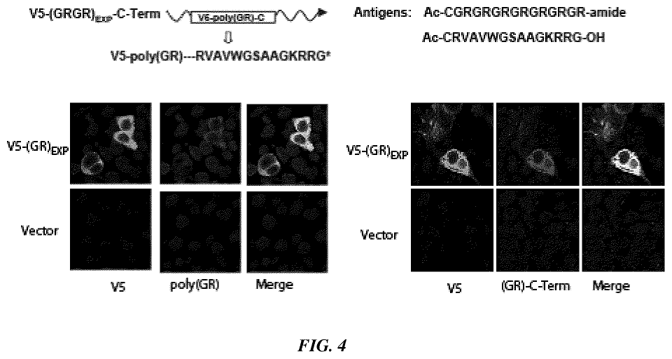

Antibodies to a poly-(GR) sequence or to the C-terminus of the poly-(GR)-repeat protein were generated. Fluorescent staining using these antibodies showed that these antibodies were capable of detecting the poly-(GR) repeat protein (FIG. 4).

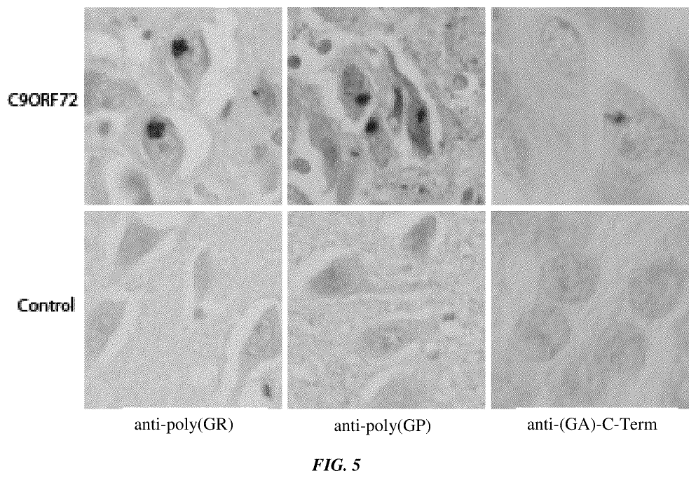

Antibodies were further generated to a poly-(GP) sequence and the C-terminus of the poly-(GA)-repeat protein. The anti-poly-(GR), anti-poly-(GP), and anti-poly-(GA)-C-term antibodies were then used to stain sections of brain tissue from patients with C9ORF72 ALS or controls (FIG. 5).

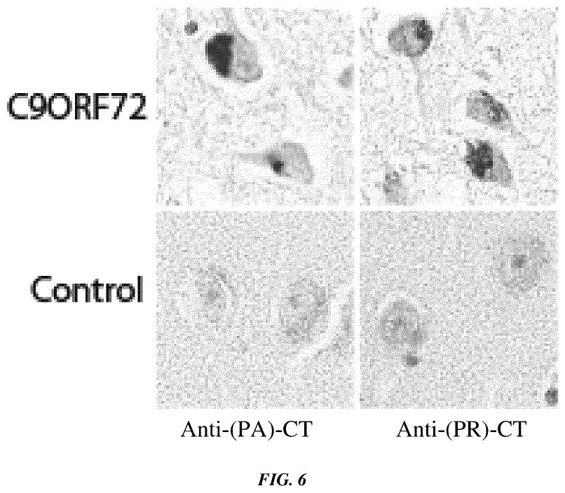

It was then hypothesized that transcripts of C9ORF72 may be produced in both a sense and anti-sense direction (see FIG. 1). It was further hypothesized that these anti-sense transcripts may also undergo RAN translation to produce further repeat proteins from the 5′-GGCCCC-3′ repeats present in the anti-sense transcript. As shown in FIG. 6, both poly-(PA) and poly-(PR) proteins were detectable in brain tissue samples from patients with C9ORF72 ALS but not in controls. These results indicate that di-amino acid-repeat-containing proteins, such as RAN proteins are produced from both a sense and anti-sense transcript produced from the C90RF72 locus.

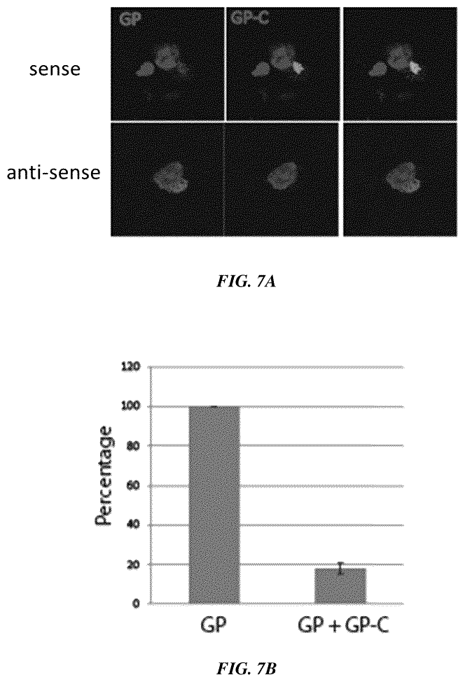

FIG. 7 shows that approximately 20% of aggregates detected with the anti-GP antibody (GP) also co-localize with antibodies directed against the unique C-terminus of the sense GP protein (GP-C). Consistent with the increases levels of antisense transcripts that seen in affected brains, these co-localization data suggest the more ˜80 percent of the GP dipeptide aggregates are expressed from C9ORF72 antisense transcripts.

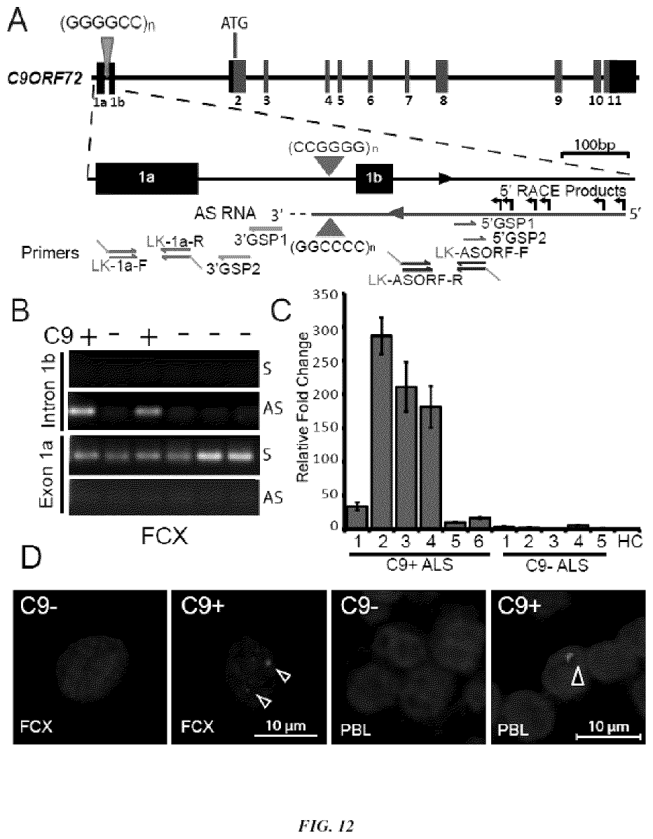

Additionally, the anti-sense transcript was found to be dramatically elevated in subjects with ALS compared to controls (FIG. 12). The primers for the qPCR assay for detecting the anti-sense transcript levels are shown in the table below.

| | | |
|---|---|---|
| ORF F2 | AGTCGCTAGAGGCGAAAGC | primer in |
| | (SEQ ID NO: 36) | c9orf72 |
| | | antisense orf |
| ORF R2 | CGAGTGGGTGAGTGAGGAG | |
| | (SEQ ID NO: 37) | |
| ORF F2 + | CGACTGGAGCACGAGGACACT | |
| IK | GAAGTCGCTAGAGGCGAAAGC | |
| | (SEQ ID NO: 38) | |
| ORF R2 + | CGACTGGAGCACGAGGACACT | for RT 1st |
| Ik | GACGAGTGGGTGAGTGAGGAG | strand |
| | (SEQ ID NO: 39) | |
| Linker | CGACTGGAGCACGAGGACACT | for RT-pcr |
| | GA (SEQ ID NO: 40) | with ORF F1 |
| | | and F2 |

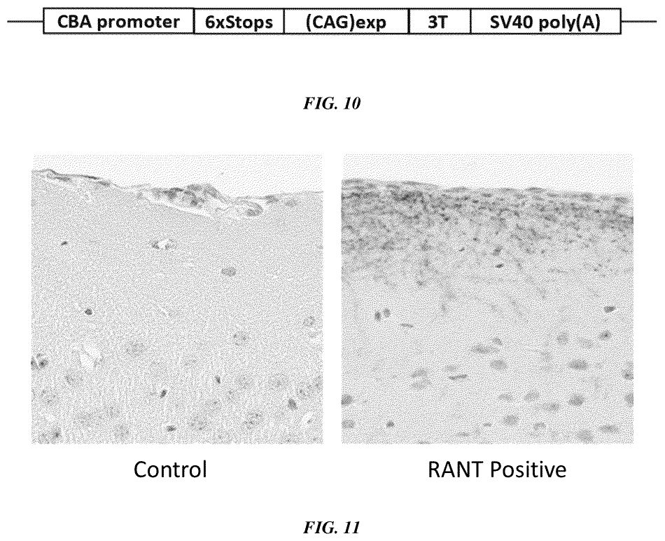

Further, di-amino acid repeat-containing proteins were found to be present in the blood (including in the serum and plasma) and in the brain of subjects with ALS (FIGS. 9 and 10) but not in control subjects.

### Example 2

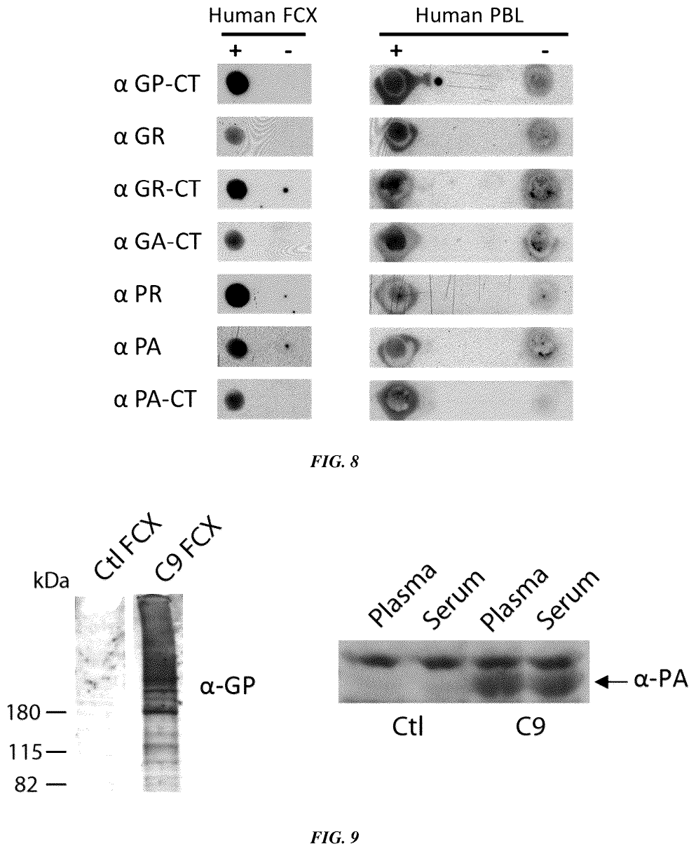

According to some aspects of the disclosure, di-amino acid repeat-containing protein (such as RAN protein) accumulation in blood and cerebral spinal fluid (CSF) substantively contribute to C9ORF72 ALS/FTD and that plasmapheresis and bone marrow transplantation will reverse progression of the disease. According to some aspects of the disclosure, di-amino acid repeat-containing protein accumulation in blood and circulating CSF infiltrates the brain parenchyma and leads to protein accumulation, neuroinflammatory changes, CNS dysfunction and neuronal death. Aspects of the disclosure are based in part on the following. First, blood brain barrier (BBB) impairment is an early feature of disease in ALS patients (4, 5) and higher rates of ALS and other neurological diseases are found in patients who have had traumatic brain injuries (6). In some embodiments, without wishing to be bound by theory, ALS is in part caused by BBB disruptions that allow for the CNS entry of immune cells and other harmful substances that accelerate ALS/FTD. Secondly, as described herein di-amino acid repeat-containing proteins were found to accumulate in ALS patient blood samples (FIGS. 8 and 9).

Although plasmapheresis and bone marrow transplants have been tested as therapeutic strategies for ALS in the past, it is not clear if any of these cases were C9ORF72 positive or if treatment was early enough to have an effect. Accordingly, in some embodiments, ALS treatment (e.g., plasmapheresis or BMT) is initiated when above-normal levels of one or more di-amino acid repeat-containing proteins are detected in the blood of a subject.

The data presented herein on di-amino acid repeat-containing protein accumulation in C9ORF72 ALS patient tissues and blood indicates that reduction of blood (and perhaps also CSF) di-amino acid repeat-containing-protein load may help treat ALS in C9ORF72 ALS patients. According to some aspects of the disclosure, reduction may be achieved, for example, using plasmapheresis or a bone marrow transplant.

### Methods

A detailed evaluation is performed on gene carriers from a C9ORF72 family (CNSA-1) and patients in the clinic including a gene-positive patient with early signs of motor neuron disease or fronto-temporal cognitive dysfunction, or both. Di-amino acid repeat-containing protein expression is correlated with repeat length in CNSA family samples and additional samples collected in clinic. Di-amino acid repeat-containing protein expression in blood is determined in longitudinally collected samples and correlated with disease onset and clinical severity. These methods are expected to characterize di-amino acid repeat-containing protein expression in C9ORF72 positive expansion study subjects and to determine if di-amino acid-repeat-containing protein expression occurs throughout life or increases with age and if di-amino acid repeat-containing protein levels quantitatively correlate with disease severity.

Plasmapheresis is tested to determine if lower di-amino acid repeat-containing-protein load in the blood and CSF reverses signs of the disease. Plasmapheresis is performed on five C9ORF72 positive individuals with early signs of the disease. Six plasmaphereses, each with 2-litter exchange with normal human albumin, is performed over two weeks, followed by one plasmapheresis weekly for the next six months. The study may be prolonged, if required. The primary outcome measure is the Appel ALS Rating Scale (AALSRS). Clinical evaluations including neurological examination, speech evaluation, neuropsychological testing, the ALS Functional Rating Scale (ALSFRS), EMG, and needle muscle biopsy for immunohistopathological evaluations of the vastus lateralis muscle are performed to assess disease progression immediately before and after the treatment period. Venipuncture and lumbar puncture are also performed before and after the 6-month (or if applicable, also after the prolonged) treatment period to assess the concentration of serum and CSF levels of RAN translation and ATG-translation products.

Bone marrow transplant in an animal model is tested to determine if BMT prevents di-amino acid repeat-containing-protein accumulation in blood and the brain. In a first cohort of animals, bone marrow from RANT-positive mice are ablated and replaced with wild-type donor marrow to test if protein aggregate load in the brain decreases. In a parallel set of experiments, RANT-negative animals are transplanted with RANT-positive bone marrow to test if CNS protein accumulation occurs in animals that only express the transgene in hematopoietic cells. Both groups of treated animals are compared to wild-type and untreated RANT control animals using a combination of behavioral, functional and neuropathological assessments.

A RAN translation mouse model has been generated. Transgenic mice were generated using a construct containing 6 stop codons (two in each reading frame) immediately upstream of a CAG expansion mutation and followed by 3 separate epitope tags in each reading frame (FIG. 10). The CAG repeat generates poly-Gln RAN proteins, which have been previously associated with diseases in humans such as fragile X syndrome. The RANT mouse model produced poly-Gln RAN proteins, which were found to localize at high levels under the pia surface in the brain which is exposed to the cerebral spinal fluid (FIG. 11). This RANT mouse model is used in the studies outlined in Example 2. Accordingly, detection of poly-amino acid repeat containing proteins (e.g., mono- or di-amino acid repeat containing proteins) may be indicative of a risk for a brain disorder associated with the poly-amino acid repeat containing proteins. Accordingly, methods described herein may be used to detect or treat other neurological diseases.

### Example 3

### Introduction

The chromosome 9p21-linked form of ALS/FTD, the most common cause of familial FTD and ALS identified to date, is caused by an expanded GGGGCC (G₄C₂) hexanucleotide repeat in intron 1 of chromosome 9 open reading frame 72 (C90RF72) (1, 2). The C9ORF72 mutation is found in 40% of familial and 7% of sporadic ALS cases and 21% of familial and 5% of sporadic FTD patients (3). The discovery of the C9ORF72 expansion has generated substantial excitement because it connects ALS and FTD to a large group of disorders caused by microsatellite expansion mutations (4).

Traditionally, microsatellite expansion mutations located in predicted coding- and noncoding regions were thought to cause disease by protein gain-, or loss-, of-function or RNA gain-of-function mechanisms (4). Protein loss-of-function has been proposed to underlie C90RF72-driven ALS/FTD because the expansion mutation leads to decreased levels of variant 1 transcripts and potential decreases in C9ORF72 protein expression (1, 2). Additionally, because the C9ORF72 G4C2 expansion mutation is located in an intron, several studies have pursued the hypothesis that C9-linked ALS-FTD results from a toxic RNA gain-of-function mechanism in which G4C2 expansion RNAs sequester important cellular factors in nuclear RNA foci. Multiple G4C2 RNA binding proteins have been identified, but so far there is no demonstration that any of these candidates directly bind endogenous expansion transcripts or co-localize with RNA foci observed in patient cells or autopsy tissue (5-8).

In this mechanism, hairpin-forming microsatellite expansion transcripts express proteins in one or more reading frames without an AUG-initiation codon (9). While a variety of names have recently been ascribed to these RAN translated proteins (e.g. homopolymeric, dipeptide, RANT), it is proposed that all proteins expressed across microsatellite expansion mutations in the absence of an ATG-initiation codon be referred to as RAN proteins to prevent confusion as additional expansion mutations that undergo RAN translation are identified.

Here it is shown that C9ORF72 ALS/FTD antisense transcripts containing the GGCCCC (G₂C₄) expansion accumulated in patient brains as nuclear, and infrequent cytoplasmic, foci. Additionally, a novel panel of antibodies directed to both the repeat motifs and unique C-terminal regions was developed and both sense and antisense RAN proteins were demonstrated to accumulate in C9ORF72 patient CNS autopsy tissue. The discovery of antisense G₂C₄ RNA foci and three novel antisense RAN proteins in C9ORF72 patient brains suggests that bidirectional transcription and RAN translation are fundamental pathologic features of C9ORF72 ALS/FTD.

### Results

### Antisense RNA Foci in C90RF72-Expansion Patients

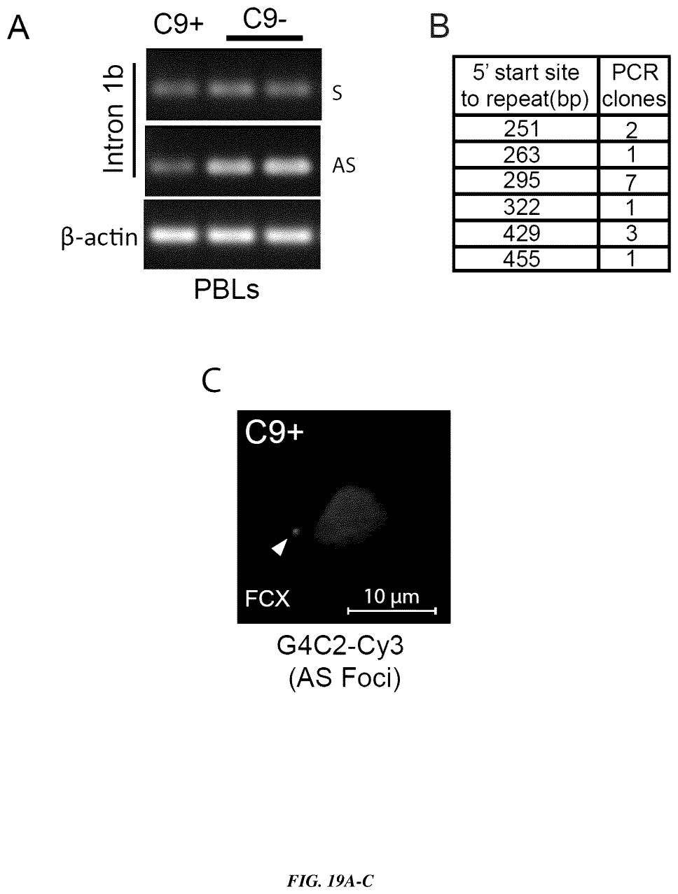

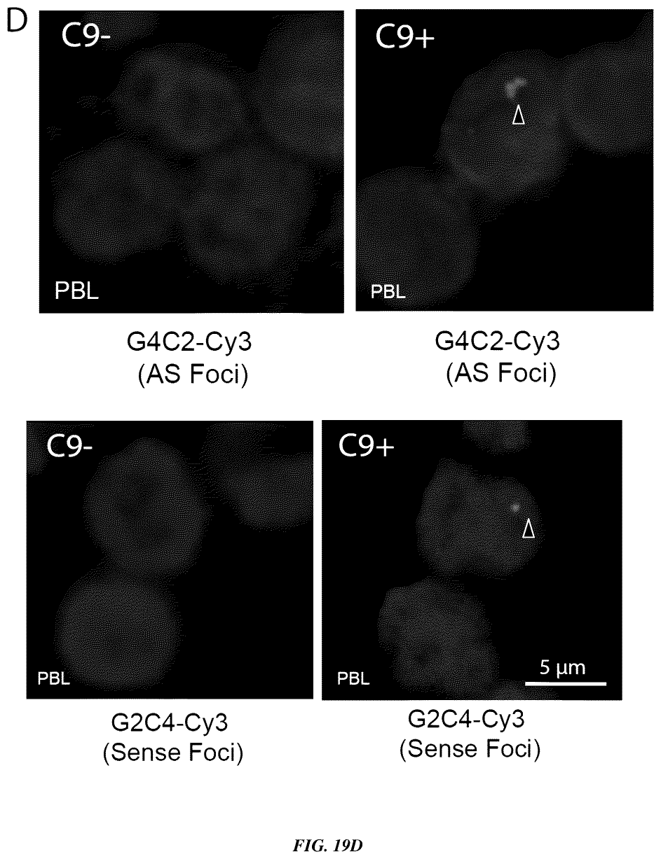

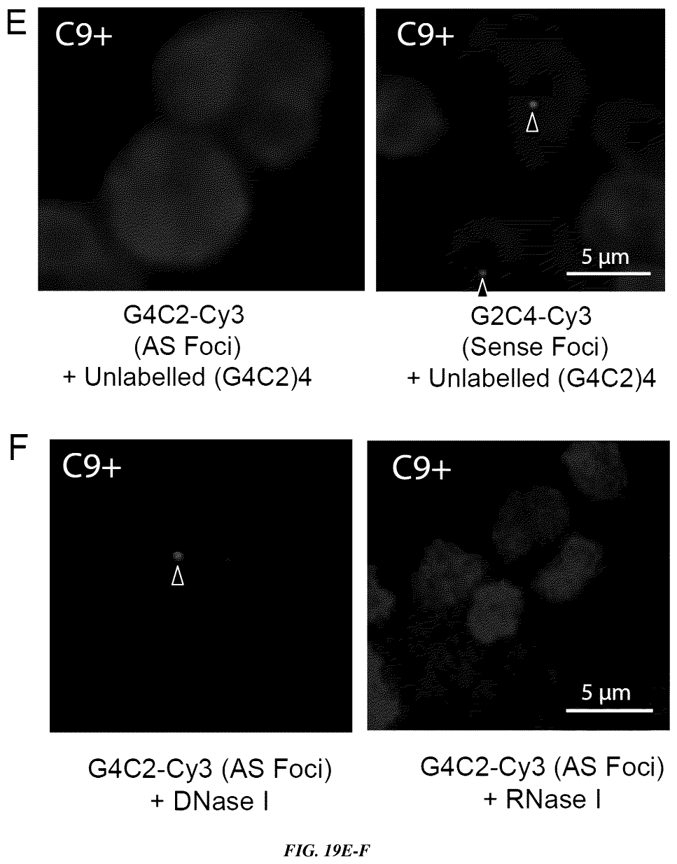

A series of experiments was performed to test the hypotheses that antisense (AS) C90RF72 expansion transcripts form AS G2C4 RNA foci and express AS proteins by RAN translation or from short AS open-reading frames (AS-ORFs). First, it was confirmed that C9ORF72 antisense transcripts are expressed using a linkered strand-specific RT-PCR strategy to compare expression of the sense and antisense transcripts in intron 1b, 5′ of the antisense G₂C₄ expansion, and exon 1a. For the antisense strand in intron 1b, strand-specific RT-PCR was performed using LK-ASORF-R primer for the RT reaction and ASORF-F and the LK for PCR to specifically amplify antisense-cDNAs (FIG. 12A). Similar strategies were used to amplify sense transcripts from the same region of intron 1b and sense and antisense transcripts in exon 1a. Intron 1b antisense transcripts were detected by RT-PCR in frontal cortex from C9 (+) ALS/PTD patients but not C9 (−) ALS/FTD or normal controls (FIG. 12B) and qRT-PCR shows these transcripts are dramatically increased among six C9 (+) ALS/FTD cases (FIG. 12C). In contrast, intron 1b sense transcripts were not detected by RT-PCR (FIG. 12B) in frontal cortex. In blood, both intron 1b sense and antisense transcripts are detectable and the dramatic C9 (+) elevation of the intron 1b antisense transcripts was not observed. 5′ RACE showed intron 1b AS transcripts begin at varying sites 251-455 basepairs (bp) upstream of the G2C4 repeat (FIGS. 12A, 19B). In contrast, 3′RACE, using 3′GSP1 or 3′GSP2 primers located 40 and 90 bp 3′ of the G2C4 repeat, did not detect transcripts. These data showed that the 3′ end of the AS transcript does not overlap the sense exon 1a region, located 170 bp 3′ of the antisense (2C4 repeat. Consistent with this result, sense but not antisense transcripts are detected by strand specific linkered-RT-PCR using primers overlapping exon 1a (FIG. 12B). To determine if antisense transcripts include the G2C4 repeat expansion, RNA fluorescence in situ hybridization (FISH) was performed using a Cy3-labelled (G4C2) 4 probe to detect putative antisense G2C4 RNA foci. The results showed nuclear (FIG. 12D) and rare cytoplasmic (FIG. 19C) G2C4 RNA foci accumulate in C9 (+) but not C9 (−) ALS frontal cortex. The detection of foci in the cytoplasm showed that antisense expansion transcripts can be found in the same cellular compartment as the protein translation machinery, presumably where RAN translation occurs. Because RNA foci in peripheral tissues may provide biomarkers of the disease, peripheral blood leukocytes (PBLs) were examined and both sense and antisense RNA foci were detected in C9 (+) but not C9 (−) PBLs (FIG. 12D, FIG. 19D). It was discovered that the RNA-FISH signal from the Cy3-G4C2 probe detecting AS-foci may be competed with excess unlabeled G4C2 oligo, and these foci were resistant to DNase I and sensitive to RNase I digestion (FIG. 19E, F). Taken together, this shows that C9ORF72 antisense transcripts are elevated in the frontal cortex in C9 (+) ALS but not C9 (−) ALS or normal controls. It was also shown for the first time that antisense transcripts containing the G₂C₄ expansion mutation are expressed and accumulate in nuclear and rare cytoplasmic RNA foci in C9 (+) frontal cortex. Additionally, it was shown that sense and antisense foci accumulate in blood, providing potential biomarkers of C90RF72 ALS/FTD in a readily accessible tissue.

### RAN Translation of GGCCCC Repeat Expansion In Vitro

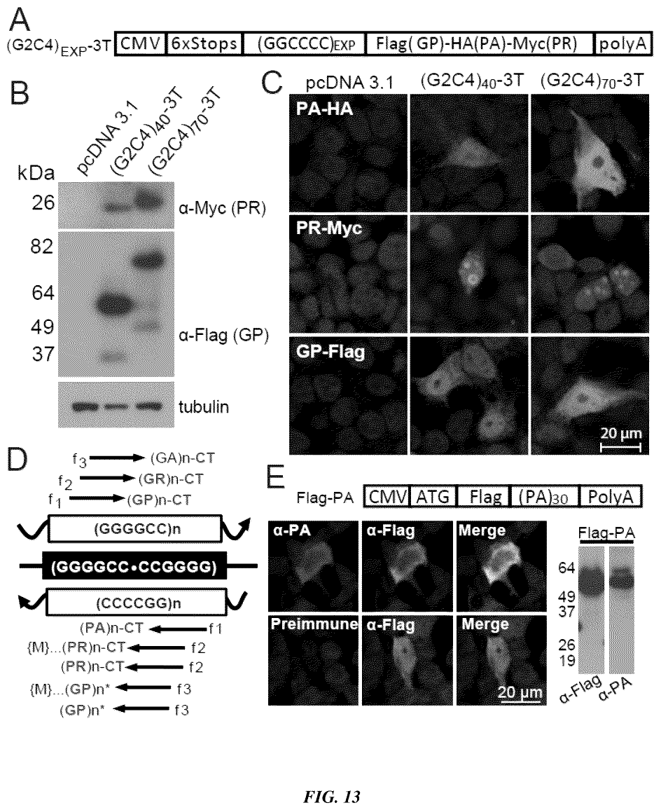

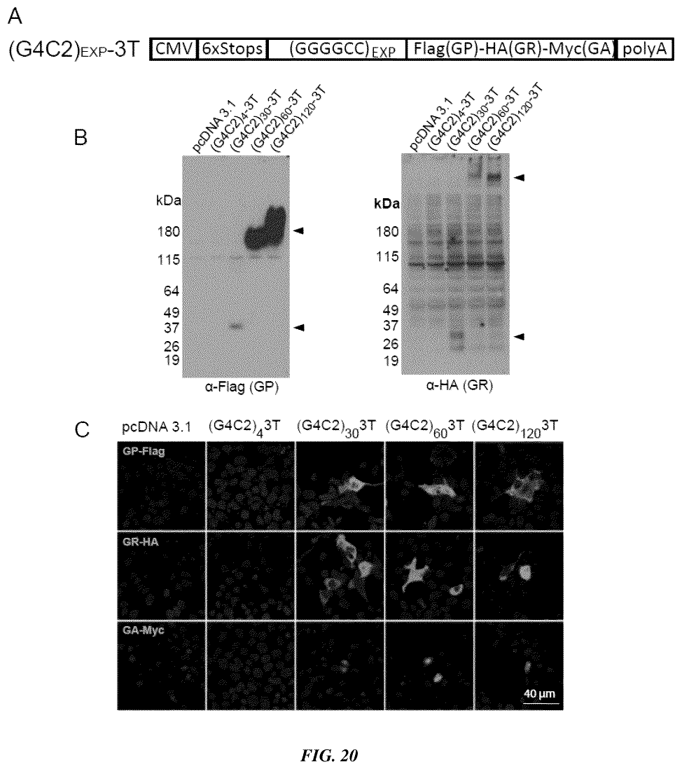

To test if the antisense G₂C₄ expansions undergo RAN translation, a triply tagged G₂C4 minigene was generated, (G₂C₄)_{EXP}-3T, lacking an ATG initiation codon, by inserting a 6X STOP codon cassette (two stops in each frame) upstream of G₂C₄ expansions of 40 or 70 repeats and three different C-terminal epitope 8 tags to monitor protein expression in all reading frames [e.g., (G₂C₄EXP transcripts translated in three frames results in Gly-Pro (GP), Pro-Ala (PA) and Pro-Arg (PR) RAN proteins] (FIG. 13A). Immunoblotting detected two epitope-tagged RAN proteins, PR-Myc and GP-Flag, but not PAHA (FIG. 13B). The (PR) 40- and (PR) 70-3xMyc proteins migrated at approximately their predicted sizes of 20 and 27 kDa, respectively. In contrast, the (GP) 40- and (GP) 70-3xFlag proteins migrated substantially higher than their predicted sizes (10-15 kDa) at 50 and 75 kDa, respectively (FIG. 13B). The faint lower molecular weight bands on this blot may result from repeat contractions seen during bacterial culture or differences in translational start site. Immunofloresence (IF) showed antisense RAN proteins are expressed in all three reading frames (FIG. 13C). The detection of PA-HA by IF but not western blotting may be caused by a lower frequency of cells expressing RAN PA-HA from these constructs. Additionally, recombinant GP-Flag and PA-HA proteins had a cytoplasmic localization whereas PR-Myc proteins were distributed in both the nucleus and cytoplasm. These localization differences may result from different properties of the repeat motifs or the C-terminal flanking sequences found in this epitope tagged construct. In an additional series of experiments also it was shown that sense G4C2-expansion constructs containing 30, 60 and 120 repeats express GP-Flag, GR-HA and GA-Myc RAN proteins (FIG. 20). In summary, these data showed that recombinant G2C4 and G₄C₂ expansion transcripts express RAN proteins in all six reading frames.

### Dual Immunological Strategy to Detect RAN Proteins

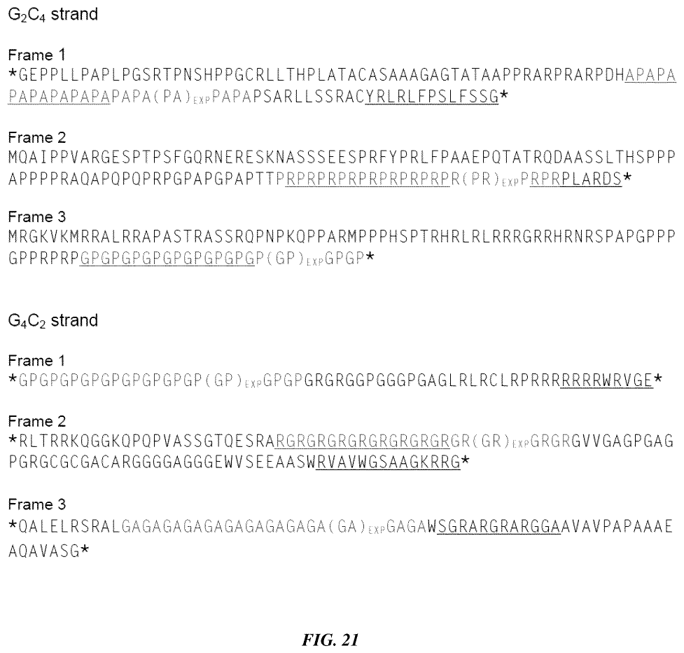

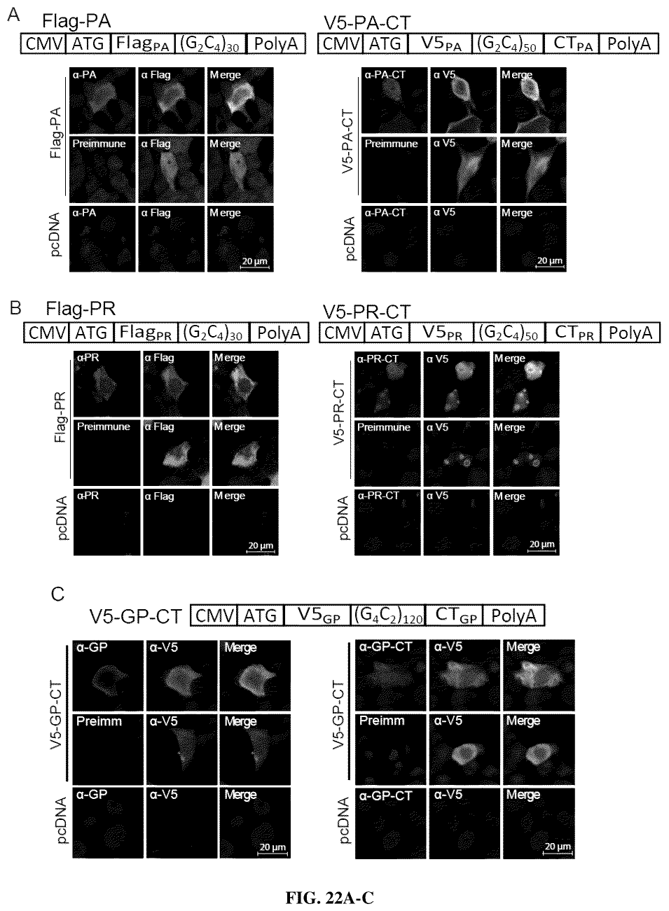

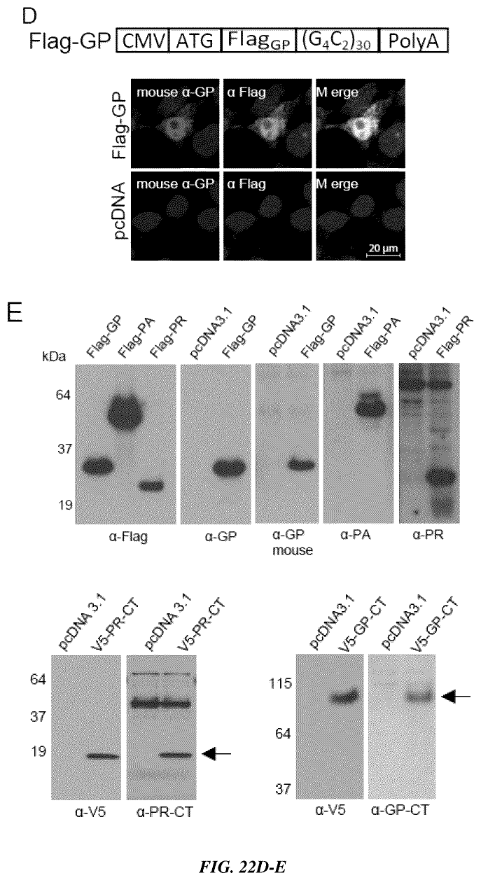

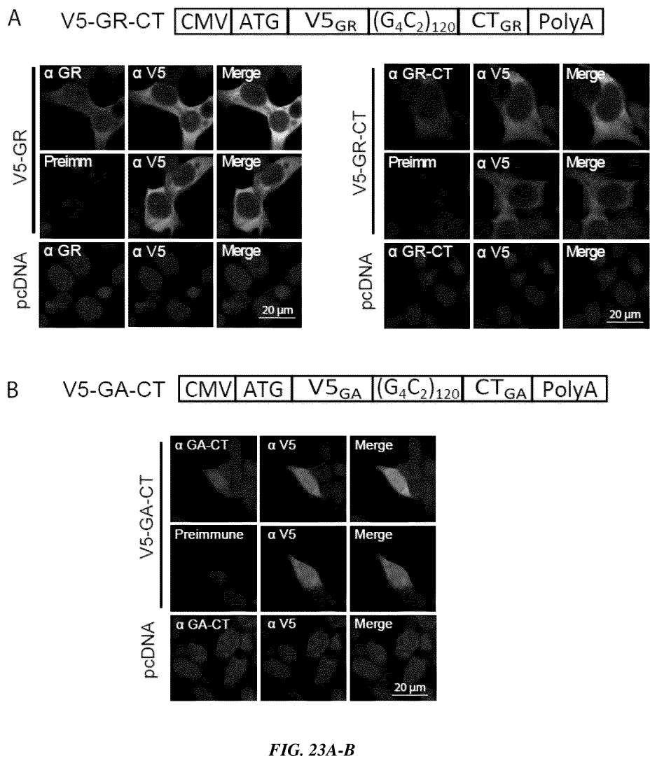

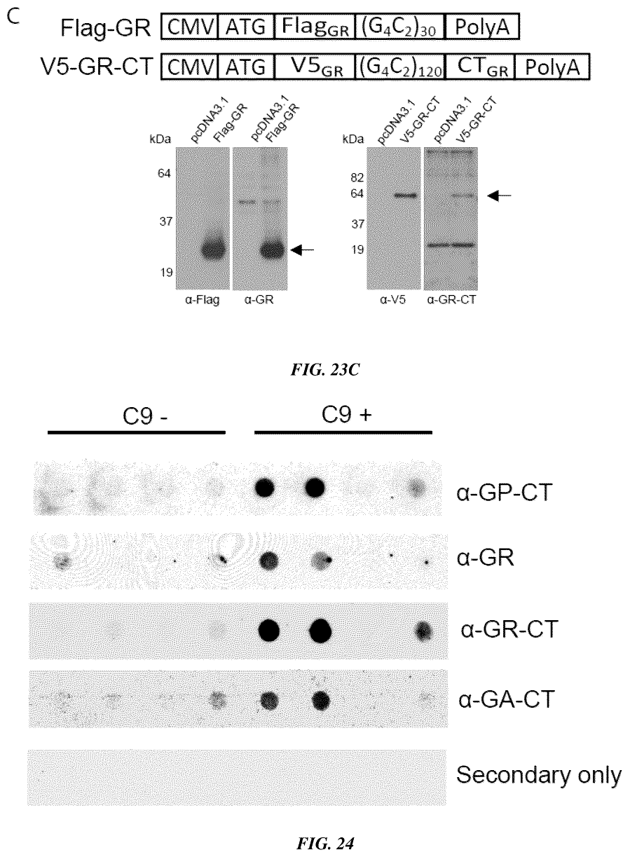

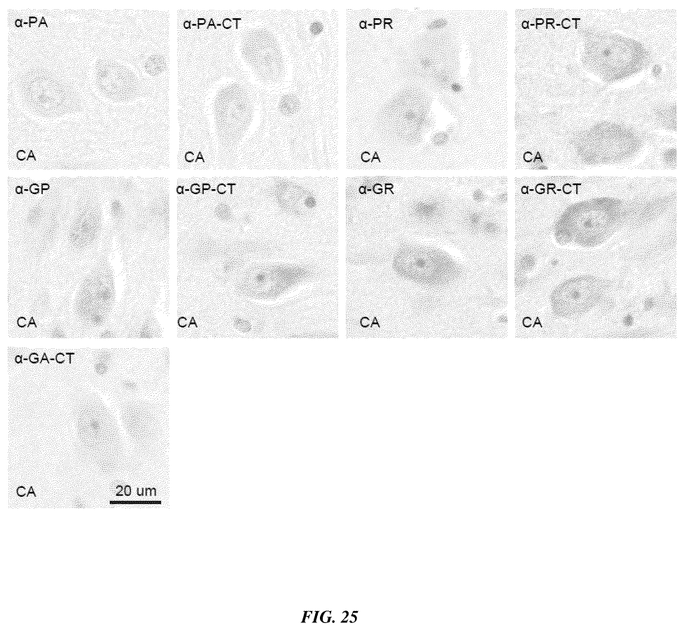

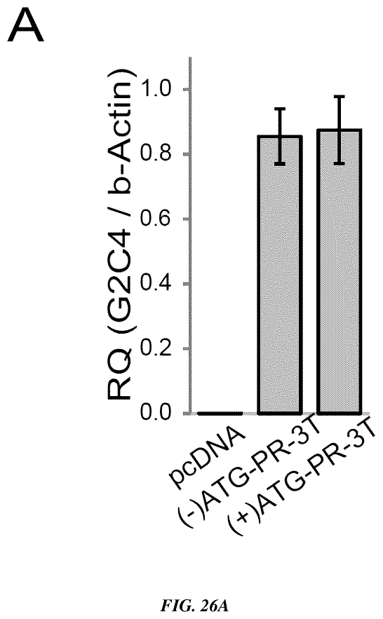

Since amino acid repeats can be found in a range of different proteins, a dual immunological strategy was used and antibodies that recognize the predicted repeat motifs described herein or their corresponding unique C-terminal regions were developed. A schematic diagram showing eight putative C9ORF72 RAN proteins is shown in FIGS. 13D and 21. Predicted proteins include six putative RAN proteins and two putative proteins with additional ATG-initiated N-terminal sequence. Unique C-terminal regions are predicted in five of the six predicted reading frames. To test for the accumulation of these proteins in vivo a series of polyclonal antibodies against the predicted repeat motifs or available corresponding C-terminal regions, were developed (FIGS. 13D, 21). Antibodies to test for putative antisense proteins [rabbit α-PA, α-PA-CT, α-PR, α-PR-CT, α-GP α-GP-CT (sense), and mouse α-GP] were generated and their specificities demonstrated in cells transfected with constructs expressing epitope-tagged recombinant protein by western blot and IF detection (FIGS. 13E, 22). Additional antibodies detecting repeat and C-terminal regions expressed in the sense direction are characterized in FIG. 23.

Antisense G₂C₄ RAN Proteins Accumulate in Brain

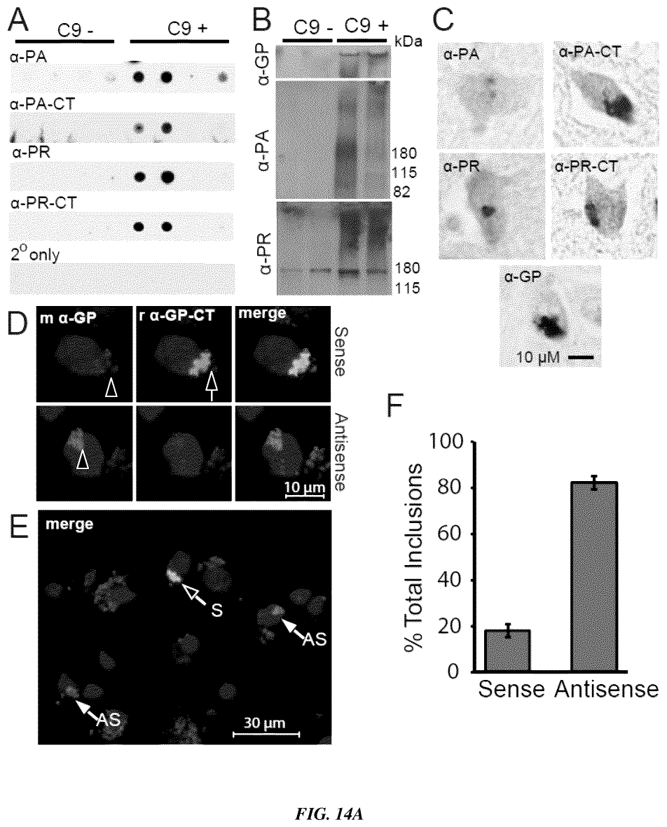

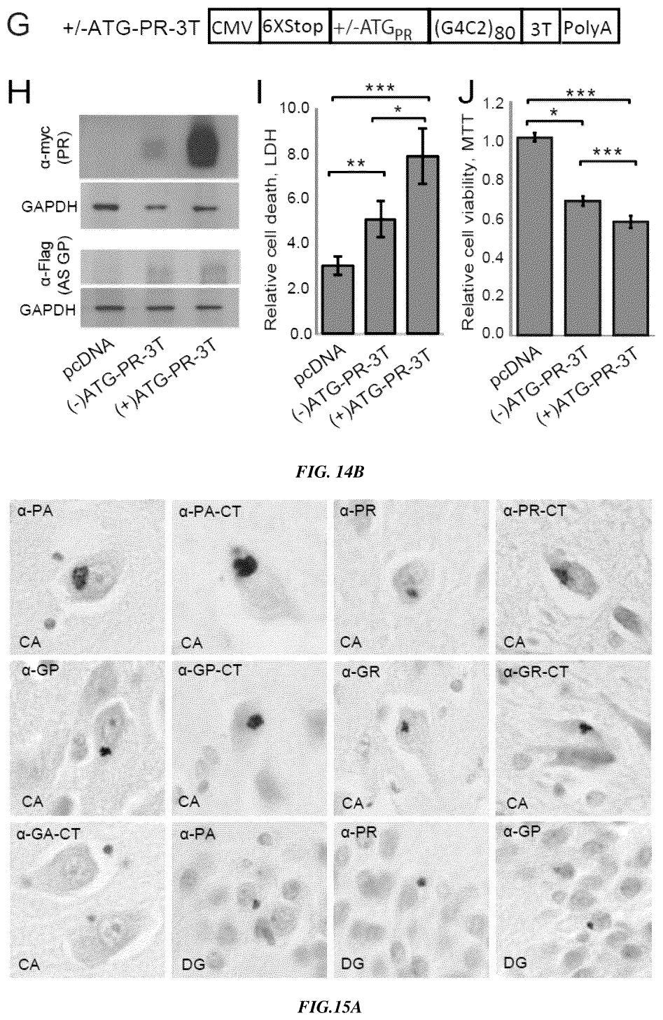

Several approaches were used to determine if novel antisense (AS) proteins are expressed in C9ORF72 expansion positive autopsy tissue. To overcome the obstacle that aggregated proteins are difficult to isolate from human brain, a sequential protein extraction protocol (23) was used on frozen C9 (+) and C9 (−) ALS frontal cortex autopsy samples. Antisense PA and PR proteins were detected with α-PA, α-PA-CT, α-PR, α-PR-CT on immuno-dot blots of 1% Triton-X100 insoluble, 2% SDS soluble extracts from a subset of C9 (+) but not C9 (−) ALS patients (FIG. 14A). Additional immuno-dot blots showing evidence for sense-RAN protein (GP, GR, GA) 10 accumulation in C9 (+) ALS/FTD frontal cortex are shown in FIG. 24. α-PA, α-PR and α-GP antibodies also detected high molecular weight smears in 2% SDS insoluble fractions from C9 (+) ALS frontal cortex samples after resuspending the pellets in sample buffer containing 8% SDS (23) (FIG. 3B). The differences in migration pattern seen for the recombinant proteins (FIG. 13B), which migrate as one or more bands, and the smears observed in patient tissue extracts (FIG. 14B) reflect differences in the RAN proteins due to much longer repeat tracts in patient samples and their extraction from highly insoluble aggregates. Immunohistochemistry (IHC) was next used to show that protein aggregates were detectable in the perikaryon of hippocampal neurons from C9 (+) ALS/FTD autopsy tissue but not in C9 (−) ALS patients or control subjects using antibodies against the repeat motifs (α-PA, α-PR, α-GP) as well as antibodies directed to predicted C-terminal sequences beyond the PA and PR repeat tracts (α-PA-CT and α-PR-CT) (FIG. 14C, 25). Previous studies using antibodies directed against the GP repeat motif, detected aggregates, which were assumed to be expressed from the sense strand (10, 11). It is noted that GP repeat-containing proteins are predicted to be expressed from both sense and antisense transcripts (FIG. 13D) In the sense direction the predicted RAN GP protein contains a unique C-terminal (CT) sequence. In contrast, the antisense GP protein has a stop codon immediately after the repeat. To distinguish sense-GP RAN proteins from antisense-GP proteins, a double label IF experiments was performed on C9 (+) human hippocampal autopsy sections using rabbit α-GP-CT to detect the CT region of the sense-GP protein and mouse α-GP to detect both sense and antisense GP expansion proteins. Double labeling showed two types of inclusions: a) putative sense inclusions double labeled with mouse α-GP and rabbit α-GP-CT sense and; b) putative antisense inclusions singly labeled with mouse-α-GP (FIG. 14D). Approximately 18% of inclusions showed the sense pattern with double labeling and 82% 11 of inclusions showed the antisense pattern and were positive for α-GP and negative for α-GP-CTsense (FIG. 14E,F). These data showed the importance of characterizing protein aggregates with both repeat and C-terminal antibodies. Taken together, these results show that insoluble, aggregate-forming antisense-RAN proteins are expressed from all three antisense reading frames.

### G2C4 Expansions and RAN Proteins are Toxic to Cells

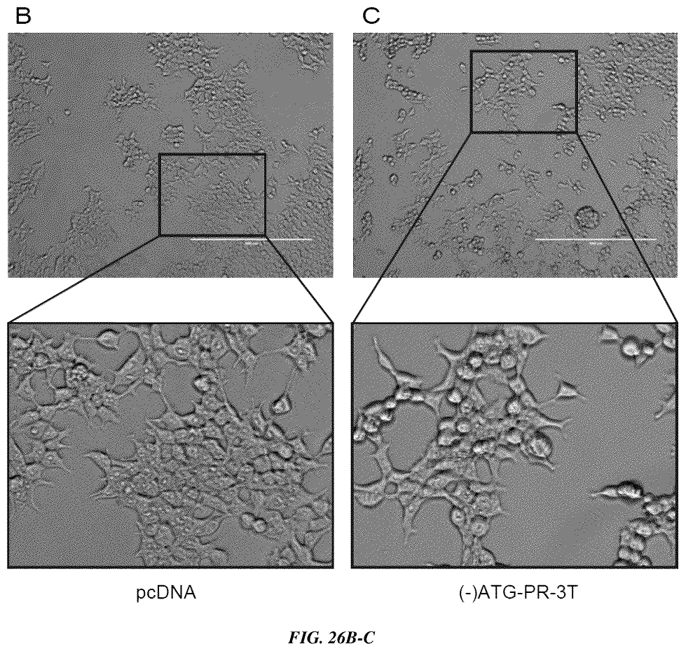

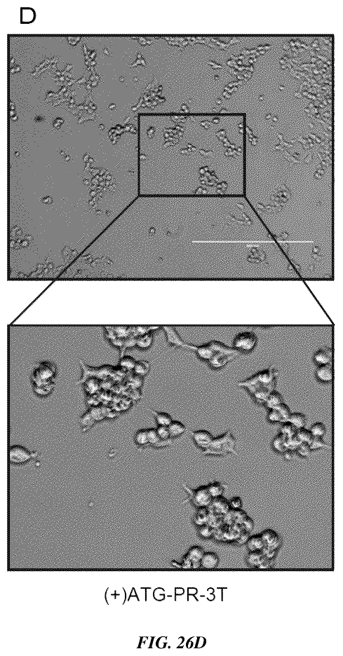

In addition to antisense GP and PR RAN proteins expressed by RAN translation, two of the antisense reading frames have upstream ATG initiation codons that may result in both ATG-initiated GP and PR proteins (M-GPAS and M-PRAS) (FIGS. 13D and 21). It was shown that the presence of an ATG-initiation codon does not prevent RAN translation from also occurring in all three reading frames (9). Therefore antisense GP and PR proteins may be expressed by both AUG-initiated and/or RAN translation. To explore the effects that an ATG-initiation codon has on RAN protein expression for the G₂C₄ expansion, an additional minigene construct was generated by placing an ATG initiation codon in front of the G₂C₄ repeat (FIG. 14G). The PR frame was selected for analysis because an ATG initiation codon naturally occurs in this reading frame. Western blotting shows that HEK293T cells transfected with (+) ATG-PR-3T express substantially higher levels of PR protein compared to (−) ATG-PR-3T transfected cells (FIG. 14H). In contrast, qRT-PCR and Western blotting showed transcript levels (FIG. 26A) and levels of RAN-translated GP (FIG. 14H) were comparable. Similar to FIG. 13, RAN-translated PA was not detectable by Western blot. The effects of these constructs on cell viability was then tested using complementary assays; lactate dehydrogenase (LDH) detection and methylthiazol tetrazolium (MTT). For the LDH assay, cells transfected with the (−) ATG-PR-3T or (+) ATG-PR-3T construct showed 1.9 and 2.9 fold increases in cell death compared to vector control cells (p=0.008 and 0.001), respectively. Additionally, (+) ATG-PR-3T transfected cells, which express elevated levels of PR protein showed a 1.5 fold increase in cell 12 death compared to cells transfected with the (−) ATG-PR-3T construct (p=0.034). The MTT assay showed similar results. Cells transfected with (−) ATG-PR-3T and +ATG-PR-3T constructs showed dramatic decreases in the number of metabolically active cells, 33% (p<0.00001) and 43% (p<0.00001), respectively compared to untreated cells or empty vector controls (FIG. 14.1). Additionally, elevated PR expression in cells transfected with (+) ATG-PR-3T had significantly lower levels of metabolic activity compared to (−) ATG-PR-3T cells (p<0.05). By light microscopy cell detachment and changes in cell morphology were evident in-ATG-PR-3T compared to control cells and these phenotypes worsened in (+) ATG-PR-3T cells which express elevated levels of PR (FIG. 26B-D). Taken together, these data demonstrated that: 1) the G₂C₄ expansion mutation is toxic to cells—this toxicity may be caused by effects of the DNA. G₂C₄ RNA and/or RAN-translated PR, GP or PA proteins; 2) increased PR protein expressed in cells transfected with the (+) ATG-PR-3T construct increases cell toxicity and death above levels caused by the DNA, G₂C₄ RNA and RAN protein effects. Therefore the PR protein was shown to be intrinsically toxic to cells.

### All Six RAN Proteins Form Aggregates in the Brain

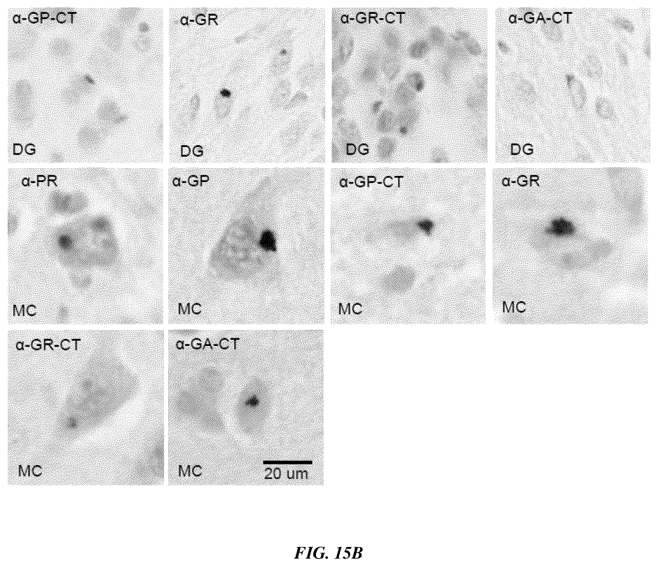

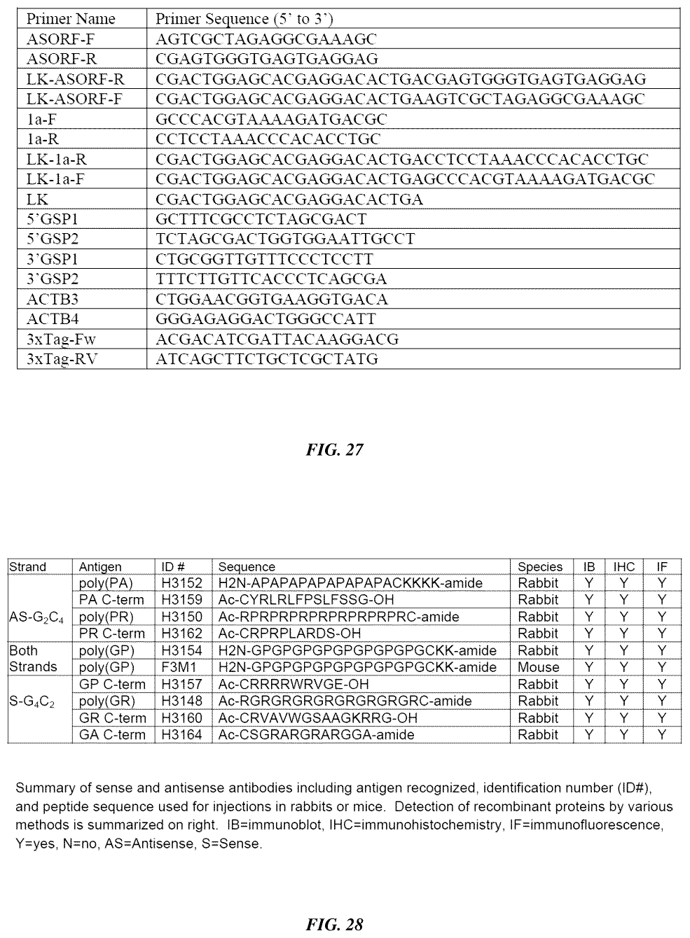

To determine if all six RAN proteins from both sense and antisense RNA strands are expressed in C9 (+) ALS patients, IHC staining was performed on sections of paraffin-embedded brain tissues using nine polyclonal antibodies against repeat-expansion and/or C-terminal sequences of these proteins. In C9 (+) cases there were abundant globular and irregular-shaped neuronal cytoplasmic inclusions (NCIs) in the hippocampus, the majority of which were in the dentate gyrus and in pyramidal cells in the CA regions. These RAN inclusions were also detected in C9 (+) motor cortex (FIG. 15). GP positive inclusions were detected in all examined C9 (+) cases but not in C9 (−) cases or normal control sections in the hippocampus as well as in the motor cortex using α-GP. In the CA regions of the 13 hippocampus and in the motor cortex, clusters of aggregates were frequently found in C9 (+) cases with aggregates in >20% of neurons (FIG. 27). Fewer aggregates were detected with the α-GP-CT sense antibody, consistent with double labeling experiments (FIG. 14D-F) that showed most GP aggregates are translated from C90RF72 antisense strand. PA inclusions were detected in hippocampus in four out of six C9 (+) cases tested and in one out of two motor cortex samples (FIG. 27). In C9 (+) cases, the frequency of PA inclusions were significantly lower in the hippocampus and motor cortex compared with GP inclusions, but high-intensity regional staining with extremely large PA inclusions found in >50% of neurons were found in one patient (FIG. 27). PR positive inclusions were also seen in hippocampus in all C9 (+) cases examined and in motor cortex in one out of two C9 (+) cases tested. Similar to the PA staining, PR inclusions are less frequent but intense regional staining was occasionally observed. In the sense direction, GR positive inclusions were found in the hippocampus and motor cortex in all C9 (+) cases examined, but appeared less frequent than the GP aggregates. GA inclusions were only occasionally detected by IHC as small perinuclear inclusions in hippocampus and in motor cortex (FIG. 15, 27). The apparent differences in the frequency of various types of aggregates may result from differences in protein conformation and epitope availability or differences in the affinities of these antibodies, which were designed to different epitopes. Taken together, this data showed that all six RAN proteins form aggregates in the C9 (+) autopsy brains.

### Inclusions of RAN Proteins in Upper and Lower Motor Neurons

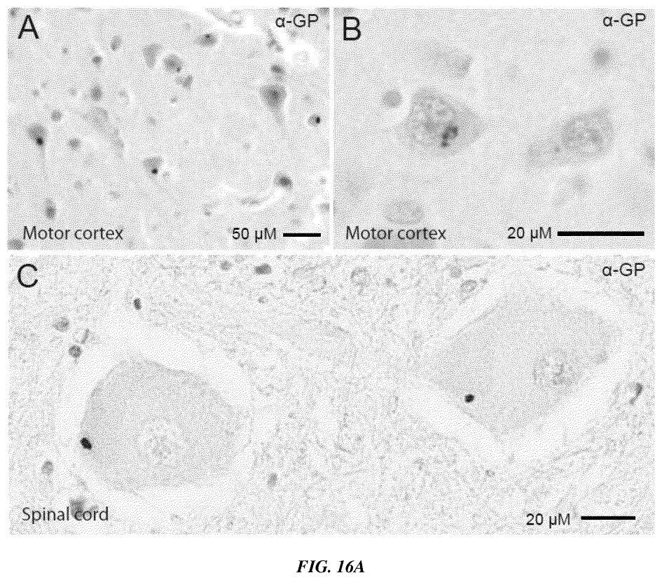

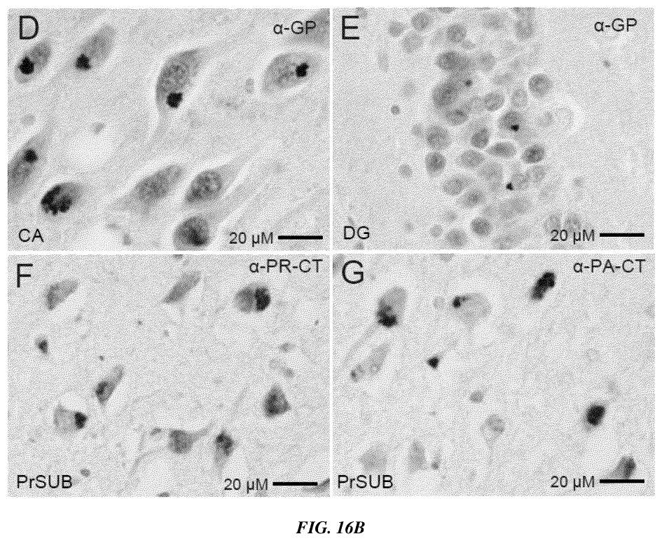

A central feature of ALS is the gradual degeneration and death of upper motor neurons in motor cortex and lower motor neurons in the brain stem and spinal cord. To test if RAN proteins accumulate in upper and lower motor neurons, IHC was performed using all nine antibodies against predicted proteins in both sense and antisense directions. In C9 (+) cases, abundant GP-positive neuronal cytoplasmic inclusions were seen in all layers of motor cortex, with frequent GP aggregates in pyramidal neurons of layer III and throughout layer V (FIG. 16A). Although cell death and atrophy made motor-neurons in layer V difficult to identify, GP inclusions in remaining upper motor neurons were found (FIG. 16B). Additionally, PA-, PR-, GR- and GA-positive inclusions were also found in the motor cortex (FIG. 15, 27). Using a similar series of experiments performed in spinal cord sections, GP aggregates in all three cases examined and aggregates in lower motor neurons in two out of three C9 (+) patients were detected, but not in C9 (−) ALS cases or normal controls (FIG. 16C). This is the first report of RAN protein accumulation in motor neurons. The discovery of GP-aggregates in both upper and lower motor neurons links C9 RAN-protein accumulation to the neurons selectively vulnerable in ALS.

### High Density Clustering of RAN-Protein Aggregates

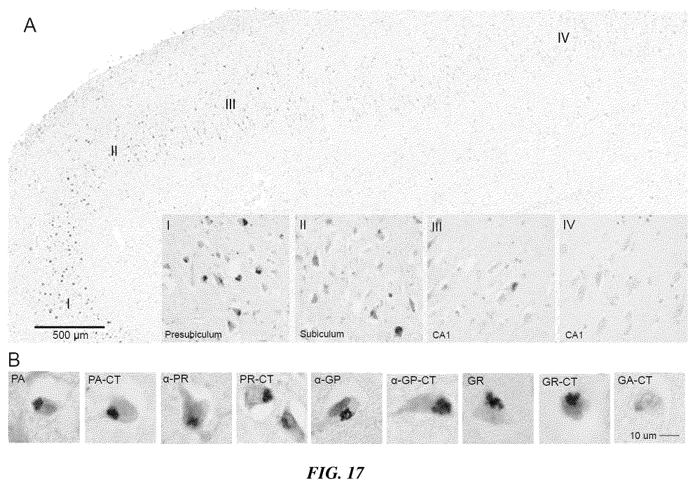

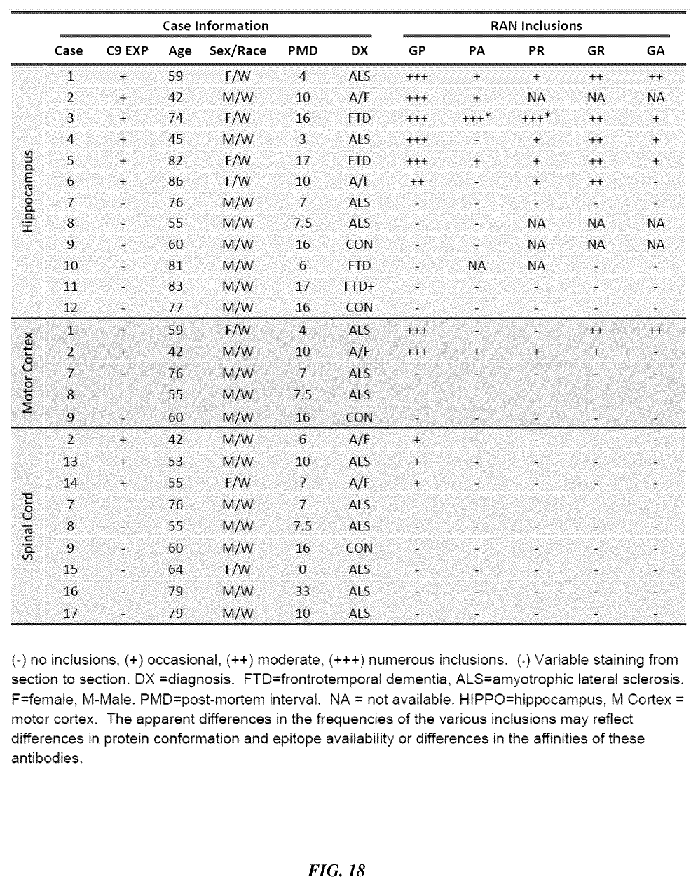

Both sense and antisense proteins accumulated in neurons of C9ORF72 autopsy brains. In general, two types of aggregation patterns were observed: 1) isolated cytoplasmic aggregates and 2) high-density clustered cytoplasmic aggregates in which ˜10 to more than. 50% of neurons were positive. Clustered aggregates were most frequently detected for GP and were found in the dentate gyrus (DG) and CA1-4 of the hippocampus (FIG. 16D, E). The clustered GP aggregates in DG were smaller and less frequent than the large cytoplasmic aggregates in CA regions. Additional clustered GP aggregates were frequently found in subiculum and presubiculum of the hippocampus as well as 15 the motor cortex. Immunostaining of serial sections showed that multiple proteins are often found in the same region. For example, intense clustered staining for PA, PR, GP, GA and GR proteins was found in the same region of the presubiculum in serial sections from one C9 (+) patient (see FIG. 16F,G). Immunostaining for PA showed that some brain regions have abundant aggregates whereas other regions in the same section are relatively spared. For example, FIG. 17A illustrates a gradient of PA inclusions (presubiculm>subiculum>CA1) across hippocampal regions in a single section in one patient. PA inclusions in this patient were numerous (>50% of neurons) in presubiculum (I), moderate in subiculum (II), and rare in CA1 hippocampal regions (Ill and IV). Consistent with the focal regional staining seen in this section, PA staining was not detected in sections from a separate block of hippocampal tissue taken from the same patient. These data shows that expression of the PA RAN protein is variable from cell to cell or that aggregation of PA in one cell triggers aggregation in neighboring cells as has been proposed in a mouse model of Parkinson's disease (24). Next, serial sections from this C9 (+) case were used to show that antibodies directed against both the repeat motifs (α-PA, α-PR, α-GP, α-GR) and corresponding C-terminal regions (α-PA-CT, α-PR-CT, α-GP-CT, α-GR-CT α-GA-CT) detect aggregates in the same densely staining region of the presubiculum (region I) (FIG. 17B). These results showed that both sense and antisense RAN protein aggregates accumulate in this region. The detection of similar aggregates in using antibodies that recognize either the repeat motifs or specific C-terminal regions confirms that these antibodies are recognizing proteins expressed across both the G2C4 and G₄C₂ expansion transcripts and provides new tools to understand the biological impact of RAN translation in C9ORF72 ALS/FTD.

### Discussion

There has been much excitement about the discovery that an intronic microsatellite expansion mutation in C9ORF72 causes a common form of both familial and sporadic ALS/FTD (1, 2). The three major pathological mechanisms being considered for this disease include haploinsufficiency (1, 2), RNA gain-of-function (5-8), and RAN translation (9, 11-13). To date, efforts to understand the molecular mechanisms of this disease have focused exclusively on understanding the consequences of the C9ORF72 expansion mutation in the sense direction. The results reported here show that C9ORF72 expansion mutation is also expressed in the antisense direction and show that antisense RNA foci and antisense RAN proteins contribute to C9ORF72 ALS/FTD. We show for the first time: 1) antisense C90RF72 but not sense transcripts are elevated in C9 (+) autopsy tissue; 2) antisense G2C4 expansion transcripts form RNA foci that accumulate in C9+brain and blood; 3) RAN translation occurs across antisense G₂C₄ expansion constructs in cell culture; 4) that sense and antisense RAN proteins accumulate in C9 (+) autopsy brains using a dual immunological approach with both repeat and C-terminal antibodies; 5) RAN protein aggregates accumulate in upper and lower motor neurons linking RAN translation directly to the key pathologic feature of ALS. Since the initial report that G₄C₂ RNA foci accumulate in C90RF72 ALS/FTD patient tissues (1, 2), a leading hypothesis is that G₄C₂ sense transcripts sequester and dysregulate RNA binding proteins similar to the sequestration of MBNL proteins in DM1, DM2 and SCA8 (4). Several groups have already reported G₄C₂ binding proteins and are testing their potential role in disease (5-8). The discovery that antisense G₂C₄ foci also accumulate in patient cells shows that G2C4 antisense RNAs and binding proteins may play a role. Additionally, the discovery of sense and antisense foci in C9 (+) peripheral blood may prove useful as an easily accessible biomarker of C90RF72 ALS/FTD. Biomarkers that monitor both sense and antisense transcripts may be particularly important as therapies that decrease expression of one strand may increase expression of the other strand. Using a dual immunological approach it was shown that G₂C₄ antisense transcripts express novel antisense proteins (PA, PR, GP) by RAN translation and/or from two short ORFs (Met-AS-PR and Met-AS-GP).

### Materials and Methods

cDNA constructs. CCCGGGGCC(GGGGCC)₂GGGGCCC (SEQ ID NO: 64) and CCCGGGGCC(GGGGCC)₂₈GGGGCCC (SEQ ID NO: 65) fragments that contain upstream 6xStop codons were synthesized and cloned into pIDTSmart vector by Integrated DNA Technologies. 6xStops-(GGGGCC)₄-3T and 6xStops-(GGGGCC)₃₀-3T constructs were generated by subcloning NheI/XhoI fragment into pcDNA3.1 vector containing triple epitopes. To expand the size of the GGGGCC repeats, SmaI/XhoI fragment was subcloned into PspOMI blunted with T4 DNA polymerase/XhoI of pcDNA-6xStops-(GGGGCC) EXP-3T. To reverse the orientation of GGGGCC repeats in pcDNA-6xStop-3T construct, SmaI/I/ClaI fragment was subcloned into pBluescript SK+ to generate pBluescript-(GGGGCC) Exp. The AfeI/XhoI fragment pBluescript-(GGGGCC) EXP was subcloned into pcDNA-6xStop-3T to make pcDNA-6xStop-(GGCCCC)_{EXP}-3T construct.

RT-PCR. 1) Strand-specific RT-PCR in autopsy tissues: Total RNA was isolated from Frontal cortex autopsy tissues and peripheral blood lymphocytes (PBL) of ALS patients and healthy controls with TRIzol (Invitrogen). To detect transcripts from both strands, cDNA was generated from 0.25 μg of total RNA using the SuperScript III system (Invitrogen) with linkered strandspecific reverse primers and PCR with strand specific forward and linker (LK) primers. The PCR reactions were done as follows: 94° C. for 3 min, then 35 cycles of 94° C. for 45 s, 58° C. for 45 s and 72° C. for 1 min followed by 6 min at 72° C. Bands were cloned and sequence to verify their specificity of the PCR amplification. 2) RT-PCR for toxicity assay in 293T cells: Total RNA from cells was extracted using miRNeasy Mini kit (Qiagen) according to the manufacturer's protocol. Total RNA was reverse transcribed using the Superscript III RT kit (Invitrogen) and random-hexamer primers. The expression of the different G4C2-3XTag constructs were analyzed by RT-PCR and qPCR using primer set: 3xTag-Fw and 3xTag-Rv. β-Actin expression was used as a reference gene amplified with primer set ACTB3 and ACTB4. Primer sequences are listed in FIG. 27.

Real time RT-PCR. Two step quantitative PCR was performed on a MyCycler Thermal Cycler system (Bio-Rad) using SYBER Green PCR Master Mix (Bio-Rad) and ASORF strand-specific cDNA and primer sets. Control reactions were performed with human beta-actin primers ACTB3 and ACTB4 using oligo d′T synthesized total cDNA as template. Two stage PCR was performed for 40 cycles (95° C. 30 s. 60° C. 30 s) in an optical 96 well plate with each sample cDNA/primer pair done in triplicate. The relative fold changes were generated by first normalizing each experimental Ct value to their beta actin Ct value and then normalized to the healthy control antisense ΔΔCt. Primer sequences are listed in FIG. 28.

Rapid Ampliciation of 5′ and 3′ cDNA ends (5′ and 3′ RACE). Four μg of total RNA from 2 C9 (+) ALS patients and 2 C9 (−) ALS patients frontal cortex autopsy tissues were used for 5′ and 3′ RACE (5′ RACE systems and 3′ RACE; Life Technologies). In 5′RACE, Primer ASORF R was used for gene specific first strand cDNA synthesis and nested reverse primers are 5′GSP1 and 5′GSP2. In 3′RACE, nested forward primers are 3′GSP1 and 3′GSP2. The 3′ RACE and 5′ RACE products were gel-extracted, cloned with TOPO TA Cloning (Invitrogen) and sequenced. Primer sequences are listed in FIG. 28.

Production of polyclonal antibodies. The polyclonal rabbit antibodies were generated by New England Peptide and the polyclonal mouse antibody was generated by the Interdisciplinary Center for Biotechnology Research (ICBR) at the University of Florida. In sense strand (GGGGCC)ₙ antisera were raised against synthetic poly (GP), poly (GR) peptides and C terminal regions of predicted GP, GR, and GA RAN proteins (FIG. 21). In antisense strand (GGCCCC), antisera were raised against synthetic poly (PA), poly (PR) peptides and the C terminal regions of predicted PA and PR RAN proteins. Peptides used to generate antibodies to both antisense and sense proteins and their use for Western blot, immunofluorescence (IF) and immunohistochemistry (IHC) is summarized in Table S3.

Cell culture and transfection. HEK293T cells were cultured in DMEM medium supplemented with 10% fetal bovine serum and incubated at 37° C., in a humid atmosphere containing 5% CO2. DNA transfections were performed using Lipofectamine 2000 Reagent (Invitrogen) according to the manufacturer's instructions.

Human Samples. Frozen frontal cortex tissue samples for biochemical and histological analysis included samples from six C9 (+) ALS, five C9 (−) ALS controls and one normal control were used in this research. Additionally, paraffin embedded fixed tissues from C9 (+) ALS/FTD and C9 (−) ALS/FTD cases as well as a normal control. Peripheral blood lymphocytes (PBL) were isolated from the buffy coat of freshly collected whole blood following brief centrifugation at 2000×g. Red blood cells (RBC) were preferentially lysed and removed using RBC Lysis Buffer (Roche), PBLs centrifuged, washed once with PBS and dried on slides. This study was conducted in compliance with the Declaration of Helsinki. Institutional review boards of the University of Florida and Johns Hopkins University approved the study. Written, informed consent was obtained from participants or relevant parties at the time of enrollment.

Immunofluorescence. The subcellular distribution of polymeric proteins was assessed in transfected HEK293T cells by immunofluorescence. Cells were plated on 8 well tissue-culture chambers and transfected with plasmids the next day. Forty-eight hours post-transfection, cells were fixed in 4% paraformaldehyde (PFA) in PBS for 30 min and permeabilized in 0.5% triton X-100 in PBS for 15 min on ice. The cells were blocked in 1% normal goat serum in PBS for 30 min. After blocking, the cells were incubated for 1 hour at RT in blocking solution containing the rabbit anti-Myc (Abcam), mouse anti-HA (Covance), mouse anti-Flag (Sigma), rabbit anti-GR and rabbit anti-GR-CT primary antibodies at a dilution of 1:400. The slides were washed three times in PBS and incubated for 1 hour at RT in blocking solution containing Goat anti-rabbit conjugated to Cy3 (Jackson ImmunoResearch, PA) and goat anti-mouse conjugated to Alexa Fluor 488 (Invitrogen) secondary antibodies at a dilution of 1:200. The slides were washed three times in PBS and mounted with mounting medium containing DAPI (Invitrogen).

RNA-FISH. Slides with cells were fixed in 4% PFA in PBS for 10 min and incubated in prechilled 70% ethanol for 30 min on ice. Following rehydration in 40% formamide in 2×SSC for 10 min, the slides were blocked with hybridization solution (40% formamide, 2×SSC, 10 mg/ml BSA, 100 mg/ml dextran sulfate and 10 mg/ml yeast (RNA) for 10 minutes at 55° C., and then incubated with 200 ng/ml denatured RNA probe in hybridization solution at 55° C. for 2 hours. After hybridization the slides were washed 3 times with 40% formaminde in 2×SSC and briefly washed one time in PBS. Autofluorescence of lipofuscin was quenched by 0.25% of Sudan Black B in 70% ethanol and the slides were mounted with mounting medium containing DAPI (Invitrogen). The specificity of the RNA foci was determined by treating cells prior to FISH detection with either RNAse (100 μg/mL in 2×SSC), DNase (1 U/ul in DNaseI buffer) or Protease K (120 ug/mL in 2 mM CaCl2, 20 mM Tris, pH 7.5). Treated cells were incubated at 37° C. for 30 minutes, washed 3 times with PBS then 3 times with 2×SSC. Subsequent FISH detected was performed as described above. Antisense foci specificity was determined using standard FISH detection to first hybridize slides with 10-fold excess unlabeled (G4C2) 4 oligo followed by hybridization with either G4C2-cy3 (antisense probe) or G₂C₄-cy3 (sense probe). Subsequent treatment and detection were performed as described above.

Western blotting. Transfected cells in each well of a six-well tissue-culture plate were rinsed with PBS and lysed in 300 μL RIPA buffer with protease inhibitor cocktail for 45 min on ice. DNA was sheared by passage through a 21-gauge needle. The cell lysates were centrifuged at 16,000×g for 15 min at 4° C., and the supernatant was collected. The protein concentration of the cell lysate was determined using the protein assay dye reagent (Bio-Rad). Twenty micrograms of protein were separated in a 4-12% NuPAGE Bis-Tris gel (Invitrogen) and transferred to a nitrocellulose membrane (Amersham). The membrane was blocked in 5% dry milk in PBS containing 0.05% Tween-20 (PBS-T) and probed with the anti-Flag (1:2000), anti-Myc (1:1000), anti-HA (1:1000), or rabbit polyclonal antibodies (1:1000) in blocking solution. After the membrane was incubated with anti-rabbit or anti-mouse HRP-conjugated secondary antibody (Amersham), bands were visualized by the ECL plus Western Blotting Detection System (Amersham). Sequential extraction of patient frontal cortex autopsy tissue was performed as follows: tissue was homogenized in PBS containing 1% Triton-X100, 15 mM MgCl₂, 0.2 mg/ml DNase I and protease inhibitor cocktail and centrifuged at 16,000×g for 15 min at 4° C. The supernatant was collected. The pellet was resuspended in 2% SDS and incubated at room temperature for 1 hour, then centrifuged at 16,000×g for 15 min at 4° C. The supernatant was collected and the 2% SDS insoluble pellet was resuspended in 8% SDS. 62.5 mM Tris-HCl pH 6.8, 10% glycerol and 20% 2-Mercaptoethanol for protein blotting (25).

Protein slot blot. 1% Triton-X100 soluble fraction and 2% SDS soluble fraction from the sequential extraction was immobilized onto nitrocellulose membranes with Bio-Dot 96-well microfiltration system (Bio-Rad) under vacuum. The membranes were washed in PBS-T and blotted with each rabbit polyclonal antibody (1:2000) using the same protocol as western blotting.

Immunohistochemistry. Ten-micrometer sections were deparaffinized in xylene and rehydrated through graded alcohol, incubated with 95-100% formic acid for 5 min, and washed with distilled water for 10 min. HIER was performed by steaming sections in citrate buffer, pH 6.0, at 90° C. for 30 min. To block nonspecific immunoglobulin binding, a serum-free block (Biocare Medical) was applied for 30 min. Rabbit polyclonal antibodies were applied at a dilution of from 1:5000 to 1:15,000 in serum-free block (Biocare Medical) and incubated overnight at 4° C. Linking reagent (streptavidin and/or alkaline phosphatase, Covance) was applied for 30 min at room temperature. These sections were incubated in 3% H₂O₂ for 15 min to bleach endogenous peroxidase activity. Then labeling reagent (HRP, Covance) was applied for 30 min at room temperature. Peroxidase activity was developed with NovaRed substrate (Vector) and sections were counterstained with hematoxylin.

Cell toxicity assays. All the transfection experiments were performed using Lipofectamine 2000 (Invitrogen), according to the manufacturer's instruction and at a 60% cell confluence. 500 ng of each vector was transfected in 35 mm wells. Cell death was determined by measuring Lactate dehydrogenase (LDH) cell release, using CytoTox 96 non-radioactive cytotoxicity assay (Promega) according to the manufacturer's instructions. Absorbance was recorded at 490 nm and total LDH release was measured by lysing the cells with 1% Triton X-100. In each experiment, determinations were performed in quintuplicates for each experimental condition and average data calculated. Statistical significance was determined using the two tailed unpaired Student t test for single comparisons (p<0.05) and the analysis of variance (ANOVA) when multiple pairwise conditions were compared.

Cell viability assays. HeK293T cells were transfected in 96 well plates and cell viability was determined 42 hours post-transfection with the 3-(4,5-dimethythiazol-2-yl)-2,5-diphenyl tetrazolium bromide (MTT) assay. MTT was added to cell culture media at 0.5 mg/mL final concentration and incubated for 45 minutes at 37° C. Cells were then lysed with 100 μL of DMSO upon medium removal and absorbance was measured at 595 nm. In each experiment, determinations were performed in quintuplicates. Statistical significance was determined using Student's t test (p<0.05).

### Example 4. BAC Transgenic Mouse Model of C9ORF72 ALS to Test the Hypothesis that Both Sense and Antisense Transcripts Contribute to ALS/FTD

Rationale: A mouse model of C9ORF72 ALS/FTD that recapitulates the sense and antisense transcripts is critical for modeling this disease. BAC clones were isolated from a human patient which contain ˜800 G4C2 repeats. These BAC clones were used to generate 8 founder lines. These mice are useful, for example, to answer the following questions: Does both RAN protein expression and RNA gain of function contribute to C9ORF72 ALS/FTD? Are sense and antisense mechanisms both important in C9ORF72 pathogenesis?

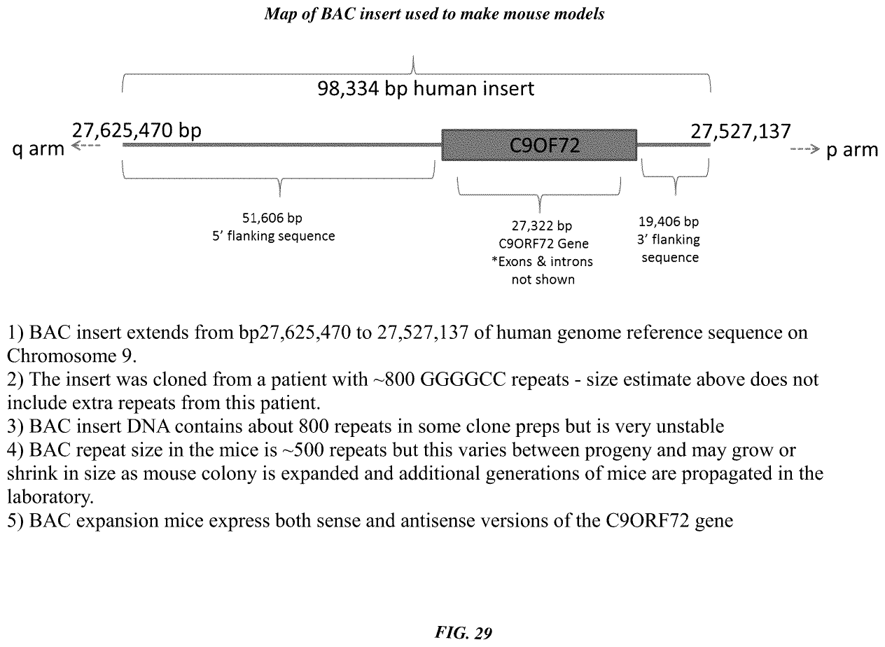

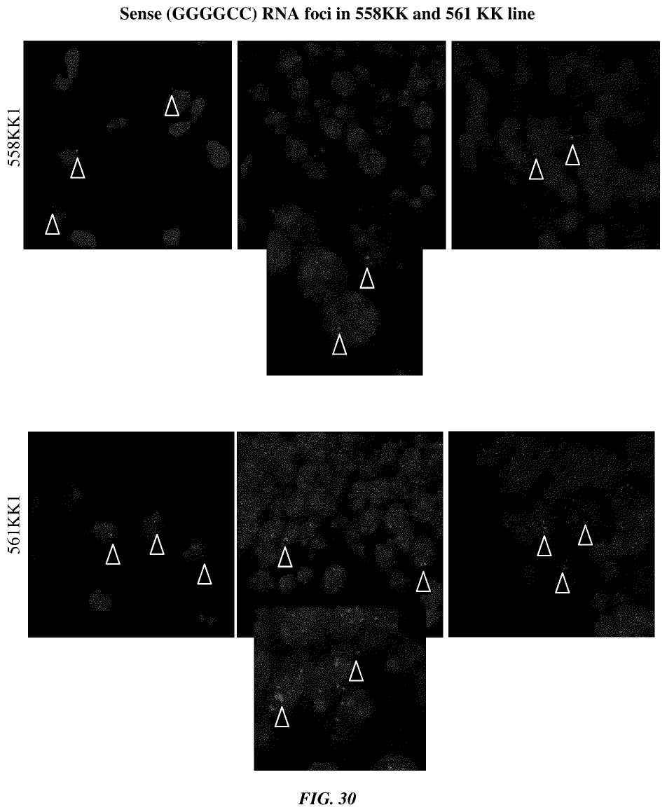

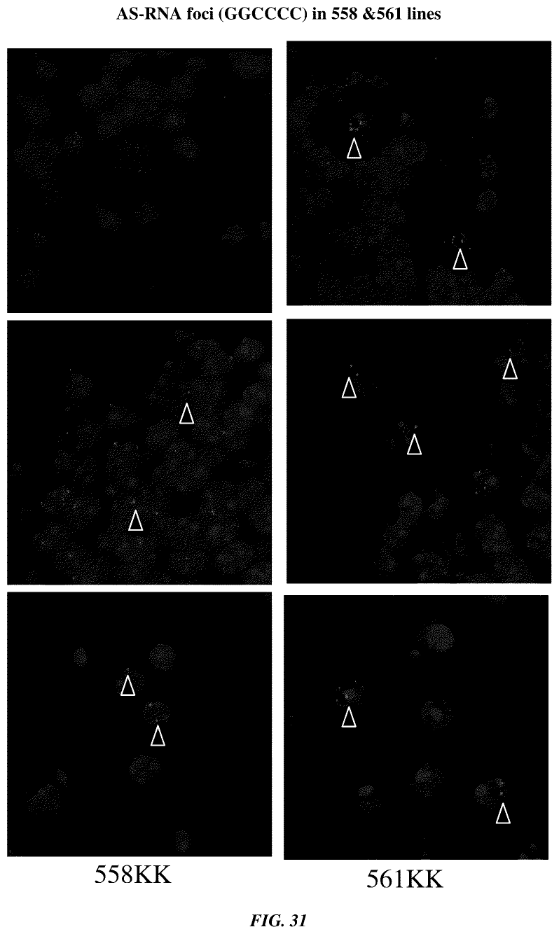

Approach: BAC clones containing the full human C9ORF72 gene plus flanking sequences were isolated from a human patient with ˜800 GGGGCC repeats and inserted into the pCCIBAC™ plasmid (Epicentre®)). The BAC insert chosen for use in the mouse extended from bp27,625,470 to 27,527,137 of human genome reference sequence on Chromosome 9 (FIG. 29). The coordinates above do not include extra repeats from this patient. It was found that the BAC insert DNA contained about 800 repeats in some clone preps but was very unstable. Pronuclear injections were performed and 8 FVB founder lines were generated-2 independent lines which were confirmed expansion mutations. The BAC repeat size in the mice was ˜500 repeats but varied between progeny and may grow or shrink in size as the mouse colony is expanded and additional generations of mice are propagated in the laboratory. BAC expansion mice expressed both sense and antisense versions of the C90RF72 gene. Sense and anti-sense GGGCC RNA foci were present in mice that had the GGGGCC repeats, but not in control mice (FIGS. 30-31).

At least two expansion and two control lines are selected for detailed characterization. Behavioral characterization includes rotorod analysis, grip strength, balance beam and open field assessments. Molecular characterization of sense and antisense transcripts and RAN proteins are performed by RT-PCR, RACE, immunoblot, immunohistochemistry and immunofluorescence. Immunohistochemistry, immunofluorescence and FISH studies are performed to correlate sites of RNA foci and C9-RAN proteins accumulation with pathological changes. RAN-protein accumulation in the CNS, CSF, muscle, blood and other tissues are examined at various times during development.

Relevance: Results from these studies will lead to a better understanding of the role that RAN translation plays in C9ORF72 ALS/FTD. Additionally, these studies will help to prioritize individual protein targets by determining which proteins are found most frequently in autopsy tissue and identifying overt differences in the toxicities of individual RAN proteins. Information from cellular and mouse models will also inform future studies on the effectiveness of various treatment strategies.

### REFERENCES

1. DeJesus-Hernandez M, et al. (2011) Expanded GGGGCC hexanucleotide repeat in noncoding region of C9ORF72 causes chromosome 9p-linked FTD and ALS. Neuron 72 (2): 245-256.
2. Renton A E, et al. (2011) A hexanucleotide repeat expansion in C9ORF72 is the cause of chromosome 9p21-linked ALS-FTD. Neuron 72 (2): 257-268.
3. Majounie E, et al. (2012) Frequency of the C9orf72 hexanucleotide repeat expansion in patients with amyotrophic lateral sclerosis and frontotemporal dementia: a cross-sectional study. Lancet Neurol 11 (4): 323-330.
4. Nelson D L, Orr H T, & Warren S T (2013) The unstable repeats—three evolving faces of neurological disease. Neuron 77 (5): 825-843.
5. Reddy K, Zamiti B, Stanley S Y, Macgregor R B, Jr., & Pearson CE (2013) The disease-associated r (GGGGCC) n repeat from the C9orf72 gene forms tract length-dependent uni and multimolecular RNA G-quadruplex structures. J Biol Chem 288 (14): 9860-9866.
6. Mori K, et al. (2013) hnRNP A3 binds to GGGGCC repeats and is a constituent of p62-positive/TDP43-negative inclusions in the hippocampus of patients with C9orf72 mutations. Acta Neuropathol 125 (3): 413-423.
7. Xu Z, et al. (2013) Expanded GGGGCC repeat RNA associated with amyotrophic lateral sclerosis and frontotemporal dementia causes neurodegeneration. Proc Natl Acad Sci USA 110 (19): 7778-7783.
8. Almeida S, et al. (2013) Modeling key pathological features of frontotemporal dementia with C9ORF72 repeat expansion in iPSC-derived human neurons. Acta Neuropathol.
9. Zu T, et al. (2011) Non-ATG-initiated translation directed by microsatellite expansions. Proc Natl Acad Sci USA 108 (1): 260-265.
10. Ash P E. et al. (2013) Unconventional translation of C9ORF72 GGGGCC expansion generates insoluble polypeptides specific to c9FTD/ALS. Neuron 77 (4): 639-646.
11. Mori K, et al. (2013) The C9orf72 GGGGCC Repeat Is Translated into Aggregating Dipeptide-Repeat Proteins in FTLD/ALS. Science.
12. Todd P K, et al. (2013) CGG repeat-associated translation mediates neurodegeneration in fragile X tremor ataxia syndrome. Neuron 78 (3): 440-455.
13. Ash PEA, et al. (2013) Unconventional Translation of C9ORF72 GGGGCC Expansion Generates Insoluble Polypeptides Specific to c9FTD/ALS Neuron.
14. Strausberg R L, et al. (2002) Generation and initial analysis of more than 15,000 full-length human and mouse cDNA sequences. Proc Natl Acad Sci USA 99 (26): 16899-16903.
15. Venter J C, et al. (2001) The sequence of the human genome. Science 291 (5507): 1304-1351.
16. Beausoleil S A, Villen J, Gerber S A, Rush J, & Gygi S P (2006) A probability-based approach for high-throughput protein phosphorylation analysis and site localization. Nat Biotechnol 24 (10): 1285-1292.
17. Sopher B L, et al. (2011) CTCF regulates ataxin-7 expression through promotion of a convergently transcribed, antisense noncoding RNA. Neuron 70 (6): 1071-1084.
18. Chung D W, Rudnicki D D, Yu L, & Margolis R L (2011) A natural antisense transcript at the Huntington's disease repeat locus regulates HTT expression. Hum Mol Genet 20(17): 3467-3477,
19. Wilburn B, et al. (2011) An antisense CAG repeat transcript at JPH3 locus mediates expanded polyglutamine protein toxicity in Huntington's disease-like 2 mice. Neuron 70 (3): 427-440.
20. Ladd P D, et al. (2007) An antisense transcript spanning the CGG repeat region of FMRI is upregulated in premutation carriers but silenced in full mutation individuals. Hum Mol Genet 16 (24): 3174-3187.
21. Moseley M L, et al. (2006) Bidirectional expression of CUG and CAG expansion transcripts and intranuclear polyglutamine inclusions in spinocerebellar ataxia type 8. Nat Genet 38 (7): 758-769.
22. Cho D H, et al. (2005) Antisense transcription and heterochromatin at the DM1 CTG repeats are constrained by CTCF. Mol Cell 20 (3): 483-489.
23. Li H, Wyman T, Yu Z X, Li S H, & Li X J (2003) Abnormal association of mutant huntingtin with synaptic vesicles inhibits glutamate release. Hum Mol Genet 12 (16): 2021-2030.
24. Luk K C, et al. (2012) Pathological alpha-synuclein transmission initiates Parkinson-like neurodegeneration in nontransgenic mice. Science 338 (6109): 949-953.

Without further elaboration, it is believed that one skilled in the art can, based on the above description, utilize the present disclosure to its fullest extent. The following specific embodiments are, therefore, to be construed as merely illustrative, and not limitative of the remainder of the disclosure in any way whatsoever. All publications cited herein are incorporated by reference for the purposes or subject matter referenced herein.

### OTHER EMBODIMENTS

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present disclosure, and without departing from the spirit and scope thereof, can make various changes and modifications of the disclosure to adapt it to various usages and conditions. Thus, other embodiments are also within the claims.

What is claimed is:

1-24. (canceled)

25. A method of producing an antibody, the method comprising administering to a subject a poly-(Gly-Ala) di-amino acid repeat-containing protein.

26. The method of claim 25, wherein the poly-(Gly-Ala) di-amino acid repeat-containing protein comprises 2-10,000 di-amino acid repeats.

27. The method of claim 25, wherein the poly-(Gly-Ala) di-amino acid repeat-containing protein comprises:
(i) the amino acid sequence (GA)ₓWSGRARGRARGGAAVAVPAPAAAEAQAVASG (SEQ ID NO: 8); or
(ii) the amino acid sequence (AG)ₓAWSGRARGRARGGAAVAVPAPAAAEAQA VASG (SEQ ID NO: 9),
wherein X represents the number of di-amino acid repeats and is 2-10,000.

28. The method of claim 25, wherein the poly-(Gly-Ala) di-amino acid repeat-containing protein comprises the sequence set forth in SEQ ID NO: 26.

29. The method of claim 25, further comprising the step of isolating the antibody.

30. The method of claim 25, wherein the antibody specifically binds an antigen comprising poly-(Gly-Ala) di-amino acid repeats.

31. The method of claim 25, wherein the antibody specifically binds an antigen comprising a C-terminal sequence of the poly-(Gly-Ala) di-amino acid repeat-containing protein.

32. The method of claim 25, wherein the poly-(Gly-Ala) di-amino acid repeating-containing protein comprises 10-200 di-amino acid repeats.

33. The method of claim 25, wherein the antibody is a polyclonal antibody.

34. The method of claim 25, wherein the antibody is a monoclonal antibody.

35. The method of claim 25, wherein the antibody is a humanized antibody.

36. The method of claim 25, wherein the subject is a mammalian subject.

37. The method of claim 25, wherein the subject is a mouse.

38. The method of claim 25, wherein the subject is a rabbit.
